SEQUENCE LISTING (76 sequences)

SEQ ID NO: 1 - protein, 5 aa, synthetic construct
EAAAK

SEQ ID NO: 2 - protein, 5 aa, synthetic construct
GGGGS

SEQ ID NO: 3 - DNA, 6958 bp, synthetic construct
  source 1..6958: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatggctctgcctgtgacagctctgctgctgcctcttgctctgcttctgcatgccgccagacctgacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaagagaacaggcggaggctctggcggaggtggaagcggaggcggagcttctgaagtgcagctggttgaaagtggcggcggactggttcaacctggcggatctctgagactgagctgtgccgccagcggcttcaatgtgtatgcctctggcatgcactgggtccgacaggcccctggaaaaggccttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagcagcttctactacgtgtacgccatggactactggggccagggaacactggtcaccgtgtccagcgccgccgctatcgaagtgatgtaccctcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgattacatgaacatgacccctcggaggcccggaccaaccagaaagcactaccagccttacgctcctcctagagatttcgccgcctaccggtccagagtgaagttctccagatctgccgacgctcctgcctatcagcagggccagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaagacgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 4 - protein, 793 aa, synthetic construct
MALPVTALLLPLALLLHAARPDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKRTGGGSGGGGSGGGASEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 5 - DNA, 6976 bp, synthetic construct
  source 1..6976: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatggctctgcctgtgacagctctgctgctgcctcttgctctgcttctgcatgccgccagacctgacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaagagaacaggcggaggctctggcggaggtggaagcggaggcggagcttctgaagtgcagctggttgaaagtggcggcggactggttcaacctggcggatctctgagactgagctgtgccgccagcggcttcaatgtgtatgcctctggcatgcactgggtccgacaggcccctggaaaaggccttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagcagcttctactacgtgtacgccatggactactggggccagggaacactggtcaccgtgtccagcacaacaacccctgctcctagacctcctacaccagctcctacaatcgccagccagcctctgtctctgaggccagaggcttgtagacctgctgctggcggagccgtgcatacaagaggactggatttcgcctgcgatatctacttctgggtgctcgtggttgtcggcggagtgctggcctgttatagcctgctggtcacagtggccttcatcatcttttgggtccgaagcaagcggagccggctgctgcacagcgattacatgaacatgacccctcggaggcccggacctaccagaaagcactaccagccttacgctcctcctagagatttcgccgcctaccggtccagagtgaagttctccagatctgccgacgctcctgcctatcagcagggccagaaccagctgtacaacgagctgaacctggggagaagagaagagtacgacgtgctggataagcggagaggcagagatcctgagatgggcggaaagccccagcggagaaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagacgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 6 - protein, 799 aa, synthetic construct
MALPVTALLLPLALLLHAARPDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKRTGGGSGGGGSGGGASEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSTTTPAPRPPTPAPTIASQPLSLRPEACRPAAGGAVHTRGLDFACDIYFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 7 - DNA, 7300 bp, synthetic construct
  source 1..7300: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgggcactcgattgctctgttgggccgcgctgtgcttgctgggtgccgaattgacagaagcgggtgtagctcaaagtcctcggtacaagattattgagaaacgccagtctgttgccttctggtgtaaccctatctctggtcacgcaactctctattggtatcagcaaatcctgggtcaaggtcctaaacttctgattcaatttcagaataatggcgttgtggacgactctcaactgcctaaagacagattcagtgcggaaaggttgaaaggcgtcgatagcacactcaaaattcagccggcaaagcttgaagactccgccgtttatctgtgcgcgagttctcttgatcctggggacacaggggaactgttctttggcgagggatcacgattgaccgtcctggaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagcagggccaagagaagtggaagcggcgccacaaacttcagcctgttgaaacaggccggggacgtcgaagagaatcctggaccaatggaatcctttctcggaggtgtactgctcatactctggctgcaagtggattgggtaaagagtcaaaaaattgaacaaaacagcgaggctttgaatatccaagagggcaagactgccacgctgacctgtaactatacaaactatagtccggcatacctccagtggtaccggcaagatcctggtcgcgggccggttttcctgttgcttattagagagaatgaaaaggaaaaaagaaaagaacgcttgaaggtcactttcgacacgacgcttaaacagagtcttttccacattaccgcgtcacagcctgcggactcagctacatacctgtgtgctctcgacatctatccccacgacatgaggtttggcgcaggcactcgactcacggtaaaacccgacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 8 - protein, 907 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDPGDTGELFFGEGSRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALDIYPHDMRFGAGTRLTVKPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 9 - DNA, 7309 bp, synthetic construct
  source 1..7309: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgcatcctcagctgctgggctacgtggtgctgtgtttgcttggagccggacctctggaagcccaagtgacacagaaccccagatacctgatcaccgtgaccggcaagaaactgaccgtgacctgcagccagaacatgaaccacgagtacatgagctggtacagacaggaccctggcctgggcctgagacagatctactacagcatgaacgtggaagtgaccgacaagggcgacgtgcccgagggctacaaggtgtccagaaaagagaagcggaacttcccactgatcctggaaagcccatctcctaaccagaccagcctgtacttctgcgccagctctattcagcagggcgccgacacacagtacttcggccctggaacaagactgacagtgctggaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagcagggccaagagaagtggaagcggcgccacaaacttcagcctgttgaaacaggccggggacgtcgaagagaatcctggaccaatggccaagagcctgagagtgctgctggtcatcctgtggctgcagctgtcttgggtctggtcccagcagaaagaggtggaacaggaccctggacctctgtctgttcctgagggcgccatcgtgtccctgaactgcacctacagcaacagcgccttccagtacttcatgtggtacagacagtacagccggaagggccccgagctgctgatgtacacatacagcagcggcaacaaagaggacggccggtttacagcccaggtggacaagagcagcaagtacatctccctgttcatccgggacagccagcctagcgatagcgccacatatctgtgtgccatgagcggcctgaaagaggacagcagctacaagctgatcttcggcagcggcaccagactgctcgttagacctgacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 10 - protein, 910 aa, synthetic construct
MHPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSIQQGADTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMAKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSGLKEDSSYKLIFGSGTRLLVRPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 11 - DNA, 7300 bp, synthetic construct
  source 1..7300: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgtctatcggcctgctgtgctgcgtggcctttagtctgctttgggcctctcctgtgaatgccggcgtgacccagacacctaagttccaggtgctgaaaaccggccagagcatgaccctgcagtgcgcccaggacatgaaccacaacagcatgtactggtacagacaggaccccggcatgggcctgagactgatctactactctgccagcgagggcaccaccgacaaaggcgaagtgcccaatggctacaacgtgtcccggctgaacaagagagagttcagcctgcggctggaaagcgccgctccttctcagacctccgtgtacttttgcgccagcagcgaaggcctgtggcaagtgggagatgagcagtactttggccctggcaccagactgaccgtgacagaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagcagggccaagagaagtggaagcggcgccacaaacttcagcctgttgaaacaggccggggacgtcgaagagaatcctggaccaatgatcagcctgagagtgctgctggtcatcctgtggctgcagctgtcttgggtctggtcccagcggaaagaggtggaacaggaccccggacctttcaatgtgcctgaaggcgccaccgtggccttcaactgcacctacagcaatagcgccagccagagcttcttctggtacagacaggattgccggaaagaacccaagctgctgatgagcgtgtacagcagcggcaacgaggacggcagattcacagcccagctgaacagagccagccagtacatcagcctgctgatccgggatagcaagctgagcgatagcgccacctacctgtgtgtggttcagcctggcggctaccagaaagtgacctttggcaccggcaccaagctgcaagtgatccctgacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 12 - protein, 907 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEGLWQVGDEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVQPGGYQKVTFGTGTKLQVIPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 13 - DNA, 7315 bp, synthetic construct
  source 1..7315: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgggcaccagactgttcttctacgtggccctgtgtctgctgtggaccggacatatggacgccggcattacacagagccccagacacaaagtgaccgagacaggcacccctgtgacactgagatgtcaccagaccgagaaccaccgctacatgtactggtacagacaggaccctggccacggcctgagactgatccactacagctacggcgtgaaggacaccgacaagggcgaagtgtctgacggctacagcgtgtccagaagcaagaccgaggacttcctgctgaccctggaaagcgccacaagcagccagaccagcgtgtacttctgtgccatcagcgagctggttaccggcgatagccctctgcactttggcaatggcaccaggctgaccgtgacagaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagcagggccaagagaagtggaagcggcgccacaaacttcagcctgttgaaacaggccggggacgtcgaagagaatcctggaccaatgaccagagtgtctctgctgtgggccgtcgtggtgtccacatgtctggaatctggcatggcccagaccgtgacacagagccagcctgagatgtctgtgcaagaggccgagacagtgaccctgagctgcacctacgataccagcgagaacaactactacctgttctggtacaagcagcctcctagccggcagatgatcctggtcatcagacaagaggcctataagcagcagaacgccaccgagaacagattcagcgtgaacttccagaaggccgccaagagcttcagcctgaagatcagcgatagccagctgggcgacaccgccatgtacttttgcgccttcatgggctactctggcgccggaagctaccagctgacatttggcaagggcaccaaactgagcgtgatccctgacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 14 - protein, 912 aa, synthetic construct
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISELVTGDSPLHFGNGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFMGYSGAGSYQLTFGKGTKLSVIPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 15 - DNA, 7330 bp, synthetic construct
  source 1..7330: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgggcaccagactgctgtgttgggctgctctgtgtctgctgggagccgaacttactgaagctggcgtggcccagtctcctcggtacaagatcatcgagaagcggcagagcgtggccttctggtgcaatcctatcagcggacacgccacactgtactggtatcagcagatcctcggacagggccccaagctgctgattcagttccagaacaacggcgtggtggacgacagccagctgcctaaggatagattcagcgccgagcggctgaaaggcgtggacagcacactgaagatccagcctgccaagctggaagatagcgccgtgtacctgtgtgccagctctctggatcctggcgataccggcgagctgttctttggcgagggcagcagactgaccgtgctcgaggacctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcacagatccccagcctctgaaagagcagccagctctgaacgacagccggtactgtctgagcagcaggctgagagtgtccgccaccttctggcagaaccccagaaaccacttcagatgccaggtgcagttctacggcctgagcgagaacgatgagtggacacaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggactctagaggcagagccaagagatctggcagcggcgccacaaactttagcctgctgaaacaggccggcgacgtggaagagaaccccggacctatggaatcctttctcggtggcgtgctgctcatcctgtggctgcaagtggattgggtcaagagccagaagatcgagcagaacagcgaggccctgaacatccaagagggcaagaccgccactctgacctgcaactacaccaactacagccccgcctacctgcagtggtacagacaggaccctggaagaggccctgtgttcctgctgctgatccgcgagaatgagaaagagaagcgcaaagagcgcctgaaagtcaccttcgacacaaccctgaagcagagcctgttccacatcaccgcctctcagccagccgatagcgccacatatctgtgcgccctggacatctaccctcacgacatgagattcggagccggcaccaggctgacagtgaagcccgatattcagaaccccgatcctgccgtctatcagctgagagacagcaagagcagcgacaagagcgtgtgcctgttcaccgacttcgactcccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctcctgaaagtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 16 - protein, 917 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDPGDTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGATNFSLLKQAGDVEENPGPMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALDIYPHDMRFGAGTRLTVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 17 - DNA, 7330 bp, synthetic construct
  source 1..7330: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgggcactcgattgctctgttgggccgcgctgtgcttgctgggtgccgaattgacagaagcgggtgtagctcaaagtcctcggtacaagattattgagaaacgccagtctgttgccttctggtgtaaccctatctctggtcacgcaactctctattggtatcagcaaatcctgggtcaaggtcctaaacttctgattcaatttcagaataatggcgttgtggacgactctcaactgcctaaagacagattcagtgcggaaaggttgaaaggcgtcgatagcacactcaaaattcagccggcaaagcttgaagactccgccgtttatctgtgcgcgagttctcttgatcctggggacacaggggaactgttctttggcgagggatcacgattgaccgtcctggaggacctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacacaggatagagccaagccagtgacacagatcgtgtctgccgaagcctggggcagagccgattgtggctttaccagcgagagctaccagcaaggcgtgctgtctgccaccatcctgtacgagatcctgctgggcaaagccactctgtacgccgtgctggtgtctgccctggtgctgatggccatggtcaagcggaaggacagcagaggcagggccaagagaagtggaagcggcgccacaaacttcagcctgttgaaacaggccggggacgtcgaagagaatcctggaccaatggaatcctttctcggaggtgtactgctcatactctggctgcaagtggattgggtaaagagtcaaaaaattgaacaaaacagcgaggctttgaatatccaagagggcaagactgccacgctgacctgtaactatacaaactatagtccggcatacctccagtggtaccggcaagatcctggtcgcgggccggttttcctgttgcttattagagagaatgaaaaggaaaaaagaaaagaacgcttgaaggtcactttcgacacgacgcttaaacagagtcttttccacattaccgcgtcacagcctgcggactcagctacatacctgtgtgctctcgacatctatccccacgacatgaggtttggcgcaggcactcgactcacggtaaaacccgacatccagaatcctgatcctgccgtgtaccagctgcgggacagcaagagcagcgataagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggctttagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 18 - protein, 917 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDPGDTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGATNFSLLKQAGDVEENPGPMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALDIYPHDMRFGAGTRLTVKPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 19 - DNA, 7330 bp, synthetic construct
  source 1..7330: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgggcactcgattgctctgttgggccgcgctgtgcttgctgggtgccgaattgacagaagcgggtgtagctcaaagtcctcggtacaagattattgagaaacgccagtctgttgccttctggtgtaaccctatctctggtcacgcaactctctattggtatcagcaaatcctgggtcaaggtcctaaacttctgattcaatttcagaataatggcgttgtggacgactctcaactgcctaaagacagattcagtgcggaaaggttgaaaggcgtcgatagcacactcaaaattcagccggcaaagcttgaagactccgccgtttatctgtgcgcgagttctcttgatcctggggacacaggggaactgttctttggcgagggatcacgattgaccgtcctggaggacctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacacaggatagagccaagccagtgacacagatcgtgtctgccgaagcctggggcagagccgattgtggctttaccagcgagagctaccagcaaggcgtgctgtctgccaccatcctgtacgagatcctgctgggcaaagccactctgtacgccgtgctggtgtctgccctggtgctgatggccatggtcaagcggaaggacagcagaggcagggccaagagaagtggaagcggcgccacaaacttcagcctgttgaaacaggccggggacgtcgaagagaatcctggaccaatggaatcctttctcggaggtgtactgctcatactctggctgcaagtggattgggtaaagagtcaaaaaattgaacaaaacagcgaggctttgaatatccaagagggcaagactgccacgctgacctgtaactatacaaactatagtccggcatacctccagtggtaccggcaagatcctggtcgcgggccggttttcctgttgcttattagagagaatgaaaaggaaaaaagaaaagaacgcttgaaggtcactttcgacacgacgcttaaacagagtcttttccacattaccgcgtcacagcctgcggactcagctacatacctgtgtgctctcgacatctatccccacgacatgaggtttggcgcaggcactcgactcacggtaaaacccgacatccagaatcctgatcctgccgtgtaccagctgcgggacagcaagagcagcgataagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgctggtgatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 20 - protein, 917 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDPGDTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGATNFSLLKQAGDVEENPGPMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALDIYPHDMRFGAGTRLTVKPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 21 - DNA, 6975 bp, synthetic construct
  source 1..6975: mol_type = other DNA
acgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatatacaggtaagaaactgctaacagcataccagatgatgagatcactttgggtagaatagatgaactaaagagaaggattgacagagactaacgtcaggatcccaggtgacgccaatacctactggttcatgggtcacagtttgagatgtgtccctagctactagggtcctgcttctcctgtaagagaccagttaaatgcttagctacaggcaagctgcccggaacaggttatgtcaggtcactgatttggttgacaatagagacatcacctctctatccccgtcatttcattcattctaaactgttcgctcctgtaggactactgttgtaggaagccaaatttccgtcaatattctgagtctgagtatagaaagaagccatttctcctacctttgattgactgtcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatggctctgcctgtgacagctctgctgctgcctctggctctgcttctgcatgccgccagacctgacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaagagaacaggcggcggatctggcggcggaggaagcggaggcggagcttctgaagttcagctggtggaatctggcggaggcctggttcaacctggcggatctctgagactgagctgtgccgccagcggcttcaatgtgtacgcctctggaatgcactgggtccgacaggcccctggcaaaggacttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagctccttctactacgtgtacgccatggactactggggccagggaacactggtcacagtttctagtcagagcatcaagggcaatcacctggtcaaggtgtacgactaccaagaggacggcagcgtgctgctgacatgtgatgccgaggccaagaacatcacatggttcaaggacggcaagatgatcggcttcctgaccgaggacaagaagaagtggaacctgggcagcaacgccaaggatcccagaggcatgtatcagtgcaagggcagccagaacaagagcaagcccctgcaggtctactaccggatgtgccagaactgcatcgagctgaacgccgctaccatctccggctttctgttcgccgagatcgtgtccatcttcgtgctggccgtgggcgtgtactttatcgctggacaggatggcgtcagacagagcagagccagcgacaagcaaaccctgctgcctaacgaccagctgtaccagcctctgaaggacagagaggacgaccagtacagccatctgcagggcaaccagctgcggagaaacagagccaagagatctggcagcggcgccaccaatttcagcctgctgaaacaggctggcgacgtggaagagaaccctggacctatgggagctggcgctacaggcagagctatggatggacctagactgctgctcctgctgctgctcggagtttctcttggcggagccaaagaggcctgtcctaccggcctgtatacacactctggcgagtgctgcaaggcctgcaatcttggagaaggcgtggcacagccttgcggcgctaatcagacagtgtgcgagccttgcctggacagcgtgacctttagcgacgtggtgtctgccaccgagccatgcaagccttgtaccgagtgtgtgggcctgcagagcatgtctgccccttgtgtggaagccgacgatgccgtgtgtagatgcgcctacggctactaccaggacgagacaacaggcagatgcgaggcctgtagagtgtgtgaagccggctctggactggtgttcagctgccaagacaagcagaacaccgtgtgcgaggaatgccccgatggcacctatagcgacgaggccaaccatgtggatccctgcctgccttgtactgtgtgcgaagataccgagcggcagctgcgcgagtgtacaagatgggctgatgccgagtgcgaagagatccccggcagatggatcaccagaagcacacctccagagggcagcgatagcacagccccttctacacaagagcccgaggctcctcctgagcaggatctgattgcctctacagtggccggcgtggtcacaacagtgatgggatcttctcagcccgtggtcaccagaggcaccaccgacaatctgatccccgtgtactgtagcatcctggccgccgtggttgtgggactcgtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttggccaaagtacctgtaaagacagaagagagatagagtattagctgggcatggagttgttcagatgccctgaagtatttccatggttatttgaggccaagatgaagaaagatccaaatccatgaaaatggtagtatctatacttagttttccggattgtagactaaatcactcaattgaattcaaggtgataataaacaatttgtctcatctttgtcctggtatcctgggagggtgtttctaaccgttggaatttcatgagtgacaggagtggctttgttatctgtggtggatccttagaccatacctgagtttatgcatggcaatgactcaggatggtgctggtcaagaccaaccatatggtaagaggattgcagctttgaaactagtggtatcagcccccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggtgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttag

SEQ ID NO: 22 - protein, 730 aa, synthetic construct
MALPVTALLLPLALLLHAARPDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKRTGGGSGGGGSGGGASEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNRAKRSGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 23 - DNA, 7366 bp, synthetic construct
  source 1..7366: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatggctctgcctgtgacagctctgctgctgcctcttgctctgcttctgcatgccgccagacctgaagtgcagctggttgaaagtggcggcggactggttcaacctggcggatctctgagactgagctgtgccgccagcggcttcaatgtgtatgcctctggcatgcactgggtccgacaggcccctggaaaaggccttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagcagcttctactacgtgtacgccatggactactggggccagggaacactggtcaccgtgtccagcagaacaggcggaggctctggcggaggtggaagcggaggcggagcttctgacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaaggacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagcagggccaagagaagtggaagcggcgccacaaacttcagcctgttgaaacaggccggggacgtcgaagagaatcctggaccaatggcactgcccgtcactgccttgttgctgccacttgcactgctgctccatgccgccaggccagaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 24 - protein, 929 aa, synthetic construct
MALPVTALLLPLALLLHAARPEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSRTGGGSGGGGSGGGASDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGATNFSLLKQAGDVEENPGPMALPVTALLLPLALLLHAARPEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 25 - DNA, 7366 bp, synthetic construct
  source 1..7366: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatggctctgcctgtgacagctctgctgctgcctcttgctctgcttctgcatgccgccagacctgaagtgcagctggttgaaagtggcggcggactggttcaacctggcggatctctgagactgagctgtgccgccagcggcttcaatgtgtatgcctctggcatgcactgggtccgacaggcccctggaaaaggccttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagcagcttctactacgtgtacgccatggactactggggccagggaacactggtcaccgtgtccagcagaacaggcggaggctctggcggaggtggaagcggaggcggagcttctgacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaaggaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagcagggccaagagaagtggaagcggcgccacaaacttcagcctgttgaaacaggccggggacgtcgaagagaatcctggaccaatggcactgcccgtcactgccttgttgctgccacttgcactgctgctccatgccgccaggccagacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 26 - protein, 929 aa, synthetic construct
MALPVTALLLPLALLLHAARPEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSRTGGGSGGGGSGGGASDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMALPVTALLLPLALLLHAARPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 27 - DNA, 7366 bp, synthetic construct
  source 1..7366: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatggctctgcctgtgacagctctgctgctgcctcttgctctgcttctgcatgccgccagacctgacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaagagaacaggcggaggctctggcggaggtggaagcggaggcggagcttctgaagtgcagctggttgaaagtggcggcggactggttcaacctggcggatctctgagactgagctgtgccgccagcggcttcaatgtgtatgcctctggcatgcactgggtccgacaggcccctggaaaaggccttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagcagcttctactacgtgtacgccatggactactggggccagggaacactggtcaccgtgtccagcgacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagcagggccaagagaagtggaagcggcgccacaaacttcagcctgttgaaacaggccggggacgtcgaagagaatcctggaccaatggcactgcccgtcactgccttgttgctgccacttgcactgctgctccatgccgccaggccagaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 28 - protein, 929 aa, synthetic construct
MALPVTALLLPLALLLHAARPDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKRTGGGSGGGGSGGGASEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGATNFSLLKQAGDVEENPGPMALPVTALLLPLALLLHAARPEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 29 - DNA, 7366 bp, synthetic construct
  source 1..7366: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatggctctgcctgtgacagctctgctgctgcctcttgctctgcttctgcatgccgccagacctgacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaagagaacaggcggaggctctggcggaggtggaagcggaggcggagcttctgaagtgcagctggttgaaagtggcggcggactggttcaacctggcggatctctgagactgagctgtgccgccagcggcttcaatgtgtatgcctctggcatgcactgggtccgacaggcccctggaaaaggccttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagcagcttctactacgtgtacgccatggactactggggccagggaacactggtcaccgtgtccagcgaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagcagggccaagagaagtggaagcggcgccacaaacttcagcctgttgaaacaggccggggacgtcgaagagaatcctggaccaatggcactgcccgtcactgccttgttgctgccacttgcactgctgctccatgccgccaggccagacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 30 - protein, 929 aa, synthetic construct
MALPVTALLLPLALLLHAARPDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKRTGGGSGGGGSGGGASEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMALPVTALLLPLALLLHAARPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 31 - DNA, 8038 bp, synthetic construct
  source 1..8038: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgtctatcggcctgctgtgttgtgccgctctgtctctgctttgggccggacctgttaatgccggcgtgacccagacacctaagttccaggtgctgaaaaccggccagagcatgaccctgcagtgcgcccaggatatgaaccacgagtacatgagctggtacagacaggaccctggcatgggcctgagactgatccactattctgtcggagccggcatcaccgaccagggcgaagttcctaatggctacaacgtgtccagaagcaccaccgaggacttcccactgagactgctgtctgccgctcctagccagaccagcgtgtacttctgtgcctctagacctggactggctggcggcagacctgagcagtattttggccctggcaccagactgaccgtgaccgaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagcagggccaagagaagtggaagcggcgccacaaacttcagcctgttgaaacaggccggggacgtcgaagagaatcctggaccaatggctctgcctgtgacagctctgctgctgcctcttgctctgcttctgcatgccgccagacctgaagtgcagctggttgaaagtggcggcggactggttcaacctggcggatctctgagactgagctgtgccgccagcggcttcaatgtgtatgcctctggcatgcactgggtccgacaggcccctggaaaaggccttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagcagcttctactacgtgtacgccatggactactggggccagggaacactggtcaccgtgtccagcagaacaggcggaggctctggcggaggtggaagcggaggcggagcttctgacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaagcagaaagaggtggaacagaacagcggccctctgtctgttcctgaaggcgctatcgccagcctgaactgcacctacagcgatagaggcagccagagcttcttctggtacagacagtacagcggcaagagccccgagctgatcatgagcatctacagcaacggcgacaaagaggacggccggtttacagcccagctgaacaaggccagccagtacgtgtccctgctgatcagagatagccagcctagcgacagcgccacctatctgtgtgccgtgaccacagatagctggggcaagctgcagtttggcgccggaacacaggtggtggttacacctgacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 32 - protein, 1153 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRPGLAGGRPEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMALPVTALLLPLALLLHAARPEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSRTGGGSGGGGSGGGASDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKQKEVEQNSGPLSVPEGAIASLNCTYSDRGSQSFFWYRQYSGKSPELIMSIYSNGDKEDGRFTAQLNKASQYVSLLIRDSQPSDSATYLCAVTTDSWGKLQFGAGTQVVVTPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 33 - DNA, 8044 bp, synthetic construct
  source 1..8044: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatggctctgcctgtgacagctctgctgctgcctcttgctctgcttctgcatgccgccagacctgaagtgcagctggttgaaagtggcggcggactggttcaacctggcggatctctgagactgagctgtgccgccagcggcttcaatgtgtatgcctctggcatgcactgggtccgacaggcccctggaaaaggccttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagcagcttctactacgtgtacgccatggactactggggccagggaacactggtcaccgtgtccagcagaacaggcggaggctctggcggaggtggaagcggaggcggagcttctgacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaagaatgccggcgtgacccagacacctaagttccaggtgctgaaaaccggccagagcatgaccctgcagtgcgcccaggatatgaaccacgagtacatgagctggtacagacaggaccctggcatgggcctgagactgatccactattctgtcggagccggcatcaccgaccagggcgaagttcctaatggctacaacgtgtccagaagcaccaccgaggacttcccactgagactgctgtctgccgctcctagccagaccagcgtgtacttctgtgcctctagacctggactggctggcggcagacctgagcagtattttggccctggcaccagactgaccgtgaccgaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagcagggccaagagaagtggaagcggcgccacaaacttcagcctgttgaaacaggccggggacgtcgaagagaatcctggaccaatgaagtccctgagagtgctgctggtcatcctgtggctgcagctgtcttgggtctggtcccagcagaaagaggtggaacagaacagcggccctctgtctgttcctgaaggcgctatcgccagcctgaactgcacctacagcgatagaggcagccagagcttcttctggtacagacagtacagcggcaagagccccgagctgatcatgagcatctacagcaacggcgacaaagaggacggccggtttacagcccagctgaacaaggccagccagtacgtgtccctgctgatcagagatagccagcctagcgacagcgccacctatctgtgtgccgtgaccacagatagctggggcaagctgcagtttggcgccggaacacaggtggtggttacacctgacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 34 - protein, 1155 aa, synthetic construct
MALPVTALLLPLALLLHAARPEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSRTGGGSGGGGSGGGASDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRPGLAGGRPEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVTTDSWGKLQFGAGTQVVVTPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 35 - DNA, 8038 bp, synthetic construct
  source 1..8038: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgtctatcggcctgctgtgttgtgccgctctgtctctgctttgggccggacctgttaatgccggcgtgacccagacacctaagttccaggtgctgaaaaccggccagagcatgaccctgcagtgcgcccaggatatgaaccacgagtacatgagctggtacagacaggaccctggcatgggcctgagactgatccactattctgtcggagccggcatcaccgaccagggcgaagttcctaatggctacaacgtgtccagaagcaccaccgaggacttcccactgagactgctgtctgccgctcctagccagaccagcgtgtacttctgtgcctctagacctggactggctggcggcagacctgagcagtattttggccctggcaccagactgaccgtgaccgaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagcagggccaagagaagtggaagcggcgccacaaacttcagcctgttgaaacaggccggggacgtcgaagagaatcctggaccaatggctctgcctgtgacagctctgctgctgcctcttgctctgcttctgcatgccgccagacctgacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaagagaacaggcggaggctctggcggaggtggaagcggaggcggagcttctgaagtgcagctggttgaaagtggcggcggactggttcaacctggcggatctctgagactgagctgtgccgccagcggcttcaatgtgtatgcctctggcatgcactgggtccgacaggcccctggaaaaggccttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagcagcttctactacgtgtacgccatggactactggggccagggaacactggtcaccgtgtccagccagaaagaggtggaacagaacagcggccctctgtctgttcctgaaggcgctatcgccagcctgaactgcacctacagcgatagaggcagccagagcttcttctggtacagacagtacagcggcaagagccccgagctgatcatgagcatctacagcaacggcgacaaagaggacggccggtttacagcccagctgaacaaggccagccagtacgtgtccctgctgatcagagatagccagcctagcgacagcgccacctatctgtgtgccgtgaccacagatagctggggcaagctgcagtttggcgccggaacacaggtggtggttacacctgacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 36 - protein, 1153 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRPGLAGGRPEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMALPVTALLLPLALLLHAARPDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKRTGGGSGGGGSGGGASEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSQKEVEQNSGPLSVPEGAIASLNCTYSDRGSQSFFWYRQYSGKSPELIMSIYSNGDKEDGRFTAQLNKASQYVSLLIRDSQPSDSATYLCAVTTDSWGKLQFGAGTQVVVTPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 37 - DNA, 8044 bp, synthetic construct
  source 1..8044: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatggctctgcctgtgacagctctgctgctgcctcttgctctgcttctgcatgccgccagacctgacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaagagaacaggcggaggctctggcggaggtggaagcggaggcggagcttctgaagtgcagctggttgaaagtggcggcggactggttcaacctggcggatctctgagactgagctgtgccgccagcggcttcaatgtgtatgcctctggcatgcactgggtccgacaggcccctggaaaaggccttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagcagcttctactacgtgtacgccatggactactggggccagggaacactggtcaccgtgtccagcaatgccggcgtgacccagacacctaagttccaggtgctgaaaaccggccagagcatgaccctgcagtgcgcccaggatatgaaccacgagtacatgagctggtacagacaggaccctggcatgggcctgagactgatccactattctgtcggagccggcatcaccgaccagggcgaagttcctaatggctacaacgtgtccagaagcaccaccgaggacttcccactgagactgctgtctgccgctcctagccagaccagcgtgtacttctgtgcctctagacctggactggctggcggcagacctgagcagtattttggccctggcaccagactgaccgtgaccgaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagcagggccaagagaagtggaagcggcgccacaaacttcagcctgttgaaacaggccggggacgtcgaagagaatcctggaccaatgaagtccctgagagtgctgctggtcatcctgtggctgcagctgtcttgggtctggtcccagcagaaagaggtggaacagaacagcggccctctgtctgttcctgaaggcgctatcgccagcctgaactgcacctacagcgatagaggcagccagagcttcttctggtacagacagtacagcggcaagagccccgagctgatcatgagcatctacagcaacggcgacaaagaggacggccggtttacagcccagctgaacaaggccagccagtacgtgtccctgctgatcagagatagccagcctagcgacagcgccacctatctgtgtgccgtgaccacagatagctggggcaagctgcagtttggcgccggaacacaggtggtggttacacctgacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 38 - protein, 1155 aa, synthetic construct
MALPVTALLLPLALLLHAARPDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKRTGGGSGGGGSGGGASEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRPGLAGGRPEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVTTDSWGKLQFGAGTQVVVTPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 39 - DNA, 7318 bp, synthetic construct
  source 1..7318: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatggctctgcctgtgacagctctgctgctgcctcttgctctgcttctgcatgccgccagacctgacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaaggaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagcagggccaagagaagtggaagcggcgccacaaacttcagcctgttgaaacaggccggggacgtcgaagagaatcctggaccaatggcactgcccgtcactgccttgttgctgccacttgcactgctgctccatgccgccaggccagaagtgcagctggttgaaagtggcggcggactggttcaacctggcggatctctgagactgagctgtgccgccagcggcttcaatgtgtatgcctctggcatgcactgggtccgacaggcccctggaaaaggccttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagcagcttctactacgtgtacgccatggactactggggccagggaacactggtcaccgtgtccagcgacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 40 - protein, 913 aa, synthetic construct
MALPVTALLLPLALLLHAARPDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMALPVTALLLPLALLLHAARPEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 41 - DNA, 7318 bp, synthetic construct
  source 1..7318: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatggctctgcctgtgacagctctgctgctgcctcttgctctgcttctgcatgccgccagacctgaagtgcagctggttgaaagtggcggcggactggttcaacctggcggatctctgagactgagctgtgccgccagcggcttcaatgtgtatgcctctggcatgcactgggtccgacaggcccctggaaaaggccttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagcagcttctactacgtgtacgccatggactactggggccagggaacactggtcaccgtgtccagcgaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagcagggccaagagaagtggaagcggcgccacaaacttcagcctgttgaaacaggccggggacgtcgaagagaatcctggaccaatggcactgcccgtcactgccttgttgctgccacttgcactgctgctccatgccgccaggccagacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaaggacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 42 - protein, 913 aa, synthetic construct
MALPVTALLLPLALLLHAARPEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMALPVTALLLPLALLLHAARPDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 43 - DNA, 7348 bp, synthetic construct
  source 1..7348: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatggctctgcctgtgacagctctgctgctgcctcttgctctgcttctgcatgccgccagacctgacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaaggaagccgccgctaaagaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagcagggccaagagaagtggaagcggcgccacaaacttcagcctgttgaaacaggccggggacgtcgaagagaatcctggaccaatggcactgcccgtcactgccttgttgctgccacttgcactgctgctccatgccgccaggccagaagtgcagctggttgaaagtggcggcggactggttcaacctggcggatctctgagactgagctgtgccgccagcggcttcaatgtgtatgcctctggcatgcactgggtccgacaggcccctggaaaaggccttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagcagcttctactacgtgtacgccatggactactggggccagggaacactggtcaccgtgtccagcgaagccgccgctaaagacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 44 - protein, 923 aa, synthetic construct
MALPVTALLLPLALLLHAARPDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKEAAAKEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMALPVTALLLPLALLLHAARPEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSEAAAKDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 45 - DNA, 7348 bp, synthetic construct
  source 1..7348: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatggctctgcctgtgacagctctgctgctgcctcttgctctgcttctgcatgccgccagacctgaagtgcagctggttgaaagtggcggcggactggttcaacctggcggatctctgagactgagctgtgccgccagcggcttcaatgtgtatgcctctggcatgcactgggtccgacaggcccctggaaaaggccttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagcagcttctactacgtgtacgccatggactactggggccagggaacactggtcaccgtgtccagcgaagccgccgctaaagaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagcagggccaagagaagtggaagcggcgccacaaacttcagcctgttgaaacaggccggggacgtcgaagagaatcctggaccaatggcactgcccgtcactgccttgttgctgccacttgcactgctgctccatgccgccaggccagacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaaggaagccgccgctaaagacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 46 - protein, 923 aa, synthetic construct
MALPVTALLLPLALLLHAARPEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSEAAAKEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMALPVTALLLPLALLLHAARPDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKEAAAKDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 47 - DNA, 8026 bp, synthetic construct
  source 1..8026: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatggctctgcctgtgacagctctgctgctgcctcttgctctgcttctgcatgccgccagacctgacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaagggcggtggcggttctaatgccggcgtgacccagacacctaagttccaggtgctgaaaaccggccagagcatgaccctgcagtgcgcccaggatatgaaccacgagtacatgagctggtacagacaggaccctggcatgggcctgagactgatccactattctgtcggagccggcatcaccgaccagggcgaagttcctaatggctacaacgtgtccagaagcaccaccgaggacttcccactgagactgctgtctgccgctcctagccagaccagcgtgtacttctgtgcctctagacctggactggctggcggcagacctgagcagtattttggccctggcaccagactgaccgtgaccgaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagcagggccaagagaagtggaagcggcgccacaaacttcagcctgttgaaacaggccggggacgtcgaagagaatcctggaccaatggcactgcccgtcactgccttgttgctgccacttgcactgctgctccatgccgccaggccagaagtgcagctggttgaaagtggcggcggactggttcaacctggcggatctctgagactgagctgtgccgccagcggcttcaatgtgtatgcctctggcatgcactgggtccgacaggcccctggaaaaggccttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagcagcttctactacgtgtacgccatggactactggggccagggaacactggtcaccgtgtccagcggcggtggcggttctcagaaagaggtggaacagaacagcggccctctgtctgttcctgaaggcgctatcgccagcctgaactgcacctacagcgatagaggcagccagagcttcttctggtacagacagtacagcggcaagagccccgagctgatcatgagcatctacagcaacggcgacaaagaggacggccggtttacagcccagctgaacaaggccagccagtacgtgtccctgctgatcagagatagccagcctagcgacagcgccacctatctgtgtgccgtgaccacagatagctggggcaagctgcagtttggcgccggaacacaggtggtggttacacctgacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 48 - protein, 1149 aa, synthetic construct
MALPVTALLLPLALLLHAARPDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKGGGGSNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRPGLAGGRPEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMALPVTALLLPLALLLHAARPEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSGGGGSQKEVEQNSGPLSVPEGAIASLNCTYSDRGSQSFFWYRQYSGKSPELIMSIYSNGDKEDGRFTAQLNKASQYVSLLIRDSQPSDSATYLCAVTTDSWGKLQFGAGTQVVVTPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 49 - DNA, 8026 bp, synthetic construct
  source 1..8026: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatggctctgcctgtgacagctctgctgctgcctcttgctctgcttctgcatgccgccagacctgaagtgcagctggttgaaagtggcggcggactggttcaacctggcggatctctgagactgagctgtgccgccagcggcttcaatgtgtatgcctctggcatgcactgggtccgacaggcccctggaaaaggccttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagcagcttctactacgtgtacgccatggactactggggccagggaacactggtcaccgtgtccagcggcggtggcggttctaatgccggcgtgacccagacacctaagttccaggtgctgaaaaccggccagagcatgaccctgcagtgcgcccaggatatgaaccacgagtacatgagctggtacagacaggaccctggcatgggcctgagactgatccactattctgtcggagccggcatcaccgaccagggcgaagttcctaatggctacaacgtgtccagaagcaccaccgaggacttcccactgagactgctgtctgccgctcctagccagaccagcgtgtacttctgtgcctctagacctggactggctggcggcagacctgagcagtattttggccctggcaccagactgaccgtgaccgaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagcagggccaagagaagtggaagcggcgccacaaacttcagcctgttgaaacaggccggggacgtcgaagagaatcctggaccaatggcactgcccgtcactgccttgttgctgccacttgcactgctgctccatgccgccaggccagacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaagggcggtggcggttctcagaaagaggtggaacagaacagcggccctctgtctgttcctgaaggcgctatcgccagcctgaactgcacctacagcgatagaggcagccagagcttcttctggtacagacagtacagcggcaagagccccgagctgatcatgagcatctacagcaacggcgacaaagaggacggccggtttacagcccagctgaacaaggccagccagtacgtgtccctgctgatcagagatagccagcctagcgacagcgccacctatctgtgtgccgtgaccacagatagctggggcaagctgcagtttggcgccggaacacaggtggtggttacacctgacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 50 - protein, 1149 aa, synthetic construct
MALPVTALLLPLALLLHAARPEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSGGGGSNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRPGLAGGRPEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMALPVTALLLPLALLLHAARPDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKGGGGSQKEVEQNSGPLSVPEGAIASLNCTYSDRGSQSFFWYRQYSGKSPELIMSIYSNGDKEDGRFTAQLNKASQYVSLLIRDSQPSDSATYLCAVTTDSWGKLQFGAGTQVVVTPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 51 - DNA, 7957 bp, synthetic construct
  source 1..7957: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgggcaccagactgctgtgttgggctgctctgtgtctgctgggagccgaacttactgaagctggcgtggcccagtctcctcggtacaagatcatcgagaagcggcagagcgtggccttctggtgcaatcctatcagcggacacgccacactgtactggtatcagcagatcctcggacagggccccaagctgctgattcagttccagaacaacggcgtggtggacgacagccagctgcctaaggatagattcagcgccgagcggctgaaaggcgtggacagcacactgaagatccagcctgccaagctggaagatagcgccgtgtacctgtgtgccagctctctggatcctggcgataccggcgagctgttctttggcgagggcagcagactgaccgtgctcgaggacctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcacagatccccagcctctgaaagagcagccagctctgaacgacagccggtactgtctgagcagcaggctgagagtgtccgccaccttctggcagaaccccagaaaccacttcagatgccaggtgcagttctacggcctgagcgagaacgatgagtggacacaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatgatggctgctggtggacctggtgctggatctgctgcccctgtgtctagcacatctagcctgcctctggccgctctgaacatgagagtcagaagaaggctgagcctgttcctgaacgtgcggactcaggtggccgctgattggacagctctggccgaggaaatggacttcgagtacctggaaatccggcagctggaaacccaggccgatcctacaggcagactgctggatgcttggcaaggcagacctggcgcttctgtggggagactgcttgagctgctgacaaagctgggcagagatgacgtgctgctggaactgggccctagcatcgaggaagattgccagaagtacatcctgaagcagcagcaagaggaagccgagaagcctctgcaagtggccgccgtggatagcagcgttccaagaacagctgagctggccggcatcaccacactggatgatcctctgggaaagaaggtggccaagaagcccaccaacaaggcccctcatcctaagcaagagccccaagagatcaacttccccgacgatctgcccggcagcaatactgctgctcccgtgcaagaaaccctgcacggttgtcagcccgtgacacaagaggacggcaaagaaagccggatcagcgtgcaagagagacagagagccaagagatctggcagcggcgccacaaactttagcctgctgaaacaggccggcgacgtggaagagaaccccggacctatggaatcctttctcggtggcgtgctgctcatcctgtggctgcaagtggattgggtcaagagccagaagatcgagcagaacagcgaggccctgaacatccaagagggcaagaccgccactctgacctgcaactacaccaactacagccccgcctacctgcagtggtacagacaggaccctggaagaggccctgtgttcctgctgctgatccgcgagaatgagaaagagaagcgcaaagagcgcctgaaagtcaccttcgacacaaccctgaagcagagcctgttccacatcaccgcctctcagccagccgatagcgccacatatctgtgcgccctggacatctaccctcacgacatgagattcggagccggcaccaggctgacagtgaagcccgatattcagaaccccgatcctgccgtctatcagctgagagacagcaagagcagcgacaagagcgtgtgcctgttcaccgacttcgactcccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctcctgaaagtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 52 - protein, 1126 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDPGDTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGKKVAKKPTNKAPHPKQEPQEINFPDDLPGSNTAAPVQETLHGCQPVTQEDGKESRISVQERQRAKRSGSGATNFSLLKQAGDVEENPGPMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALDIYPHDMRFGAGTRLTVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 53 - DNA, 7720 bp, synthetic construct
  source 1..7720: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgggcaccagactgctgtgttgggctgctctgtgtctgctgggagccgaacttactgaagctggcgtggcccagtctcctcggtacaagatcatcgagaagcggcagagcgtggccttctggtgcaatcctatcagcggacacgccacactgtactggtatcagcagatcctcggacagggccccaagctgctgattcagttccagaacaacggcgtggtggacgacagccagctgcctaaggatagattcagcgccgagcggctgaaaggcgtggacagcacactgaagatccagcctgccaagctggaagatagcgccgtgtacctgtgtgccagctctctggatcctggcgataccggcgagctgttctttggcgagggcagcagactgaccgtgctcgaggacctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcacagatccccagcctctgaaagagcagccagctctgaacgacagccggtactgtctgagcagcaggctgagagtgtccgccaccttctggcagaaccccagaaaccacttcagatgccaggtgcagttctacggcctgagcgagaacgatgagtggacacaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggactctagaggcagagccaagagatctggcagcggcgccacaaactttagcctgctgaaacaggccggcgacgtggaagagaaccccggacctatggaatcctttctcggtggcgtgctgctcatcctgtggctgcaagtggattgggtcaagagccagaagatcgagcagaacagcgaggccctgaacatccaagagggcaagaccgccactctgacctgcaactacaccaactacagccccgcctacctgcagtggtacagacaggaccctggaagaggccctgtgttcctgctgctgatccgcgagaatgagaaagagaagcgcaaagagcgcctgaaagtcaccttcgacacaaccctgaagcagagcctgttccacatcaccgcctctcagccagccgatagcgccacatatctgtgcgccctggacatctaccctcacgacatgagattcggagccggcaccaggctgacagtgaagcccgatattcagaaccccgatcctgccgtctatcagctgagagacagcaagagcagcgacaagagcgtgtgcctgttcaccgacttcgactcccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctcctgaaagtggccggcttcaacctgctgatgaccctgagactgtggtccagccgggccaaacgatctggctctggccagtgtaccaactatgccctgctcaagctcgctggcgacgtcgagtctaaccctggaccaatgctcggcatctggacactgctgcctctggtgctgacaagcgtggccagactgagcagcaagagcgtgaacgcccaagtgaccgacatcaacagcaaaggcctggaactgagaaagaccgtgaccaccgtggaaacccagaacctggaaggcctgcaccacgacggccagttctgtcacaaaccttgtccacctggcgagcggaaggccagagattgcacagtgaatggcgacgagcctgactgcgtgccctgtcaagagggcaaagagtacaccgacaaggcccacttcagcagcaagtgcagacggtgcagactgtgcgacgaaggccacggactggaagtggaaatcaactgcacccggacacagaacaccaagtgccggtgcaagcccaacttcttctgcaacagcaccgtgtgcgagcactgcgacccttgtaccaagtgcgaacacggcatcatcaaagagtgcaccctgacctccaacacgaagtgcaaagaggaaggcagcagaagcaacctcggctggctgtgtctgctgctgctccccattcctctgatcgtgtgggttatggctgctggcggacctggtgctggatctgctgcacctgtgtccagcacatctagcctgccactggccgctctgaacatgagagtcagaagaaggctgagcctgttcctgaacgtgcgcactcaggtggccgctgattggacagctctggccgaggaaatggacttcgagtacctggaaatccggcagctggaaacacaggccgatcctacaggcagactgctggatgcttggcaaggcagacctggcgcttctgtggggagactgcttgagctgctgacaaagctgggcagagatgacgtgctgctggaactgggacccagcatcgaggaagattgccagaagtacatcctgaagcagcaacaagaggaagccgagaagcctctgcaggttgccgccgtggatagcagcgttccaagaacagctgagctggccggcatcaccacactggatgatcctctgggaaagaaggtggccaagaagcccacaaacaaggcccctcatcctaagcaagagccccaagagatcaacttccccgacgatctgcccggcagcaatactgcagctcccgtgcaagaaaccctgcacggttgtcagcccgtgacacaagaggacggcaaagaaagccggatcagcgtgcaagagagacagtgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 54 - protein, 1047 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDPGDTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGATNFSLLKQAGDVEENPGPMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALDIYPHDMRFGAGTRLTVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGQCTNYALLKLAGDVESNPGPMLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGKKVAKKPTNKAPHPKQEPQEINFPDDLPGSNTAAPVQETLHGCQPVTQEDGKESRISVQERQ

SEQ ID NO: 55 - DNA, 7195 bp, synthetic construct
  source 1..7195: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgggcaccagactgctgtgttgggctgctctgtgtctgctgggagccgaacttactgaagctggcgtggcccagtctcctcggtacaagatcatcgagaagcggcagagcgtggccttctggtgcaatcctatcagcggacacgccacactgtactggtatcagcagatcctcggacagggccccaagctgctgattcagttccagaacaacggcgtggtggacgacagccagctgcctaaggatagattcagcgccgagcggctgaaaggcgtggacagcacactgaagatccagcctgccaagctggaagatagcgccgtgtacctgtgtgccagctctctggatcctggcgataccggcgagctgttctttggcgagggcagcagactgaccgtgctcgaggacctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcacagatccccagcctctgaaagagcagccagctctgaacgacagccggtactgtctgagcagcaggctgagagtgtccgccaccttctggcagaaccccagaaaccacttcagatgccaggtgcagttctacggcctgagcgagaacgatgagtggacacaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggactctagaggcagagccaagagatctggcagcggcgccacaaactttagcctgctgaaacaggccggcgacgtggaagagaaccccggacctatggaatcctttctcggtggcgtgctgctcatcctgtggctgcaagtggattgggtcaagagccagaagatcgagcagaacagcgaggccctgaacatccaagagggcaagaccgccactctgacctgcaactacaccaactacagccccgcctacctgcagtggtacagacaggaccctggaagaggccctgtgttcctgctgctgatccgcgagaatgagaaagagaagcgcaaagagcgcctgaaagtcaccttcgacacaaccctgaagcagagcctgttccacatcaccgcctctcagccagccgatagcgccacatatctgtgcgccctggacatctaccctcacgacatgagattcggagccggcaccaggctgacagtgaagcccgatattcagaaccccgatcctgccgtctatcagctgagagacagcaagagcagcgacaagagcgtgtgcctgttcaccgacttcgactcccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctcctgaaagtggccggcttcaacctgctgatgaccctgagactgtggtccagccgggccaaacgatctggctctggccagtgtaccaactatgccctgctcaagctcgctggcgacgtcgagtctaaccctggaccaatgctcggcatctggacactgctgcctctggtgctgacaagcgtggccagactgagcagcaagagcgtgaacgcccaagtgaccgacatcaacagcaaaggcctggaactgagaaagaccgtgaccaccgtggaaacccagaacctggaaggcctgcaccacgacggccagttctgtcacaaaccttgtccacctggcgagcggaaggccagagattgcacagtgaatggcgacgagcctgactgcgtgccctgtcaagagggcaaagagtacaccgacaaggcccacttcagcagcaagtgcagacggtgcagactgtgcgacgaaggccacggactggaagtggaaatcaactgcacccggacacagaacaccaagtgccggtgcaagcccaacttcttctgcaacagcaccgtgtgcgagcactgcgacccttgtaccaagtgcgaacacggcatcatcaaagagtgcaccctgacctccaacacgaagtgcaaagaggaaggcagcagaagcaacctcggctggctgtgtctgctgctgctccccattcctctgatcgtgtgggtcgccctgtacctgctgagaagggatcagagactgcctcctgacgctcacaaaccaccaggcggaggcagcttcagaacccctatccaagaggaacaggctgacgcccacagcaccctggccaaaatttgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 56 - protein, 872 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDPGDTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGATNFSLLKQAGDVEENPGPMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALDIYPHDMRFGAGTRLTVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGQCTNYALLKLAGDVESNPGPMLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVALYLLRRDQRLPPDAHKPPGGGSFRTPIQEEQADAHSTLAKI

SEQ ID NO: 57 - DNA, 7195 bp, synthetic construct
  source 1..7195: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgggcaccagactgctgtgttgggctgctctgtgtctgctgggagccgaacttactgaagctggcgtggcccagtctcctcggtacaagatcatcgagaagcggcagagcgtggccttctggtgcaatcctatcagcggacacgccacactgtactggtatcagcagatcctcggacagggccccaagctgctgattcagttccagaacaacggcgtggtggacgacagccagctgcctaaggatagattcagcgccgagcggctgaaaggcgtggacagcacactgaagatccagcctgccaagctggaagatagcgccgtgtacctgtgtgccagctctctggatcctggcgataccggcgagctgttctttggcgagggcagcagactgaccgtgctcgaggacctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcacagatccccagcctctgaaagagcagccagctctgaacgacagccggtactgtctgagcagcaggctgagagtgtccgccaccttctggcagaaccccagaaaccacttcagatgccaggtgcagttctacggcctgagcgagaacgatgagtggacacaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggactctagaggcagagccaagagatctggcagcggcgccacaaactttagcctgctgaaacaggccggcgacgtggaagagaaccccggacctatggaatcctttctcggtggcgtgctgctcatcctgtggctgcaagtggattgggtcaagagccagaagatcgagcagaacagcgaggccctgaacatccaagagggcaagaccgccactctgacctgcaactacaccaactacagccccgcctacctgcagtggtacagacaggaccctggaagaggccctgtgttcctgctgctgatccgcgagaatgagaaagagaagcgcaaagagcgcctgaaagtcaccttcgacacaaccctgaagcagagcctgttccacatcaccgcctctcagccagccgatagcgccacatatctgtgcgccctggacatctaccctcacgacatgagattcggagccggcaccaggctgacagtgaagcccgatattcagaaccccgatcctgccgtctatcagctgagagacagcaagagcagcgacaagagcgtgtgcctgttcaccgacttcgactcccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctcctgaaagtggccggcttcaacctgctgatgaccctgagactgtggtccagccgggccaaacgatctggctctggccagtgtaccaactatgccctgctcaagctcgctggcgacgtcgagtctaaccctggaccaatgctcggcatctggacactgctgcctctggtgctgacaagcgtggccagactgagcagcaagagcgtgaacgcccaagtgaccgacatcaacagcaaaggcctggaactgagaaagaccgtgaccaccgtggaaacccagaacctggaaggcctgcaccacgacggccagttctgtcacaaaccttgtccacctggcgagcggaaggccagagattgcacagtgaatggcgacgagcctgactgcgtgccctgtcaagagggcaaagagtacaccgacaaggcccacttcagcagcaagtgcagacggtgcagactgtgcgacgaaggccacggactggaagtggaaatcaactgcacccggacacagaacaccaagtgccggtgcaagcccaacttcttctgcaacagcaccgtgtgcgagcactgcgacccttgtaccaagtgcgaacacggcatcatcaaagagtgcaccctgacctccaacacgaagtgcaaagaggaaggcagcagaagcaacctcggctggctgtgtctgctgctgctccccattcctctgatcgtgtgggtcaagcggggcagaaagaagctgctgtacatcttcaagcagcccttcatgcggcccgtgcagaccacacaagaggaagatggctgctcctgcagattccccgaggaagaagaaggcggctgcgaactttgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 58 - protein, 872 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDPGDTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGATNFSLLKQAGDVEENPGPMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALDIYPHDMRFGAGTRLTVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGQCTNYALLKLAGDVESNPGPMLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRGRKKLLYIFKQPFMRPVQTTQEEDGCSCRFPEEEEGGCEL

SEQ ID NO: 59 - DNA, 7654 bp, synthetic construct
  source 1..7654: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgggcaccagactgctgtgttgggctgctctgtgtctgctgggagccgaacttactgaagctggcgtggcccagtctcctcggtacaagatcatcgagaagcggcagagcgtggccttctggtgcaatcctatcagcggacacgccacactgtactggtatcagcagatcctcggacagggccccaagctgctgattcagttccagaacaacggcgtggtggacgacagccagctgcctaaggatagattcagcgccgagcggctgaaaggcgtggacagcacactgaagatccagcctgccaagctggaagatagcgccgtgtacctgtgtgccagctctctggatcctggcgataccggcgagctgttctttggcgagggcagcagactgaccgtgctcgaggacctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcacagatccccagcctctgaaagagcagccagctctgaacgacagccggtactgtctgagcagcaggctgagagtgtccgccaccttctggcagaaccccagaaaccacttcagatgccaggtgcagttctacggcctgagcgagaacgatgagtggacacaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggactctagaggcagagccaagagatctggcagcggcgccacaaactttagcctgctgaaacaggccggcgacgtggaagagaaccccggacctatggaatcctttctcggtggcgtgctgctcatcctgtggctgcaagtggattgggtcaagagccagaagatcgagcagaacagcgaggccctgaacatccaagagggcaagaccgccactctgacctgcaactacaccaactacagccccgcctacctgcagtggtacagacaggaccctggaagaggccctgtgttcctgctgctgatccgcgagaatgagaaagagaagcgcaaagagcgcctgaaagtcaccttcgacacaaccctgaagcagagcctgttccacatcaccgcctctcagccagccgatagcgccacatatctgtgcgccctggacatctaccctcacgacatgagattcggagccggcaccaggctgacagtgaagcccgatattcagaaccccgatcctgccgtctatcagctgagagacagcaagagcagcgacaagagcgtgtgcctgttcaccgacttcgactcccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctcctgaaagtggccggcttcaacctgctgatgaccctgagactgtggtccagccgggccaaacgatctggctctggccagtgtaccaactatgccctgctcaagctcgctggcgacgtcgagtctaaccctggaccaatgctcggcatctggacactgctgcctctggtgctgacaagcgtggccagactgagcagcaagagcgtgaacgcccaagtgaccgacatcaacagcaaaggcctggaactgagaaagaccgtgaccaccgtggaaacccagaacctggaaggcctgcaccacgacggccagttctgtcacaaaccttgtccacctggcgagcggaaggccagagattgcacagtgaatggcgacgagcctgactgcgtgccctgtcaagagggcaaagagtacaccgacaaggcccacttcagcagcaagtgcagacggtgcagactgtgcgacgaaggccacggactggaagtggaaatcaactgcacccggacacagaacaccaagtgccggtgcaagcccaacttcttctgcaacagcaccgtgtgcgagcactgcgacccttgtaccaagtgcgaacacggcatcatcaaagagtgcaccctgacctccaacacgaagtgcaaagaggaaggcagcagaagcaacctcggctggctgtgtctgctgctgctccccattcctctgatcgtgtgggtcaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggatgaagtcgagggctttctgcaggacacctttccacagcagctggaagagagcgagaagcagagactcggcggagatgtgcagagccctaattgccctagcgaggacgtggtcatcacccctgagagcttcggcagagatagcagcctgacatgtctggccggcaatgtgtccgcctgtgatgcccctatcctgagcagctccagaagcctggattgcagagagagcggcaagaacggccctcacgtgtaccaggatctgctcctgtctctgggcaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaaccagtgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 60 - protein, 1025 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDPGDTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGATNFSLLKQAGDVEENPGPMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALDIYPHDMRFGAGTRLTVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGQCTNYALLKLAGDVESNPGPMLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 61 - DNA, 8809 bp, synthetic construct
  source 1..8809: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgggcaccagactgctgtgttgggctgctctgtgtctgctgggagccgaacttactgaagctggcgtggcccagtctcctcggtacaagatcatcgagaagcggcagagcgtggccttctggtgcaatcctatcagcggacacgccacactgtactggtatcagcagatcctcggacagggccccaagctgctgattcagttccagaacaacggcgtggtggacgacagccagctgcctaaggatagattcagcgccgagcggctgaaaggcgtggacagcacactgaagatccagcctgccaagctggaagatagcgccgtgtacctgtgtgccagctctctggatcctggcgataccggcgagctgttctttggcgagggcagcagactgaccgtgctcgaggacctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcacagatccccagcctctgaaagagcagccagctctgaacgacagccggtactgtctgagcagcaggctgagagtgtccgccaccttctggcagaaccccagaaaccacttcagatgccaggtgcagttctacggcctgagcgagaacgatgagtggacacaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggactctagaggcagagccaagagatctggcagcggcgccacaaactttagcctgctgaaacaggccggcgacgtggaagagaaccccggacctatggaatcctttctcggtggcgtgctgctcatcctgtggctgcaagtggattgggtcaagagccagaagatcgagcagaacagcgaggccctgaacatccaagagggcaagaccgccactctgacctgcaactacaccaactacagccccgcctacctgcagtggtacagacaggaccctggaagaggccctgtgttcctgctgctgatccgcgagaatgagaaagagaagcgcaaagagcgcctgaaagtcaccttcgacacaaccctgaagcagagcctgttccacatcaccgcctctcagccagccgatagcgccacatatctgtgcgccctggacatctaccctcacgacatgagattcggagccggcaccaggctgacagtgaagcccgatattcagaaccccgatcctgccgtctatcagctgagagacagcaagagcagcgacaagagcgtgtgcctgttcaccgacttcgactcccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctcctgaaagtggccggcttcaacctgctgatgaccctgagactgtggtccagccgggccaaacgatctggctctggccagtgtaccaactatgccctgctcaagctcgctggcgacgtcgagtctaaccctggaccaatggcccagtggaatcagctgcagcagctcgacaccagatacctcgaacagctccaccagctgtactccgacagcttccctatggaactgcggcagtttctggccccttggatcgagtctcaggattgggcctacgccgccagcaaagagtctcacgccacactggtgttccacaacctgctgggcgagatcgaccagcagtacagccggtttctgcaagagtccaacgtgctgtaccagcacaacctgaggcggatcaagcagttcctgcagagcagatacctggaaaagcccatggaaatcgcccggatcgtggccagatgtctgtgggaagaaagcagactgctgcagaccgccgctacagctgctcaacaaggcggacaggccaaccatcctacagccgccgtggttacagagaagcagcagatgctggaacagcatctccaggacgtgcggaagagagtgcaggacctggaacagaaaatgaaggtggtggaaaacctgcaggacgacttcgacttcaactacaagaccctgaagtcccagggcgacatgcaggatctgaacggcaacaaccagagcgtgacccggcagaagatgcagcaactcgaacagatgctgacagccctggaccagatgaggcggagcattgtgtctgaactggccggactgctgagcgccatggaatacgtgcagaaaaccctgaccgacgaggaactggctgactggaaaagacggcagcagatcgcctgtatcggcggacctcctaacatctgcctggaccggctggaaaactggatcacaagcctggccgaaagccagctgcagacaagacagcagatcaagaagctggaagaactgcagcagaaggtgtcctacaagggcgaccccatcgtgcagcacagacccatgctcgaggaacgcatcgtggaactgttccggaatctgatgaagtccgccttcgtggtggaacggcagccctgtatgcctatgcaccccgatagacccctggtcatcaagaccggcgtgcagttcaccaccaaagtgcggctgctggtcaagttccccgagctgaactaccagctgaagatcaaagtgtgcatcgacaaggacagcggcgacgtggcagctctgagaggcagcagaaagttcaacatcctgggcaccaacaccagggtcatgaacatggaagagagcaacaacggctccctgagcgccgagttcaagcacctgacactgcgcgagcagagatgcggaaatggcggcagagccaactgtgacgccagcctgattgtgacagaggaactgcacctgatcaccttcgagacagaggtgtaccaccagggcctgaagatcgacctggaaacccactctctgcccgtggtggtcatcagcaacatctgtcagatgcccaacgcctgggccagcatcctgtggtacaacatgctgaccaacaatcccaagaacgtgaacttcttcaccaagcctccaatcggcacctgggaccaagtggccgaagttctgtcctggcagttcagcagcaccaccaagagaggcctgagcatcgagcagctgacaacactggccgagaagctgcttggacccggcgtgaactatagcggctgtcagatcacatgggccaagttctgcaaagaaaacatggccggcaagggcttcagcttctgggtctggctggacaacatcatcgacctcgtcaagaagtacattctggccctgtggaacgagggctacatcatgggcttcatctccaaagagagagagcgggccatcctgagcacaaagcctccaggcacattcctgctgcggttcagcgagagcagcaaagaaggcggcgtcacctttacctgggtcgagaaggatatcagcggcaagacccagatccagtccgtggaaccctacaccaagcagcagctgaacaatatgagcttcgccgagatcatcatggggtacaagatcatggacgccaccaatatcctggtgtccccactggtgtacctgtatcctgacatccccaaagaggaagccttcggcaagtactgcagacccgagagccaagagcaccctgaagccgatcctggaagcgccgctccttacctgaaaacaaagttcatctgcgtgacccctaccacctgtagcaacaccatcgatctgcctatgagccccagaacactggacagcctgatgcagttcggcaacaatggcgaaggcgccgaaccatctgctggcggacagtttgagagcctgaccttcgacatggaactgacctccgagtgcgccacctctcctatgtgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 62 - protein, 1410 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDPGDTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGATNFSLLKQAGDVEENPGPMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALDIYPHDMRFGAGTRLTVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGQCTNYALLKLAGDVESNPGPMAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTRVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 63 - DNA, 8878 bp, synthetic construct
  source 1..8878: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgggcaccagactgctgtgttgggctgctctgtgtctgctgggagccgaacttactgaagctggcgtggcccagtctcctcggtacaagatcatcgagaagcggcagagcgtggccttctggtgcaatcctatcagcggacacgccacactgtactggtatcagcagatcctcggacagggccccaagctgctgattcagttccagaacaacggcgtggtggacgacagccagctgcctaaggatagattcagcgccgagcggctgaaaggcgtggacagcacactgaagatccagcctgccaagctggaagatagcgccgtgtacctgtgtgccagctctctggatcctggcgataccggcgagctgttctttggcgagggcagcagactgaccgtgctcgaggacctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcacagatccccagcctctgaaagagcagccagctctgaacgacagccggtactgtctgagcagcaggctgagagtgtccgccaccttctggcagaaccccagaaaccacttcagatgccaggtgcagttctacggcctgagcgagaacgatgagtggacacaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggactctagaggcagagccaagagatctggcagcggcgccacaaactttagcctgctgaaacaggccggcgacgtggaagagaaccccggacctatggaatcctttctcggtggcgtgctgctcatcctgtggctgcaagtggattgggtcaagagccagaagatcgagcagaacagcgaggccctgaacatccaagagggcaagaccgccactctgacctgcaactacaccaactacagccccgcctacctgcagtggtacagacaggaccctggaagaggccctgtgttcctgctgctgatccgcgagaatgagaaagagaagcgcaaagagcgcctgaaagtcaccttcgacacaaccctgaagcagagcctgttccacatcaccgcctctcagccagccgatagcgccacatatctgtgcgccctggacatctaccctcacgacatgagattcggagccggcaccaggctgacagtgaagcccgatattcagaaccccgatcctgccgtctatcagctgagagacagcaagagcagcgacaagagcgtgtgcctgttcaccgacttcgactcccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctcctgaaagtggccggcttcaacctgctgatgaccctgagactgtggtccagccgggccaaacgatctggctctggccagtgtaccaactatgccctgctcaagctcgctggcgacgtcgagtctaaccctggaccaatggccggatggattcaggctcagcagcttcaaggcgacgccctgagacagatgcaggtcctgtatggccagcacttccccatcgaagtgcggcactatctggcccagtggatcgagtctcagccctgggatgccatcgacctggacaaccctcaggatagaggacaggccacacagctgctggaaggcctggttcaagagctgcagaagaaagccgagcaccaagtgggcgaagatggcttcctgctgaagatcaagctgggccactacgccactcagctccagaacacctacgacagatgccccatggaactcgtgcggtgcatccggcacatcctgtacaacgagcagaggctcgtcagagaggccaacaactgttcttctcctgccggcgtgctggtggatgccatgagccagaaacacctccagatcaaccagcgcttcgaggaactgcggctgatcacccaggacaccgagaacgagctgaagaagctgcagcagacccaagagtacttcatcatccagtaccaagagagcctgcgcattcaggcccagtttgctcagctgggacagctgaaccctcaagagcggatgagcagagagacagccctccagcagaaacaggtgtccctggaaacctggctgcagagagaggctcagaccctgcagcagtacagagtggaactggccgagaagcaccagaaaacactgcagctgctgcggaagcagcagacaatcatcctggacgacgagctgatccagtggaagcggagacagcagctggctggaaatggcggacctcctgagggatctctggacgtgttgcagtcttggtgcgagaagctggccgaaatcatctggcagaaccggcagcagattcggagagccgagagactgtgtcagcagctccctattcctggacctgtggaagagatgctggctgaagtgaacgccaccatcaccgacatcatcagcgccctggtcaccagcacctttatcatcgagaagcagcctccacaggtgctgaaaacccagaccaagtttgccgccacagtgcggttgctcgttggcggcaagctgaacgtgcacatgaaccctccacaagtgaaggccacaattatcagcgagcagcaggccaagagcctgctgaaaaacgagaacacccggaacgagtgcagcggcgagatcctgaacaactgctgcgtgatggaataccaccaggccaccggaacactgagcgcccacttcagaaatatgtccctgaagcggatcaagcgcgccgatagaagaggcgccgagtctgtgaccgaagagaagttcaccgtgctgttcgagagccagttcagcgtgggcagcaacgagctggtgttccaagtgaaaaccctgagcctgcctgtggtggtcatcgtgcacggaagccaggatcacaatgccaccgctacagtgctgtgggacaacgcctttgccgagcctggcagagtgccttttgccgtgcctgataaggtcctgtggcctcagctttgcgaggccctgaacatgaagttcaaggccgaggtgcagagcaaccggggcctgaccaaagaaaacctggtgtttcttgcccagaagctgttcaacatcagcagcaaccacctcgaggactacaacagcatgtccgtgtcttggtcccagttcaacagagagaacttgcccggctggaactacaccttctggcagtggttcgacggcgtcatggaagtcctgaagaaacatcacaagccccactggaacgacggcgccatcctgggctttgtgaacaaacagcaggcccacgatctgctgatcaacaagcccgatggcacctttctgctgagattcagcgacagcgagatcggcggcatcacaatcgcctggaagttcgacagccccgaccggaatctgtggaacctgaagccattcaccaccagagacttcagcatccgcagcctggctgacagactgggcgacctgaactacctgatctacgtgttccccgatcggcccaaggatgaggtgttcgccaagtactacacccctgtgctggctaaggccgtggacggctatgtgaagccccagatcaaacaggtggtgcccgagttcgtgaacgccttcactgatgctggcgcctccgccacctatatggatcaggcaccttctccagtcgtgtgccctcagcctcactacaacatgtacccacctaatcctgatcctgtgctcgaccaggacggcgagttcgatctggacgagtctatggacgtggcccggcacgttgaagaactgctcagacggcctatggacagcctggacgctagactgtctcctccagccggcctgtttacaagcgccagaagctctctgagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 64 - protein, 1433 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDPGDTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGATNFSLLKQAGDVEENPGPMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALDIYPHDMRFGAGTRLTVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGQCTNYALLKLAGDVESNPGPMAGWIQAQQLQGDALRQMQVLYGQHFPIEVRHYLAQWIESQPWDAIDLDNPQDRGQATQLLEGLVQELQKKAEHQVGEDGFLLKIKLGHYATQLQNTYDRCPMELVRCIRHILYNEQRLVREANNCSSPAGVLVDAMSQKHLQINQRFEELRLITQDTENELKKLQQTQEYFIIQYQESLRIQAQFAQLGQLNPQERMSRETALQQKQVSLETWLQREAQTLQQYRVELAEKHQKTLQLLRKQQTIILDDELIQWKRRQQLAGNGGPPEGSLDVLQSWCEKLAEIIWQNRQQIRRAERLCQQLPIPGPVEEMLAEVNATITDIISALVTSTFIIEKQPPQVLKTQTKFAATVRLLVGGKLNVHMNPPQVKATIISEQQAKSLLKNENTRNECSGEILNNCCVMEYHQATGTLSAHFRNMSLKRIKRADRRGAESVTEEKFTVLFESQFSVGSNELVFQVKTLSLPVVVIVHGSQDHNATATVLWDNAFAEPGRVPFAVPDKVLWPQLCEALNMKFKAEVQSNRGLTKENLVFLAQKLFNISSNHLEDYNSMSVSWSQFNRENLPGWNYTFWQWFDGVMEVLKKHHKPHWNDGAILGFVNKQQAHDLLINKPDGTFLLRFSDSEIGGITIAWKFDSPDRNLWNLKPFTTRDFSIRSLADRLGDLNYLIYVFPDRPKDEVFAKYYTPVLAKAVDGYVKPQIKQVVPEFVNAFTDAGASATYMDQAPSPVVCPQPHYNMYPPNPDPVLDQDGEFDLDESMDVARHVEELLRRPMDSLDARLSPPAGLFTSARSSLS

SEQ ID NO: 65 - DNA, 8005 bp, synthetic construct
  source 1..8005: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgggcaccagactgctgtgttgggctgctctgtgtctgctgggagccgaacttactgaagctggcgtggcccagtctcctcggtacaagatcatcgagaagcggcagagcgtggccttctggtgcaatcctatcagcggacacgccacactgtactggtatcagcagatcctcggacagggccccaagctgctgattcagttccagaacaacggcgtggtggacgacagccagctgcctaaggatagattcagcgccgagcggctgaaaggcgtggacagcacactgaagatccagcctgccaagctggaagatagcgccgtgtacctgtgtgccagctctctggatcctggcgataccggcgagctgttctttggcgagggcagcagactgaccgtgctcgaggacctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcacagatccccagcctctgaaagagcagccagctctgaacgacagccggtactgtctgagcagcaggctgagagtgtccgccaccttctggcagaaccccagaaaccacttcagatgccaggtgcagttctacggcctgagcgagaacgatgagtggacacaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggactctagaggcagagccaagagatctggcagcggcgccacaaactttagcctgctgaaacaggccggcgacgtggaagagaaccccggacctatggaatcctttctcggtggcgtgctgctcatcctgtggctgcaagtggattgggtcaagagccagaagatcgagcagaacagcgaggccctgaacatccaagagggcaagaccgccactctgacctgcaactacaccaactacagccccgcctacctgcagtggtacagacaggaccctggaagaggccctgtgttcctgctgctgatccgcgagaatgagaaagagaagcgcaaagagcgcctgaaagtcaccttcgacacaaccctgaagcagagcctgttccacatcaccgcctctcagccagccgatagcgccacatatctgtgcgccctggacatctaccctcacgacatgagattcggagccggcaccaggctgacagtgaagcccgatattcagaaccccgatcctgccgtctatcagctgagagacagcaagagcagcgacaagagcgtgtgcctgttcaccgacttcgactcccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctcctgaaagtggccggcttcaacctgctgatgaccctgagactgtggtccagccgggccaaacgatctggctctggccagtgtaccaactatgccctgctcaagctcgctggcgacgtcgagtctaaccctggaccaatgaccatcctgggcaccaccttcggcatggtgtttagcctgctgcaggtcgtgtctggcagcctggataacaacggcacagccacacctgagctgcctacacagggcaccttcagcaacgtgtccaccaatgtgtcctaccaagagacaaccacacctagcacactgggcagcacatctctgcaccctgtgtctcagcacggcaatgaggccaccaccaacatcaccgagacaaccgtgaagttcaccagcaccagcgtgatcacctccgtgtacggcaacaccaacagcagcgtgcagagccagacctccgtgatcagcaccgtgtttacaacccctgccaatgtctctacccctgagacaactctgaagcccagcctgtctcctggaaacgtgtccgatctgagcaccacctctaccagcctggccacctctcctacaaagccctacacaagcagcagccccatcctgagcgatatcaaggccgagatcaagtgcagcggcatccgggaagtgaagctgacccagggcatctgcctggaacagaacaagaccagcagctgcgccgagttcaagaaggacagaggcgaaggactggccagagtgctgtgtggcgaagaacaggccgatgctgatgctggcgctcaagtgtgctctctgctgctggctcagtctgaagtgcggcctcagtgtctgcttctggtcctggccaacagaaccgagatcagcagcaaactgcagctgatgaagaagcaccagagcgacctgaagaagctgggcatcctggacttcaccgagcaggatgtggccagccaccagagctacagccagaaaacccctatcctgctgacatgccccacaatcagcatcctgtcctttttcagcgtggccctgctcgtgatcctggcctgtgtgctgtggaagaagcggatcaagcccatcgtgtggcctagcctgcctgaccacaagaaaaccctggaacacctgtgcaagaagccccggaagaacctgaacgtgtccttcaatcccgagagcttcctggactgccagatccacagagtggacgacatccaggccagagatgaggtggaaggctttctgcaggacactttcccacagcagctggaagagagcgagaagcagagactcggcggagatgtgcagtcccctaattgccctagcgaggacgtggtcatcaccccagagagcttcggcagagatagcagcctgacatgtctggccggcaatgtgtctgcctgtgacgcccctattctgagcagctccagaagcctggattgcagagagagcggcaagaacggccctcacgtgtaccaggatctgctcctgtctctgggcactaccaatagcaccctgcctccaccattcagcctgcagagcggaatcctgacactgaacccagtggctcagggccagcctatcctgacaagcctgggctccaatcaagaagaggcctacgtcaccatgagcagcttctaccagaaccagtgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 66 - protein, 1142 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDPGDTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGATNFSLLKQAGDVEENPGPMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALDIYPHDMRFGAGTRLTVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGQCTNYALLKLAGDVESNPGPMTILGTTFGMVFSLLQVVSGSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTPILLTCPTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 67 - DNA, 7885 bp, synthetic construct
  source 1..7885: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgggcaccagactgctgtgttgggctgctctgtgtctgctgggagccgaacttactgaagctggcgtggcccagtctcctcggtacaagatcatcgagaagcggcagagcgtggccttctggtgcaatcctatcagcggacacgccacactgtactggtatcagcagatcctcggacagggccccaagctgctgattcagttccagaacaacggcgtggtggacgacagccagctgcctaaggatagattcagcgccgagcggctgaaaggcgtggacagcacactgaagatccagcctgccaagctggaagatagcgccgtgtacctgtgtgccagctctctggatcctggcgataccggcgagctgttctttggcgagggcagcagactgaccgtgctcgaggacctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcacagatccccagcctctgaaagagcagccagctctgaacgacagccggtactgtctgagcagcaggctgagagtgtccgccaccttctggcagaaccccagaaaccacttcagatgccaggtgcagttctacggcctgagcgagaacgatgagtggacacaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggactctagaggcagagccaagagatctggcagcggcgccacaaactttagcctgctgaaacaggccggcgacgtggaagagaaccccggacctatggaatcctttctcggtggcgtgctgctcatcctgtggctgcaagtggattgggtcaagagccagaagatcgagcagaacagcgaggccctgaacatccaagagggcaagaccgccactctgacctgcaactacaccaactacagccccgcctacctgcagtggtacagacaggaccctggaagaggccctgtgttcctgctgctgatccgcgagaatgagaaagagaagcgcaaagagcgcctgaaagtcaccttcgacacaaccctgaagcagagcctgttccacatcaccgcctctcagccagccgatagcgccacatatctgtgcgccctggacatctaccctcacgacatgagattcggagccggcaccaggctgacagtgaagcccgatattcagaaccccgatcctgccgtctatcagctgagagacagcaagagcagcgacaagagcgtgtgcctgttcaccgacttcgactcccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctcctgaaagtggccggcttcaacctgctgatgaccctgagactgtggtccagccgggccaaacgatctggctctggccagtgtaccaactatgccctgctcaagctcgctggcgacgtcgagtctaaccctggaccaatgaccatcctgggcaccaccttcggcatggtgtttagcctgctgcaggtcgtgtctggcgagtctggctatgcccagaatggcgacctggaagatgccgagctggacgactacagcttcagctgctacagccagctggaagtgaacggcagccagcactctctgacctgcgccttcgaagatcccgacgtgaacatcaccaacctggaattcgagatctgtggcgccctggtggaagtcaagtgcctgaacttcagaaagctgcaagagatctactttatcgagacaaagaagttcctgctgatcggcaagagcaacatctgcgtgaaagtgggcgagaagtccctgacctgcaagaagatcgacctgaccaccatcgtgaagcccgaggctcctttcgatctgagcgtggtgtatagagagggcgccaacgacttcgtggtcaccttcaacaccagccacctccaaaagaaatacgtgaaggtgctgatgcacgacgtggcctaccggcaagagaaggacgagaacaagtggacccacgtgaacctgagcagcaccaagctgacactgctgcagaggaaactgcagcctgccgctatgtacgagatcaaagtgcggagcatccccgaccactacttcaaaggcttttggagcgagtggtccccgagctactactttcggacccctgagatcaacaacagcagcggcgagatggaccccatcctgctgacctgtcctacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctcgaggaaagcgagaagcagagactcggcggagatgtgcagagccctaattgccctagcgaggacgtggtcatcaccccagagagcttcggcagagatagcagcctgacatgtctggccggcaatgtgtccgcctgtgatgcccctatcctgtccagcagcagaagcctggattgcagagagagcggcaagaacggccctcacgtgtaccaggatctgctcctgtctctgggaaccaccaacagcacactgcctccaccattcagcctgcagagcggcatcctgacactgaaccctgttgctcagggccagccaatcctgacaagcctgggcagcaatcaagaagaggcctacgtcaccatgagcagcttctaccagaaccagtgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 68 - protein, 1102 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDPGDTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGATNFSLLKQAGDVEENPGPMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALDIYPHDMRFGAGTRLTVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGQCTNYALLKLAGDVESNPGPMTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTCPTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 69 - DNA, 7936 bp, synthetic construct
  source 1..7936: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgggcactcgattgctctgttgggccgcgctgtgcttgctgggtgccgaattgacagaagcgggtgtagctcaaagtcctcggtacaagattattgagaaacgccagtctgttgccttctggtgtaaccctatctctggtcacgcaactctctattggtatcagcaaatcctgggtcaaggtcctaaacttctgattcaatttcagaataatggcgttgtggacgactctcaactgcctaaagacagattcagtgcggaaaggttgaaaggcgtcgatagcacactcaaaattcagccggcaaagcttgaagactccgccgtttatctgtgcgcgagttctcttgatcctggggacacaggggaactgttctttggcgagggatcacgattgaccgtcctggaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcatggctgctggtggacctggtgctggatctgctgcccctgtgtctagcacatctagcctgcctctggccgctctgaacatgagagtcagaagaaggctgagcctgttcctgaacgtgcggactcaggtggccgctgattggacagctctggccgaggaaatggacttcgagtacctggaaatccggcagctggaaacccaggccgatcctacaggcagactgctggatgcttggcaaggcagacctggcgcttctgtggggagactgcttgagctgctgacaaagctgggcagagatgacgtgctgctggaactgggccctagcatcgaggaagattgccagaagtacatcctgaagcagcagcaagaggaagccgagaagcctctgcaagtggccgccgtggatagcagcgttccaagaacagctgagctggccggcatcaccacactggatgatcctctgggaaagaaggtggccaagaagcccaccaacaaggcccctcatcctaagcaagagccccaagagatcaacttccccgacgatctgcccggcagcaatactgctgctcccgtgcaagaaaccctgcacggttgtcagcccgtgacacaagaggacggcaaagaaagccggatcagcgtgcaagagagacagagggccaagagaagtggaagcggcgccacaaacttcagcctgttgaaacaggccggggacgtcgaagagaatcctggaccaatggaatcctttctcggaggtgtactgctcatactctggctgcaagtggattgggtaaagagtcaaaaaattgaacaaaacagcgaggctttgaatatccaagagggcaagactgccacgctgacctgtaactatacaaactatagtccggcatacctccagtggtaccggcaagatcctggtcgcgggccggttttcctgttgcttattagagagaatgaaaaggaaaaaagaaaagaacgcttgaaggtcactttcgacacgacgcttaaacagagtcttttccacattaccgcgtcacagcctgcggactcagctacatacctgtgtgctctcgacatctatccccacgacatgaggtttggcgcaggcactcgactcacggtaaaacccgacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 70 - protein, 1119 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDPGDTGELFFGEGSRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGKKVAKKPTNKAPHPKQEPQEINFPDDLPGSNTAAPVQETLHGCQPVTQEDGKESRISVQERQRAKRSGSGATNFSLLKQAGDVEENPGPMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALDIYPHDMRFGAGTRLTVKPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 71 - DNA, 7690 bp, synthetic construct
  source 1..7690: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatgggcactcgattgctctgttgggccgcgctgtgcttgctgggtgccgaattgacagaagcgggtgtagctcaaagtcctcggtacaagattattgagaaacgccagtctgttgccttctggtgtaaccctatctctggtcacgcaactctctattggtatcagcaaatcctgggtcaaggtcctaaacttctgattcaatttcagaataatggcgttgtggacgactctcaactgcctaaagacagattcagtgcggaaaggttgaaaggcgtcgatagcacactcaaaattcagccggcaaagcttgaagactccgccgtttatctgtgcgcgagttctcttgatcctggggacacaggggaactgttctttggcgagggatcacgattgaccgtcctggaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagcagggccaagagaagtggaagcggcgccacaaacttcagcctgttgaaacaggccggggacgtcgaagagaatcctggaccaatggaatcctttctcggaggtgtactgctcatactctggctgcaagtggattgggtaaagagtcaaaaaattgaacaaaacagcgaggctttgaatatccaagagggcaagactgccacgctgacctgtaactatacaaactatagtccggcatacctccagtggtaccggcaagatcctggtcgcgggccggttttcctgttgcttattagagagaatgaaaaggaaaaaagaaaagaacgcttgaaggtcactttcgacacgacgcttaaacagagtcttttccacattaccgcgtcacagcctgcggactcagctacatacctgtgtgctctcgacatctatccccacgacatgaggtttggcgcaggcactcgactcacggtaaaacccgacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgctcggcatctggacactgctgcctctggtgctgacaagcgtggccagactgagcagcaagagcgtgaacgcccaagtgaccgacatcaacagcaaaggcctggaactgagaaagaccgtgaccaccgtggaaacccagaacctggaaggcctgcaccacgacggccagttctgtcacaaaccttgtccacctggcgagcggaaggccagagattgcacagtgaatggcgacgagcctgactgcgtgccctgtcaagagggcaaagagtacaccgacaaggcccacttcagcagcaagtgcagacggtgcagactgtgcgacgaaggccacggactggaagtggaaatcaactgcacccggacacagaacaccaagtgccggtgcaagcccaacttcttctgcaacagcaccgtgtgcgagcactgcgacccttgtaccaagtgcgaacacggcatcatcaaagagtgcaccctgacctccaacacgaagtgcaaagaggaaggcagcagaagcaacctcggctggctgtgtctgctgctgctccccattcctctgatcgtgtgggttatggctgctggcggacctggtgctggatctgctgcacctgtgtccagcacatctagcctgccactggccgctctgaacatgagagtcagaagaaggctgagcctgttcctgaacgtgcgcactcaggtggccgctgattggacagctctggccgaggaaatggacttcgagtacctggaaatccggcagctggaaacacaggccgatcctacaggcagactgctggatgcttggcaaggcagacctggcgcttctgtggggagactgcttgagctgctgacaaagctgggcagagatgacgtgctgctggaactgggacccagcatcgaggaagattgccagaagtacatcctgaagcagcaacaagaggaagccgagaagcctctgcaggttgccgccgtggatagcagcgttccaagaacagctgagctggccggcatcaccacactggatgatcctctgggaaagaaggtggccaagaagcccacaaacaaggcccctcatcctaagcaagagccccaagagatcaacttccccgacgatctgcccggcagcaatactgcagctcccgtgcaagaaaccctgcacggttgtcagcccgtgacacaagaggacggcaaagaaagccggatcagcgtgcaagagagacagtgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 72 - protein, 1037 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDPGDTGELFFGEGSRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALDIYPHDMRFGAGTRLTVKPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGKKVAKKPTNKAPHPKQEPQEINFPDDLPGSNTAAPVQETLHGCQPVTQEDGKESRISVQERQ

SEQ ID NO: 73 - DNA, 7984 bp, synthetic construct
  source 1..7984: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatggctctgcctgtgacagctctgctgctgcctcttgctctgcttctgcatgccgccagacctgacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaaggaagccgccgctaaagaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcatggctgctggtggacctggtgctggatctgctgcccctgtgtctagcacatctagcctgcctctggccgctctgaacatgagagtcagaagaaggctgagcctgttcctgaacgtgcggactcaggtggccgctgattggacagctctggccgaggaaatggacttcgagtacctggaaatccggcagctggaaacccaggccgatcctacaggcagactgctggatgcttggcaaggcagacctggcgcttctgtggggagactgcttgagctgctgacaaagctgggcagagatgacgtgctgctggaactgggccctagcatcgaggaagattgccagaagtacatcctgaagcagcagcaagaggaagccgagaagcctctgcaagtggccgccgtggatagcagcgttccaagaacagctgagctggccggcatcaccacactggatgatcctctgggaaagaaggtggccaagaagcccaccaacaaggcccctcatcctaagcaagagccccaagagatcaacttccccgacgatctgcccggcagcaatactgctgctcccgtgcaagaaaccctgcacggttgtcagcccgtgacacaagaggacggcaaagaaagccggatcagcgtgcaagagagacagagggccaagagaagtggaagcggcgccacaaacttcagcctgttgaaacaggccggggacgtcgaagagaatcctggaccaatggcactgcccgtcactgccttgttgctgccacttgcactgctgctccatgccgccaggccagaagtgcagctggttgaaagtggcggcggactggttcaacctggcggatctctgagactgagctgtgccgccagcggcttcaatgtgtatgcctctggcatgcactgggtccgacaggcccctggaaaaggccttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagcagcttctactacgtgtacgccatggactactggggccagggaacactggtcaccgtgtccagcgaagccgccgctaaagacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgggagctggcgctacaggcagagctatggatggacctagactgctgctgctcctgcttctgggagtgtctcttggcggagccaaagaggcctgtcctaccggcctgtatacacactctggcgagtgctgcaaggcctgcaatcttggagaaggcgtcgcacagccttgcggcgctaatcagacagtgtgcgagccttgcctggacagcgtgacctttagcgacgtggtgtcagccaccgagccatgcaagccatgtaccgagtgtgtgggcctgcagagcatgtctgccccttgcgtggaagctgacgatgccgtgtgtagatgcgcctacggctactaccaggacgagacaacaggcagatgcgaggcctgtagagtgtgcgaagctggatctggcctggtgttcagctgccaagacaagcagaacaccgtgtgcgaggaatgccccgatggcacctatagcgacgaggccaaccatgtggatccctgcctgccttgtaccgtgtgtgaagataccgagcggcagctgcgcgagtgtacaagatgggctgatgccgagtgcgaagagatccctggcagatggatcaccagaagcacacctccagagggcagcgatagcacagccccttctacacaagagcccgaggctcctcctgagcaggacctgattgcttctacagtggctggcgtcgtgaccaccgtgatgggatctagtcagcccgtggtcaccagaggcaccaccgacaatctgatccccgtgtactgtagcatcctggccgccgtggttgttggactggtggcctatatcgccttcaagcggtggaacagctgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 74 - protein, 1135 aa, synthetic construct
MALPVTALLLPLALLLHAARPDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKEAAAKEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGKKVAKKPTNKAPHPKQEPQEINFPDDLPGSNTAAPVQETLHGCQPVTQEDGKESRISVQERQRAKRSGSGATNFSLLKQAGDVEENPGPMALPVTALLLPLALLLHAARPEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSEAAAKDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNS

SEQ ID NO: 75 - DNA, 7738 bp, synthetic construct
  source 1..7738: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccatggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagccaccatggctctgcctgtgacagctctgctgctgcctcttgctctgcttctgcatgccgccagacctgacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgcctacttcctgtacagcggcgtgcccagcagattcagcggctctagaagcggcaccgacttcaccctgaccataagcagtctgcagcccgaggacttcgccacctactactgtcagcagtacagccggtacagccccgtgacatttggccagggcaccaaggtggaaatcaaggaagccgccgctaaagaggacctgagaaacgtgacccctcctaaggtgtccctgttcgagcctagcaaggccgagatcgccaacaagcagaaagccacactcgtgtgcctggccagaggcttctttcccgatcacgtggaactgtcttggtgggtcaacggcaaagaggtgcacagcggcgtctgtactgatccccaggcctacaaagagagcaactacagctactgcctgagcagcagactgagagtgtccgccaccttctggcacaaccccagaaaccacttcagatgccaggtgcagtttcacggcctgagcgaagaggacaagtggcctgagggaagccccaagccagtgacacagaatatctctgccgaagcctggggcagagccgattgtggaattaccagcgccagctaccagcaaggcgtgctgtctgccacaatcctgtacgagatcctgctgggcaaagccactctgtacgccgtgctggtgtctaccctggtcgtgatggccatggtcaagcggaagaacagcagggccaagagaagtggaagcggcgccacaaacttcagcctgttgaaacaggccggggacgtcgaagagaatcctggaccaatggcactgcccgtcactgccttgttgctgccacttgcactgctgctccatgccgccaggccagaagtgcagctggttgaaagtggcggcggactggttcaacctggcggatctctgagactgagctgtgccgccagcggcttcaatgtgtatgcctctggcatgcactgggtccgacaggcccctggaaaaggccttgagtgggtcgccaagatctaccccgacagcgactacacctactatgccgacagcgtgaagggcagattcaccatcagcgccgacaccagcaagaacaccgcctacctgcagatgaacagcctgagagccgaggacaccgccgtgtactactgctccagagacagcagcttctactacgtgtacgccatggactactggggccagggaacactggtcaccgtgtccagcgaagccgccgctaaagacattcagaatcctgagcctgccgtgtaccagctgaaggaccctagaagccaggacagcaccctgtgcctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacgtgccaggacatcttcaaagagacaaacgccacctatcctagcagcgacgtgccctgtgatgccacactgaccgagaagtccttcgagacagacatgaacctgaacttccagaacctgctggtcatcgtgctgagaatcctgctgctgaaggtggccggcttcaacctgctgatgaccctgagactgtggtccagccgggccaaacgatctggctctggccagtgtaccaactatgccctgctcaagctcgctggcgacgtcgagtctaaccctggaccaatgctcggcatctggacactgctgcctctggtgctgacaagcgtggccagactgagcagcaagagcgtgaacgcccaagtgaccgacatcaacagcaaaggcctggaactgagaaagaccgtgaccaccgtggaaacccagaacctggaaggcctgcaccacgacggccagttctgtcacaaaccttgtccacctggcgagcggaaggccagagattgcacagtgaatggcgacgagcctgactgcgtgccctgtcaagagggcaaagagtacaccgacaaggcccacttcagcagcaagtgcagacggtgcagactgtgcgacgaaggccacggactggaagtggaaatcaactgcacccggacacagaacaccaagtgccggtgcaagcccaacttcttctgcaacagcaccgtgtgcgagcactgcgacccttgtaccaagtgcgaacacggcatcatcaaagagtgcaccctgacctccaacacgaagtgcaaagaggaaggcagcagaagcaacctcggctggctgtgtctgctgctgctccccattcctctgatcgtgtgggttatggctgctggcggacctggtgctggatctgctgcacctgtgtccagcacatctagcctgccactggccgctctgaacatgagagtcagaagaaggctgagcctgttcctgaacgtgcgcactcaggtggccgctgattggacagctctggccgaggaaatggacttcgagtacctggaaatccggcagctggaaacacaggccgatcctacaggcagactgctggatgcttggcaaggcagacctggcgcttctgtggggagactgcttgagctgctgacaaagctgggcagagatgacgtgctgctggaactgggacccagcatcgaggaagattgccagaagtacatcctgaagcagcaacaagaggaagccgagaagcctctgcaggttgccgccgtggatagcagcgttccaagaacagctgagctggccggcatcaccacactggatgatcctctgggaaagaaggtggccaagaagcccacaaacaaggcccctcatcctaagcaagagccccaagagatcaacttccccgacgatctgcccggcagcaatactgcagctcccgtgcaagaaaccctgcacggttgtcagcccgtgacacaagaggacggcaaagaaagccggatcagcgtgcaagagagacagtgatgattcgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttacacggcagggtcagggttctggatatctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaaccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 76 - protein, 1053 aa, synthetic construct
MALPVTALLLPLALLLHAARPDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKEAAAKEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMALPVTALLLPLALLLHAARPEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSEAAAKDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGQCTNYALLKLAGDVESNPGPMLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGKKVAKKPTNKAPHPKQEPQEINFPDDLPGSNTAAPVQETLHGCQPVTQEDGKESRISVQERQ

(19) United States
(12) Patent Application Publication
(10) Pub. No.: US 2026/0286301 A1
(43) Pub. Date: Sep. 24, 2026

(54) ENGINEERED T-CELL RECEPTORS AND METHODS OF MAKING AND USING THE SAME

(71) Applicant: The Johns Hopkins University, Baltimore, MD (US)

(72) Inventors: Kenneth W. Kinzler, Frankford, DE (US); Brian J. Mog, Baltimore, MD (US); Nickolas Papadopoulos, Towson, MD (US); Bert Vogelstein, Baltimore, MD (US); Shibin Zhou, Owings Mills, MD (US); Michael S. Hwang, Seattle, WA (US); Jacqueline Douglass, Baltimore, MD (US); Emily Han-Chung Hsiue, Baltimore, MD (US); Sarah DiNapoli, Baltimore, MD (US); Drew M. Pardoll, Brookeville, MD (US)

(21) Appl. No.: 19/473,591

(22) PCT Filed: Apr. 12, 2024
(86) PCT No.: PCT/US2024/024379

§ 371 Date: Oct. 8, 2025

Related U.S. Application Data
(60) Provisional application No. 63/459,056, filed on Apr. 13, 2023.
(60) Provisional application No. 63/512,021, filed on Jul. 5, 2023.

(51) Int. Cl. C12N 5/0783; A61K 40/11; A61K 40/32; A61P 35/00; C07K 14/705

(52) U.S. Cl. CPC C12N 5/0636; A61K 40/11; A61K 40/32; A61P 35/00; C07K 14/7051; C12N 2510/00

(57) ABSTRACT

Provided herein are immune cells comprising an engineered T cell receptor (TCR) comprising (a) a chimeric TCR alpha chain comprising a TCR alpha constant (TCR Ca) domain, wherein the chimeric TCR alpha chain lacks a TCR alpha variable (TCR Va) domain; (b) a chimeric TCR beta chain comprising a TCR beta constant (TCR Cβ) domain, wherein the chimeric TCR beta chain lacks a TCR beta variable (TCR Vβ) domain; (c) a scFv VH domain; (d) a scFv VL domain; and (e) a co-stimulatory domain.

### CROSS-REFERENCE TO RELATED APPLICATIONS

This application claims priority to U.S. Provisional Patent Application No. 63/459,056, filed on Apr. 13, 2023, and U.S. Provisional Patent Application No. 63/512,021, filed on Jul. 5, 2023. The disclosures of the prior applications are considered part of the disclosure of this application and are incorporated herein by reference in their entireties.

### FEDERALLY SPONSORED RESEARCH OR DEVELOPMENT

This invention was made with government support under grants CA006973, CA152753, CA228991, and CA062924 awarded by the National Institutes of Health. The government has certain rights in the invention.

### SEQUENCE LISTING

This application contains a Sequence Listing that has been submitted electronically as an XML file named 44807-0453WO1_ST26_SL.XML.” The XML file, created on Apr. 9, 2024, is 6,769,565 bytes in size. The material in the XML file is hereby incorporated by reference in its entirety.

### TECHNICAL FIELD

The present disclosure relates to a cellular therapy that uses engineered T-cell receptors to produce robust T-cell expansion and induce long-term regression of tumors bearing low densities of antigen. In particular, it relates to compositions of TCR embedded scFv for long-term activation (“TESLA”), nucleic acids encoding TESLAs, and recombinant cells expressing at least one TESLA. The disclosure also includes methods of generating and using such modified T cells (or other immune cells) expressing at least one TESLA to treat a disease (e.g., a cancer).

### BACKGROUND

T cell-based therapeutics are one of the promising approaches to treat advanced cancers and are now the subject of intense research. Naturally-occurring, tumor-targeted T cells from patients with cancer, and more recently T cells engineered to express the T cell receptors (TCRs) from these T cells, have been shown to induce remissions in a subset of patients with solid tumors that have failed multiple previous therapies. T cells modified with chimeric antigen receptors (CARs) have produced substantial improvements in clinical outcomes for patients with leukemias, lymphomas, and multiple myeloma and are being developed for other tumor types. However, many challenges remain. For example, the lack of persistence of engineered T cells following their administration is often the cause of eventual treatment failure and is an active area of investigation. Another challenge is that the treatment of common solid tumors with engineered T cells has yet to come to fruition in the clinic. The identification of suitable tumor-specific antigens on solid tumors for T cells to target will be critical for future success in this area.

### SUMMARY

Provided herein are immune cells comprising an engineered T cell receptor (TCR) comprising: (a) a chimeric TCR alpha chain comprising a TCR alpha constant (TCR Cα) domain, wherein the chimeric TCR alpha chain lacks a TCR alpha variable (TCR Vα) domain; (b) a chimeric TCR beta chain comprising a TCR beta constant (TCR Cβ) domain, wherein the chimeric TCR beta chain lacks a TCR beta variable (TCR Vβ) domain; (c) a scFv VH domain; (d) a scFv VL domain; and (e) a co-stimulatory domain.

In some embodiments, (i) the scFv VH domain is linked to the TCR alpha constant (TCR Cα) domain, and (ii) the scFv VL domain is linked to the TCR beta constant (TCR Cβ) domain. In some embodiments, the scFv VH domain is linked to the TCR alpha constant (TCR Cα) domain via a linker, and the scFv VL domain is linked to the TCR beta constant (TCR Cβ) domain via the linker.

In some embodiments, (i) the scFv VL domain is linked to the TCR alpha constant (TCR Cα) domain, and (ii) the scFv VH domain is linked to the TCR beta constant (TCR Cβ) domain. In some embodiments, the scFv VL domain is linked to the TCR Cα domain via a linker, and the scFv VH domain is linked to the TCR CB domain via the linker.

In some embodiments, the scFv VH domain and the scFv VL domain are linked to the TCR alpha constant (TCR Cα) domain. In some embodiments, the scFv VH domain and the scFv VL domain are linked to the TCR beta constant (TCR Cβ) domain. In some embodiments, the immune cell further comprises a TCR Cγ domain and a TCR Cδ domain.

Also provided herein are immune cells comprising an engineered T cell receptor (TCR) comprising: (a) a chimeric TCR gamma chain comprising a TCR gamma constant (TCR Cγ) domain, wherein the chimeric TCR gamma chain lacks a TCR gamma variable (TCR Vγ) domain; (b) a chimeric TCR delta chain comprising a TCR delta constant (TCR C8) domain, wherein the chimeric TCR delta chain lacks a TCR delta variable (TCR Vδ) domain; (c) a scFv VH domain; (d) a scFv VL domain; and (e) a co-stimulatory domain.

In some embodiments, (i) the scFv VH domain is linked to the TCR Cγ domain, and (ii) the scFv VL domain is linked to the TCR Cδ domain. In some embodiments, the scFv VH domain is linked to the TCR Cγ domain via a linker, and the scFv VL domain is linked to the TCR Cδ domain via the linker.

In some embodiments, (i) the scFv VL domain is linked to the TCR Cγ domain, and (ii) the scFv VH domain is linked to the TCR Cδ domain. In some embodiments, the scFv VL domain is linked to the TCR Cγ domain via a linker, and the scFv VH domain is linked to the TCR Cδ domain via the linker.

In some embodiments, the scFv VH domain and the scFv VL domain are linked to the TCR Cγ domain. In some embodiments, the scFv VH domain and the scFv VL domain are linked to the TCR Cδ domain. In some embodiments, the immune cell further comprises a TCR Cα domain and a TCR CB domain.

In some embodiments, the engineered TCR specifically binds to a neoantigen. In some embodiments, the neoantigen comprises EGFRvIII. In some embodiments, the neoantigen is presented on a HLA molecule. In some embodiments, the neoantigen comprises a peptide derived from a mutant oncogene. In some embodiments, the neoantigen comprises a p53RH antigen.

In some embodiments, the linker comprises SEQ ID NO: 1 or SEQ ID NO: 2.

In some embodiments, the co-stimulatory domain comprises a single co-stimulatory domain. In some embodiments, the co-stimulatory domain comprises a co-stimulatory domain of CD28, 4-1BB, OX40, ICOS, MYD88, 2B4, BTLA, CD2, CD22, CD27, CD30, CD4, CD40, CD79a, CD79b, CD84, CD8a, CD8b, CRACC, CRTAM, CTLA-4, DAP10, DNAM-1, DAP12, DR3, FCER1G, FCGR1A, FCGR2A, FCGR2B, FCGR3A, FCRL1, FCRL2, FCRL3, FCRL4, 5 FCRL5, FCRL6, GITR, HVEM, ITAM, KIR2DL1, KIR2DL2, KIR2DL3, KIR2DL4, KIR2DL5A, KIR2DL5B, KIR3DL1, KIR3DL2, KIR3DL3, LAG3, LAT, LILRB1, LILRB2, Ly9, NKG2A, NKG2C, NKG2D, NKp30, NKp44, NKp46, PD-1, PILRB, SIRPa, SLAMF1, SLAMF6, SLAMF7, TIGIT, TIM1, TIM3, TNFL6, TLR1, TLR10, TLR2, TLR3, TLR4, TLR5, TLR6, TLR7, TLR8, or TLR9. In some embodiments, the co-stimulatory domain comprises a co-stimulatory domain of MyD88 or a co-stimulatory domain of CD40.

In some embodiments, the co-stimulatory domain comprises a first co-stimulatory domain and a second co-stimulatory domain. In some embodiments, the first co-stimulatory domain and the second co-stimulatory domain are independently selected from CD28, 4-1BB, OX40, ICOS, MYD88, 2B4, BTLA, CD2, CD22, CD27, CD30, CD4, CD40, CD79a, CD79b, CD84, CD8a, CD8b, CRACC, CRTAM, CTLA-4, DAP10, DNAM-1, DAP12, DR3, FCER1G, FCGR1A, FCGR2A, FCGR2B, FCGR3A, FCRL1, FCRL2, FCRL3, FCRL4, 5 FCRL5, FCRL6, GITR, HVEM, ITAM, KIR2DL1, KIR2DL2, KIR2DL3, KIR2DL4, KIR2DL5A, KIR2DL5B, KIR3DL1, KIR3DL2, KIR3DL3, LAG3, LAT, LILRB1, LILRB2, Ly9, NKG2A, NKG2C, NKG2D, NKp30, NKp44, NKp46, PD-1, PILRB, SIRPa, SLAMF1, SLAMF6, SLAMF7, TIGIT, TIM1, TIM3, TNFL6, TLR1, TLR10, TLR2, TLR3, TLR4, TLR5, TLR6, TLR7, TLR8, or TLR9. In some embodiments, the first co-stimulatory domain comprises a co-stimulatory domain of MyD88 and the second co-stimulatory domain comprises a co-stimulatory domain of CD40.

In some embodiments, the co-stimulatory domain is linked to the TCR Cβ domain. In some embodiments, the co-stimulatory domain is linked to the TCR Cγ domain. In some embodiments, the co-stimulatory domain is linked to an extracellular and transmembrane domain of Fas.

In some embodiments, the engineered TCR is expressed from an expression cassette placed in an endogenous T cell receptor alpha constant (TRAC) locus of the immune cell. In some embodiments, the immune cell is a human immune cell. In some embodiments, the immune cell is a T cell.

Also provided herein are nucleic acid sequences encoding an engineered TCR, wherein the engineered TCR comprises: (a) a chimeric TCR alpha chain comprising a TCR alpha constant (TCR Cα domain, wherein the chimeric TCR alpha chain lacks a TCR alpha variable (TCR Vα) domain; (b) a chimeric TCR beta chain comprising a TCR beta constant (TCR Cβ) domain, wherein the chimeric TCR beta chain lacks a TCR beta variable (TCR Vβ) domain; (c) a scFv VH domain; (d) a scFv VL domain; and (e) a co-stimulatory domain.

In some embodiments, (i) the scFv VH domain is linked to the TCR alpha constant (TCR Cα) domain, and (ii) the scFv VL domain is linked to the TCR beta constant (TCR Cβ) domain. In some embodiments, the scFv VH domain is linked to the TCR alpha constant (TCR Cα) domain via a linker, and the scFv VL domain is linked to the TCR beta constant (TCR Cβ) domain via the linker.

In some embodiments, (i) the scFv VL domain is linked to the TCR alpha constant (TCR Cα) domain, and (ii) the scFv VH domain is linked to the TCR beta constant (TCR Cβ) domain. In some embodiments, the scFv VL domain is linked to the TCR Cα domain via a linker, and the scFv VH domain is linked to the TCR Cβ domain via the linker.

In some embodiments, the scFv VH domain and the scFv VL domain are linked to the TCR alpha constant (TCR Cα) domain. In some embodiments, the scFv VH domain and the scFv VL domain are linked to the TCR beta constant (TCR Cβ) domain. In some embodiments, the nucleic acid sequence further comprises a TCR Cγ domain and a TCR Cδ domain.

Also provided herein are nucleic acid sequences encoding an engineered TCR, wherein the engineered TCR comprises: (a) a chimeric TCR gamma chain comprising a TCR gamma constant (TCR Cγ) domain, wherein the chimeric TCR gamma chain lacks a TCR gamma variable (TCR Vγ) domain; (b) a chimeric TCR delta chain comprising a TCR delta constant (TCR Cδ) domain, wherein the chimeric TCR delta chain lacks a TCR delta variable (TCR Vδ) domain; (c) a scFv VH domain; (d) a scFv VL domain; and (e) a co-stimulatory domain.

In some embodiments, (i) the scFv VH domain is linked to the TCR Cγ domain, and (ii) the scFv VL domain is linked to the TCR Cδ domain. In some embodiments, the scFv VH domain is linked to the TCR Cγ domain via a linker, and the scFv VL domain is linked to the TCR Cδ domain via the linker.

In some embodiments, (i) the scFv VL domain is linked to the TCR Cγ domain, and (ii) the scFv VH domain is linked to the TCR Cδ domain. In some embodiments, the scFv VL domain is linked to the TCR Cγ domain via a linker, and the scFv VH domain is linked to the TCR Cδ domain via the linker.

In some embodiments, the scFv VH domain and the scFv VL domain are linked to the TCR Cγ domain. In some embodiments, the scFv VH domain and the scFv VL domain are linked to the TCR Cδ domain. In some embodiments, the nucleic acid sequence further comprises a TCR Cα domain and a TCR Cβ domain.

In some embodiments, the linker comprises SEQ ID NO: 1 or SEQ ID NO: 2.

In some embodiments, the co-stimulatory domain comprises a single co-stimulatory domain. In some embodiments, the co-stimulatory domain comprises a co-stimulatory domain of CD28, 4-1BB, OX40, ICOS, MYD88, 2B4, BTLA, CD2, CD22, CD27, CD30, CD4, CD40, CD79a, CD79b, CD84, CD8a, CD8b, CRACC, CRTAM, CTLA-4, DAP10, DNAM-1, DAP12, DR3, FCER1G, FCGR1A, FCGR2A, FCGR2B, FCGR3A, FCRL1, FCRL2, FCRL3, FCRL4, 5 FCRL5, FCRL6, GITR, HVEM, ITAM, KIR2DL1, KIR2DL2, KIR2DL3, KIR2DL4, KIR2DL5A, KIR2DL5B, KIR3DL1, KIR3DL2, KIR3DL3, LAG3, LAT, LILRB1, LILRB2, Ly9, NKG2A, NKG2C, NKG2D, NKp30, NKp44, NKp46, PD-1, PILRB, SIRPa, SLAMF1, SLAMF6, SLAMF7, TIGIT, TIM1, TIM3, TNFL6, TLR1, TLR10, TLR2, TLR3, TLR4, TLR5, TLR6, TLR7, TLR8, or TLR9. In some embodiments, the co-stimulatory domain comprises a co-stimulatory domain of MyD88 or a co-stimulatory domain of CD40.

In some embodiments, the co-stimulatory domain comprises a first co-stimulatory domain and a second co-stimulatory domain. In some embodiments, the first co-stimulatory domain and the second co-stimulatory domain are independently selected from CD28, 4-1BB, OX40, ICOS, MYD88, 2B4, BTLA, CD2, CD22, CD27, CD30, CD4, CD40, CD79a, CD79b, CD84, CD8a, CD8b, CRACC, CRTAM, CTLA-4, DAP10, DNAM-1, DAP12, DR3, FCER1G, FCGR1A, FCGR2A, FCGR2B, FCGR3A, FCRL1, FCRL2, FCRL3, FCRL4, 5 FCRL5, FCRL6, GITR, HVEM, ITAM, KIR2DL1, KIR2DL2, KIR2DL3, KIR2DL4, KIR2DL5A, KIR2DL5B, KIR3DL1, KIR3DL2, KIR3DL3, LAG3, LAT, LILRB1, LILRB2, Ly9, NKG2A, NKG2C, NKG2D, NKp30, NKp44, NKp46, PD-1, PILRB, SIRPa, SLAMF1, SLAMF6, SLAMF7, TIGIT, TIM1, TIM3, TNFL6, TLR1, TLR10, TLR2, TLR3, TLR4, TLR5, TLR6, TLR7, TLR8, or TLR9. In some embodiments, the first co-stimulatory domain comprises a co-stimulatory domain of MyD88 and the second co-stimulatory domain comprises a co-stimulatory domain of CD40.

In some embodiments, the co-stimulatory domain is linked to the TCR Cβ domain. In some embodiments, the co-stimulatory domain is linked to the TCR Cγ domain. In some embodiments, the co-stimulatory domain is linked to an extracellular and transmembrane domain of Fas.

Also provided herein are vectors comprising any one of the nucleic acid sequences described herein. In some embodiments, the vector further comprises a promoter. In some embodiments, the promoter is a TRAC promoter or EF1-alpha promoter. In some embodiments, the vector is a viral vector.

Also provided herein are methods of producing an engineered immune cell, the method comprising: introducing into an immune cell any one of the nucleic acids or any one of the vectors described herein, thereby producing the engineered immune cell. In some embodiments, the nucleic acid is introduced into the immune cell by using a gene-editing agent. In some embodiments, the gene-editing agent comprises CRISPR components.

Also provided herein are engineered immune cells produced by any one of the methods described herein.

Also provided herein are pharmaceutical compositions comprising any one of the engineered immune cells described herein and a pharmaceutically acceptable carrier.

Also provided herein are methods of treating a disease in a subject, the method comprising administering to the subject any one of the engineered immune cells or any one of the pharmaceutical compositions described herein. In some embodiments, the disease is a cancer comprising a p53 mutation. In some embodiments, the p53 mutation comprises a p53R175H mutation. In some embodiments, the cancer is an ovary cancer, colorectum cancer, esophagus cancer, head and neck cancer, larynx cancer, lung cancer, skin cancer, pancreas cancer, stomach cancer, liver cancer, brain cancer, bladder cancer, breast cancer, uterus cancer, soft tissue cancer, lymph node cancer, prostate cancer, bone cancer, endocrine gland cancer, or cervix cancer.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A-1F show potencies of conventional CARs and TCRs vary with antigen density. (FIG. 1A) Diagrams of TCR-1, CAR-1, and CAR-2. CAR-1 employs a CD28 hinge, while CAR-2 employs a CD8α hinge. Both CARs use a CD28 transmembrane domain and an intracellular signaling domain followed by a CD3ζ intracellular domain. Vα, Vβ, Cα and Cβ denote TCR variable α, variable β, constant α and constant β chains, respectively; ε, δ, γ and ζ denote the ε, δ, γ and ζ CD3 subunits, respectively; VH and VL, variable heavy and light chains of scFv, respectively. (FIG. 1B) T2 cells pulsed with decreasing concentrations of the p53RH peptide (HMTEVVRHC) were incubated with modified T cells at an ET ratio of 1:5 for 24 hrs. Conditioned supernatant was assessed for IFN-γ by ELISA. (FIGS. 1C-1D) Modified T cells were cultured with KMS26 and NALM6 isogenic cell sets at an ET ratio of 1:5 for 20 hrs. Conditioned supernatant was assayed for IFN-γ by ELISA. (FIGS. 1E-1F) Modified T cells were cultured with KMS26 and NALM6 isogenic cell sets at an ET ratio of 1:5 for 20 hrs. Cytotoxicity was quantified by bioluminescence. Data are shown as means±SD of three technical replicates, except for the T cells only conditions, which represent two technical replicates. Data are representative of two independent experiments. **** P<0.0001, *** P<0.001 by two-way ANOVA with Tukey's multiple comparison test.

FIGS. 2A-2E show hybrid TCR/CARs can signal at endogenous p53RH antigen levels. (FIG. 2A) Diagrams of TCR-1 and TCR/CAR-1 through-9 demonstrate the attachment of the H2-scFv to the N-terminus of CD3γ (TCR/CAR-1), the TCRα or β constant domains (Cα, Cβ) (TCR/CAR-2 to 5), or the TCRα or β variable domains (Vα, Vβ) of the full length TCR (TCR/CAR-6 to 9). The H2-scFv was attached in the VLVH orientation (TCR/CAR-1, 4, 5, 8, 9) or VHVL orientation (TCR/CAR-2, 3, 6, 7). (FIGS. 2B-2E) Modified T cells (1×10⁴) were incubated with either KMS26 or NALM6 isogenic cell sets (5×10⁴) for 20 hrs. Conditioned supernatant was analyzed for IFN-γ by ELISA (FIGS. 2B-2C). A bioluminescence assay was used to quantify cytotoxicity (FIGS. 2D-2E). Data are shown as means±SD of three technical replicates for all conditions except T Cells Only, which represent two technical replicates. Comparisons between TCR-1, TCR/CAR-1, TCR/CAR-4, and TCR/CAR-5 are representative of at least two independent experiments. TCR constructs in the left panels of B-E utilize human TCRα and β constant domains, while all constructs in the right panels of FIGS. 2B-2E utilize modified murine constant domains. For TCR-1, human and modified murine constant domains are equivalent (FIG. 18). TCR/CAR-1 was introduced into the CD3G locus, while all other constructs were introduced into the TRAC locus. **** P<0.0001, *P<0.05. ns, not significant, by two-way ANOVA with Tukey's multiple comparison test.

FIGS. 3A-3I show Split/CARs have comparable potency to conventional TCRs. (FIG. 3A) Diagrams of TCR-1 and Split/CAR-1 through-6 depict the attachment of the H2-scFv VL and VH domains to the TCRα or β constant domains (Cα, Cβ) without a linker (Split/CAR-1, -2) or with a 5 amino acid “EAAAK” linker (Split/CAR-3, -4). Split/CAR-5 and -6 show attachment of VL and VH domains to the N-termini of the TCRα and β variable domains (Vα, Vβ) of the full length TCR through a 5 amino acid “GGGGS” (G4S) linker. All depicted constructs utilize modified murine constant domains. (FIGS. 3B-31) Modified T cells (1×10⁴) were cultured with KMS26 or NALM6 isogenic cell sets (5×10⁴) for 20-21 hrs. Conditioned supernatant was analyzed for IFN-γ by ELISA (FIGS. 3B, 3C, 3F, 3G). A bioluminescence assay was used to quantify cytotoxicity (FIGS. 3D, 3E, 3H, 3I). Data are shown as means±SD of three technical replicates for all conditions except T Cells Only, which represent two technical replicates. Comparisons of TCR-1, Split/CAR-1 and Split/CAR-3 are representative of four independent experiments. **** P<0.0001, ** P<0.01, *P<0.05. ns, not significant, by two-way ANOVA with Tukey's multiple comparison test.

FIGS. 4A-4H show Split/CAR and TCR demonstrate in vivo activity. (FIG. 4A) Schematic of KMS26-MUT in vivo model timeline. NSG mice were injected via tail vein with 3.5×10⁵ KMS26-MUT cells on day −6. Mice were randomized based on bioluminescence imaging (BLI) signal on day −1. Either 2×10⁶ knock-in (KI+) T cells normalized to 18% KI frequency with TCR-Control T cells or 11.1×10⁶ TCR-Control T cells were injected via tail vein on day 0. (FIG. 4B) BLI measurements of treated mice. Data represent mean±SD of measurements from five mice. (FIG. 4C) Kaplan-Meier survival curves for mice in the KMS26-MUT in vivo model. (FIG. 4D) Schematic of NALM6-MUT in vivo model timeline. NSG mice were injected via tail vein with 5×10⁵ NALM6-MUT cells on day −3. Mice were randomized based on BLI signal on day −1. Either 3×10⁶ KI+ T cells normalized to 16% KI+ frequency with TCR-Control T cells or 18.5×10⁶ TCR-Control T cells were injected via tail vein on day 0. (FIG. 4E) BLI measurements of treated mice. Data represent mean±SD of five mice. No T cells and TCR-Control curves are truncated because all mice died before the day 30 timepoint. (FIG. 4F) Kaplan-Meier survival curves for mice in the NALM6-MUT in vivo model. (FIGS. 4G-4H) Numbers of total T cells (FIG. 4G) and KI+ T cells (FIG. 4H) in the peripheral blood of mice were quantified by flow cytometry on days 8 and 17 after T cell injection. Data represent mean±SD of measurements from five mice. **** P<0.0001, ** P<0.01, *P<0.05. ns, not significant, by paired t-tests with Holm-Šídak multiple comparison correction for FIG. 4G-4H or by log-rank Mantel-Cox test with Bonferroni correction for FIGS. 4C and 4F.

FIGS. 5A-5E show co-stimulation improves long-term in vitro function of TESLA and TCR. (FIG. 5A) Schematic depicting co-stimulation modified Split/CAR-3 and TCR-1. MyD88 and CD40 (MC) domains linked to the transmembrane domain of the TCRβ chain generate TESLA-1 and TCR/CoS-1, while MC domains linked to the transmembrane domain of Fas generate TESLA-2 and TCR/CoS-2. (FIGS. 5B-5C) Modified T cells (1×10⁴) were cultured with NALM6-MUT cells (5×10⁴) in the absence of exogenous cytokines. Every 48 hrs, 5×10⁴ NALM6-MUT cells were added to the co-culture. Live cell imaging was used to quantify cancer cells. Data are representative of means±SEM of three technical replicates (FIG. 5B) or four technical replicates (FIG. 5C). (FIGS. 5D-5E) Flow cytometric quantification of knock-in (KI+) T cells (FIG. 5D) and NALM6-MUT cells (FIG. 5E) on day 25 of the multiple stimulation assay shown in (FIG. 5C). Data are shown as means±SD of four technical replicates. All data are representative of two independent experiments. **** P<0.0001. ns, not significant, by one-way ANOVA with Tukey's multiple comparison test.

FIGS. 6A-6H show TESLAs demonstrate prolonged activity in vivo. (FIG. 6A) Schematic showing the design of the in vivo experiment. NSG mice were inoculated via tail vein with 3.5×10⁵ KMS26-MUT cells on day −6. Mice were randomized based on BLI signal on day −1. Tail vein injection of modified T cells (either 1×10⁶ knock-in+ (KI+) T cells normalized to 10% KI frequency with TCR-Control T cells or 1×10⁷ TCR-Control T Cells) was performed on day 0. (FIGS. 6B-6D) Approximately weekly BLI imaging was used to track cancer cell growth. Data points are values from individual mice. Curves are truncated when the mouse died before the specified timepoint. (FIG. 6E) Kaplan-Meier survival curves of seven treatment groups, n=5 mice per group. (FIGS. 6F-6H) Quantification of KI+ T cells in peripheral blood of mice using flow cytometry. Each data point represents one mouse. Curves are truncated when either a mouse died or when no KI+ T cells could be detected in peripheral blood by flow cytometry.

FIGS. 7A-7B show CRISPR knock-in and knock-out strategy in primary human T cells. (FIG. 7A) Diagram showing the simultaneous TRAC knock-in (KI) and TRBC1 and TRBC2 knock-out (KO) approach. TCR-1 and CAR-1 homology directed repair templates (HDRTs) are shown specifically. Double stranded DNA (dsDNA) HDRTs include an EF1α promoter (EF1α), the receptor domain(s), a tNGFR tag, and a poly A terminator sequence following the stop codon of the tNGFR tag. Independent protein domains are separated by furin-2A sequences (2A). Homology arms (HAs) are approximately 300 base pairs (bps) in length. (FIG. 7B) Flow cytometric characterization of modified T cells with the p53RH tetramer and CD3 staining reagents four days after nucleofection. The top row displays all live T cells, while the middle row displays the CD4+ T cell subset and the bottom row displays the CD8+ T cell subset (defined as CD4 staining negative).

FIGS. 8A-8C show generation of the NALM6-MUT cell line. (FIG. 8A) Sanger sequencing of the TP53 locus near codon 175 of both the NALM6-WT line and the NALM6-MUT line. The homology directed repair template included five synonymous mutations between the substitution encoding the R175H mutation and the CRISPR Cas9 cut site to minimize template switching during double strand break repair. (FIG. 8B) Flow cytometric staining for HLA-A*02:01 with BB7.2, an antibody for HLA-A*02:01, or an isotype control antibody labeled with brilliant violet 785 on the NALM6-WT and NALM6-MUT lines. (FIG. 8C) Mass spectrometry quantification of the p53RH peptide (HMTEVVRHC) eluted from the HLA molecules of the NALM6-WT and NALM6-MUT lines.

FIGS. 9A-9B show flow cytometric characterization of Tier 2 T cells. (FIG. 9A) Flow cytometric staining of tNGFR+ modified T cells with p53RH tetramer labeled with phycoerythrin (p53RH-tetramer-PE) and SK7, an antibody for CD3, labeled with brilliant violet 421 (CD3-BV421) ten days after nucleofection. (FIG. 9B) Comparison of p53RH tetramer and anti-CD3 staining on tNGFR+ CD4+ and tNGFR+ CD8+ T cells 11 days after nucleofection. TCR-Control and Unmodified T cells show all live, single cells (FIGS. 9A-9B). FIGS. 10A-10B show flow cytometric characterization of Tier 3 T cells. (FIG. 10A) Flow cytometric staining of tNGFR+ modified T cells with p53RH tetramer and anti-CD3 ten days after nucleofection. (FIG. 10B) Comparison of p53RH tetramer and anti-CD3 staining on tNGFR+ CD4+ and tNGFR+ CD8+ T cells 11 days after nucleofection. TCR-Control and Unmodified T cells show all live, single cells (FIGS. 10A-10B).

FIGS. 11A-11D show comparing Split/CAR-3 to four p53RH reactive TCRs. (FIGS. 11A-11B) Modified T cells were cultured with KMS26 and NALM6 isogenic cell sets at an E:T ratio of 1:5 for 20 hrs. Conditioned supernatant was assayed for IFN-γ by ELISA. (FIGS. 11C-11D) The cytotoxicity of modified T cells in the same co-cultures was quantified by bioluminescence. Data are shown as means±SD of three technical replicates, except for the T Cells Only conditions, which are two technical replicates. Data are representative of two independent experiments.

FIGS. 12A-12C show screening approach for candidate co-stimulatory domains. (FIG. 12A) Schematic showing the TRAC knock-in and TRBC knock-out strategy for testing multiple co-stimulatory constructs alongside TCR-1. When the co-stimulatory construct encodes an independent protein domain, the tNGFR domain is replaced by the co-stimulatory sequence. For this screen, the TCR-1 construct encodes the human TCRα and TCRβ constant domains. (FIG. 12B) Diagrams depicting each of the nine co-stimulatory designs tested. For TCR/CoS-1, the MC domains are connected to the intracellular side of the TCRβ transmembrane domain. TCR/Cos-2 through-5 link the Fas extracellular (Fas EC) and transmembrane domains to MC, OX40, 4-1BB, and IL7Rα signaling domains. TCR/CoS-6 and -7 co-express STAT3 (CASTAT3) and STAT5 (CASTAT5) molecules bearing mutations which impart constitutive activity. TCR/CoS-8 and -9 co-express the IL7Rα intracellular domain linked to the IL7Rα transmembrane domain with a CPT insertion which drives dimerization and constitutive activity. TCR/CoS-8 utilizes a CD34 extracellular domain (CD34 EC) and TCR/CoS-9 utilizes the IL7Rα extracellular domain (IL7Rα EC). (FIG. 12C) Flow cytometric quantification of p53RH and CD3 staining levels in CD8+ tetramer+ and CD4+ tetramer+ T cells ten days after nucleofection. TCR-Control and Unmodified T cells show all live, single cells.

FIGS. 13A-13I show functional assessment of co-stimulatory candidates. (FIG. 13A) NALM6-MUT cells (5×10⁴) were co-incubated with modified T cells (1×10⁴) with exogenous IL-2. Every 48 hrs, 5×10⁴ NALM6-MUT cells were added to the co-culture with additional IL-2. Conditioned supernatant collected every two days was assayed for IFN-γ by ELISA. Data shown are means±SD of three technical replicates. (FIG. 13B) Live cell imaging was used to quantify cancer cell growth during the assay described in (FIG. 13A). Data are representative of means±SEM of four technical replicates. (FIGS. 13C-13E) Flow cytometric analysis at the end of the multiple stimulation assay (MSA) quantifying NALM6-MUT cells (FIG. 13C), knock-in positive (KI+) T cell numbers (FIG. 13D), and co-inhibitory receptor expression on KI+ T cells (FIG. 13E). Data show means±SD of four technical replicates. (FIGS. 13F-13G) Modified T cells (1×10⁴) were incubated with the KMS26 isogenic cell set at an E:T ratio of 1:5 for 20 hrs. IFN-γ in conditioned supernatant was measured by ELISA (FIG. 13F). Cytotoxicity was measured by a bioluminescence assay (FIG. 13G). Data show means±SD of three technical replicates, except for T Cells Only, which have only two technical replicates. (FIGS. 13H-131) Modified T cells (1×10⁴) were incubated with the KMS26 and NALM6 isogenic cell sets (5×10⁴) for 24 hrs. Conditioned supernatant was analyzed for IFN-γ by ELISA. Data are shown as means±SD of three technical replicates and represent three independent experiments. Constructs in FIGS. 13A-13G were designed with human TCRα and TCRβ constant domains, while constructs in FIGS. 13H and 13I used modified murine constant domains as described herein.

FIG. 14 shows markers of TESLA activation in vitro. Modified T cells (1×10⁴) were incubated with the NALM6 isogenic cell set at an E:T ratio of 1:5 for 22 hrs in the absence of exogenous IL-2. Conditioned supernatant was analyzed by Luminex for 15 analytes. Cytotoxicity was analyzed by bioluminescence assay (lower right panel). Data show means±SD of three technical replicates.

FIGS. 15A-15H show NALM6-MUT in vivo model. (FIG. 15A) Schematic showing the NALM6-MUT in vivo model. NSG mice were inoculated via tail vein with 5×10⁵ NALM6-MUT cells on day −3 followed by randomization based on BLI signal on day −1. Tail vein injection of modified T cells (either 0.4×10⁶ KI+ T cells normalized to 13.6% KI frequency with TCR-Control T cells or 2.9×10⁶ TCR-Control T Cells) was performed on day 0. (FIGS. 15B-15D) Approximately weekly BLI imaging was used to track cancer cell growth. Data points are values from individual mice. Curves are truncated when mice died. (FIG. 15E) Kaplan-Meier survival curves of seven treatment groups, n=5 mice per group. (FIGS. 15F-15H) Flow cytometric quantification of KI+ T cells detected in peripheral blood of mice. Data points show individual mice. Curves are truncated either when mice died or when no KI+ T cells were detected by flow cytometry.

FIGS. 16A-16H show NALM6-WT in vivo model. (FIG. 16A) Schematic showing the NALM6-WT in vivo model. NSG mice were inoculated via tail vein with 5×10⁵ NALM6-WT cells on day −3 followed by randomization on day −1 based on BLI signal. Tail vein injection of modified T cells (either 0.4×10⁶ KI+ T cells normalized to 8.3% KI frequency with TCR-Control T cells or 4.8×10⁶ TCR-Control T Cells) was performed on day 0. (FIG. 16B) Approximately weekly BLI imaging was used to track cancer cell growth. Data are shown as means±SD of values from five mice. (FIG. 16C) Kaplan-Meier survival curves of seven treatment groups, n=5 mice per group. (FIGS. 16D-16F) Flow cytometric quantification of KI+ T cells detected in peripheral blood of mice. Data are values from individual mice. Curves are truncated either when mice died or when no KI+ T cells were detected by flow cytometry. (FIGS. 16G-16H) Weight measurements of all mice in the NALM6-WT experiment (FIG. 16G) or NALM6-MUT experiment (FIG. 16H), plotted as means±SD of values from five mice.

FIG. 17 shows HLA-A*02:01 expression on cancer cells in NALM6-MUT in vivo model. Flow cytometric staining of HLA-A*02:01 on NALM6-MUT cells detected in peripheral blood of mice on days 50 or 67 after T cell injection. Each row shows live NALM6-MUT cells from a different mouse or from in vitro culture.

FIGS. 18A-18B show comparison of TCR-1 with human vs. modified murine TCRα and TCRβ constant domains. (FIG. 18A) Modified T cells were cultured with the KMS26 isogenic cell sets at an E:T ratio of 1:5 for 21 hrs. “Human” vs. “murine” designates the species of origin of the TCRα and TCRβ constant domains. “Disulfide” indicates the presence of an additional engineered disulfide bond between the TCRα and TCRβ constant domains. “LVL” indicates the presence of hydrophobic amino acid substitutions in the transmembrane domain of the TCRα chain. Conditioned supernatant was assayed for IFN-γ by ELISA. (FIG. 18B) The cytotoxicity of modified T cells in the same co-culture was quantified by bioluminescence. Data are shown as means±SD of three technical replicates, except for the T Cells Only conditions, which are two technical replicates. Data are representative of one experiment. **** P<0.0001, *P<0.05. ns, not significant, by two-way ANOVA with Tukey's multiple comparison test.

FIGS. 19A-19C show comparison of endogenous TRAC promoter with exogenous EF1α promoter for expression of Split/CAR-3. CRISPR strategies were designed to insert a Split/CAR-3 construct at the TRAC locus either under control of the endogenous TRAC promoter or with an exogenous EF1α promoter. The endogenous TRAC promoter strategy utilized a Cas9 ribonucleoprotein (RNP) while the exogenous EF1α promoter strategy utilized a Cpf1 RNP. Both Cas9 and Cpf1 RNPs targeted overlapping regions of exon 1 of the TRAC locus and included concomitant KO of the TRBC loci. The Split/CAR-3 construct utilized human TCR constant domains as opposed to the murine constant domains. (FIGS. 19A-19B) Modified T cells were cultured with the KMS26 and NALM6 isogenic cell sets at an E:T ratio of 1:5 for 20 hrs. Conditioned supernatant was assayed for IFN-γ by ELISA. Data are shown as means±SD of three technical replicates, except for the T Cells Only conditions, which are two technical replicates. Data are representative of one experiment. **** P<0.0001 by two-way ANOVA with Tukey's multiple comparison test. (FIG. 19C) Modified T cells (4×10³) labeled with Cell Trace Violet were incubated with KMS26-MUT cells (1.6×10⁴) in 16 replicates. Every 48 hrs, the number of cancer cells in four replicates was quantified by flow cytometry. Additional KMS26-MUT cells (3.2×10⁴) were added to the remaining replicates. This process was repeated 3 more times for a total of 4 time points. Data are shown as means±SD of four technical replicates.

FIG. 20 shows co-inhibitory receptor expression after 12 day multiple stimulation assay. Modified T cells (1×10⁴) were incubated with NALM6-MUT cells (5×10⁴) in the presence of exogenous IL-2. Every 2 days for 12 days, 5×10⁴ NALM6-MUT cells in IL-2 media were added to the culture. At the end of 12 days, the expression of co-inhibitory receptors on modified T cells were quantified by flow cytometry. Data shown are means of four technical replicates.

FIGS. 21A-21F show MyD88-CD40 promotes expansion and persistence of CD4 T cells. (FIGS. 21A-21D) Modified T cells (1×10⁴) were incubated with KMS26 and NALM6 isogenic cell sets (5×10⁴) in the presence of exogenous IL-2 for 5 days, after which CD4+ and CD8+ knock-in+ (KI+) T cells numbers were quantified (FIGS. 21A-21B). The percent CD4+ KI+ T cells with and without stimulation were determined (FIGS. 21C-21D). (FIGS. 21E-21F) Modified T cells (1×10⁴) were incubated with NALM6-MUT cells (5×10⁴) in the presence of exogenous IL-2. Every 2 days for 12 days, 5×10⁴ NALM6-MUT cells in IL-2 media were added to the culture. At the end of 12 days, the number of total KI+ T cells (FIG. 21E) as well as CD4+ and CD8+ KI+ T cells (FIG. 21F) were quantified by flow cytometry. In all panels, when applicable data shown are means±SD of three to four technical replicates.

FIG. 22 show normalized counts of selected genes modulated in TESLA-1 T cells. TESLA-1, Split/CAR-3 and unmodified T cells were incubated with KMS26-MUT, KMS26-NULL, or no target cells at an ET ratio of 1:5 for 18 hrs in the absence of exogenous IL-2 followed by flow sorting and transcriptomic analysis of CD4+ and CD8+ T cells. Normalized transcript counts from the DEseq2 analysis are displayed. Data show means±SD of 6 technical replicates.

FIGS. 23A-23B show volcano plots for TESLA-1 T cells compared to Split/CAR-3 T cells. (FIG. 23A) Genes expressed differentially between TESLA-1 and Split/CAR-3 after co-culture with KMS26-MUT cells for 18 hours, with CD4+ T cells displayed on the left and CD8+ T cells on the right. (FIG. 23B) Genes expressed differentially between TESLA-1 and Split/CAR-3 after culture without target cells for 18 hours, with CD4+ T cells displayed on the left and CD8+ T cells on the right.

FIGS. 24A-24B show heatmaps of differentially expressed TESLA-1 T cells. (FIGS. 24A and 24B) The 25 most upregulated genes and downregulated genes by log 2 fold change between TESLA-1 and Split/CAR-3 after co-culture with KMS26-MUT cells for 18 hours, with genes from CD4+ T cells (FIG. 24A) and CD8+ T cells (FIG. 24B) displayed independently. Differentially expressed genes displayed on heatmaps were filtered for base-mean expression above 10 and scaled by row.

FIGS. 25A-25D show Pathway Analysis of TESLA-1 T cells. (FIGS. 25A and 25B) STRING analysis of genes upregulated in CD4+ (FIG. 25A) or CD8+ (FIG. 25B) TESLA-1 T cells compared to Split/CAR-3 T cells after co-culture with KMS26-MUT cells. (FIGS. 25C and 25D) STRING analysis of genes upregulated in CD4+ (FIG. 25C) or CD8+ (FIG. 25D) TESLA-1 T cells compared to Split/CAR-3 T cells after culture in the absence of target cells. Genes input into STRING analysis were identified through DEseq2 with adjusted p-values less than 0.05 and any positive log 2 fold change. No pathways were identified in down-regulated genes.

### DETAILED DESCRIPTION

As with any cancer therapeutic agent, success may depend on specificity. If the agent is directed against a target that is also expressed in normal tissues, life-threatening on-target, off-tumor toxicities can occur. In some embodiments, clinically-approved T cell-based therapeutic agents are not tumor-specific-they can be used because they target antigens on normal B cells and plasma cells that are dispensable for patient survival. Antigens derived from mutant oncogenes or tumor suppressor genes are present only in cancer cells and, thus, can be leveraged to unequivocally distinguish cancer cells from normal cells. Although most of these genes produce intracellular proteins, proteolytically processed peptides derived from these mutant genes can be presented on the tumor cell surface by binding to human leukocyte antigen (HLA) molecules.

TCRs as the recognition component of T cells have the advantage over CARs in that TCR-equipped T cells can bind and kill target cells containing a few antigen copies per cell. On the other hand, CARs have the advantage of leveraging antibody-based recognition domains, which have much higher affinities (nM vs. μM) and are much easier to discover and improve than TCRs using techniques such as phage display, yeast display, ribosome display, and a host of others originally designed to improve soluble antibody therapeutics rather than cell-based TCRs. Moreover, CARs directly integrate co-stimulatory signaling with antigen-recognition, allowing for more robust T cell activation.

In some embodiments, described herein are immune cells comprising an engineered T cell receptor (TCR), wherein the engineered TCR includes features that combine the advantages of antibody-based CARs with the advantages of naturally occurring TCRs to create anew type of receptor called a TESLA (TCR Embedded ScFv for Long-term Activation), which generates sustained T cell activity against a low-density antigen both in vitro and in vivo. In some embodiments, an immune cell described herein can be referred to as a highly persistent and sensitive CAR-T cell (“Hyper-CAR”) or a co-stimulatory synthetic TCR and antigen receptor T cell (“Co-STAR”).

In some embodiments, provided herein are immune cells comprising an engineered T cell receptor (TCR) that includes (a) a chimeric TCR alpha chain comprising a TCR alpha constant (TCR Cα) domain, wherein the chimeric TCR alpha chain lacks a TCR alpha variable (TCR Vα) domain; (b) a chimeric TCR beta chain comprising a TCR beta constant (TCR Cβ) domain, wherein the chimeric TCR beta chain lacks a TCR beta variable (TCR Vβ) domain; (c) a scFv VH domain; (d) a scFv VL domain; and (e) a co-stimulatory domain.

Also provided herein are nucleic acid sequences encoding an engineered TCR, wherein the engineered TCR includes (a) a chimeric TCR alpha chain comprising a TCR alpha constant (TCR Cα) domain, wherein the chimeric TCR alpha chain lacks a TCR alpha variable (TCR Vα) domain; (b) a chimeric TCR beta chain comprising a TCR beta constant (TCR Cβ) domain, wherein the chimeric TCR beta chain lacks a TCR beta variable (TCR Vβ) domain; (c) a scFv VH domain; (d) a scFv VL domain; and (e) a co-stimulatory domain.

This disclosure also provides vectors, compositions, methods of generating, and methods of treatment using the engineered immune cells described herein.

Various non-limiting aspects of these immune cells are described herein, and can be used in any combination without limitation. Additional aspects of various components of methods of making and using the immune cells are known in the art.

It must be noted that, as used in the specification and the appended claims, the singular forms “a,” “an” and “the” include plural referents unless the context clearly dictates otherwise.

As used herein, the term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be intravenous, intra-arterial, intra-tumor, intraperitoneal, intrathecal, or intraventricular. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

As used herein, the term “antigen” refers to a molecule or molecular structure that binds to a specific antibody, B-cell receptor, or T-cell receptor. In general, an antigen may be or include any chemical entity such as, for example, a small molecule, a nucleic acid sequence (e.g., DNA or RNA), a peptide, a polypeptide, a protein, a carbohydrate, a glycoprotein, a lipid or phospholipid, a lipoprotein, a polymer (including biologic polymers [e.g., nucleic acid and/or amino acid polymers] and polymers other than biologic polymers [e.g., other than a nucleic acid or amino acid polymer]), etc. In some embodiments, an antigen is or comprises a polypeptide. In some embodiments, an antigen is or comprises a glycan. In some embodiments, an antigen is or comprises a phospholipid. In some embodiments, an antigen is or comprises a phospholipid-protein complex. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some certain embodiments, an antigen is present in a cellular context (e.g., an antigen is expressed on the surface of a cell or expressed in a cell). In some embodiments, an antigen is present on the surface of a cell in a complex with HLA. In some embodiments, an antigen is a recombinant antigen.

As used herein, the term “binding” typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell).

As used herein, the terms “cancer”, “malignancy”, “neoplasm”, “tumor”, and “carcinoma”, refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

As used herein, in general, the term “engineered” refers to the aspect of having been manipulated by the hand of man. For example, a polypeptide is considered to be “engineered” when the polypeptide sequence manipulated by the hand of man. For example, in some embodiments of the present disclosure, an engineered polypeptide comprises a sequence that includes one or more amino acid mutations, truncations, deletions, and/or insertions that have been introduced by the hand of man into a reference polypeptide sequence. In some embodiments, an engineered polypeptide includes a polypeptide that has been fused (e.g., covalently linked) to one or more additional polypeptides by the hand of man, to form a fusion polypeptide that would not naturally occur in vivo. Comparably, a cell or organism is considered to be “engineered” if it has been manipulated so that its genetic information is altered (e.g., new genetic material not previously present has been introduced, for example by transformation, mating, somatic hybridization, transfection, transduction, electroporation, or other mechanism, or previously present genetic material is altered or removed, for example by substitution or deletion mutation, or by mating protocols). As is common practice and is understood by those in the art, derivatives and/or progeny of an engineered polypeptide or cell are typically still referred to as “engineered” even though the actual manipulation was performed on a prior entity.

As used herein, the term “pharmaceutical composition” refers to a composition in which an active agent is formulated together with one or more pharmaceutically acceptable carriers. In some embodiments, the composition is suitable for administration to a human or animal subject. In some embodiments, the active agent is present in unit dose amount appropriate for administration in a therapeutic regimen that shows a statistically significant probability of achieving a predetermined therapeutic effect when administered to a relevant population.

As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

As used herein, the term “subject” refers to an organism, typically a mammal (e.g., a human). In some embodiments, a subject is suffering from a relevant disease, disorder, or condition. In some embodiments, a subject is susceptible to a disease, disorder, or condition. In some embodiments, a subject displays one or more signs or symptoms or characteristics of a disease, disorder, or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

As used herein, a “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual 2^{nd} ed., Cold Spring Harbor Laboratory Press, Cold Spring Harbor, N.Y. (1989)), which is incorporated herein by reference for any purpose.

### Engineered Immune Cells

Provided herein are immune cells comprising an engineered T cell receptor (TCR) that include (a) a chimeric TCR alpha chain comprising a TCR alpha constant (TCR Cα) domain, wherein the chimeric TCR alpha chain lacks a TCR alpha variable (TCR Vα) domain; (b) a chimeric TCR beta chain comprising a TCR beta constant (TCR Cβ) domain, wherein the chimeric TCR beta chain lacks a TCR beta variable (TCR Vβ) domain; (c) a scFv VH domain; (d) a scFv VL domain; and (e) a co-stimulatory domain.

As used herein, an “immune cell” refer to a cell of the immune system which can be categorized as a lymphocyte (e.g., T cells, B cells, and natural killer (NK) cells), dendritic cells, monocytes/macrophages, granulocytes (e.g., neutrophils, eosinophils, basophils), mast cells, and their subsets. In some embodiments, the immune cell is a T cell. In some embodiments, the immune cell is a αβ-T cell. In some embodiments, the immune cell is a γδ-T cell. In some embodiments, the immune cell is an NK cell. In some embodiments, the immune cell is an NKT cell. In some embodiments, the immune cell is a monocyte or macrophage. In some embodiments, the cell is a precursor to these cells (e.g., a pluripotent stem cell) that is subsequently differentiated. In some embodiments, an immune cell is an engineered immune cell, which means the immune cell has been genetically modified to express a non-naturally occurring protein (e.g., TESLA (TCR Embedded ScFv for Long-term Activation)), or modified to include a non-coded amino acid, or modified to include posttranslational modifications, or modified to include an exogenous nucleic acid.

The immune cells (e.g., T cells) may be modified in one or more than one manner. The modified immune cells (e.g., T cells) may express at least one non-natural molecule that is a receptor (e.g., a TESLA) for an antigen that is present on the surface of one or more types of cells. In some embodiments, immune cells include immune cells (e.g., T cells) that are not found in nature because they are engineered to comprise or express at least one synthetic molecule that is not found in nature. In specific embodiments, the immune cell can be a T cell, e.g., a αβ-T cell, a γδ-T cell, a CD4+ T cell, a CD8+ T cell, a CD3+CD4−CD8− double-negative (DN) T cell, a Treg cell, a Th1 T cell, a Th2 T cell, a Th17 T cell, another type of T cell, or a population of T cells that comprises a combination of any of the foregoing.

### Engineered T Cell Receptor (TCR)

As used herein, an “engineered T cell receptor” refers to a T-cell receptor that has been genetically engineered to produce an artificial T-cell receptor for use in immunotherapy. A T-cell receptor (TCR) is a protein complex found on the surface of T cells or T lymphocytes, wherein the TCR is responsible for recognizing a presented antigen, immune synapse formation. inducing intracellular signaling, and initiating target cell killing. The TCR can include an extracellular antigen-binding domain, a transmembrane domain, and an intracellular signaling domain. In some embodiments, the TCR includes a CD3 gamma (CD3γ) chain, a CD3 epsilon (CD3ε) chain, a CD3 delta (CD3δ) chain, a T cell receptor (TCR) alpha chain (with variable [V] and/or constant [C] regions), a T cell receptor (TCR) beta chain (with variable [V] and/or constant [C] regions), a T cell receptor (TCR) gamma chain (with variable [V] and/or constant [C] regions), and a T cell receptor (TCR) delta chain (with variable [V] and/or constant [C] regions). In some embodiments, the TCR can further include a CD3 zeta (CD3ζ) chain.

Engineered TCRs with Engineered Alpha Chains and/or Engineered Beta Chains

In some embodiments, an engineered TCR can include a chimeric TCR alpha chain comprising a TCR alpha constant (TCR Cα) domain, wherein the chimeric TCR alpha chain lacks a TCR alpha variable (TCR Vα) domain. In some embodiments, the engineered TCR can include a chimeric TCR beta chain comprising a TCR beta constant (TCR Cβ) domain, wherein the chimeric TCR beta chain lacks a TCR beta variable (TCR Vβ) domain. In some embodiments, the engineered TCR can include both a chimeric TCR alpha chain comprising a TCR alpha constant (TCR Cα) domain, wherein the chimeric TCR alpha chain lacks a TCR alpha variable (TCR Vα) domain, and a chimeric TCR beta chain comprising a TCR beta constant (TCR Cβ) domain, wherein the chimeric TCR beta chain lacks a TCR beta variable (TCR Vβ) domain. In some embodiments, the engineered TCR includes both a chimeric TCR alpha chain comprising a TCR alpha constant (TCR Cα) domain, wherein the chimeric TCR alpha chain lacks a TCR alpha variable (TCR Vα) domain, and a chimeric TCR beta chain comprising a TCR beta constant (TCR Cβ) domain, wherein the chimeric TCR beta chain lacks a TCR beta variable (TCR Vβ) domain, wherein the engineered TCR is expressed in an immune cell. In some embodiments, the engineered TCR can include a TCR Cγ domain. In some embodiments, the engineered TCR can include a TCR Cδ domain. In some embodiments, a TCR Cα domain is derived from a human gene. In some embodiments, a TCR Cα domain is derived from a murine gene. In some embodiments, a TCR Cβ domain is derived from a human gene. In some embodiments, a TCR Cβ domain is derived from a murine gene. In some embodiments, a TCR Cα domain and/or a TCR Cβ domain can include a point mutation to improve membrane stability or to introduce a disulfide bond between a TCR alpha chain and a TCR beta chain.

Engineered TCRs with Engineered Gamma Chains and/or Engineered Delta Chains

In some embodiments, an engineered TCR can include a chimeric TCR gamma chain comprising a TCR gamma constant (TCR Cγ) domain, wherein the chimeric TCR gamma chain lacks a TCR gamma variable (TCR Vγ) domain. In some embodiments, the engineered TCR can include a chimeric TCR delta chain comprising a TCR delta constant (TCR Cδ) domain, wherein the chimeric TCR delta chain lacks a TCR delta variable (TCR Vδ) domain. In some embodiments, the engineered TCR can include both a chimeric TCR gamma chain comprising a TCR gamma constant (TCR Cγ) domain, wherein the chimeric TCR gamma chain lacks a TCR gamma variable (TCR Vγ) domain, and a chimeric TCR delta chain comprising a TCR delta constant (TCR Cδ) domain, wherein the chimeric TCR delta chain lacks a TCR delta variable (TCR Vδ) domain. In some embodiments, the engineered TCR includes both a chimeric TCR gamma chain comprising a TCR gamma constant (TCR Cγ) domain, wherein the chimeric TCR gamma chain lacks a TCR gamma variable (TCR Vγ) domain, and a chimeric TCR delta chain comprising a TCR delta constant (TCR Cδ) domain, wherein the chimeric TCR delta chain lacks a TCR delta variable (TCR Vδ) domain, wherein the engineered TCR is expressed in an immune cell. In some embodiments, the engineered TCR can include a TCR Cα domain. In some embodiments, the engineered TCR can include a TCR Cβ domain. In some embodiments, a TCR Cδ domain is derived from a human gene. In some embodiments, a TCR Cδ domain is derived from a murine gene. In some embodiments, a TCR Cγ domain is derived from a human gene. In some embodiments, a TCR Cγ domain is derived from a murine gene.

### Co-Stimulated TCRs

In some embodiments, an engineered TCR can include a wild-type TCR antigen binding domain and a co-stimulatory domain. In some embodiments, an engineered TCR can include a wild-type TCR antigen binding domain and a co-stimulatory domain, wherein the co-stimulatory domain comprises a single co-stimulatory domain. In some embodiments, a co-stimulatory domain comprises a co-stimulatory domain of CD28, OX40, CD27, ICAM-1, ICOS (CD278), MyD88, CD40, or 4-1BB/CD137. In some embodiments, a co-stimulatory domain comprises a co-stimulatory domain of CD28, 4-1BB, OX40, ICOS, MYD88, 2B4, BTLA, CD2, CD22, CD27, CD30, CD4, CD40, CD79a, CD79b, CD84, CD8a, CD8b, CRACC, CRTAM, CTLA-4, DAP10, DNAM-1, DAP12, DR3, FCER1G, FCGR1A, FCGR2A, FCGR2B, FCGR3A, FCRL1, FCRL2, FCRL3, FCRL4, 5 FCRL5, FCRL6, GITR, HVEM, ITAM, KIR2DL1, KIR2DL2, KIR2DL3, KIR2DL4, KIR2DL5A, KIR2DL5B, KIR3DL1, KIR3DL2, KIR3DL3, LAG3, LAT, LILRB1, LILRB2, Ly9, NKG2A, NKG2C, NKG2D, NKp30, NKp44, NKp46, PD-1, PILRB, SIRPa, SLAMF1, SLAMF6, SLAMF7, TIGIT, TIM1, TIM3, TNFL6, TLR1, TLR10, TLR2, TLR3, TLR4, TLR5, TLR6, TLR7, TLR8, or TLR9. In some embodiments, a co-stimulatory domain comprises a co-stimulatory domain of MyD88 or a co-stimulatory domain of CD40.

In some embodiments, an engineered TCR can include a wild-type TCR antigen binding domain and a co-stimulatory domain, wherein the co-stimulatory domain comprises a first co-stimulatory domain and a second co-stimulatory domain. In some embodiments, the first co-stimulatory domain and the second co-stimulatory domain are independently selected from CD28, OX40, CD27, ICAM-1, ICOS (CD278), MyD88, CD40, or 4-1BB/CD137. In some embodiments, the first co-stimulatory domain and the second co-stimulatory domain are independently selected from CD28, 4-1BB, OX40, ICOS, MYD88, 2B4, BTLA, CD2, CD22, CD27, CD30, CD4, CD40, CD79a, CD79b, CD84, CD8a, CD8b, CRACC, CRTAM, CTLA-4, DAP10, DNAM-1, DAP12, DR3, FCER1G, FCGR1A, FCGR2A, FCGR2B, FCGR3A, FCRL1, FCRL2, FCRL3, FCRL4, 5 FCRL5, FCRL6, GITR, HVEM, ITAM, KIR2DL1, KIR2DL2, KIR2DL3, KIR2DL4, KIR2DL5A, KIR2DL5B, KIR3DL1, KIR3DL2, KIR3DL3, LAG3, LAT, LILRB1, LILRB2, Ly9, NKG2A, NKG2C, NKG2D, NKp30, NKp44, NKp46, PD-1, PILRB, SIRPa, SLAMF1, SLAMF6, SLAMF7, TIGIT, TIM1, TIM3, TNFL6, TLR1, TLR10, TLR2, TLR3, TLR4, TLR5, TLR6, TLR7, TLR8, or TLR9. In some embodiments, the first co-stimulatory domain comprises a co-stimulatory domain of MyD88 and the second co-stimulatory domain comprises a co-stimulatory domain of CD40.

In some embodiments, an engineered TCR can include a wild-type TCR antigen binding domain and a co-stimulatory domain, wherein the co-stimulatory domain comprises a first co-stimulatory domain, a second co-stimulatory domain, and a third co-stimulatory domain. In some embodiments, the first co-stimulatory domain, the second co-stimulatory domain, and the third co-stimulatory domain are independently selected from CD28, OX40, CD27, ICAM-1, ICOS (CD278), MyD88, CD40, or 4-1BB/CD137. In some embodiments, the first co-stimulatory domain, the second co-stimulatory domain, and the third co-stimulatory domain are independently selected from CD28, 4-1BB, OX40, ICOS, MYD88, 2B4, BTLA, CD2, CD22, CD27, CD30, CD4, CD40, CD79a, CD79b, CD84, CD8a, CD8b, CRACC, CRTAM, CTLA-4, DAP10, DNAM-1, DAP12, DR3, FCER1G, FCGR1A, FCGR2A, FCGR2B, FCGR3A, FCRL1, FCRL2, FCRL3, FCRL4, 5 FCRL5, FCRL6, GITR, HVEM, ITAM, KIR2DL1, KIR2DL2, KIR2DL3, KIR2DL4, KIR2DL5A, KIR2DL5B, KIR3DL1, KIR3DL2, KIR3DL3, LAG3, LAT, LILRB1, LILRB2, Ly9, NKG2A, NKG2C, NKG2D, NKp30, NKp44, NKp46, PD-1, PILRB, SIRPa, SLAMF1, SLAMF6, SLAMF7, TIGIT, TIM1, TIM3, TNFL6, TLR1, TLR10, TLR2, TLR3, TLR4, TLR5, TLR6, TLR7, TLR8, or TLR9. In some embodiments, an engineered TCR can include a wild-type TCR antigen binding domain and a co-stimulatory domain, wherein the co-stimulatory domain comprises a plurality (e.g., 2, 3, 4, 5, or 6) of co-stimulatory domains.

In some embodiments, a co-stimulatory domain is linked to the TCR Cα domain. In some embodiments, a co-stimulatory domain is linked to the TCR Cβ domain. In some embodiments, a co-stimulatory domain is linked to the TCR Cγ domain. In some embodiments, a co-stimulatory domain is linked to the TCR Cδ domain. In some embodiments, a co-stimulatory domain is linked to a CD3ε domain. In some embodiments, a co-stimulatory domain is linked to a CD3γ domain. In some embodiments, a co-stimulatory domain is linked to a CD3δ domain. In some embodiments, a co-stimulatory domain is linked to a CD3zeta domain. In some embodiments, one or more co-stimulatory domains is linked to one or more of the TCR Cα domain, the TCR Cβ domain, the TCR Cγ domain, the TCR Cδ domain, the CD3ε domain, the CD3γ domain, the CD3δ domain, the CD3zeta domain, or any combination thereof. In some embodiments, a co-stimulatory domain is linked to an extracellular and transmembrane domain of Fas.

### Antigen Binding Domain

In some embodiments, the engineered TCR can include an antigen binding domain. As used herein, an “antigen binding domain” can refer to an antibody agent or portion thereof that specifically binds to a target moiety or entity. In some embodiments, the antigen binding domain can comprise an antibody fragment. In some embodiments, the antigen binding domain can comprise a high-affinity antibody fragment. In some embodiments, the antigen binding domain can comprise a single chain variable fragment (scFv).

As used herein, “single chain variable fragment, scFv” refers to a fragment of antibody defined as a recombinant protein comprising a heavy chain variable domain (VH) and a light chain variable domain (VL) connected by a linker, which brings the two domains together into association such that an antigen-binding site is formed. In some embodiments, the engineered TCR can include a scFv VH domain. In some embodiments, the engineered TCR can include a scFv VL domain. In some embodiments, the engineered TCR can include both a scFv VH domain and a scFv VL domain.

In some embodiments, a scFv VL domain is linked to a TCR alpha constant (TCR Cα) domain, and a scFv VH domain is linked to a TCR beta constant (TCR Cβ) domain. In some embodiments, a scFv VH domain is linked to a TCR alpha constant (TCR Cα) domain, and a scFv VL domain is linked to a TCR beta constant (TCR Cβ) domain.

In some embodiments, a scFv VH domain is linked to a TCR Cγ domain, and a scFv VL domain is linked to a TCR Cδ domain. In some embodiments, a scFv VL domain is linked to a TCR Cγ domain, and a scFv VH domain is linked to a TCR Cδ domain.

In some embodiments, a scFv VH domain and a scFv VL domain are both linked to a TCR alpha constant (TCR Cα) domain. In some embodiments, a scFv VH domain and a scFv VL domain are both linked to a TCR alpha constant (TCR Cα) domain, wherein a TCR beta constant (TCR Cβ) domain is co-expressed. In some embodiments, a scFv VH domain and a scFv VL domain are both linked to a TCR beta constant (TCR Cβ) domain. In some embodiments, a scFv VH domain and a scFv VL domain are both linked to a TCR beta constant (TCR Cβ) domain, wherein a TCR alpha constant (TCR Cα) domain is co-expressed.

In some embodiments, a scFv VH domain and a scFv VL domain are both linked to a TCR Cγ domain. In some embodiments, a scFv VH domain and a scFv VL domain are both linked to a TCR Cγ domain, wherein a TCR Cδ domain is co-expressed. In some embodiments, a scFv VH domain and a scFv VL domain are both linked to a TCR Cδ domain. In some embodiments, a scFv VH domain and a scFv VL domain are both linked to a TCR Cδ domain, wherein a TCR Cγ domain is co-expressed.

### Co-Stimulatory Domain

In some embodiments, the engineered TCR can include a co-stimulatory domain. In some embodiments, the co-stimulatory domain comprises a single co-stimulatory domain. In some embodiments, the co-stimulatory domain comprises a co-stimulatory domain of CD28, OX40, CD27, ICAM-1, ICOS (CD278), MyD88, CD40, or 4-1BB/CD137. In some embodiments, the co-stimulatory domain comprises a co-stimulatory domain of CD28, 4-1BB, OX40, ICOS, MYD88, 2B4, BTLA, CD2, CD22, CD27, CD30, CD4, CD40, CD79a, CD79b, CD84, CD8a, CD8b, CRACC, CRTAM, CTLA-4, DAP10, DNAM-1, DAP12, DR3, FCER1G, FCGR1A, FCGR2A, FCGR2B, FCGR3A, FCRL1, FCRL2, FCRL3, FCRL4, 5 FCRL5, FCRL6, GITR, HVEM, ITAM, KIR2DL1, KIR2DL2, KIR2DL3, KIR2DL4, KIR2DL5A, KIR2DL5B, KIR3DL1, KIR3DL2, KIR3DL3, LAG3, LAT, LILRB1, LILRB2, Ly9, NKG2A, NKG2C, NKG2D, NKp30, NKp44, NKp46, PD-1, PILRB, SIRPa, SLAMF1, SLAMF6, SLAMF7, TIGIT, TIM1, TIM3, TNFL6, TLR1, TLR10, TLR2, TLR3, TLR4, TLR5, TLR6, TLR7, TLR8, or TLR9. In some embodiments, the co-stimulatory domain comprises a co-stimulatory domain of MyD88 or a co-stimulatory domain of CD40.

In some embodiments, the co-stimulatory domain comprises a first co-stimulatory domain and a second co-stimulatory domain. In some embodiments, the first co-stimulatory domain and the second co-stimulatory domain are independently selected from CD28, OX40, CD27, ICAM-1, ICOS (CD278), MyD88, CD40, or 4-1BB/CD137. In some embodiments, the first co-stimulatory domain and the second co-stimulatory domain are independently selected from CD28, 4-1BB, OX40, ICOS, MYD88, 2B4, BTLA, CD2, CD22, CD27, CD30, CD4, CD40, CD79a, CD79b, CD84, CD8a, CD8b, CRACC, CRTAM, CTLA-4, DAP10, DNAM-1, DAP12, DR3, FCER1G, FCGR1A, FCGR2A, FCGR2B, FCGR3A, FCRL1, FCRL2, FCRL3, FCRL4, 5 FCRL5, FCRL6, GITR, HVEM, ITAM, KIR2DL1, KIR2DL2, KIR2DL3, KIR2DL4, KIR2DL5A, KIR2DL5B, KIR3DL1, KIR3DL2, KIR3DL3, LAG3, LAT, LILRB1, LILRB2, Ly9, NKG2A, NKG2C, NKG2D, NKp30, NKp44, NKp46, PD-1, PILRB, SIRPa, SLAMF1, SLAMF6, SLAMF7, TIGIT, TIM1, TIM3, TNFL6, TLR1, TLR10, TLR2, TLR3, TLR4, TLR5, TLR6, TLR7, TLR8, or TLR9. In some embodiments, the first co-stimulatory domain comprises a co-stimulatory domain of MyD88 and the second co-stimulatory domain comprises a co-stimulatory domain of CD40. In some embodiments, the co-stimulatory domain is linked to the TCR Cα domain. In some embodiments, the co-stimulatory domain is linked to the TCR Cβ domain. In some embodiments, the co-stimulatory domain is linked to the TCR Cγ domain. In some embodiments, the co-stimulatory domain is linked to the TCR Cδ domain. In some embodiments, the co-stimulatory domain is linked to a CD38 domain. In some embodiments, the co-stimulatory domain is linked to a CD3γ domain. In some embodiments, the co-stimulatory domain is linked to a CD38 domain. In some embodiments, the co-stimulatory domain is linked to a CD3zeta domain. In some embodiments, the co-stimulatory domain is linked to the TCR Cα domain, the TCR Cβ domain, the TCR Cγ domain, the TCR Cδ domain, the CD3ε domain, the CD3γ domain, the CD3δ domain, the CD3zeta domain, or any combination thereof. In some embodiments, the co-stimulatory domain is linked to an extracellular and transmembrane domain of Fas.

In some embodiments, the co-stimulatory domain comprises a first co-stimulatory domain, a second co-stimulatory domain, and a third co-stimulatory domain. In some embodiments, the first co-stimulatory domain, the second co-stimulatory domain, and the third co-stimulatory domain are independently selected from CD28, OX40, CD27, ICAM-1, ICOS (CD278), MyD88, CD40, or 4-1BB/CD137. In some embodiments, the first co-stimulatory domain, the second co-stimulatory domain, and the third co-stimulatory domain are independently selected from CD28, 4-1BB, OX40, ICOS, MYD88, 2B4, BTLA, CD2, CD22, CD27, CD30, CD4, CD40, CD79a, CD79b, CD84, CD8a, CD8b, CRACC, CRTAM, CTLA-4, DAP10, DNAM-1, DAP12, DR3, FCER1G, FCGR1A, FCGR2A, FCGR2B, FCGR3A, FCRL1, FCRL2, FCRL3, FCRL4, 5 FCRL5, FCRL6, GITR, HVEM, ITAM, KIR2DL1, KIR2DL2, KIR2DL3, KIR2DL4, KIR2DL5A, KIR2DL5B, KIR3DL1, KIR3DL2, KIR3DL3, LAG3, LAT, LILRB1, LILRB2, Ly9, NKG2A, NKG2C, NKG2D, NKp30, NKp44, NKp46, PD-1, PILRB, SIRPa, SLAMF1, SLAMF6, SLAMF7, TIGIT, TIM1, TIM3, TNFL6, TLR1, TLR10, TLR2, TLR3, TLR4, TLR5, TLR6, TLR7, TLR8, or TLR9. In some embodiments, the co-stimulatory domain comprises a plurality (e.g., 2, 3, 4, 5, or 6) of co-stimulatory domains.

### Linker

As used herein, a “linker” can refer to any natural or synthetic linker sequence or binding/coupling domain that result in covalent or non-covalent linkage of an antigenic peptide to any part of the engineered TCR complex. In some embodiments, the linker can comprise a (G4S)ₙ linker. In some embodiments, the linker can comprise a (EAAAK)ₙ linker. In some embodiments, the linker can comprise a sequence of SEQ ID NO: 1. In some embodiments, the linker can comprise a sequence of SEQ ID NO: 2. In some embodiments, the linker can include a sequence of about 30 amino acids in length (e.g., about 25 amino acids, about 20 amino acids, about 15 amino acids, about 10 amino acids, about 8 amino acids, about 4 amino acids). In some embodiments, the linker can be derived from a hinge sequence of CD8, CD28, IgG1, or IgG4. In some embodiments, the linker can comprise a full-length hinge, or a truncation thereof.

Linker sequence
SEQ ID NO: 1
EAAAK
Linker sequence
SEQ ID NO: 2
GGGGS

In some embodiments, an immune cell includes an engineered TCR wherein (i) a scFv VH domain is linked to a TCR alpha constant (TCR Cα) domain, and (ii) a scFv VL domain is linked to a TCR beta constant (TCR Cβ) domain. In some embodiments, a scFv VH domain is directly linked to a TCR alpha constant (TCR Cα) domain, and/or a scFv VL domain is directly linked to a TCR beta constant (TCR Cβ) domain. In some embodiments, a scFv VH domain is indirectly linked to a TCR alpha constant (TCR Cα) domain, and/or a scFv VL domain is indirectly linked to a TCR beta constant (TCR Cβ) domain, via a linker, hinge, or dimerization sequence. In some embodiments, a scFv VH domain is linked to a TCR alpha constant (TCR Cα) domain via a linker, and a scFv VL domain is linked to a TCR beta constant (TCR Cβ) domain via the linker.

In some embodiments, the immune cell includes an engineered TCR wherein (i) a scFv VL domain is linked to a TCR alpha constant (TCR Cα) domain, and (ii) a scFv VH domain is linked to a TCR beta constant (TCR Cβ) domain. In some embodiments, a scFv VL domain is directly linked to a TCR Cα domain, and/or a scFv VH domain is directly linked to a TCR Cβ domain. In some embodiments, a scFv VL domain is indirectly linked to a TCR Cα domain, and/or a scFv VH domain is indirectly linked to a TCR Cβ domain, via a linker, hinge, or dimerization sequence. In some embodiments, a scFv VL domain is linked to a TCR Cα domain via a linker, and a scFv VH domain is linked to a TCR Cβ domain via the linker.

In some embodiments, a scFv VH domain and a scFv VL domain are linked to a TCR alpha constant (TCR Cα) domain. In some embodiments, a scFv VH domain and a scFv VL domain are directly linked to a TCR alpha constant (TCR Cα) domain. In some embodiments, a scFv VH domain and a scFv VL domain are indirectly linked to a TCR alpha constant (TCR Cα) domain, via a linker, hinge, or dimerization sequence.

In some embodiments, a scFv VH domain and a scFv VL domain are linked to a TCR beta constant (TCR Cβ) domain. In some embodiments, a scFv VH domain and a scFv VL domain are directly linked to a TCR beta constant (TCR Cβ) domain. In some embodiments, a scFv VH domain and a scFv VL domain are indirectly linked to a TCR beta constant (TCR Cβ) domain, via a linker, hinge, or dimerization sequence.

In some embodiments, the immune cell includes an engineered TCR wherein (i) a scFv VH domain is linked to a TCR Cγ domain, and (ii) a scFv VL domain is linked to a TCR Cδ domain. In some embodiments, a scFv VH domain is directly linked to a TCR Cγ domain, and/or a scFv VL domain is directly linked to a TCR Cδ domain. In some embodiments, a scFv VH domain is indirectly linked to a TCR Cγ domain, and/or a scFv VL domain is indirectly linked to a TCR Cδ domain, via a linker, hinge, or dimerization sequence. In some embodiments, a scFv VH domain is linked to a TCR Cγ domain via a linker, and a scFv VL domain is linked to a TCR Cδ domain via the linker.

In some embodiments, the immune cell includes an engineered TCR wherein (i) a scFv VL domain is linked to a TCR Cγ domain, and (ii) a scFv VH domain is linked to a TCR Cδ domain. In some embodiments, a scFv VL domain is directly linked to a TCR Cγ domain, and/or a scFv VH domain is directly linked to a TCR Cδ domain. In some embodiments, a scFv VL domain is indirectly linked to a TCR Cγ domain, and/or a scFv VH domain is indirectly linked to a TCR Cδ domain, via a linker, hinge, or dimerization sequence. In some embodiments, a scFv VL domain is linked to a TCR Cγ domain via a linker, and a scFv VH domain is linked to a TCR Cδ domain via the linker.

In some embodiments, a scFv VH domain and a scFv VL domain are linked to a TCR Cγ domain. In some embodiments, a scFv VH domain and a scFv VL domain are directly linked to a TCR Cγ domain. In some embodiments, a scFv VH domain and a scFv VL domain are indirectly linked to a TCR Cγ domain, via a linker, hinge, or dimerization sequence. In some embodiments, a scFv VH domain and a scFv VL domain are linked to a TCR Cδ domain. In some embodiments, a scFv VH domain and a scFv VL domain are directly linked to a TCR Cδ domain. In some embodiments, a scFv VH domain and a scFv VL domain are indirectly linked to a TCR Cδ domain, via a linker, hinge, or dimerization sequence.

In some embodiments, a scFv VH domain and a scFv VL domain are linked to a CD3ε domain. In some embodiments, a scFv VH domain and a scFv VL domain are directly linked to a CD3ε domain. In some embodiments, a scFv VH domain and a scFv VL domain are indirectly linked to a CD3ε domain, via a linker, hinge, or dimerization sequence. In some embodiments, a scFv VH domain and a scFv VL domain are linked to a CD3γ domain. In some embodiments, a scFv VH domain and a scFv VL domain are directly linked to a CD3γ domain. In some embodiments, a scFv VH domain and a scFv VL domain are indirectly linked to a CD3γ domain, via a linker, hinge, or dimerization sequence. In some embodiments, a scFv VH domain and a scFv VL domain are linked to a CD3δ domain. In some embodiments, a scFv VH domain and a scFv VL domain are directly linked to a CD3δ domain. In some embodiments, a scFv VH domain and a scFv VL domain are indirectly linked to a CD3δ domain, via a linker, hinge, or dimerization sequence.

In some embodiments, the engineered TCR specifically binds to a neoantigen. As used herein, a “neoantigen” refers to a cancer-associated protein that are presented on cancer cells harboring certain mutations that occur in tumor DNA. In some embodiments, the neoantigen comprises a tumor-specific neoantigen. In some embodiments, the neoantigen comprises a non-HLA antigen. In some embodiments, the neoantigen comprises EGFRvIII. In some embodiments, the neoantigen is presented on a HLA molecule. In some embodiments, the neoantigen comprises a peptide derived from a mutant oncogene. In some embodiments, the neoantigen comprises a p53RH antigen. In some embodiments, the neoantigen can comprise a BRAF p.Val600Glu/A*02 antigen, a KRAS p.Gly12Asp/A*03 antigen, a KRAS p.Gly12Val/A*03:01 antigen, a TP53 p.Arg175His/A*02:01 (p53RH) antigen, a KRAS p.Gly12Asp/A*11:01 antigen, a KRAS p.Gly12Val/B*35 antigen, a HRAS/KRAS/NRAS p.Gln61Arg/A*01:01 antigen, a KRAS p.Gly 12Val/A*11:01 antigen, a BRAF p. Val600Glu/B*27:05 antigen, a KRAS p.Gly12Asp/C*08:02 antigen, or a KRAS p.Gly12Val/A*03:01 antigen. In some embodiments, the neoantigen can comprise a BRAF p. Val600Glu antigen, a BRAF p.Val600Met antigen, a FBXW7 p.Arg465Cys antigen, a FBXW7 p.Arg479GIn antigen, a FGFR3 p.Ser249Cys antigen, a IDH1 p.Arg132Cys antigen, a KRAS p.Gly12Cys antigen, a KRAS p.Gly 12Asp antigen, a KRAS p.Gly 12Arg antigen, a KRAS p.Gly 12Val antigen, a KRAS p.Gly 13Asp antigen, a MUC4 p.Asp3157Asn antigen, a NRAS p.GIn61Lys antigen, a NRAS p.GIn61 Arg antigen, a PIK3CA p.Glu545Lys antigen, a PIK3CA p.His1047Arg antigen, a PIK3CA p.Arg88Gln antigen, a PPP2RIA p.Pro179Arg antigen, a PTEN p.Arg130Gly antigen, a PTEN p.Arg130Gin antigen, a TP53 p.Arg175His antigen, a TP53 p.Arg248Gln antigen, a TP53 p.Arg273His antigen, a TP53 p.Arg282Trp antigen, or a TP53 p.Ser241Tyr antigen. In some embodiments, the neoantigen can be presented on a A*02:01 HLA allele, a C*07:01 HLA allele, a C*07:02 HLA allele, a A*01:01 HLA allele, a C*04:01 HLA allele, a A*03:01 HLA allele, a B*07:02 HLA allele, a B*08:01 HLA allele, a A*24:02 HLA allele, or a C*06:02 HLA allele.

In some embodiments, the engineered TCR specifically binds to a tumor associated antigen. In some embodiments, the tumor associated antigen can include a cancer germline antigen (e.g. NY-ESO-1, MAGE-A family members), human endogenous retroviruses, cancer associated viruses (e.g. EBV, HPV), LINE-1 (ORF1p and ORF2p), tissue differentiation antigens (e.g. CD19, CD20, CD22, CD7, CD123), or over-expressed antigens (e.g. CEA, WT1). In some embodiments, the tumor associated antigen is presented on a HLA molecule. In some embodiments, the tumor associated antigen is presented on a cell surface. In some embodiments, the engineered TCR specifically binds to a target post-translational modification on a cell surface (e.g., CA19-9 or other carbohydrate alterations).

In some embodiments, the engineered TCR is expressed from an expression cassette placed in an endogenous T cell receptor alpha constant (TRAC) locus of the immune cell. In some embodiments, the immune cell is a human immune cell. In some embodiments, the immune cell is a T cell.

### Nucleic Acid Sequences and Vectors

Also provided herein are nucleic acid sequences encoding any one of the engineered TCRs described herein. As used herein, “nucleic acid” is used to include any compound and/or substance that comprise a polymer of nucleotides. In some embodiments, a polymer of nucleotides is referred to as polynucleotides. Exemplary nucleic acids or polynucleotides can include, but are not limited to, ribonucleic acids (RNAs) and/or deoxyribonucleic acids (DNAs).

In some embodiments, nucleic acid constructs may be inserted into a recombinant vector or viral vector by methods known to the art, and nucleic acid molecules may be operably linked to an expression control sequence. Non-limiting examples of recombinant vectors include plasmid vectors, transposon vectors, cosmid vectors, and viral vectors (e.g., any adenoviral vectors, cytomegaloviral [CMV] vectors, simian viral [SV40] vectors, adeno-associated virus vectors, lentiviral vectors, and retroviral vectors). In some embodiments, the recombinant vector is a viral vector.

Additional sequences can be added to such cloning and/or expression sequences to optimize their function in cloning and/or expression, to aid in isolation of the polynucleotide, or to improve the introduction of the polynucleotide into a cell. Use of cloning vectors, recombinant vectors, adapters, and linkers is well known in the art.

Also provided herein are recombinant vectors comprising any one the nucleic acid molecules or comprising nucleic acid sequences encoding for any of the amino acid sequences described herein. In some embodiments, the recombinant vector can further include a promoter. In some embodiments, the promoter is a TRAC promoter or EF1-alpha promoter.

Also provided herein are methods of producing any one of the engineered immune cells described herein, comprising introducing into an immune cell any one of the nucleic acid sequences or any one of the vectors described herein. In some embodiments, nucleic acid sequences are inserted into a vector that is able to express a single-chain chimeric polypeptide or a multi-chain chimeric polypeptide of the present disclosure when introduced into an appropriate cell. In some embodiments, the cell can be a eukaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as chordate, mammalian, avian, plant, or insect cells.

Methods of introducing nucleic acids and expression vectors into a cell (e.g., an eukaryotic cell) are known in the art. Non-limiting examples of methods that can be used to introduce a nucleic acid into a cell include electroporation, microinjection, any form of transfection, lipofection, calcium phosphate transfection, dendrimer-based transfection, cationic polymer transfection, cell squeezing, sonoporation, optical transfection, impalefection, hydrodynamic delivery, magnetofection, nanoparticle transfection, cell-penetrating peptides, or viral transduction.

In some embodiments, a nucleic acid sequence can be introduced into an immune cell by using a gene-editing agent. As used herein, a “gene-editing agent” can refer to an agent that can target and bind to a specific sequence in DNA. In some embodiments, a gene-editing agent comprises CRISPR/Cas9 components. In some embodiments, the gene-editing agent comprises CRISPR components. As used herein, the term “CRISPR” refers to a technique of sequence specific genetic manipulation relying on the clustered regularly interspaced short palindromic repeats pathway, which unlike RNA interference regulates gene expression at a transcriptional level. As used herein, a “Cas effector” or “CRISPR-associated protein” can refer to an enzyme or protein that uses CRISPR sequences as a guide to recognize and cleave specific nucleic acid strands that are complementary to the CRISPR sequence. A gene-editing Cas effector can associate with a CRISPR RNA sequence to bind to, and alter DNA or RNA target sequences. In some embodiments, the gene-editing agent comprises a gene-editing Cas effector. In some embodiments, the gene-editing Cas effector comprises a Cas9 protein, a Cas13b protein, or a Cas13d protein. In some embodiments, a gene-editing Cas effector can be a Cas9 endonuclease that makes a double-stranded break in a target DNA sequence. In some embodiments, a gene-editing Cas effector can be a Cas12a nuclease that also makes a double-stranded break in a target DNA sequence. In some embodiments, a gene-editing Cas effector can be a Cas13 nuclease which targets RNA. In some embodiments, a gene-editing Cas effector comprises a Cas9 protein, a Cas13b protein, or a Cas13d protein. In some embodiments, the gene-editing Cas effector comprises a nuclease dead Cas9 (dCas9) protein. In some embodiments, the gene-editing Cas effector comprises a Cas13b protein. In some embodiments, the gene-editing Cas effector comprises a Cas13d protein.

In some embodiments, the gene-editing agent further comprises a guide RNA (gRNA), wherein the gRNA is targeted to an individual gene of a cell. The term “guide RNA” or “gRNA” is a specific type of gRNA that combines tracrRNA (transactivating RNA), which binds to Cas9 to activate the complex to create the necessary strand breaks, and crRNA (CRISPR RNA), comprising complimentary nucleotides to the tracrRNA, into a single RNA construct. Exemplary methods of employing the CRISPR technique are described in WO 2017/091630, which is incorporated by reference in its entirety.

In some embodiments, the guide RNA can recognize a target RNA, for example, by hybridizing to the target RNA. In some embodiments, the guide RNA comprises a sequence that is complementary to the target RNA. In some embodiments, the gRNA can include one or more modified nucleotides. In some embodiments, the gRNA has a length that is about 10 nt (e.g., about 20 nt, about 30 nt, about 40 nt, about 50 nt, about 60 nt, about 70 nt, about 80 nt, about 90 nt, about 100 nt, about 120 nt, about 140 nt, about 160 nt, about 180 nt, about 200 nt, about 300 nt, about 400 nt, about 500 nt, about 600 nt, about 700 nt, about 800 nt, about 900 nt, about 1000 nt, or about 2000 nt).

### Therapeutic Applications

Provided herein are methods of treating a disease in a subject, comprising administering to the subject a composition that comprises or delivers any one of the engineered immune cell described herein or a pharmaceutical composition that includes the engineered immune cell and a pharmaceutically acceptable carrier. As used herein, the term “pharmaceutical composition” refers to a composition in which an active agent is formulated together with one or more pharmaceutically acceptable carriers. In some embodiments, a pharmaceutical composition can include a buffer, a diluent, solubilizer, emulsifier, preservative, adjuvant, an excipient, or any combination thereof. In some embodiments, a composition, if desired, can also contain one or more additional therapeutically active substances. In some embodiments, the composition is suitable for administration to a human or animal subject. In some embodiments, the active agent is present in unit dose amount appropriate for administration in a therapeutic regimen that shows a statistically significant probability of achieving a predetermined therapeutic effect when administered to a relevant population.

In some embodiments, the disease is a cancer. In some embodiments, the disease is a cancer comprising a p53 mutation. In some embodiments, the p53 mutation comprises a p53R175H mutation. In some embodiments, the cancer is an ovary cancer, colorectum cancer, esophagus cancer, head and neck cancer, larynx cancer, lung cancer, skin cancer, pancreas cancer, stomach cancer, liver cancer, brain cancer, bladder cancer, breast cancer, uterus cancer, soft tissue cancer, lymph node cancer, prostate cancer, bone cancer, endocrine gland cancer, or cervix cancer.

### EXAMPLES

The disclosure is further described in the following examples, which do not limit the scope of the disclosure described in the claims.

### Materials and Methods

### Cell Lines

KMS26 was obtained from the Japanese Collection of Research Bioresources Cell Bank (JCRB). T2 cells were obtained from American Type Culture Collection (ATCC). KMS26, NALM6, and T2 cells were cultured in RPMI-1640 (ATCC, 30-2001) with 10% FBS (Cytiva, SH30070.03) and 1% penicillin-streptomycin (Thermo Fisher Scientific, 15140163). Cells were grown in a humidified incubator at 37° C. in 5% CO₂. The KMS26-WT (parental) and KMS26-NULL (TP53 KO) cell lines were modified with GFP and luciferase.

### Generation of NALM6-MUT Cell Line

The IDT Alt-R CRISPR system (IDT) was used to convert the wildtype TP53 allele of NALM6 to the R175H mutant to generate the NALM6-MUT cell line. A homology directed repair template (HDRT) encoded the R175H mutation as well as 5 synonymous mutations to abrogate the PAM site and prevent template switching during repair. A TP53 targeted Cas9 crRNA was duplexed with tracrRNA at a 1:1 molar ratio at 95° C. for 5 mins. After cooling to room temperature, 100 pmols of cr: tracrRNA duplex was complexed with 50 μmol of Cas9 nuclease for 15 minutes at room temperature. The resulting ribonucleoprotein was mixed with 60 μmol of a single stranded HDRT (IDT, Alt-R HDR Donor Oligo) encoding the R175H mutation and 5×10⁵ NALM6 cells in 20 μl of OptiMEM (Thermo Fisher Scientific, 31985062) in a 0.1 cm electroporation cuvette (Bio-Rad, 1652089). The mixture was then electroporated at 100 V for 10 ms using an ECM 2001 (BTX). Cells were immediately recovered with warm culture medium. After 1 week of culture, the electroporated cells were plated by limiting dilution. Individual clones were screened by Sanger sequencing a PCR amplicon encompassing the edit site to assess the TP53 R175H mutation status. Sequences of the gRNA, HDRT, and primers are included in Table 1.

TABLE 1

| Name | Description | Sequence |
|---|---|---|
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |

[symbol]

indicates data missing or illegible when filed

Detection and Quantification of p53RH Antigen

Neoantigen detection and quantification was performed through Valid-NEO pipeline by Complete Omics. In brief, a total of 500 million cells were lysed and pHLA complexes were immunoprecipitated using Valid-NEO enrichment column (Complete Omics Inc) packed by matrix conjugated with anti-human HLA-A, B, C antibody clone W6/32 (BioXcell, BE0079). After elution, dissociation, filtration, size exclusion separation, cleanup and fractionation procedures, peptides were subsequently analyzed on a triple quadrupole mass spectrometer. Transition parameters were manually examined and curated to exclude ions with excessive noise due to co-elution with impurities. Absolute copy numbers of neoantigen peptides presented on the cell surface were calculated based on the Valid-NEO quantification using the AQUA heavy isotope labeled peptides.

### HDRT Template Design and Generation

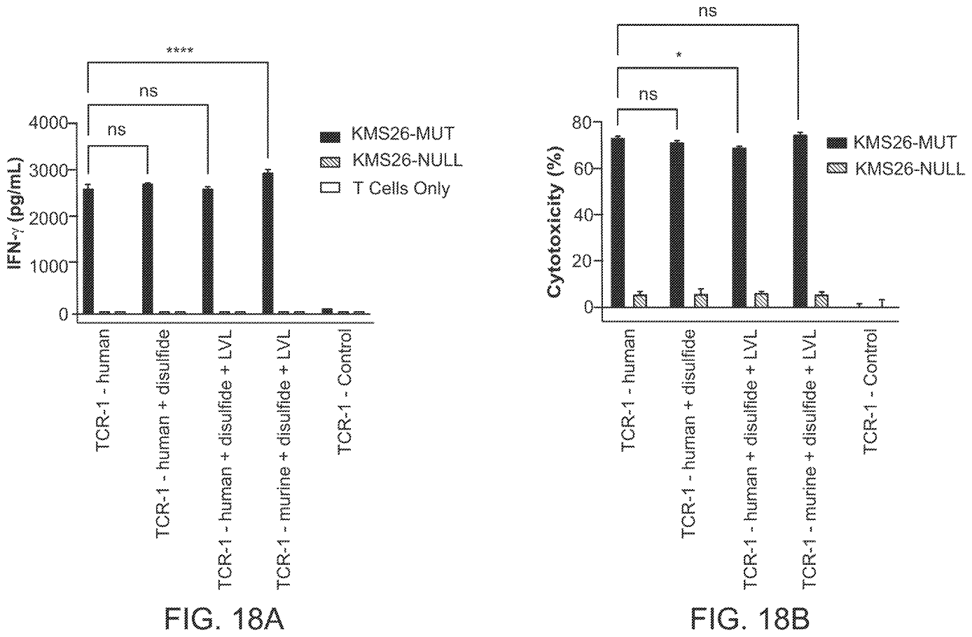

Double stranded DNA HDRTs were generated by PCR amplification from plasmid templates. Plasmid templates were cloned with NEBuilder DNA Assembly HiFi (NEB, E2621L) by mixing synthesized DNA fragments (IDT gBlocks or GeneArt Strings) with a linearized pUC19 derived vector after excising the original contents with EcoRI and HindIII (Addgene, 112021). HDRTs are designed with an EF1a promoter to drive expression of the encoded receptor and a truncated nerve growth factor receptor (tNGFR) tag. Independent protein domains are separated by furin-2A sequences. A poly A terminator sequence is included after the stop codon. Homology arms (HAs) are approximately 300 bps in length. Unless specified in figure legends, all constructs encoding TCRα and TCRβ constant domains use murine chains modified with an additional disulfide bond and stabilizing mutations in the transmembrane of the alpha chain. For TCR-1, fully human TCRα and TCRβ chains without modifications were found to be functionally similar to the modified murine constant domains (FIGS. 18A-18B).

To produce the HDRTs, plasmid templates were PCR amplified with primers specific to the M13 forward and reverse sites using the Q5 Hot Start High-Fidelity 2× Master Mix (New England BioLabs, M0494L). For HDRTs used with a Cas9 nuclease, tCTS sites corresponding to the sgRNA sequence were employed. For HDRTs used with a Cpf1 nuclease, an irrelevant tCTS site was used. HDRT PCR amplicons were purified with 1× AMPure XP Reagent (Beckman Coulter Life Sciences, A63880), eluted in sterile water, and quantified with a NanoDrop Spectrophotometer (Thermo Fisher Scientific). Sequences of the T cell HDRT plasmids are included in Table 2.

TABLE 2

| | | | | | Amino Acid |
|---|---|---|---|---|---|
| Tier | | | | Plasmid | Sequence |
| Number | Tier Name | Designation | Description | Sequence | of CDS |
| 1 | Conventionally | CAR-1 | H2-CAR with CD28 hinge | SEQ ID NO: 3 | SEQ ID NO: 4 |
| | Engineered | CAR-2 | H2-CAR with CD8α hinge | SEQ ID NO: 5 | SEQ ID NO: 6 |
| | T Cells | TCR-1 | AV6/BV11 TCR | SEQ ID NO: 7 | SEQ ID NO: 8 |
| | | TCR-2 | 1a2 TCR | SEQ ID NO: 9 | SEQ ID NO: 10 |
| | | TCR-3 | AV12/BV6 TCR | SEQ ID NO: 11 | SEQ ID NO: 12 |
| | | TCR-4 | AV38/BV10 TCR | SEQ ID NO: 13 | SEQ ID NO: 14 |
| | | TCR-1 - | AV6/BV11 TCR with | SEQ ID NO: 15 | SEQ ID NO: 16 |
| | | human | human constant | | |
| | | | domains | | |
| | | TCR-1 - | AV6/BV11 TCR with | SEQ ID NO: 17 | SEQ ID NO: 18 |
| | | human + | human constant | | |
| | | disulfide | domains and an | | |
| | | | engineered | | |
| | | | disulfide bond | | |
| | | TCR-1 - | AV6/BV11 TCR with | SEQ ID NO: 19 | SEQ ID NO: 20 |
| | | human + | human constant | | |
| | | disulfide + | domains and an | | |
| | | LVL | engineered disulfide | | |
| | | | bond and stabilizing | | |
| | | | mutations in the | | |
| | | | TCRα | | |
| | | | transmembrane | | |
| | | | domain | | |
| 2 | Hybrid TCR- | TCR/CAR-1 | H2-CD3γ | SEQ ID NO: 21 | SEQ ID NO: 22 |
| | CAR T Cells | TCR/CAR-2 | H2-VHVL-Cα-2A-Cβ | SEQ ID NO: 23 | SEQ ID NO: 24 |
| | | TCR/CAR-3 | H2-VHVL-Cβ-2A-Cα | SEQ ID NO: 25 | SEQ ID NO: 26 |
| | | TCR/CAR-4 | H2-VLVH-Cα-2A-Cβ | SEQ ID NO: 27 | SEQ ID NO: 28 |
| | | TCR/CAR-5 | H2-VLVH-Cβ-2A-Cα | SEQ ID NO: 29 | SEQ ID NO: 30 |
| | | TCR/CAR-6 | H2-VHVL-A6-TCRα- | SEQ ID NO: 31 | SEQ ID NO: 32 |
| | | | 2A-A6-TCRβ | | |
| | | TCR/CAR-7 | H2-VHVL-A6-TCRβ- | SEQ ID NO: 33 | SEQ ID NO: 34 |
| | | | 2A-A6-TCRα | | |
| | | TCR/CAR-8 | H2-VLVH-A6-TCRα- | SEQ ID NO: 35 | SEQ ID NO: 36 |
| | | | 2A-A6-TCRβ | | |
| | | TCR/CAR-9 | H2-VLVH-A6-TCRβ- | SEQ ID NO: 37 | SEQ ID NO: 38 |
| | | | 2A-A6-TCRα | | |
| 3 | Split/CAR | Split/CAR-1 | H2-VL-Cβ-2A-VH-Cα | SEQ ID NO: 39 | SEQ ID NO: 40 |
| | T Cells | Split/CAR-2 | H2-VH-Cβ-2A-VL-Cα | SEQ ID NO: 41 | SEQ ID NO: 42 |
| | | Split/CAR-3 | H2-VL-EAAAK-Cβ-2A- | SEQ ID NO: 43 | SEQ ID NO: 44 |
| | | | VH-EAAAK-Cα | | |
| | | Split/CAR-4 | H2-VH-EAAAK-Cβ- | SEQ ID NO: 45 | SEQ ID NO: 46 |
| | | | 2A-VL-EAAAK-Cα | | |
| | | Split/CAR-5 | H2-VL-A6-TCRβ-2A- | SEQ ID NO: 47 | SEQ ID NO: 48 |
| | | | VH-A6-TCRα | | |
| | | Split/CAR-6 | H2-VH-A6-TCRβ-2A- | SEQ ID NO: 49 | SEQ ID NO: 50 |
| | | | VL-A6-TCRα | | |
| 4 | Co-stimulated | TCR/CoS-1 | TCR-1 + Cβ-MC | SEQ ID NO: 51 | SEQ ID NO: 52 |
| | T Cells | (human | | | |
| | | constant | | | |
| | | domains) | | | |
| | | TCR/CoS-2 | TCR-1 + Fas-MC | SEQ ID NO: 53 | SEQ ID NO: 54 |
| | | (human | | | |
| | | constant | | | |
| | | domains) | | | |
| | | TCR/CoS-3 | TCR-1 + Fas-OX40 | SEQ ID NO: 55 | SEQ ID NO: 56 |
| | | (human | | | |
| | | constant | | | |
| | | domains) | | | |
| | | TCR/CoS-4 | TCR-1 + Fas-41BB | SEQ ID NO: 57 | SEQ ID NO: 58 |
| | | (human | | | |
| | | constant | | | |
| | | domains) | | | |
| | | TCR/CoS-5 | TCR-1 + Fas-IL7Rα | SEQ ID NO: 59 | SEQ ID NO: 60 |
| | | (human | | | |
| | | constant | | | |
| | | domains) | | | |
| | | TCR/CoS-6 | TCR-1 + CASTAT3 | SEQ ID NO: 61 | SEQ ID NO: 62 |
| | | (human | | | |
| | | constant | | | |
| | | domains) | | | |
| | | TCR/CoS-7 | TCR-1 + CASTAT5 | SEQ ID NO: 63 | SEQ ID NO: 64 |
| | | (human | | | |
| | | constant | | | |
| | | domains) | | | |
| | | TCR/CoS-8 | TCR-1 + C7R-1 | SEQ ID NO: 65 | SEQ ID NO: 66 |
| | | (human | | | |
| | | constant | | | |
| | | domains) | | | |
| | | TCR/CoS-9 | TCR-1 + C7R-2 | SEQ ID NO: 67 | SEQ ID NO: 68 |
| | | (human | | | |
| | | constant | | | |
| | | domains) | | | |
| | | TCR/CoS-1 | TCR-1 + Cβ-MC | SEQ ID NO: 69 | SEQ ID NO: 70 |
| | | TCR/CoS-2 | TCR-1 + Fas-MC | SEQ ID NO: 71 | SEQ ID NO: 72 |
| | | TESLA-1 | Split/CAR-3 + Cβ-MC | SEQ ID NO: 73 | SEQ ID NO: 74 |
| | | TESLA-2 | Split/CAR-3 + Fas-MC | SEQ ID NO: 75 | SEQ ID NO: 76 |

-H2-CAR with CD28 hinge (plasmid sequence)
SEQ ID NO: 3
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcggggggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
CTCTGCCTGTGACAGCTCTGCTGCTGCCTCTTGCTCTGCTTCTGCATGCCGCCAGA
CCTGACATCCAGATGACACAGAGCCCTAGCAGCCTGTCTGCCAGCGTGGGAGAC
AGAGTGACCATCACCTGTAGAGCCAGCCAGGACGTGAACACAGCCGTGGCTTGG
TATCAGCAGAAGCCTGGCAAGGCCCCTAAGCTGCTGATCTACAGCGCCTACTTCC
TGTACAGCGGCGTGCCCAGCAGATTCAGCGGCTCTAGAAGCGGCACCGACTTCA
CCCTGACCATAAGCAGTCTGCAGCCCGAGGACTTCGCCACCTACTACTGTCAGCA
GTACAGCCGGTACAGCCCCGTGACATTTGGCCAGGGCACCAAGGTGGAAATCAA
GAGAACAGGCGGAGGCTCTGGCGGAGGTGGAAGCGGAGGCGGAGCTTCTGAAG
TGCAGCTGGTTGAAAGTGGCGGCGGACTGGTTCAACCTGGCGGATCTCTGAGAC
TGAGCTGTGCCGCCAGCGGCTTCAATGTGTATGCCTCTGGCATGCACTGGGTCCG
ACAGGCCCCTGGAAAAGGCCTTGAGTGGGTCGCCAAGATCTACCCCGACAGCGA
CTACACCTACTATGCCGACAGCGTGAAGGGCAGATTCACCATCAGCGCCGACAC
CAGCAAGAACACCGCCTACCTGCAGATGAACAGCCTGAGAGCCGAGGACACCGC
CGTGTACTACTGCTCCAGAGACAGCAGCTTCTACTACGTGTACGCCATGGACTAC
TGGGGCCAGGGAACACTGGTCACCGTGTCCAGCGCCGCCGCTATCGAAGTGATG
TACCCTCCTCCTTACCTGGACAACGAGAAGTCCAACGGCACCATCATCCACGTGA
AGGGAAAGCACCTGTGTCCTTCTCCACTGTTCCCCGGACCTAGCAAGCCTTTCTG
GGTGCTCGTTGTTGTTGGCGGCGTGCTGGCCTGTTACAGCCTGCTGGTTACCGTG
GCCTTCATCATCTTTTGGGTCCGAAGCAAGCGGAGCCGGCTGCTGCACAGCGATT
ACATGAACATGACCCCTCGGAGGCCCGGACCAACCAGAAAGCACTACCAGCCTT
ACGCTCCTCCTAGAGATTTCGCCGCCTACCGGTCCAGAGTGAAGTTCTCCAGATC
TGCCGACGCTCCTGCCTATCAGCAGGGCCAGAACCAGCTGTACAACGAGCTGAA
CCTGGGGAGAAGAGAAGAGTACGACGTGCTGGACAAGCGGAGAGGCAGAGATC
CTGAGATGGGCGGCAAGCCCAGACGGAAGAATCCTCAAGAGGGCCTGTATAATG
AGCTGCAGAAAGACAAGATGGCCGAGGCCTACAGCGAGATCGGAATGAAGGGC
GAGCGCAGAAGAGGCAAGGGACACGATGGACTGTACCAGGGACTGAGCACCGC
CACCAAGGATACCTATGACGCCCTGCACATGCAGGCCCTGCCTCCAAGACGGGC
CAAACGATCTGGCTCTGGCCAGTGTACCAACTATGCCCTGCTCAAGCTCGCTGGC
GACGTCGAGTCTAACCCTGGACCAATGGGAGCTGGCGCTACAGGCAGAGCTATG
GATGGACCTAGACTGCTGCTGCTCCTGCTTCTGGGAGTGTCTCTTGGCGGAGCCA
AAGAGGCCTGTCCTACCGGCCTGTATACACACTCTGGCGAGTGCTGCAAGGCCTG
CAATCTTGGAGAAGGCGTCGCACAGCCTTGCGGCGCTAATCAGACAGTGTGCGA
GCCTTGCCTGGACAGCGTGACCTTTAGCGACGTGGTGTCAGCCACCGAGCCATGC
AAGCCATGTACCGAGTGTGTGGGCCTGCAGAGCATGTCTGCCCCTTGCGTGGAA
GCTGACGATGCCGTGTGTAGATGCGCCTACGGCTACTACCAGGACGAGACAACA
GGCAGATGCGAGGCCTGTAGAGTGTGCGAAGCTGGATCTGGCCTGGTGTTCAGC
TGCCAAGACAAGCAGAACACCGTGTGCGAGGAATGCCCCGATGGCACCTATAGC
GACGAGGCCAACCATGTGGATCCCTGCCTGCCTTGTACCGTGTGTGAAGATACCG
AGCGGCAGCTGCGCGAGTGTACAAGATGGGCTGATGCCGAGTGCGAAGAGATCC
CTGGCAGATGGATCACCAGAAGCACACCTCCAGAGGGCAGCGATAGCACAGCCC
CTTCTACACAAGAGCCCGAGGCTCCTCCTGAGCAGGACCTGATTGCTTCTACAGT
GGCTGGCGTCGTGACCACCGTGATGGGATCTAGTCAGCCCGTGGTCACCAGAGG
CACCACCGACAATCTGATCCCCGTGTACTGTAGCATCCTGGCCGCCGTGGTTGTT
GGACTGGTGGCCTATATCGCCTTCAAGCGGTGGAACAGCTGATGATTCGAAGCG
GCCGCTTCGAGCAGACATGATAAGATACATTGATGAGTTTGGACAAACCACAAC
TAGAATGCAGTGAAAAAAATGCTTTATTTGTGAAATTTGTGATGCTATTGCTTTA
TTTGTAACCATTATAAGCTGCAATAAACAAGTTACACGGCAGGGTCAGGGTTCTG
GATATctgtgggacaagaggatcagggttaggacatgatctcatttccctctttgccccaacccaggctggagtccagatgccagt
gatggacaagggcggggctctgtggggctggcaagtcacggtctcatgctttatacgggaaatagcatcttagaaaccagctgctcgt
gatggactgggactcagggacaggcacaagctatcaatcttggccaagaggccatgatttcagtgaacgttcacggccaggcctggc
ctgccactcaaggaaacCACTGGCCGTCGTTTTACAACGTCGTGACTgggaaaaccctggcgttacccaa
cttaatcgccttgcagcacatccccctttcgccagctggcgtaatagcgaagaggcccgcaccgatcgcccttcccaacagttgcgca
gcctgaatggcgaatggcgcctgatgcggtattttctccttacgcatctgtgcggtatttcacaccgcatatggtgcactctcagtacaatc
tgctctgatgccgcatagttaagccagccccgacacccgccaacacccgctgacgcgccctgacgggcttgtctgctcccggcatcc
gcttacagacaagctgtgaccgtctccgggagctgcatgtgtcagaggttttcaccgtcatcaccgaaacgcgcga
-H2-CAR with CD28 hinge (amino acid sequence)
SEQ ID NO: 4
MALPVTALLLPLALLLHAARPDIQMTQSPSSLSASVGDRVTITCRASQDVNTAVAWY
QQKPGKAPKLLIYSAYFLYSGVPSRFSGSRSGTDFTLTISSLQPEDFATYYCQQYSRYS
PVTFGQGTKVEIKRTGGGSGGGGSGGGASEVQLVESGGGLVQPGGSLRLSCAASGF
NVYASGMHWVRQAPGKGLEWVAKIYPDSDYTYYADSVKGRFTISADTSKNTAYLQ
MNSLRAEDTAVYYCSRDSSFYYVYAMDYWGQGTLVTVSSAAAIEVMYPPPYLDNE
KSNGTIIHVKGKHLCPSPLFPGPSKPFWVLVVVGGVLACYSLLVTVAFIIFWVRSKRS
RLLHSDYMNMTPRRPGPTRKHYQPYAPPRDFAAYRSRVKFSRSADAPAYQQGQNQ
LYNELNLGRREEYDVLDKRRGRDPEMGGKPRRKNPQEGLYNELQKDKMAEAYSEI
GMKGERRRGKGHDGLYQGLSTATKDTYDALHMQALPPRRAKRSGSGQCTNYALL
KLAGDVESNPGPMGAGATGRAMDGPRLLLLLLLGVSLGGAKEACPTGLYTHSGECC
KACNLGEGVAQPCGANQTVCEPCLDSVTFSDVVSATEPCKPCTECVGLQSMSAPCV
EADDAVCRCAYGYYQDETTGRCEACRVCEAGSGLVFSCQDKQNTVCEECPDGTYS
DEANHVDPCLPCTVCEDTERQLRECTRWADAECEEIPGRWITRSTPPEGSDSTAPSTQ
EPEAPPEQDLIASTVAGVVTTVMGSSQPVVTRGTTDNLIPVYCSILAAVVVGLVAYIA
FKRWNS
-H2-CAR with CD8a hinge (plasmid sequence)
SEQ ID NO: 5
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
CTCTGCCTGTGACAGCTCTGCTGCTGCCTCTTGCTCTGCTTCTGCATGCCGCCAGA
CCTGACATCCAGATGACACAGAGCCCTAGCAGCCTGTCTGCCAGCGTGGGAGAC
AGAGTGACCATCACCTGTAGAGCCAGCCAGGACGTGAACACAGCCGTGGCTTGG
TATCAGCAGAAGCCTGGCAAGGCCCCTAAGCTGCTGATCTACAGCGCCTACTTCC
TGTACAGCGGCGTGCCCAGCAGATTCAGCGGCTCTAGAAGCGGCACCGACTTCA
CCCTGACCATAAGCAGTCTGCAGCCCGAGGACTTCGCCACCTACTACTGTCAGCA
GTACAGCCGGTACAGCCCCGTGACATTTGGCCAGGGCACCAAGGTGGAAATCAA
GAGAACAGGCGGAGGCTCTGGCGGAGGTGGAAGCGGAGGCGGAGCTTCTGAAG
TGCAGCTGGTTGAAAGTGGCGGCGGACTGGTTCAACCTGGCGGATCTCTGAGAC
TGAGCTGTGCCGCCAGCGGCTTCAATGTGTATGCCTCTGGCATGCACTGGGTCCG
ACAGGCCCCTGGAAAAGGCCTTGAGTGGGTCGCCAAGATCTACCCCGACAGCGA
CTACACCTACTATGCCGACAGCGTGAAGGGCAGATTCACCATCAGCGCCGACAC
CAGCAAGAACACCGCCTACCTGCAGATGAACAGCCTGAGAGCCGAGGACACCGC
CGTGTACTACTGCTCCAGAGACAGCAGCTTCTACTACGTGTACGCCATGGACTAC
TGGGGCCAGGGAACACTGGTCACCGTGTCCAGCACAACAACCCCTGCTCCTAGA
CCTCCTACACCAGCTCCTACAATCGCCAGCCAGCCTCTGTCTCTGAGGCCAGAGG
CTTGTAGACCTGCTGCTGGCGGAGCCGTGCATACAAGAGGACTGGATTTCGCCTG
CGATATCTACTTCTGGGTGCTCGTGGTTGTCGGCGGAGTGCTGGCCTGTTATAGC
CTGCTGGTCACAGTGGCCTTCATCATCTTTTGGGTCCGAAGCAAGCGGAGCCGGC
TGCTGCACAGCGATTACATGAACATGACCCCTCGGAGGCCCGGACCTACCAGAA
AGCACTACCAGCCTTACGCTCCTCCTAGAGATTTCGCCGCCTACCGGTCCAGAGT
GAAGTTCTCCAGATCTGCCGACGCTCCTGCCTATCAGCAGGGCCAGAACCAGCTG
TACAACGAGCTGAACCTGGGGAGAAGAGAAGAGTACGACGTGCTGGATAAGCG
GAGAGGCAGAGATCCTGAGATGGGCGGAAAGCCCCAGCGGAGAAAGAATCCTC
AAGAGGGCCTGTATAATGAGCTGCAGAAAGACAAGATGGCCGAGGCCTACAGC
GAGATCGGAATGAAGGGCGAGCGCAGAAGAGGCAAGGGACACGATGGACTGTA
CCAGGGCCTGAGCACCGCCACCAAGGATACCTATGATGCCCTGCACATGCAGGC
CCTGCCTCCAAGACGGGCCAAACGATCTGGCTCTGGCCAGTGTACCAACTATGCC
CTGCTCAAGCTCGCTGGCGACGTCGAGTCTAACCCTGGACCAATGGGAGCTGGC
GCTACAGGCAGAGCTATGGATGGACCTAGACTGCTGCTGCTCCTGCTTCTGGGAG
TGTCTCTTGGCGGAGCCAAAGAGGCCTGTCCTACCGGCCTGTATACACACTCTGG
CGAGTGCTGCAAGGCCTGCAATCTTGGAGAAGGCGTCGCACAGCCTTGCGGCGC
TAATCAGACAGTGTGCGAGCCTTGCCTGGACAGCGTGACCTTTAGCGACGTGGTG
TCAGCCACCGAGCCATGCAAGCCATGTACCGAGTGTGTGGGCCTGCAGAGCATG
TCTGCCCCTTGCGTGGAAGCTGACGATGCCGTGTGTAGATGCGCCTACGGCTACT
ACCAGGACGAGACAACAGGCAGATGCGAGGCCTGTAGAGTGTGCGAAGCTGGA
TCTGGCCTGGTGTTCAGCTGCCAAGACAAGCAGAACACCGTGTGCGAGGAATGC
CCCGATGGCACCTATAGCGACGAGGCCAACCATGTGGATCCCTGCCTGCCTTGTA
CCGTGTGTGAAGATACCGAGCGGCAGCTGCGCGAGTGTACAAGATGGGCTGATG
CCGAGTGCGAAGAGATCCCTGGCAGATGGATCACCAGAAGCACACCTCCAGAGG
GCAGCGATAGCACAGCCCCTTCTACACAAGAGCCCGAGGCTCCTCCTGAGCAGG
ACCTGATTGCTTCTACAGTGGCTGGCGTCGTGACCACCGTGATGGGATCTAGTCA
GCCCGTGGTCACCAGAGGCACCACCGACAATCTGATCCCCGTGTACTGTAGCATC
CTGGCCGCCGTGGTTGTTGGACTGGTGGCCTATATCGCCTTCAAGCGGTGGAACA
GCTGATGATTCGAAGCGGCCGCTTCGAGCAGACATGATAAGATACATTGATGAG
TTTGGACAAACCACAACTAGAATGCAGTGAAAAAAATGCTTTATTTGTGAAATTT
GTGATGCTATTGCTTTATTTGTAACCATTATAAGCTGCAATAAACAAGTTACACG
GCAGGGTCAGGGTTCTGGATATctgtgggacaagaggatcagggttaggacatgatctcatttccctctttgcccc
aacccaggctggagtccagatgccagtgatggacaagggcggggctctgtggggctggcaagtcacggtctcatgctttatacggga
aatagcatcttagaaaccagctgctcgtgatggactgggactcagggacaggcacaagctatcaatcttggccaagaggccatgattt
cagtgaacgttcacggccaggcctggcctgccactcaaggaaacCACTGGCCGTCGTTTTACAACGTCGTG
ACTgggaaaaccctggcgttacccaacttaatcgccttgcagcacatccccctttcgccagctggcgtaatagcgaagaggcccgc
accgatcgcccttcccaacagttgcgcagcctgaatggcgaatggcgcctgatgcggtattttctccttacgcatctgtgcggtatttcac
accgcatatggtgcactctcagtacaatctgctctgatgccgcatagttaagccagccccgacacccgccaacacccgctgacgcgcc
ctgacgggcttgtctgctcccggcatccgcttacagacaagctgtgaccgtctccgggagctgcatgtgtcagaggttttcaccgtcatc
accgaaacgcgcga
-H2-CAR with CD8α hinge (amino acid sequence)
SEQ ID NO: 6
MALPVTALLLPLALLLHAARPDIQMTQSPSSLSASVGDRVTITCRASQDVNTAVAWY
QQKPGKAPKLLIYSAYFLYSGVPSRFSGSRSGTDFTLTISSLQPEDFATYYCQQYSRYS
PVTFGQGTKVEIKRTGGGSGGGGSGGGASEVQLVESGGGLVQPGGSLRLSCAASGF
NVYASGMHWVRQAPGKGLEWVAKIYPDSDYTYYADSVKGRFTISADTSKNTAYLQ
MNSLRAEDTAVYYCSRDSSFYYVYAMDYWGQGTLVTVSSTTTPAPRPPTPAPTIASQ
PLSLRPEACRPAAGGAVHTRGLDFACDIYFWVLVVVGGVLACYSLLVTVAFIIFWVR
SKRSRLLHSDYMNMTPRRPGPTRKHYQPYAPPRDFAAYRSRVKFSRSADAPAYQQG
QNQLYNELNLGRREEYDVLDKRRGRDPEMGGKPQRRKNPQEGLYNELQKDKMAE
AYSEIGMKGERRRGKGHDGLYQGLSTATKDTYDALHMQALPPRRAKRSGSGQCTN
YALLKLAGDVESNPGPMGAGATGRAMDGPRLLLLLLLGVSLGGAKEACPTGLYTHS
GECCKACNLGEGVAQPCGANQTVCEPCLDSVTFSDVVSATEPCKPCTECVGLQSMS
APCVEADDAVCRCAYGYYQDETTGRCEACRVCEAGSGLVFSCQDKQNTVCEECPD
GTYSDEANHVDPCLPCTVCEDTERQLRECTRWADAECEEIPGRWITRSTPPEGSDST
APSTQEPEAPPEQDLIASTVAGVVTTVMGSSQPVVTRGTTDNLIPVYCSILAAVVVGL
VAYIAFKRWNS
-AV6/BV11 TCR (plasmid sequence)
SEQ ID NO: 7
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgaggggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
GCACTCGATTGCTCTGTTGGGCCGCGCTGTGCTTGCTGGGTGCCGAATTGACAGA
AGCGGGTGTAGCTCAAAGTCCTCGGTACAAGATTATTGAGAAACGCCAGTCTGTT
GCCTTCTGGTGTAACCCTATCTCTGGTCACGCAACTCTCTATTGGTATCAGCAAAT
CCTGGGTCAAGGTCCTAAACTTCTGATTCAATTTCAGAATAATGGCGTTGTGGAC
GACTCTCAACTGCCTAAAGACAGATTCAGTGCGGAAAGGTTGAAAGGCGTCGAT
AGCACACTCAAAATTCAGCCGGCAAAGCTTGAAGACTCCGCCGTTTATCTGTGCG
CGAGTTCTCTTGATCCTGGGGACACAGGGGAACTGTTCTTTGGCGAGGGATCACG
ATTGACCGTCCTGGAGGACCTGAGAAACGTGACCCCTCCTAAGGTGTCCCTGTTC
GAGCCTAGCAAGGCCGAGATCGCCAACAAGCAGAAAGCCACACTCGTGTGCCTG
GCCAGAGGCTTCTTTCCCGATCACGTGGAACTGTCTTGGTGGGTCAACGGCAAAG
AGGTGCACAGCGGCGTCTGTACTGATCCCCAGGCCTACAAAGAGAGCAACTACA
GCTACTGCCTGAGCAGCAGACTGAGAGTGTCCGCCACCTTCTGGCACAACCCCA
GAAACCACTTCAGATGCCAGGTGCAGTTTCACGGCCTGAGCGAAGAGGACAAGT
GGCCTGAGGGAAGCCCCAAGCCAGTGACACAGAATATCTCTGCCGAAGCCTGGG
GCAGAGCCGATTGTGGAATTACCAGCGCCAGCTACCAGCAAGGCGTGCTGTCTG
CCACAATCCTGTACGAGATCCTGCTGGGCAAAGCCACTCTGTACGCCGTGCTGGT
GTCTACCCTGGTCGTGATGGCCATGGTCAAGCGGAAGAACAGCAGGGCCAAGAG
AAGTGGAAGCGGCGCCACAAACTTCAGCCTGTTGAAACAGGCCGGGGACGTCGA
AGAGAATCCTGGACCAATGGAATCCTTTCTCGGAGGTGTACTGCTCATACTCTGG
CTGCAAGTGGATTGGGTAAAGAGTCAAAAAATTGAACAAAACAGCGAGGCTTTG
AATATCCAAGAGGGCAAGACTGCCACGCTGACCTGTAACTATACAAACTATAGT
CCGGCATACCTCCAGTGGTACCGGCAAGATCCTGGTCGCGGGCCGGTTTTCCTGT
TGCTTATTAGAGAGAATGAAAAGGAAAAAAGAAAAGAACGCTTGAAGGTCACTT
TCGACACGACGCTTAAACAGAGTCTTTTCCACATTACCGCGTCACAGCCTGCGGA
CTCAGCTACATACCTGTGTGCTCTCGACATCTATCCCCACGACATGAGGTTTGGC
GCAGGCACTCGACTCACGGTAAAACCCGACATTCAGAATCCTGAGCCTGCCGTG
TACCAGCTGAAGGACCCTAGAAGCCAGGACAGCACCCTGTGCCTGTTCACCGAC
TTCGACAGCCAGATCAACGTGCCCAAGACCATGGAAAGCGGCACCTTCATCACC
GATAAGTGCGTGCTGGACATGAAGGCCATGGACAGCAAGAGCAACGGCGCCATT
GCCTGGTCCAACCAGACCAGCTTCACGTGCCAGGACATCTTCAAAGAGACAAAC
GCCACCTATCCTAGCAGCGACGTGCCCTGTGATGCCACACTGACCGAGAAGTCCT
TCGAGACAGACATGAACCTGAACTTCCAGAACCTGCTGGTCATCGTGCTGAGAA
TCCTGCTGCTGAAGGTGGCCGGCTTCAACCTGCTGATGACCCTGAGACTGTGGTC
CAGCCGGGCCAAACGATCTGGCTCTGGCCAGTGTACCAACTATGCCCTGCTCAAG
CTCGCTGGCGACGTCGAGTCTAACCCTGGACCAATGGGAGCTGGCGCTACAGGC
AGAGCTATGGATGGACCTAGACTGCTGCTGCTCCTGCTTCTGGGAGTGTCTCTTG
GCGGAGCCAAAGAGGCCTGTCCTACCGGCCTGTATACACACTCTGGCGAGTGCT
GCAAGGCCTGCAATCTTGGAGAAGGCGTCGCACAGCCTTGCGGCGCTAATCAGA
CAGTGTGCGAGCCTTGCCTGGACAGCGTGACCTTTAGCGACGTGGTGTCAGCCAC
CGAGCCATGCAAGCCATGTACCGAGTGTGTGGGCCTGCAGAGCATGTCTGCCCCT
TGCGTGGAAGCTGACGATGCCGTGTGTAGATGCGCCTACGGCTACTACCAGGAC
GAGACAACAGGCAGATGCGAGGCCTGTAGAGTGTGCGAAGCTGGATCTGGCCTG
GTGTTCAGCTGCCAAGACAAGCAGAACACCGTGTGCGAGGAATGCCCCGATGGC
ACCTATAGCGACGAGGCCAACCATGTGGATCCCTGCCTGCCTTGTACCGTGTGTG
AAGATACCGAGCGGCAGCTGCGCGAGTGTACAAGATGGGCTGATGCCGAGTGCG
AAGAGATCCCTGGCAGATGGATCACCAGAAGCACACCTCCAGAGGGCAGCGATA
GCACAGCCCCTTCTACACAAGAGCCCGAGGCTCCTCCTGAGCAGGACCTGATTGC
TTCTACAGTGGCTGGCGTCGTGACCACCGTGATGGGATCTAGTCAGCCCGTGGTC
ACCAGAGGCACCACCGACAATCTGATCCCCGTGTACTGTAGCATCCTGGCCGCCG
TGGTTGTTGGACTGGTGGCCTATATCGCCTTCAAGCGGTGGAACAGCTGATGATT
CGAAGCGGCCGCTTCGAGCAGACATGATAAGATACATTGATGAGTTTGGACAAA
CCACAACTAGAATGCAGTGAAAAAAATGCTTTATTTGTGAAATTTGTGATGCTAT
TGCTTTATTTGTAACCATTATAAGCTGCAATAAACAAGTTACACGGCAGGGTCAG
GGTTCTGGATATctgtgggacaagaggatcagggttaggacatgatctcatttccctctttgccccaacccaggctggagtc
cagatgccagtgatggacaagggcggggctctgtggggctggcaagtcacggtctcatgctttatacgggaaatagcatcttagaaac
cagctgctcgtgatggactgggactcagggacaggcacaagctatcaatcttggccaagaggccatgatttcagtgaacgttcacggc
caggcctggcctgccactcaaggaaacCACTGGCCGTCGTTTTACAACGTCGTGACTgggaaaaccctg
gcgttacccaacttaatcgccttgcagcacatccccctttcgccagctggcgtaatagcgaagaggcccgcaccgatcgcccttccca
acagttgcgcagcctgaatggcgaatggcgcctgatgcggtattttctccttacgcatctgtgcggtatttcacaccgcatatggtgcact
ctcagtacaatctgctctgatgccgcatagttaagccagccccgacacccgccaacacccgctgacgcgccctgacgggcttgtctgc
tcccggcatccgcttacagacaagctgtgaccgtctccgggagctgcatgtgtcagaggttttcaccgtcatcaccgaaacgcgcga
-AV6/BV11 TCR (amino acid sequence)
SEQ ID NO: 8
MGTRLLCWAALCLLGAELTEAGVAQSPRYKIIEKRQSVAFWCNPISGHATLYWYQQ
ILGQGPKLLIQFQNNGVVDDSQLPKDRFSAERLKGVDSTLKIQPAKLEDSAVYLCASS
LDPGDTGELFFGEGSRLTVLEDLRNVTPPKVSLFEPSKAEIANKQKATLVCLARGFFP
DHVELSWWVNGKEVHSGVCTDPQAYKESNYSYCLSSRLRVSATFWHNPRNHFRCQ
VQFHGLSEEDKWPEGSPKPVTQNISAEAWGRADCGITSASYQQGVLSATILYEILLGK
ATLYAVLVSTLVVMAMVKRKNSRAKRSGSGATNFSLLKQAGDVEENPGPMESFLG
GVLLILWLQVDWVKSQKIEQNSEALNIQEGKTATLTCNYTNYSPAYLQWYRQDPGR
GPVFLLLIRENEKEKRKERLKVTFDTTLKQSLFHITASQPADSATYLCALDIYPHDMR
FGAGTRLTVKPDIQNPEPAVYQLKDPRSQDSTLCLFTDFDSQINVPKTMESGTFITDK
CVLDMKAMDSKSNGAIAWSNQTSFTCQDIFKETNATYPSSDVPCDATLTEKSFETD
MNLNFQNLLVIVLRILLLKVAGFNLLMTLRLWSSRAKRSGSGQCTNYALLKLAGDV
ESNPGPMGAGATGRAMDGPRLLLLLLLGVSLGGAKEACPTGLYTHSGECCKACNLG
EGVAQPCGANQTVCEPCLDSVTFSDVVSATEPCKPCTECVGLQSMSAPCVEADDAV
CRCAYGYYQDETTGRCEACRVCEAGSGLVFSCQDKQNTVCEECPDGTYSDEANHV
DPCLPCTVCEDTERQLRECTRWADAECEEIPGRWITRSTPPEGSDSTAPSTQEPEAPPE
QDLIASTVAGVVTTVMGSSQPVVTRGTTDNLIPVYCSILAAVVVGLVAYIAFKRWNS
-1a2 TCR (plasmid sequence)
SEQ ID NO: 9
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggyggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGC
ATCCTCAGCTGCTGGGCTACGTGGTGCTGTGTTTGCTTGGAGCCGGACCTCTGGA
AGCCCAAGTGACACAGAACCCCAGATACCTGATCACCGTGACCGGCAAGAAACT
GACCGTGACCTGCAGCCAGAACATGAACCACGAGTACATGAGCTGGTACAGACA
GGACCCTGGCCTGGGCCTGAGACAGATCTACTACAGCATGAACGTGGAAGTGAC
CGACAAGGGCGACGTGCCCGAGGGCTACAAGGTGTCCAGAAAAGAGAAGCGGA
ACTTCCCACTGATCCTGGAAAGCCCATCTCCTAACCAGACCAGCCTGTACTTCTG
CGCCAGCTCTATTCAGCAGGGCGCCGACACACAGTACTTCGGCCCTGGAACAAG
ACTGACAGTGCTGGAGGACCTGAGAAACGTGACCCCTCCTAAGGTGTCCCTGTTC
GAGCCTAGCAAGGCCGAGATCGCCAACAAGCAGAAAGCCACACTCGTGTGCCTG
GCCAGAGGCTTCTTTCCCGATCACGTGGAACTGTCTTGGTGGGTCAACGGCAAAG
AGGTGCACAGCGGCGTCTGTACTGATCCCCAGGCCTACAAAGAGAGCAACTACA
GCTACTGCCTGAGCAGCAGACTGAGAGTGTCCGCCACCTTCTGGCACAACCCCA
GAAACCACTTCAGATGCCAGGTGCAGTTTCACGGCCTGAGCGAAGAGGACAAGT
GGCCTGAGGGAAGCCCCAAGCCAGTGACACAGAATATCTCTGCCGAAGCCTGGG
GCAGAGCCGATTGTGGAATTACCAGCGCCAGCTACCAGCAAGGCGTGCTGTCTG
CCACAATCCTGTACGAGATCCTGCTGGGCAAAGCCACTCTGTACGCCGTGCTGGT
GTCTACCCTGGTCGTGATGGCCATGGTCAAGCGGAAGAACAGCAGGGCCAAGAG
AAGTGGAAGCGGCGCCACAAACTTCAGCCTGTTGAAACAGGCCGGGGACGTCGA
AGAGAATCCTGGACCAATGGCCAAGAGCCTGAGAGTGCTGCTGGTCATCCTGTG
GCTGCAGCTGTCTTGGGTCTGGTCCCAGCAGAAAGAGGTGGAACAGGACCCTGG
ACCTCTGTCTGTTCCTGAGGGCGCCATCGTGTCCCTGAACTGCACCTACAGCAAC
AGCGCCTTCCAGTACTTCATGTGGTACAGACAGTACAGCCGGAAGGGCCCCGAG
CTGCTGATGTACACATACAGCAGCGGCAACAAAGAGGACGGCCGGTTTACAGCC
CAGGTGGACAAGAGCAGCAAGTACATCTCCCTGTTCATCCGGGACAGCCAGCCT
AGCGATAGCGCCACATATCTGTGTGCCATGAGCGGCCTGAAAGAGGACAGCAGC
TACAAGCTGATCTTCGGCAGCGGCACCAGACTGCTCGTTAGACCTGACATTCAGA
ATCCTGAGCCTGCCGTGTACCAGCTGAAGGACCCTAGAAGCCAGGACAGCACCC
TGTGCCTGTTCACCGACTTCGACAGCCAGATCAACGTGCCCAAGACCATGGAAA
GCGGCACCTTCATCACCGATAAGTGCGTGCTGGACATGAAGGCCATGGACAGCA
AGAGCAACGGCGCCATTGCCTGGTCCAACCAGACCAGCTTCACGTGCCAGGACA
TCTTCAAAGAGACAAACGCCACCTATCCTAGCAGCGACGTGCCCTGTGATGCCAC
ACTGACCGAGAAGTCCTTCGAGACAGACATGAACCTGAACTTCCAGAACCTGCT
GGTCATCGTGCTGAGAATCCTGCTGCTGAAGGTGGCCGGCTTCAACCTGCTGATG
ACCCTGAGACTGTGGTCCAGCCGGGCCAAACGATCTGGCTCTGGCCAGTGTACC
AACTATGCCCTGCTCAAGCTCGCTGGCGACGTCGAGTCTAACCCTGGACCAATGG
GAGCTGGCGCTACAGGCAGAGCTATGGATGGACCTAGACTGCTGCTGCTCCTGCT
TCTGGGAGTGTCTCTTGGCGGAGCCAAAGAGGCCTGTCCTACCGGCCTGTATACA
CACTCTGGCGAGTGCTGCAAGGCCTGCAATCTTGGAGAAGGCGTCGCACAGCCT
TGCGGCGCTAATCAGACAGTGTGCGAGCCTTGCCTGGACAGCGTGACCTTTAGCG
ACGTGGTGTCAGCCACCGAGCCATGCAAGCCATGTACCGAGTGTGTGGGCCTGC
AGAGCATGTCTGCCCCTTGCGTGGAAGCTGACGATGCCGTGTGTAGATGCGCCTA
CGGCTACTACCAGGACGAGACAACAGGCAGATGCGAGGCCTGTAGAGTGTGCGA
AGCTGGATCTGGCCTGGTGTTCAGCTGCCAAGACAAGCAGAACACCGTGTGCGA
GGAATGCCCCGATGGCACCTATAGCGACGAGGCCAACCATGTGGATCCCTGCCT
GCCTTGTACCGTGTGTGAAGATACCGAGCGGCAGCTGCGCGAGTGTACAAGATG
GGCTGATGCCGAGTGCGAAGAGATCCCTGGCAGATGGATCACCAGAAGCACACC
TCCAGAGGGCAGCGATAGCACAGCCCCTTCTACACAAGAGCCCGAGGCTCCTCC
TGAGCAGGACCTGATTGCTTCTACAGTGGCTGGCGTCGTGACCACCGTGATGGGA
TCTAGTCAGCCCGTGGTCACCAGAGGCACCACCGACAATCTGATCCCCGTGTACT
GTAGCATCCTGGCCGCCGTGGTTGTTGGACTGGTGGCCTATATCGCCTTCAAGCG
GTGGAACAGCTGATGATTCGAAGCGGCCGCTTCGAGCAGACATGATAAGATACA
TTGATGAGTTTGGACAAACCACAACTAGAATGCAGTGAAAAAAATGCTTTATTTG
TGAAATTTGTGATGCTATTGCTTTATTTGTAACCATTATAAGCTGCAATAAACAA
GTTACACGGCAGGGTCAGGGTTCTGGATATctgtgggacaagaggatcagggttaggacatgatctcattt
ccctctttgccccaacccaggctggagtccagatgccagtgatggacaagggcggggctctgtggggctggcaagtcacggtctcat
gctttatacgggaaatagcatcttagaaaccagctgctcgtgatggactgggactcagggacaggcacaagctatcaatcttggccaa
gaggccatgatttcagtgaacgttcacggccaggcctggcctgccactcaaggaaacCACTGGCCGTCGTTTTACA
ACGTCGTGACTgggaaaaccctggcgttacccaacttaatcgccttgcagcacatccccctttcgccagctggcgtaatagc
gaagaggcccgcaccgatcgcccttcccaacagttgcgcagcctgaatggcgaatggcgcctgatgcggtattttctccttacgcatct
gtgcggtatttcacaccgcatatggtgcactctcagtacaatctgctctgatgccgcatagttaagccagccccgacacccgccaacac
ccgctgacgcgccctgacgggcttgtctgctcccggcatccgcttacagacaagctgtgaccgtctccgggagctgcatgtgtcagag
gttttcaccgtcatcaccgaaacgcgcga
-1a2 TCR (amino acid sequence)
SEQ ID NO: 10
MHPQLLGYVVLCLLGAGPLEAQVTQNPRYLITVTGKKLTVTCSQNMNHEYMSWYR
QDPGLGLRQIYYSMNVEVTDKGDVPEGYKVSRKEKRNFPLILESPSPNQTSLYFCASS
IQQGADTQYFGPGTRLTVLEDLRNVTPPKVSLFEPSKAEIANKQKATLVCLARGFFPD
HVELSWWVNGKEVHSGVCTDPQAYKESNYSYCLSSRLRVSATFWHNPRNHFRCQV
QFHGLSEEDKWPEGSPKPVTQNISAEAWGRADCGITSASYQQGVLSATILYEILLGKA
TLYAVLVSTLVVMAMVKRKNSRAKRSGSGATNFSLLKQAGDVEENPGPMAKSLRV
LLVILWLQLSWVWSQQKEVEQDPGPLSVPEGAIVSLNCTYSNSAFQYFMWYRQYSR
KGPELLMYTYSSGNKEDGRFTAQVDKSSKYISLFIRDSQPSDSATYLCAMSGLKEDSS
YKLIFGSGTRLLVRPDIQNPEPAVYQLKDPRSQDSTLCLFTDFDSQINVPKTMESGTFI
TDKCVLDMKAMDSKSNGAIAWSNQTSFTCQDIFKETNATYPSSDVPCDATLTEKSFE
TDMNLNFQNLLVIVLRILLLKVAGFNLLMTLRLWSSRAKRSGSGQCTNYALLKLAG
DVESNPGPMGAGATGRAMDGPRLLLLLLLGVSLGGAKEACPTGLYTHSGECCKAC
NLGEGVAQPCGANQTVCEPCLDSVTFSDVVSATEPCKPCTECVGLQSMSAPCVEAD
DAVCRCAYGYYQDETTGRCEACRVCEAGSGLVFSCQDKQNTVCEECPDGTYSDEA
NHVDPCLPCTVCEDTERQLRECTRWADAECEEIPGRWITRSTPPEGSDSTAPSTQEPE
APPEQDLIASTVAGVVTTVMGSSQPVVTRGTTDNLIPVYCSILAAVVVGLVAYIAFK
RWNS
-AV12/BV6 TCR (plasmid sequence)
SEQ ID NO: 11
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggagggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcggggggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGT
CTATCGGCCTGCTGTGCTGCGTGGCCTTTAGTCTGCTTTGGGCCTCTCCTGTGAAT
GCCGGCGTGACCCAGACACCTAAGTTCCAGGTGCTGAAAACCGGCCAGAGCATG
ACCCTGCAGTGCGCCCAGGACATGAACCACAACAGCATGTACTGGTACAGACAG
GACCCCGGCATGGGCCTGAGACTGATCTACTACTCTGCCAGCGAGGGCACCACC
GACAAAGGCGAAGTGCCCAATGGCTACAACGTGTCCCGGCTGAACAAGAGAGA
GTTCAGCCTGCGGCTGGAAAGCGCCGCTCCTTCTCAGACCTCCGTGTACTTTTGC
GCCAGCAGCGAAGGCCTGTGGCAAGTGGGAGATGAGCAGTACTTTGGCCCTGGC
ACCAGACTGACCGTGACAGAGGACCTGAGAAACGTGACCCCTCCTAAGGTGTCC
CTGTTCGAGCCTAGCAAGGCCGAGATCGCCAACAAGCAGAAAGCCACACTCGTG
TGCCTGGCCAGAGGCTTCTTTCCCGATCACGTGGAACTGTCTTGGTGGGTCAACG
GCAAAGAGGTGCACAGCGGCGTCTGTACTGATCCCCAGGCCTACAAAGAGAGCA
ACTACAGCTACTGCCTGAGCAGCAGACTGAGAGTGTCCGCCACCTTCTGGCACA
ACCCCAGAAACCACTTCAGATGCCAGGTGCAGTTTCACGGCCTGAGCGAAGAGG
ACAAGTGGCCTGAGGGAAGCCCCAAGCCAGTGACACAGAATATCTCTGCCGAAG
CCTGGGGCAGAGCCGATTGTGGAATTACCAGCGCCAGCTACCAGCAAGGCGTGC
TGTCTGCCACAATCCTGTACGAGATCCTGCTGGGCAAAGCCACTCTGTACGCCGT
GCTGGTGTCTACCCTGGTCGTGATGGCCATGGTCAAGCGGAAGAACAGCAGGGC
CAAGAGAAGTGGAAGCGGCGCCACAAACTTCAGCCTGTTGAAACAGGCCGGGG
ACGTCGAAGAGAATCCTGGACCAATGATCAGCCTGAGAGTGCTGCTGGTCATCC
TGTGGCTGCAGCTGTCTTGGGTCTGGTCCCAGCGGAAAGAGGTGGAACAGGACC
CCGGACCTTTCAATGTGCCTGAAGGCGCCACCGTGGCCTTCAACTGCACCTACAG
CAATAGCGCCAGCCAGAGCTTCTTCTGGTACAGACAGGATTGCCGGAAAGAACC
CAAGCTGCTGATGAGCGTGTACAGCAGCGGCAACGAGGACGGCAGATTCACAGC
CCAGCTGAACAGAGCCAGCCAGTACATCAGCCTGCTGATCCGGGATAGCAAGCT
GAGCGATAGCGCCACCTACCTGTGTGTGGTTCAGCCTGGCGGCTACCAGAAAGT
GACCTTTGGCACCGGCACCAAGCTGCAAGTGATCCCTGACATTCAGAATCCTGAG
CCTGCCGTGTACCAGCTGAAGGACCCTAGAAGCCAGGACAGCACCCTGTGCCTG
TTCACCGACTTCGACAGCCAGATCAACGTGCCCAAGACCATGGAAAGCGGCACC
TTCATCACCGATAAGTGCGTGCTGGACATGAAGGCCATGGACAGCAAGAGCAAC
GGCGCCATTGCCTGGTCCAACCAGACCAGCTTCACGTGCCAGGACATCTTCAAAG
AGACAAACGCCACCTATCCTAGCAGCGACGTGCCCTGTGATGCCACACTGACCG
AGAAGTCCTTCGAGACAGACATGAACCTGAACTTCCAGAACCTGCTGGTCATCGT
GCTGAGAATCCTGCTGCTGAAGGTGGCCGGCTTCAACCTGCTGATGACCCTGAGA
CTGTGGTCCAGCCGGGCCAAACGATCTGGCTCTGGCCAGTGTACCAACTATGCCC
TGCTCAAGCTCGCTGGCGACGTCGAGTCTAACCCTGGACCAATGGGAGCTGGCG
CTACAGGCAGAGCTATGGATGGACCTAGACTGCTGCTGCTCCTGCTTCTGGGAGT
GTCTCTTGGCGGAGCCAAAGAGGCCTGTCCTACCGGCCTGTATACACACTCTGGC
GAGTGCTGCAAGGCCTGCAATCTTGGAGAAGGCGTCGCACAGCCTTGCGGCGCT
AATCAGACAGTGTGCGAGCCTTGCCTGGACAGCGTGACCTTTAGCGACGTGGTGT
CAGCCACCGAGCCATGCAAGCCATGTACCGAGTGTGTGGGCCTGCAGAGCATGT
CTGCCCCTTGCGTGGAAGCTGACGATGCCGTGTGTAGATGCGCCTACGGCTACTA
CCAGGACGAGACAACAGGCAGATGCGAGGCCTGTAGAGTGTGCGAAGCTGGATC
TGGCCTGGTGTTCAGCTGCCAAGACAAGCAGAACACCGTGTGCGAGGAATGCCC
CGATGGCACCTATAGCGACGAGGCCAACCATGTGGATCCCTGCCTGCCTTGTACC
GTGTGTGAAGATACCGAGCGGCAGCTGCGCGAGTGTACAAGATGGGCTGATGCC
GAGTGCGAAGAGATCCCTGGCAGATGGATCACCAGAAGCACACCTCCAGAGGGC
AGCGATAGCACAGCCCCTTCTACACAAGAGCCCGAGGCTCCTCCTGAGCAGGAC
CTGATTGCTTCTACAGTGGCTGGCGTCGTGACCACCGTGATGGGATCTAGTCAGC
CCGTGGTCACCAGAGGCACCACCGACAATCTGATCCCCGTGTACTGTAGCATCCT
GGCCGCCGTGGTTGTTGGACTGGTGGCCTATATCGCCTTCAAGCGGTGGAACAGC
TGATGATTCGAAGCGGCCGCTTCGAGCAGACATGATAAGATACATTGATGAGTTT
GGACAAACCACAACTAGAATGCAGTGAAAAAAATGCTTTATTTGTGAAATTTGT
GATGCTATTGCTTTATTTGTAACCATTATAAGCTGCAATAAACAAGTTACACGGC
AGGGTCAGGGTTCTGGATATctgtgggacaagaggatcagggttaggacatgatctcatttccctctttgccccaac
ccaggctggagtccagatgccagtgatggacaagggcggggctctgtggggctggcaagtcacggtctcatgctttatacgggaaat
agcatcttagaaaccagctgctcgtgatggactgggactcagggacaggcacaagctatcaatcttggccaagaggccatgatttcag
tgaacgttcacggccaggcctggcctgccactcaaggaaacCACTGGCCGTCGTTTTACAACGTCGTGAC
Tgggaaaaccctggcgttacccaacttaatcgccttgcagcacatccccctttcgccagctggcgtaatagcgaagaggcccgcacc
gatcgcccttcccaacagttgcgcagcctgaatggcgaatggcgcctgatgcggtattttctccttacgcatctgtgcggtatttcacacc
gcatatggtgcactctcagtacaatctgctctgatgccgcatagttaagccagccccgacacccgccaacacccgctgacgcgccctg
acgggcttgtctgctcccggcatccgcttacagacaagctgtgaccgtctccgggagctgcatgtgtcagaggttttcaccgtcatcac
cgaaacgcgcga
-AV12/BV6 TCR (amino acid sequence)
SEQ ID NO: 12
MSIGLLCCVAFSLLWASPVNAGVTQTPKFQVLKTGQSMTLQCAQDMNHNSMYWY
RQDPGMGLRLIYYSASEGTTDKGEVPNGYNVSRLNKREFSLRLESAAPSQTSVYFCA
SSEGLWQVGDEQYFGPGTRLTVTEDLRNVTPPKVSLFEPSKAEIANKQKATLVCLAR
GFFPDHVELSWWVNGKEVHSGVCTDPQAYKESNYSYCLSSRLRVSATFWHNPRNH
FRCQVQFHGLSEEDKWPEGSPKPVTQNISAEAWGRADCGITSASYQQGVLSATILYEI
LLGKATLYAVLVSTLVVMAMVKRKNSRAKRSGSGATNFSLLKQAGDVEENPGPMIS
LRVLLVILWLQLSWVWSQRKEVEQDPGPFNVPEGATVAFNCTYSNSASQSFFWYRQ
DCRKEPKLLMSVYSSGNEDGRFTAQLNRASQYISLLIRDSKLSDSATYLCVVQPGGY
QKVTFGTGTKLQVIPDIQNPEPAVYQLKDPRSQDSTLCLFTDFDSQINVPKTMESGTFI
TDKCVLDMKAMDSKSNGAIAWSNQTSFTCQDIFKETNATYPSSDVPCDATLTEKSFE
TDMNLNFQNLLVIVLRILLLKVAGENLLMTLRLWSSRAKRSGSGQCTNYALLKLAG
DVESNPGPMGAGATGRAMDGPRLLLLLLLGVSLGGAKEACPTGLYTHSGECCKAC
NLGEGVAQPCGANQTVCEPCLDSVTFSDVVSATEPCKPCTECVGLQSMSAPCVEAD
DAVCRCAYGYYQDETTGRCEACRVCEAGSGLVFSCQDKQNTVCEECPDGTYSDEA
NHVDPCLPCTVCEDTERQLRECTRWADAECEEIPGRWITRSTPPEGSDSTAPSTQEPE
APPEQDLIASTVAGVVTTVMGSSQPVVTRGTTDNLIPVYCSILAAVVVGLVAYIAFK
RWNS
-AV38/BV10 TCR (plasmid sequence)
SEQ ID NO: 13
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcggggggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
GCACCAGACTGTTCTTCTACGTGGCCCTGTGTCTGCTGTGGACCGGACATATGGA
CGCCGGCATTACACAGAGCCCCAGACACAAAGTGACCGAGACAGGCACCCCTGT
GACACTGAGATGTCACCAGACCGAGAACCACCGCTACATGTACTGGTACAGACA
GGACCCTGGCCACGGCCTGAGACTGATCCACTACAGCTACGGCGTGAAGGACAC
CGACAAGGGCGAAGTGTCTGACGGCTACAGCGTGTCCAGAAGCAAGACCGAGG
ACTTCCTGCTGACCCTGGAAAGCGCCACAAGCAGCCAGACCAGCGTGTACTTCTG
TGCCATCAGCGAGCTGGTTACCGGCGATAGCCCTCTGCACTTTGGCAATGGCACC
AGGCTGACCGTGACAGAGGACCTGAGAAACGTGACCCCTCCTAAGGTGTCCCTG
TTCGAGCCTAGCAAGGCCGAGATCGCCAACAAGCAGAAAGCCACACTCGTGTGC
CTGGCCAGAGGCTTCTTTCCCGATCACGTGGAACTGTCTTGGTGGGTCAACGGCA
AAGAGGTGCACAGCGGCGTCTGTACTGATCCCCAGGCCTACAAAGAGAGCAACT
ACAGCTACTGCCTGAGCAGCAGACTGAGAGTGTCCGCCACCTTCTGGCACAACC
CCAGAAACCACTTCAGATGCCAGGTGCAGTTTCACGGCCTGAGCGAAGAGGACA
AGTGGCCTGAGGGAAGCCCCAAGCCAGTGACACAGAATATCTCTGCCGAAGCCT
GGGGCAGAGCCGATTGTGGAATTACCAGCGCCAGCTACCAGCAAGGCGTGCTGT
CTGCCACAATCCTGTACGAGATCCTGCTGGGCAAAGCCACTCTGTACGCCGTGCT
GGTGTCTACCCTGGTCGTGATGGCCATGGTCAAGCGGAAGAACAGCAGGGCCAA
GAGAAGTGGAAGCGGCGCCACAAACTTCAGCCTGTTGAAACAGGCCGGGGACGT
CGAAGAGAATCCTGGACCAATGACCAGAGTGTCTCTGCTGTGGGCCGTCGTGGT
GTCCACATGTCTGGAATCTGGCATGGCCCAGACCGTGACACAGAGCCAGCCTGA
GATGTCTGTGCAAGAGGCCGAGACAGTGACCCTGAGCTGCACCTACGATACCAG
CGAGAACAACTACTACCTGTTCTGGTACAAGCAGCCTCCTAGCCGGCAGATGATC
CTGGTCATCAGACAAGAGGCCTATAAGCAGCAGAACGCCACCGAGAACAGATTC
AGCGTGAACTTCCAGAAGGCCGCCAAGAGCTTCAGCCTGAAGATCAGCGATAGC
CAGCTGGGCGACACCGCCATGTACTTTTGCGCCTTCATGGGCTACTCTGGCGCCG
GAAGCTACCAGCTGACATTTGGCAAGGGCACCAAACTGAGCGTGATCCCTGACA
TTCAGAATCCTGAGCCTGCCGTGTACCAGCTGAAGGACCCTAGAAGCCAGGACA
GCACCCTGTGCCTGTTCACCGACTTCGACAGCCAGATCAACGTGCCCAAGACCAT
GGAAAGCGGCACCTTCATCACCGATAAGTGCGTGCTGGACATGAAGGCCATGGA
CAGCAAGAGCAACGGCGCCATTGCCTGGTCCAACCAGACCAGCTTCACGTGCCA
GGACATCTTCAAAGAGACAAACGCCACCTATCCTAGCAGCGACGTGCCCTGTGA
TGCCACACTGACCGAGAAGTCCTTCGAGACAGACATGAACCTGAACTTCCAGAA
CCTGCTGGTCATCGTGCTGAGAATCCTGCTGCTGAAGGTGGCCGGCTTCAACCTG
CTGATGACCCTGAGACTGTGGTCCAGCCGGGCCAAACGATCTGGCTCTGGCCAGT
GTACCAACTATGCCCTGCTCAAGCTCGCTGGCGACGTCGAGTCTAACCCTGGACC
AATGGGAGCTGGCGCTACAGGCAGAGCTATGGATGGACCTAGACTGCTGCTGCT
CCTGCTTCTGGGAGTGTCTCTTGGCGGAGCCAAAGAGGCCTGTCCTACCGGCCTG
TATACACACTCTGGCGAGTGCTGCAAGGCCTGCAATCTTGGAGAAGGCGTCGCA
CAGCCTTGCGGCGCTAATCAGACAGTGTGCGAGCCTTGCCTGGACAGCGTGACCT
TTAGCGACGTGGTGTCAGCCACCGAGCCATGCAAGCCATGTACCGAGTGTGTGG
GCCTGCAGAGCATGTCTGCCCCTTGCGTGGAAGCTGACGATGCCGTGTGTAGATG
CGCCTACGGCTACTACCAGGACGAGACAACAGGCAGATGCGAGGCCTGTAGAGT
GTGCGAAGCTGGATCTGGCCTGGTGTTCAGCTGCCAAGACAAGCAGAACACCGT
GTGCGAGGAATGCCCCGATGGCACCTATAGCGACGAGGCCAACCATGTGGATCC
CTGCCTGCCTTGTACCGTGTGTGAAGATACCGAGCGGCAGCTGCGCGAGTGTACA
AGATGGGCTGATGCCGAGTGCGAAGAGATCCCTGGCAGATGGATCACCAGAAGC
ACACCTCCAGAGGGCAGCGATAGCACAGCCCCTTCTACACAAGAGCCCGAGGCT
CCTCCTGAGCAGGACCTGATTGCTTCTACAGTGGCTGGCGTCGTGACCACCGTGA
TGGGATCTAGTCAGCCCGTGGTCACCAGAGGCACCACCGACAATCTGATCCCCGT
GTACTGTAGCATCCTGGCCGCCGTGGTTGTTGGACTGGTGGCCTATATCGCCTTC
AAGCGGTGGAACAGCTGATGATTCGAAGCGGCCGCTTCGAGCAGACATGATAAG
ATACATTGATGAGTTTGGACAAACCACAACTAGAATGCAGTGAAAAAAATGCTT
TATTTGTGAAATTTGTGATGCTATTGCTTTATTTGTAACCATTATAAGCTGCAATA
AACAAGTTACACGGCAGGGTCAGGGTTCTGGATATctgtgggacaagaggatcagggttaggacat
gatctcatttccctctttgccccaacccaggctggagtccagatgccagtgatggacaagggcggggctctgtggggctggcaagtca
cggtctcatgctttatacgggaaatagcatcttagaaaccagctgctcgtgatggactgggactcagggacaggcacaagctatcaatc
ttggccaagaggccatgatttcagtgaacgttcacggccaggcctggcctgccactcaaggaaacCACTGGCCGTCGTT
TTACAACGTCGTGACTgggaaaaccctggcgttacccaacttaatcgccttgcagcacatccccctttcgccagctggc
gtaatagcgaagaggcccgcaccgatcgcccttcccaacagttgcgcagcctgaatggcgaatggcgcctgatgcggtattttctcctt
acgcatctgtgcggtatttcacaccgcatatggtgcactctcagtacaatctgctctgatgccgcatagttaagccagccccgacacccg
ccaacacccgctgacgcgccctgacgggcttgtctgctcccggcatccgcttacagacaagctgtgaccgtctccgggagctgcatgt
gtcagaggttttcaccgtcatcaccgaaacgcgcga
-AV38/BV10 TCR (amino acid sequence)
SEQ ID NO: 14
MGTRLFFYVALCLLWTGHMDAGITQSPRHKVTETGTPVTLRCHQTENHRYMYWYR
QDPGHGLRLIHYSYGVKDTDKGEVSDGYSVSRSKTEDFLLTLESATSSQTSVYFCAIS
ELVTGDSPLHFGNGTRLTVTEDLRNVTPPKVSLFEPSKAEIANKQKATLVCLARGFFP
DHVELSWWVNGKEVHSGVCTDPQAYKESNYSYCLSSRLRVSATFWHNPRNHFRCQ
VQFHGLSEEDKWPEGSPKPVTQNISAEAWGRADCGITSASYQQGVLSATILYEILLGK
ATLYAVLVSTLVVMAMVKRKNSRAKRSGSGATNFSLLKQAGDVEENPGPMTRVSL
LWAVVVSTCLESGMAQTVTQSQPEMSVQEAETVTLSCTYDTSENNYYLFWYKQPPS
RQMILVIRQEAYKQQNATENRFSVNFQKAAKSFSLKISDSQLGDTAMYFCAFMGYS
GAGSYQLTFGKGTKLSVIPDIQNPEPAVYQLKDPRSQDSTLCLFTDFDSQINVPKTME
SGTFITDKCVLDMKAMDSKSNGAIAWSNQTSFTCQDIFKETNATYPSSDVPCDATLT
EKSFETDMNLNFQNLLVIVLRILLLKVAGFNLLMTLRLWSSRAKRSGSGQCTNYALL
KLAGDVESNPGPMGAGATGRAMDGPRLLLLLLLGVSLGGAKEACPTGLYTHSGECC
KACNLGEGVAQPCGANQTVCEPCLDSVTFSDVVSATEPCKPCTECVGLQSMSAPCV
EADDAVCRCAYGYYQDETTGRCEACRVCEAGSGLVFSCQDKQNTVCEECPDGTYS
DEANHVDPCLPCTVCEDTERQLRECTRWADAECEEIPGRWITRSTPPEGSDSTAPSTQ
EPEAPPEQDLIASTVAGVVTTVMGSSQPVVTRGTTDNLIPVYCSILAAVVVGLVAYIA
FKRWNS
-AV6/BV11 TCR with human constant domains (plasmid sequence)
SEQ ID NO: 15
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
GCACCAGACTGCTGTGTTGGGCTGCTCTGTGTCTGCTGGGAGCCGAACTTACTGA
AGCTGGCGTGGCCCAGTCTCCTCGGTACAAGATCATCGAGAAGCGGCAGAGCGT
GGCCTTCTGGTGCAATCCTATCAGCGGACACGCCACACTGTACTGGTATCAGCAG
ATCCTCGGACAGGGCCCCAAGCTGCTGATTCAGTTCCAGAACAACGGCGTGGTG
GACGACAGCCAGCTGCCTAAGGATAGATTCAGCGCCGAGCGGCTGAAAGGCGTG
GACAGCACACTGAAGATCCAGCCTGCCAAGCTGGAAGATAGCGCCGTGTACCTG
TGTGCCAGCTCTCTGGATCCTGGCGATACCGGCGAGCTGTTCTTTGGCGAGGGCA
GCAGACTGACCGTGCTCGAGGACCTGAAGAACGTGTTCCCACCTGAGGTGGCCG
TGTTCGAGCCTTCTGAGGCCGAGATCAGCCACACACAGAAAGCCACACTCGTGT
GTCTGGCCACCGGCTTCTATCCCGATCACGTGGAACTGTCTTGGTGGGTCAACGG
CAAAGAGGTGCACAGCGGCGTCAGCACAGATCCCCAGCCTCTGAAAGAGCAGCC
AGCTCTGAACGACAGCCGGTACTGTCTGAGCAGCAGGCTGAGAGTGTCCGCCAC
CTTCTGGCAGAACCCCAGAAACCACTTCAGATGCCAGGTGCAGTTCTACGGCCTG
AGCGAGAACGATGAGTGGACACAGGATAGAGCCAAGCCTGTGACACAGATCGT
GTCTGCCGAAGCCTGGGGCAGAGCCGATTGTGGCTTTACCAGCGAGAGCTACCA
GCAGGGCGTGCTGTCTGCCACAATCCTGTACGAGATCCTGCTGGGCAAAGCCACT
CTGTACGCCGTGCTGGTGTCTGCCCTGGTGCTGATGGCCATGGTCAAGCGGAAGG
ACTCTAGAGGCAGAGCCAAGAGATCTGGCAGCGGCGCCACAAACTTTAGCCTGC
TGAAACAGGCCGGCGACGTGGAAGAGAACCCCGGACCTATGGAATCCTTTCTCG
GTGGCGTGCTGCTCATCCTGTGGCTGCAAGTGGATTGGGTCAAGAGCCAGAAGA
TCGAGCAGAACAGCGAGGCCCTGAACATCCAAGAGGGCAAGACCGCCACTCTGA
CCTGCAACTACACCAACTACAGCCCCGCCTACCTGCAGTGGTACAGACAGGACC
CTGGAAGAGGCCCTGTGTTCCTGCTGCTGATCCGCGAGAATGAGAAAGAGAAGC
GCAAAGAGCGCCTGAAAGTCACCTTCGACACAACCCTGAAGCAGAGCCTGTTCC
ACATCACCGCCTCTCAGCCAGCCGATAGCGCCACATATCTGTGCGCCCTGGACAT
CTACCCTCACGACATGAGATTCGGAGCCGGCACCAGGCTGACAGTGAAGCCCGA
TATTCAGAACCCCGATCCTGCCGTCTATCAGCTGAGAGACAGCAAGAGCAGCGA
CAAGAGCGTGTGCCTGTTCACCGACTTCGACTCCCAGACCAACGTGTCCCAGAGC
AAGGACAGCGACGTGTACATCACCGACAAGACCGTGCTGGACATGCGGAGCATG
GACTTCAAGAGCAACAGCGCCGTGGCCTGGTCCAACAAGAGCGATTTCGCCTGC
GCCAACGCCTTCAACAACAGCATTATCCCCGAGGACACATTCTTCCCAAGTCCTG
AGAGCAGCTGCGACGTGAAGCTGGTGGAAAAGAGCTTCGAGACAGACACCAAC
CTGAACTTCCAGAACCTGAGCGTGATCGGCTTCCGGATCCTGCTCCTGAAAGTGG
CCGGCTTCAACCTGCTGATGACCCTGAGACTGTGGTCCAGCCGGGCCAAACGATC
TGGCTCTGGCCAGTGTACCAACTATGCCCTGCTCAAGCTCGCTGGCGACGTCGAG
TCTAACCCTGGACCAATGGGAGCTGGCGCTACAGGCAGAGCTATGGATGGACCT
AGACTGCTGCTGCTCCTGCTTCTGGGAGTGTCTCTTGGCGGAGCCAAAGAGGCCT
GTCCTACCGGCCTGTATACACACTCTGGCGAGTGCTGCAAGGCCTGCAATCTTGG
AGAAGGCGTCGCACAGCCTTGCGGCGCTAATCAGACAGTGTGCGAGCCTTGCCT
GGACAGCGTGACCTTTAGCGACGTGGTGTCAGCCACCGAGCCATGCAAGCCATG
TACCGAGTGTGTGGGCCTGCAGAGCATGTCTGCCCCTTGCGTGGAAGCTGACGAT
GCCGTGTGTAGATGCGCCTACGGCTACTACCAGGACGAGACAACAGGCAGATGC
GAGGCCTGTAGAGTGTGCGAAGCTGGATCTGGCCTGGTGTTCAGCTGCCAAGAC
AAGCAGAACACCGTGTGCGAGGAATGCCCCGATGGCACCTATAGCGACGAGGCC
AACCATGTGGATCCCTGCCTGCCTTGTACCGTGTGTGAAGATACCGAGCGGCAGC
TGCGCGAGTGTACAAGATGGGCTGATGCCGAGTGCGAAGAGATCCCTGGCAGAT
GGATCACCAGAAGCACACCTCCAGAGGGCAGCGATAGCACAGCCCCTTCTACAC
AAGAGCCCGAGGCTCCTCCTGAGCAGGACCTGATTGCTTCTACAGTGGCTGGCGT
CGTGACCACCGTGATGGGATCTAGTCAGCCCGTGGTCACCAGAGGCACCACCGA
CAATCTGATCCCCGTGTACTGTAGCATCCTGGCCGCCGTGGTTGTTGGACTGGTG
GCCTATATCGCCTTCAAGCGGTGGAACAGCTGATGATTCGAAGCGGCCGCTTCGA
GCAGACATGATAAGATACATTGATGAGTTTGGACAAACCACAACTAGAATGCAG
TGAAAAAAATGCTTTATTTGTGAAATTTGTGATGCTATTGCTTTATTTGTAACCAT
TATAAGCTGCAATAAACAAGTTACACGGCAGGGTCAGGGTTCTGGATATctgtgggac
aagaggatcagggttaggacatgatctcatttccctctttgccccaacccaggctggagtccagatgccagtgatggacaagggcggg
gctctgtggggctggcaagtcacggtctcatgctttatacgggaaatagcatcttagaaaccagctgctcgtgatggactgggactcag
ggacaggcacaagctatcaatcttggccaagaggccatgatttcagtgaacgttcacggccaggcctggcctgccactcaaggaaac
CACTGGCCGTCGTTTTACAACGTCGTGACTgggaaaaccctggcgttacccaacttaatcgccttgcagca
catccccctttcgccagctggcgtaatagcgaagaggcccgcaccgatcgcccttcccaacagttgcgcagcctgaatggcgaatgg
cgcctgatgcggtattttctccttacgcatctgtgcggtatttcacaccgcatatggtgcactctcagtacaatctgctctgatgccgcatag
ttaagccagccccgacacccgccaacacccgctgacgcgccctgacgggcttgtctgctcccggcatccgcttacagacaagctgtg
accgtctccgggagctgcatgtgtcagaggttttcaccgtcatcaccgaaacgcgcga
-AV6/BV11 TCR with human constant domains (amino acid sequence)
SEQ ID NO: 16
MGTRLLCWAALCLLGAELTEAGVAQSPRYKIIEKRQSVAFWCNPISGHATLYWYQQ
ILGQGPKLLIQFQNNGVVDDSQLPKDRFSAERLKGVDSTLKIQPAKLEDSAVYLCASS
LDPGDTGELFFGEGSRLTVLEDLKNVFPPEVAVFEPSEAEISHTQKATLVCLATGFYP
DHVELSWWVNGKEVHSGVSTDPQPLKEQPALNDSRYCLSSRLRVSATFWQNPRNHF
RCQVQFYGLSENDEWTQDRAKPVTQIVSAEAWGRADCGFTSESYQQGVLSATILYEI
LLGKATLYAVLVSALVLMAMVKRKDSRGRAKRSGSGATNFSLLKQAGDVEENPGP
MESFLGGVLLILWLQVDWVKSQKIEQNSEALNIQEGKTATLTCNYTNYSPAYLQWY
RQDPGRGPVFLLLIRENEKEKRKERLKVTFDTTLKQSLFHITASQPADSATYLCALDI
YPHDMRFGAGTRLTVKPDIQNPDPAVYQLRDSKSSDKSVCLFTDFDSQTNVSQSKDS
DVYITDKTVLDMRSMDFKSNSAVAWSNKSDFACANAFNNSIIPEDTFFPSPESSCDV
KLVEKSFETDTNLNFQNLSVIGFRILLLKVAGFNLLMTLRLWSSRAKRSGSGQCTNY
ALLKLAGDVESNPGPMGAGATGRAMDGPRLLLLLLLGVSLGGAKEACPTGLYTHSG
ECCKACNLGEGVAQPCGANQTVCEPCLDSVTFSDVVSATEPCKPCTECVGLQSMSA
PCVEADDAVCRCAYGYYQDETTGRCEACRVCEAGSGLVFSCQDKQNTVCEECPDG
TYSDEANHVDPCLPCTVCEDTERQLRECTRWADAECEEIPGRWITRSTPPEGSDSTAP
STQEPEAPPEQDLIASTVAGVVTTVMGSSQPVVTRGTTDNLIPVYCSILAAVVVGLVA
YIAFKRWNS
-AV6/BV11 TCR with human constant domains and an engineered disulfide
bond (plasmid sequence)
SEQ ID NO: 17
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
GCACTCGATTGCTCTGTTGGGCCGCGCTGTGCTTGCTGGGTGCCGAATTGACAGA
AGCGGGTGTAGCTCAAAGTCCTCGGTACAAGATTATTGAGAAACGCCAGTCTGTT
GCCTTCTGGTGTAACCCTATCTCTGGTCACGCAACTCTCTATTGGTATCAGCAAAT
CCTGGGTCAAGGTCCTAAACTTCTGATTCAATTTCAGAATAATGGCGTTGTGGAC
GACTCTCAACTGCCTAAAGACAGATTCAGTGCGGAAAGGTTGAAAGGCGTCGAT
AGCACACTCAAAATTCAGCCGGCAAAGCTTGAAGACTCCGCCGTTTATCTGTGCG
CGAGTTCTCTTGATCCTGGGGACACAGGGGAACTGTTCTTTGGCGAGGGATCACG
ATTGACCGTCCTGGAGGACCTGAAGAACGTGTTCCCACCTGAGGTGGCCGTGTTC
GAGCCTTCTGAGGCCGAGATCAGCCACACACAGAAAGCCACACTCGTGTGTCTG
GCCACCGGCTTCTATCCCGATCACGTGGAACTGTCTTGGTGGGTCAACGGCAAAG
AGGTGCACAGCGGCGTCTGTACCGATCCTCAGCCTCTGAAAGAGCAGCCCGCTCT
GAACGACAGCAGATACTGCCTGAGCAGCAGACTGAGAGTGTCCGCCACCTTCTG
GCAGAACCCCAGAAACCACTTCAGATGCCAGGTGCAGTTCTACGGCCTGAGCGA
GAACGATGAGTGGACACAGGATAGAGCCAAGCCAGTGACACAGATCGTGTCTGC
CGAAGCCTGGGGCAGAGCCGATTGTGGCTTTACCAGCGAGAGCTACCAGCAAGG
CGTGCTGTCTGCCACCATCCTGTACGAGATCCTGCTGGGCAAAGCCACTCTGTAC
GCCGTGCTGGTGTCTGCCCTGGTGCTGATGGCCATGGTCAAGCGGAAGGACAGC
AGAGGCAGGGCCAAGAGAAGTGGAAGCGGCGCCACAAACTTCAGCCTGTTGAA
ACAGGCCGGGGACGTCGAAGAGAATCCTGGACCAATGGAATCCTTTCTCGGAGG
TGTACTGCTCATACTCTGGCTGCAAGTGGATTGGGTAAAGAGTCAAAAAATTGAA
CAAAACAGCGAGGCTTTGAATATCCAAGAGGGCAAGACTGCCACGCTGACCTGT
AACTATACAAACTATAGTCCGGCATACCTCCAGTGGTACCGGCAAGATCCTGGTC
GCGGGCCGGTTTTCCTGTTGCTTATTAGAGAGAATGAAAAGGAAAAAAGAAAAG
AACGCTTGAAGGTCACTTTCGACACGACGCTTAAACAGAGTCTTTTCCACATTAC
CGCGTCACAGCCTGCGGACTCAGCTACATACCTGTGTGCTCTCGACATCTATCCC
CACGACATGAGGTTTGGCGCAGGCACTCGACTCACGGTAAAACCCGACATCCAG
AATCCTGATCCTGCCGTGTACCAGCTGCGGGACAGCAAGAGCAGCGATAAGAGC
GTGTGCCTGTTCACCGACTTCGACAGCCAGACCAACGTGTCCCAGAGCAAGGAC
AGCGACGTGTACATCACCGATAAGTGCGTGCTGGACATGCGGAGCATGGACTTC
AAGAGCAACAGCGCCGTGGCCTGGTCCAACAAGAGCGATTTCGCCTGCGCCAAC
GCCTTCAACAACAGCATTATCCCCGAGGACACATTCTTCCCAAGTCCTGAGAGCA
GCTGCGACGTGAAGCTGGTGGAAAAGAGCTTCGAGACAGACACCAACCTGAACT
TCCAGAACCTGAGCGTGATCGGCTTTAGAATCCTGCTGCTGAAGGTGGCCGGCTT
CAACCTGCTGATGACCCTGAGACTGTGGTCCAGCCGGGCCAAACGATCTGGCTCT
GGCCAGTGTACCAACTATGCCCTGCTCAAGCTCGCTGGCGACGTCGAGTCTAACC
CTGGACCAATGGGAGCTGGCGCTACAGGCAGAGCTATGGATGGACCTAGACTGC
TGCTGCTCCTGCTTCTGGGAGTGTCTCTTGGCGGAGCCAAAGAGGCCTGTCCTAC
CGGCCTGTATACACACTCTGGCGAGTGCTGCAAGGCCTGCAATCTTGGAGAAGG
CGTCGCACAGCCTTGCGGCGCTAATCAGACAGTGTGCGAGCCTTGCCTGGACAG
CGTGACCTTTAGCGACGTGGTGTCAGCCACCGAGCCATGCAAGCCATGTACCGA
GTGTGTGGGCCTGCAGAGCATGTCTGCCCCTTGCGTGGAAGCTGACGATGCCGTG
TGTAGATGCGCCTACGGCTACTACCAGGACGAGACAACAGGCAGATGCGAGGCC
TGTAGAGTGTGCGAAGCTGGATCTGGCCTGGTGTTCAGCTGCCAAGACAAGCAG
AACACCGTGTGCGAGGAATGCCCCGATGGCACCTATAGCGACGAGGCCAACCAT
GTGGATCCCTGCCTGCCTTGTACCGTGTGTGAAGATACCGAGCGGCAGCTGCGCG
AGTGTACAAGATGGGCTGATGCCGAGTGCGAAGAGATCCCTGGCAGATGGATCA
CCAGAAGCACACCTCCAGAGGGCAGCGATAGCACAGCCCCTTCTACACAAGAGC
CCGAGGCTCCTCCTGAGCAGGACCTGATTGCTTCTACAGTGGCTGGCGTCGTGAC
CACCGTGATGGGATCTAGTCAGCCCGTGGTCACCAGAGGCACCACCGACAATCT
GATCCCCGTGTACTGTAGCATCCTGGCCGCCGTGGTTGTTGGACTGGTGGCCTAT
ATCGCCTTCAAGCGGTGGAACAGCTGATGATTCGAAGCGGCCGCTTCGAGCAGA
CATGATAAGATACATTGATGAGTTTGGACAAACCACAACTAGAATGCAGTGAAA
AAAATGCTTTATTTGTGAAATTTGTGATGCTATTGCTTTATTTGTAACCATTATAA
GCTGCAATAAACAAGTTACACGGCAGGGTCAGGGTTCTGGATATctgtgggacaagagga
tcagggttaggacatgatctcatttccctctttgccccaacccaggctggagtccagatgccagtgatggacaagggcggggctctgtg
gggctggcaagtcacggtctcatgctttatacgggaaatagcatcttagaaaccagctgctcgtgatggactgggactcagggacagg
cacaagctatcaatcttggccaagaggccatgatttcagtgaacgttcacggccaggcctggcctgccactcaaggaaacCACTG
GCCGTCGTTTTACAACGTCGTGACTgggaaaaccctggcgttacccaacttaatcgccttgcagcacatccccc
tttcgccagctggcgtaatagcgaagaggcccgcaccgatcgcccttcccaacagttgcgcagcctgaatggcgaatggcgcctgat
gcggtattttctccttacgcatctgtgcggtatttcacaccgcatatggtgcactctcagtacaatctgctctgatgccgcatagttaagcca
gccccgacacccgccaacacccgctgacgcgccctgacgggcttgtctgctcccggcatccgcttacagacaagctgtgaccgtctc
cgggagctgcatgtgtcagaggttttcaccgtcatcaccgaaacgcgcga
-AV6/BV11 TCR with human constant domains and an engineered disulfide
bond (amino acid sequence)
SEQ ID NO: 18
MGTRLLCWAALCLLGAELTEAGVAQSPRYKIIEKRQSVAFWCNPISGHATLYWYQQ
ILGQGPKLLIQFQNNGVVDDSQLPKDRFSAERLKGVDSTLKIQPAKLEDSAVYLCASS
LDPGDTGELFFGEGSRLTVLEDLKNVFPPEVAVFEPSEAEISHTQKATLVCLATGFYP
DHVELSWWVNGKEVHSGVCTDPQPLKEQPALNDSRYCLSSRLRVSATFWQNPRNH
FRCQVQFYGLSENDEWTQDRAKPVTQIVSAEAWGRADCGFTSESYQQGVLSATILY
EILLGKATLYAVLVSALVLMAMVKRKDSRGRAKRSGSGATNFSLLKQAGDVEENPG
PMESFLGGVLLILWLQVDWVKSQKIEQNSEALNIQEGKTATLTCNYTNYSPAYLQW
YRQDPGRGPVFLLLIRENEKEKRKERLKVTFDTTLKQSLFHITASQPADSATYLCALD
IYPHDMRFGAGTRLTVKPDIQNPDPAVYQLRDSKSSDKSVCLFTDFDSQTNVSQSKD
SDVYITDKCVLDMRSMDFKSNSAVAWSNKSDFACANAFNNSIIPEDTFFPSPESSCDV
KLVEKSFETDTNLNFQNLSVIGFRILLLKVAGFNLLMTLRLWSSRAKRSGSGQCTNY
ALLKLAGDVESNPGPMGAGATGRAMDGPRLLLLLLLGVSLGGAKEACPTGLYTHSG
ECCKACNLGEGVAQPCGANQTVCEPCLDSVTFSDVVSATEPCKPCTECVGLQSMSA
PCVEADDAVCRCAYGYYQDETTGRCEACRVCEAGSGLVFSCQDKQNTVCEECPDG
TYSDEANHVDPCLPCTVCEDTERQLRECTRWADAECEEIPGRWITRSTPPEGSDSTAP
STQEPEAPPEQDLIASTVAGVVTTVMGSSQPVVTRGTTDNLIPVYCSILAAVVVGLVA
YIAFKRWNS
-AV6/BV11 TCR with human constant domains and an engineered disulfide
bond and stabilizing mutations in the TCRα transmembrane domain (plasmid sequence)
SEQ ID NO: 19
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
GCACTCGATTGCTCTGTTGGGCCGCGCTGTGCTTGCTGGGTGCCGAATTGACAGA
AGCGGGTGTAGCTCAAAGTCCTCGGTACAAGATTATTGAGAAACGCCAGTCTGTT
GCCTTCTGGTGTAACCCTATCTCTGGTCACGCAACTCTCTATTGGTATCAGCAAAT
CCTGGGTCAAGGTCCTAAACTTCTGATTCAATTTCAGAATAATGGCGTTGTGGAC
GACTCTCAACTGCCTAAAGACAGATTCAGTGCGGAAAGGTTGAAAGGCGTCGAT
AGCACACTCAAAATTCAGCCGGCAAAGCTTGAAGACTCCGCCGTTTATCTGTGCG
CGAGTTCTCTTGATCCTGGGGACACAGGGGAACTGTTCTTTGGCGAGGGATCACG
ATTGACCGTCCTGGAGGACCTGAAGAACGTGTTCCCACCTGAGGTGGCCGTGTTC
GAGCCTTCTGAGGCCGAGATCAGCCACACACAGAAAGCCACACTCGTGTGTCTG
GCCACCGGCTTCTATCCCGATCACGTGGAACTGTCTTGGTGGGTCAACGGCAAAG
AGGTGCACAGCGGCGTCTGTACCGATCCTCAGCCTCTGAAAGAGCAGCCCGCTCT
GAACGACAGCAGATACTGCCTGAGCAGCAGACTGAGAGTGTCCGCCACCTTCTG
GCAGAACCCCAGAAACCACTTCAGATGCCAGGTGCAGTTCTACGGCCTGAGCGA
GAACGATGAGTGGACACAGGATAGAGCCAAGCCAGTGACACAGATCGTGTCTGC
CGAAGCCTGGGGCAGAGCCGATTGTGGCTTTACCAGCGAGAGCTACCAGCAAGG
CGTGCTGTCTGCCACCATCCTGTACGAGATCCTGCTGGGCAAAGCCACTCTGTAC
GCCGTGCTGGTGTCTGCCCTGGTGCTGATGGCCATGGTCAAGCGGAAGGACAGC
AGAGGCAGGGCCAAGAGAAGTGGAAGCGGCGCCACAAACTTCAGCCTGTTGAA
ACAGGCCGGGGACGTCGAAGAGAATCCTGGACCAATGGAATCCTTTCTCGGAGG
TGTACTGCTCATACTCTGGCTGCAAGTGGATTGGGTAAAGAGTCAAAAAATTGAA
CAAAACAGCGAGGCTTTGAATATCCAAGAGGGCAAGACTGCCACGCTGACCTGT
AACTATACAAACTATAGTCCGGCATACCTCCAGTGGTACCGGCAAGATCCTGGTC
GCGGGCCGGTTTTCCTGTTGCTTATTAGAGAGAATGAAAAGGAAAAAAGAAAAG
AACGCTTGAAGGTCACTTTCGACACGACGCTTAAACAGAGTCTTTTCCACATTAC
CGCGTCACAGCCTGCGGACTCAGCTACATACCTGTGTGCTCTCGACATCTATCCC
CACGACATGAGGTTTGGCGCAGGCACTCGACTCACGGTAAAACCCGACATCCAG
AATCCTGATCCTGCCGTGTACCAGCTGCGGGACAGCAAGAGCAGCGATAAGAGC
GTGTGCCTGTTCACCGACTTCGACAGCCAGACCAACGTGTCCCAGAGCAAGGAC
AGCGACGTGTACATCACCGATAAGTGCGTGCTGGACATGCGGAGCATGGACTTC
AAGAGCAACAGCGCCGTGGCCTGGTCCAACAAGAGCGATTTCGCCTGCGCCAAC
GCCTTCAACAACAGCATTATCCCCGAGGACACATTCTTCCCAAGTCCTGAGAGCA
GCTGCGACGTGAAGCTGGTGGAAAAGAGCTTCGAGACAGACACCAACCTGAACT
TCCAGAACCTGctggtgatcgtgctgAGAATCCTGCTGCTGAAGGTGGCCGGCTTCAACCT
GCTGATGACCCTGAGACTGTGGTCCAGCCGGGCCAAACGATCTGGCTCTGGCCA
GTGTACCAACTATGCCCTGCTCAAGCTCGCTGGCGACGTCGAGTCTAACCCTGGA
CCAATGGGAGCTGGCGCTACAGGCAGAGCTATGGATGGACCTAGACTGCTGCTG
CTCCTGCTTCTGGGAGTGTCTCTTGGCGGAGCCAAAGAGGCCTGTCCTACCGGCC
TGTATACACACTCTGGCGAGTGCTGCAAGGCCTGCAATCTTGGAGAAGGCGTCG
CACAGCCTTGCGGCGCTAATCAGACAGTGTGCGAGCCTTGCCTGGACAGCGTGA
CCTTTAGCGACGTGGTGTCAGCCACCGAGCCATGCAAGCCATGTACCGAGTGTGT
GGGCCTGCAGAGCATGTCTGCCCCTTGCGTGGAAGCTGACGATGCCGTGTGTAG
ATGCGCCTACGGCTACTACCAGGACGAGACAACAGGCAGATGCGAGGCCTGTAG
AGTGTGCGAAGCTGGATCTGGCCTGGTGTTCAGCTGCCAAGACAAGCAGAACAC
CGTGTGCGAGGAATGCCCCGATGGCACCTATAGCGACGAGGCCAACCATGTGGA
TCCCTGCCTGCCTTGTACCGTGTGTGAAGATACCGAGCGGCAGCTGCGCGAGTGT
ACAAGATGGGCTGATGCCGAGTGCGAAGAGATCCCTGGCAGATGGATCACCAGA
AGCACACCTCCAGAGGGCAGCGATAGCACAGCCCCTTCTACACAAGAGCCCGAG
GCTCCTCCTGAGCAGGACCTGATTGCTTCTACAGTGGCTGGCGTCGTGACCACCG
TGATGGGATCTAGTCAGCCCGTGGTCACCAGAGGCACCACCGACAATCTGATCC
CCGTGTACTGTAGCATCCTGGCCGCCGTGGTTGTTGGACTGGTGGCCTATATCGC
CTTCAAGCGGTGGAACAGCTGATGATTCGAAGCGGCCGCTTCGAGCAGACATGA
TAAGATACATTGATGAGTTTGGACAAACCACAACTAGAATGCAGTGAAAAAAAT
GCTTTATTTGTGAAATTTGTGATGCTATTGCTTTATTTGTAACCATTATAAGCTGC
AATAAACAAGTTACACGGCAGGGTCAGGGTTCTGGATATctgtgggacaagaggatcagggtt
aggacatgatctcatttccctctttgccccaacccaggctggagtccagatgccagtgatggacaagggcggggctctgtggggctgg
caagtcacggtctcatgctttatacgggaaatagcatcttagaaaccagctgctcgtgatggactgggactcagggacaggcacaagc
tatcaatcttggccaagaggccatgatttcagtgaacgttcacggccaggcctggcctgccactcaaggaaacCACTGGCCG
TCGTTTTACAACGTCGTGACTgggaaaaccctggcgttacccaacttaatcgccttgcagcacatccccctttcgcc
agctggcgtaatagcgaagaggcccgcaccgatcgcccttcccaacagttgcgcagcctgaatggcgaatggcgcctgatgcggta
ttttctccttacgcatctgtgcggtatttcacaccgcatatggtgcactctcagtacaatctgctctgatgccgcatagttaagccagcccc
gacacccgccaacacccgctgacgcgccctgacgggcttgtctgctcccggcatccgcttacagacaagctgtgaccgtctccggga
gctgcatgtgtcagaggttttcaccgtcatcaccgaaacgcgcga
-AV6/BV11 TCR with human constant domains and an engineered disulfide
bond and stabilizing mutations in the TCRα transmembrane domain (amino acid sequence)
SEQ ID NO: 20
MGTRLLCWAALCLLGAELTEAGVAQSPRYKIIEKRQSVAFWCNPISGHATLYWYQQ
ILGQGPKLLIQFQNNGVVDDSQLPKDRFSAERLKGVDSTLKIQPAKLEDSAVYLCASS
LDPGDTGELFFGEGSRLTVLEDLKNVFPPEVAVFEPSEAEISHTQKATLVCLATGFYP
DHVELSWWVNGKEVHSGVCTDPQPLKEQPALNDSRYCLSSRLRVSATFWQNPRNH
FRCQVQFYGLSENDEWTQDRAKPVTQIVSAEAWGRADCGFTSESYQQGVLSATILY
EILLGKATLYAVLVSALVLMAMVKRKDSRGRAKRSGSGATNFSLLKQAGDVEENPG
PMESFLGGVLLILWLQVDWVKSQKIEQNSEALNIQEGKTATLTCNYTNYSPAYLQW
YRQDPGRGPVFLLLIRENEKEKRKERLKVTFDTTLKQSLFHITASQPADSATYLCALD
IYPHDMRFGAGTRLTVKPDIQNPDPAVYQLRDSKSSDKSVCLFTDFDSQTNVSQSKD
SDVYITDKCVLDMRSMDFKSNSAVAWSNKSDFACANAFNNSIIPEDTFFPSPESSCDV
KLVEKSFETDTNLNFQNLLVIVLRILLLKVAGFNLLMTLRLWSSRAKRSGSGQCTNY
ALLKLAGDVESNPGPMGAGATGRAMDGPRLLLLLLLGVSLGGAKEACPTGLYTHSG
ECCKACNLGEGVAQPCGANQTVCEPCLDSVTFSDVVSATEPCKPCTECVGLQSMSA
PCVEADDAVCRCAYGYYQDETTGRCEACRVCEAGSGLVFSCQDKQNTVCEECPDG
TYSDEANHVDPCLPCTVCEDTERQLRECTRWADAECEEIPGRWITRSTPPEGSDSTAP
STQEPEAPPEQDLIASTVAGVVTTVMGSSQPVVTRGTTDNLIPVYCSILAAVVVGLVA
YIAFKRWNS
-H2-CD3γ (plasmid sequence)
SEQ ID NO: 21
acgtcaggtggcacttttcggggaaatgtgcgcggaacccctatttgtttatttttctaaatacattcaaatatgtatccgctcatgagacaa
taaccctgataaatgcttcaataatattgaaaaaggaagagtatgagtattcaacatttccgtgtcgcccttattcccttttttgcggcattttg
ccttcctgtttttgctcacccagaaacgctggtgaaagtaaaagatgctgaagatcagttgggtgcacgagtgggttacatcgaactgga
tctcaacagcggtaagatccttgagagttttcgccccgaagaacgttttccaatgatgagcacttttaaagttctgctatgtggcgcggtat
tatcccgtattgacgccgggcaagagcaactcggtcgccgcatacactattctcagaatgacttggttgagtactcaccagtcacagaa
aagcatcttacggatggcatgacagtaagagaattatgcagtgctgccataaccatgagtgataacactgcggccaacttacttctgaca
acgatcggaggaccgaaggagctaaccgcttttttgcacaacatgggggatcatgtaactcgccttgatcgttgggaaccggagctga
atgaagccataccaaacgacgagcgtgacaccacgatgcctgtagcaatggcaacaacgttgcgcaaactattaactggcgaactac
ttactctagcttcccggcaacaattaatagactggatggaggcggataaagttgcaggaccacttctgcgctcggcccttccggctggc
tggtttattgctgataaatctggagccggtgagcgtgggtctcgcggtatcattgcagcactggggccagatggtaagccctcccgtatc
gtagttatctacacgacggggagtcaggcaactatggatgaacgaaatagacagatcgctgagataggtgcctcactgattaagcattg
gtaactgtcagaccaagtttactcatatatactttagattgatttaaaacttcatttttaatttaaaaggatctaggtgaagatcctttttgata
atctcatgaccaaaatcccttaacgtgagttttcgttccactgagcgtcagaccccgtagaaaagatcaaaggatcttcttgagatccttttttt
ctgcgcgtaatctgctgcttgcaaacaaaaaaaccaccgctaccagcggtggtttgtttgccggatcaagagctaccaactctttttccg
aaggtaactggcttcagcagagcgcagataccaaatactgttcttctagtgtagccgtagttaggccaccacttcaagaactctgtagca
ccgcctacatacctcgctctgctaatcctgttaccagtggctgctgccagtggcgataagtcgtgtcttaccgggttggactcaagacga
tagttaccggataaggcgcagcggtcgggctgaacggggggttcgtgcacacagcccagcttggagcgaacgacctacaccgaac
tgagatacctacagcgtgagctatgagaaagcgccacgcttcccgaagggagaaaggcggacaggtatccggtaagcggcagggt
cggaacaggagagcgcacgagggagcttccagggggaaacgcctggtatctttatagtcctgtcgggtttcgccacctctgacttgag
cgtcgatttttgtgatgctcgtcaggggggcggagcctatggaaaaacgccagcaacgcggcctttttacggttcctggccttttgctgg
ccttttgctcacatgttctttcctgcgttatcccctgattctgtggataaccgtattaccgcctttgagtgagctgataccgctcgccgcagc
cgaacgaccgagcgcagcgagtcagtgagcgaggaagcggaagagcgcccaatacgcaaaccgcctctccccgcgcgttggcc
gattcattaatgcagctggcacgacaggtttcccgactggaaagcgggcagtgagcgcaacgcaattaatgtgagttagctcactcatt
aggcaccccaggctttacactttatgcttccggctcgtatgttgtgtggaattgtgagcggataacaatttcacacaggaaacagctatga
ccatgattacgccatatacaggtaagaaactgctaacagcataccagatgatgagatcactttgggtagaatagatgaactaaagagaa
ggattgacagagactaacgtcaggatcccaggtgacgccaatacctactggttcatgggtcacagtttgagatgtgtccctagctacta
gggtcctgcttctcctgtaagagaccagttaaatgcttagctacaggcaagctgcccggaacaggttatgtcaggtcactgatttggttg
acaatagagacatcacctctctatccccgtcatttcattcattctaaactgttcgctcctgtaggactactgttgtaggaagccaaatttccgt
caatattctgagtctgagtatagaaagaagccatttctcctacCTTTGATTGACTGTCGGCTCCGGTGCCCGT
Cagtgggcagagcgcacatcgcccacagtccccgagaagttggggggaggggtcggcaattgaaccggtgcctagagaaggtgg
cgcggggtaaactgggaaagtgatgtcgtgtactggctccgcctttttcccgagggtgggggagaaccgtatataagtgcagtagtcg
ccgtgaacgttctttttcgcaacgggtttgccgccagaacacaggtaagtgccgtgtgtggttcccgcgggcctggcctctttacgggtt
atggcccttgcgtgccttgaattacttccacctggctgcagtacgtgattcttgatcccgagcttcgggttggaagtggggggagagttc
gaggccttgcgcttaaggagccccttcgcctcgtgcttgagttgaggcctggcctgggcgctggggccgccgcgtgcgaatctggtg
gcaccttcgcgcctgtctcgctgctttcgataagtctctagccatttaaaatttttgatgacctgctgcgacgctttttttctggcaagatagtc
ttgtaaatgcgggccaagatctgcacactggtatttcggtttttggggccgcgggcggcgacggggcccgtgcgtcccagcgcacat
gttcggcgaggcggggcctgcgagcgcggccaccgagaatcggacgggggtagtctcaagctggccggcctgctctggtgcctg
gcctcgcgccgccgtgtatcgccccgccctgggcggcaaggctggcccggtcggcaccagttgcgtgagcggaaagatggccgct
tcccggccctgctgcagggagctcaaaatggaggacgcggcgctcgggagagcgggcgggtgagtcacccacacaaaggaaaa
gggcctttccgtcctcagccgtcgcttcatgtgactccacggagtaccgggcgccgtccaggcacctcgattagttctcgagcttttgga
gtacgtcgtctttaggttggggggaggggttttatgcgatggagtttccccacactgagtgggtggagactgaagttaggccagcttgg
cacttgatgtaattctccttggaatttgccctttttgagtttggatcttggttcattctcaagcctcagacagtggttcaaagtttttttCTTC
CATTTCAGGTGTCGTGAtctagaGCCACCATGGCTCTGCCTGTGACAGCTCTGCTGCT
GCCTCTGGCTCTGCTTCTGCATGCCGCCAGACCTGACATCCAGATGACACAGAGC
CCTAGCAGCCTGTCTGCCAGCGTGGGAGACAGAGTGACCATCACCTGTAGAGCC
AGCCAGGACGTGAACACAGCCGTGGCTTGGTATCAGCAGAAGCCTGGCAAGGCC
CCTAAGCTGCTGATCTACAGCGCCTACTTCCTGTACAGCGGCGTGCCCAGCAGAT
TCAGCGGCTCTAGAAGCGGCACCGACTTCACCCTGACCATAAGCAGTCTGCAGC
CCGAGGACTTCGCCACCTACTACTGTCAGCAGTACAGCCGGTACAGCCCCGTGAC
ATTTGGCCAGGGCACCAAGGTGGAAATCAAGAGAACAGGCGGCGGATCTGGCG
GCGGAGGAAGCGGAGGCGGAGCTTCTGAAGTTCAGCTGGTGGAATCTGGCGGAG
GCCTGGTTCAACCTGGCGGATCTCTGAGACTGAGCTGTGCCGCCAGCGGCTTCAA
TGTGTACGCCTCTGGAATGCACTGGGTCCGACAGGCCCCTGGCAAAGGACTTGA
GTGGGTCGCCAAGATCTACCCCGACAGCGACTACACCTACTATGCCGACAGCGT
GAAGGGCAGATTCACCATCAGCGCCGACACCAGCAAGAACACCGCCTACCTGCA
GATGAACAGCCTGAGAGCCGAGGACACCGCCGTGTACTACTGCTCCAGAGACAG
CTCCTTCTACTACGTGTACGCCATGGACTACTGGGGCCAGGGAACACTGGTCACA
GTTTCTAGTCAGAGCATCAAGGGCAATCACCTGGTCAAGGTGTACGACTACCAA
GAGGACGGCAGCGTGCTGCTGACATGTGATGCCGAGGCCAAGAACATCACATGG
TTCAAGGACGGCAAGATGATCGGCTTCCTGACCGAGGACAAGAAGAAGTGGAAC
CTGGGCAGCAACGCCAAGGATCCCAGAGGCATGTATCAGTGCAAGGGCAGCCAG
AACAAGAGCAAGCCCCTGCAGGTCTACTACCGGATGTGCCAGAACTGCATCGAG
CTGAACGCCGCTACCATCTCCGGCTTTCTGTTCGCCGAGATCGTGTCCATCTTCGT
GCTGGCCGTGGGCGTGTACTTTATCGCTGGACAGGATGGCGTCAGACAGAGCAG
AGCCAGCGACAAGCAAACCCTGCTGCCTAACGACCAGCTGTACCAGCCTCTGAA
GGACAGAGAGGACGACCAGTACAGCCATCTGCAGGGCAACCAGCTGCGGAGAA
ACAGAGCCAAGAGATCTGGCAGCGGCGCCACCAATTTCAGCCTGCTGAAACAGG
CTGGCGACGTGGAAGAGAACCCTGGACCTATGGGAGCTGGCGCTACAGGCAGAG
CTATGGATGGACCTAGACTGCTGCTCCTGCTGCTGCTCGGAGTTTCTCTTGGCGG
AGCCAAAGAGGCCTGTCCTACCGGCCTGTATACACACTCTGGCGAGTGCTGCAA
GGCCTGCAATCTTGGAGAAGGCGTGGCACAGCCTTGCGGCGCTAATCAGACAGT
GTGCGAGCCTTGCCTGGACAGCGTGACCTTTAGCGACGTGGTGTCTGCCACCGAG
CCATGCAAGCCTTGTACCGAGTGTGTGGGCCTGCAGAGCATGTCTGCCCCTTGTG
TGGAAGCCGACGATGCCGTGTGTAGATGCGCCTACGGCTACTACCAGGACGAGA
CAACAGGCAGATGCGAGGCCTGTAGAGTGTGTGAAGCCGGCTCTGGACTGGTGT
TCAGCTGCCAAGACAAGCAGAACACCGTGTGCGAGGAATGCCCCGATGGCACCT
ATAGCGACGAGGCCAACCATGTGGATCCCTGCCTGCCTTGTACTGTGTGCGAAGA
TACCGAGCGGCAGCTGCGCGAGTGTACAAGATGGGCTGATGCCGAGTGCGAAGA
GATCCCCGGCAGATGGATCACCAGAAGCACACCTCCAGAGGGCAGCGATAGCAC
AGCCCCTTCTACACAAGAGCCCGAGGCTCCTCCTGAGCAGGATCTGATTGCCTCT
ACAGTGGCCGGCGTGGTCACAACAGTGATGGGATCTTCTCAGCCCGTGGTCACC
AGAGGCACCACCGACAATCTGATCCCCGTGTACTGTAGCATCCTGGCCGCCGTGG
TTGTGGGACTCGTGGCCTATATCGCCTTCAAGCGGTGGAACAGCTGATGATTCGA
AGCGGCCGCTTCGAGCAGACATGATAAGATACATTGATGAGTTTGGACAAACCA
CAACTAGAATGCAGTGAAAAAAATGCTTTATTTGTGAAATTTGTGATGCTATTGC
TTTATTTGTAACCATTATAAGCTGCAATAAACAAGTTGGCCAAAGTACctgtaaagaca
gaagagagatagagtattagctgggcatggagttgttcagatgccctgaagtatttccatggttatttgaggccaagatgaagaaagatc
caaatccatgaaaatggtagtatctatacttagttttccggattgtagactaaatcactcaattgaattcaaggtgataataaacaatttgtct
catctttgtcctggtatcctgggagggtgtttctaaccgttggaatttcatgagtgacaggagtggctttgttatctgtggtggatccttaga
ccatacctgagtttatgcatggcaatgactcaggatggtgctggtcaagaccaaccatatggtaagaggattgcagctttgaaactagtg
gtatcagcccccactggccgtcgttttacaacgtcgtgactgggaaaaccctggcgttacccaacttaatcgccttgcagcacatcccc
ctttcgccagctggcgtaatagcgaagaggcccgcaccgatcgcccttcccaacagttgcgcagcctgaatggtgaatggcgcctga
tgcggtattttctccttacgcatctgtgcggtatttcacaccgcatatggtgcactctcagtacaatctgctctgatgccgcatagttaagcc
agccccgacacccgccaacacccgctgacgcgccctgacgggcttgtctgctcccggcatccgcttacagacaagctgtgaccgtct
ccgggagctgcatgtgtcagaggttttcaccgtcatcaccgaaacgcgcgagacgaaagggcctcgtgatacgcctatttttataggtt
aatgtcatgataataatggtttcttag
-H2-CD3γ (amino acid sequence)
SEQ ID NO: 22
MALPVTALLLPLALLLHAARPDIQMTQSPSSLSASVGDRVTITCRASQDVNTAVAWY
QQKPGKAPKLLIYSAYFLYSGVPSRFSGSRSGTDFTLTISSLQPEDFATYYCQQYSRYS
PVTFGQGTKVEIKRTGGGSGGGGSGGGASEVQLVESGGGLVQPGGSLRLSCAASGF
NVYASGMHWVRQAPGKGLEWVAKIYPDSDYTYYADSVKGRFTISADTSKNTAYLQ
MNSLRAEDTAVYYCSRDSSFYYVYAMDYWGQGTLVTVSSQSIKGNHLVKVYDYQE
DGSVLLTCDAEAKNITWFKDGKMIGFLTEDKKKWNLGSNAKDPRGMYQCKGSQNK
SKPLQVYYRMCQNCIELNAATISGFLFAEIVSIFVLAVGVYFIAGQDGVRQSRASDKQ
TLLPNDQLYQPLKDREDDQYSHLQGNQLRRNRAKRSGSGATNFSLLKQAGDVEENP
GPMGAGATGRAMDGPRLLLLLLLGVSLGGAKEACPTGLYTHSGECCKACNLGEGV
AQPCGANQTVCEPCLDSVTFSDVVSATEPCKPCTECVGLQSMSAPCVEADDAVCRC
AYGYYQDETTGRCEACRVCEAGSGLVFSCQDKQNTVCEECPDGTYSDEANHVDPC
LPCTVCEDTERQLRECTRWADAECEEIPGRWITRSTPPEGSDSTAPSTQEPEAPPEQDL
IASTVAGVVTTVMGSSQPVVTRGTTDNLIPVYCSILAAVVVGLVAYIAFKRWNS
-H2-VHVL-Cα-2A-Cβ (plasmid sequence)
SEQ ID NO: 23
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcggggggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
CTCTGCCTGTGACAGCTCTGCTGCTGCCTCTTGCTCTGCTTCTGCATGCCGCCAGA
CCTGAAGTGCAGCTGGTTGAAAGTGGCGGCGGACTGGTTCAACCTGGCGGATCT
CTGAGACTGAGCTGTGCCGCCAGCGGCTTCAATGTGTATGCCTCTGGCATGCACT
GGGTCCGACAGGCCCCTGGAAAAGGCCTTGAGTGGGTCGCCAAGATCTACCCCG
ACAGCGACTACACCTACTATGCCGACAGCGTGAAGGGCAGATTCACCATCAGCG
CCGACACCAGCAAGAACACCGCCTACCTGCAGATGAACAGCCTGAGAGCCGAGG
ACACCGCCGTGTACTACTGCTCCAGAGACAGCAGCTTCTACTACGTGTACGCCAT
GGACTACTGGGGCCAGGGAACACTGGTCACCGTGTCCAGCAGAACAGGCGGAGG
CTCTGGCGGAGGTGGAAGCGGAGGCGGAGCTTCTGACATCCAGATGACACAGAG
CCCTAGCAGCCTGTCTGCCAGCGTGGGAGACAGAGTGACCATCACCTGTAGAGC
CAGCCAGGACGTGAACACAGCCGTGGCTTGGTATCAGCAGAAGCCTGGCAAGGC
CCCTAAGCTGCTGATCTACAGCGCCTACTTCCTGTACAGCGGCGTGCCCAGCAGA
TTCAGCGGCTCTAGAAGCGGCACCGACTTCACCCTGACCATAAGCAGTCTGCAGC
CCGAGGACTTCGCCACCTACTACTGTCAGCAGTACAGCCGGTACAGCCCCGTGAC
ATTTGGCCAGGGCACCAAGGTGGAAATCAAGGACATTCAGAATCCTGAGCCTGC
CGTGTACCAGCTGAAGGACCCTAGAAGCCAGGACAGCACCCTGTGCCTGTTCAC
CGACTTCGACAGCCAGATCAACGTGCCCAAGACCATGGAAAGCGGCACCTTCAT
CACCGATAAGTGCGTGCTGGACATGAAGGCCATGGACAGCAAGAGCAACGGCGC
CATTGCCTGGTCCAACCAGACCAGCTTCACGTGCCAGGACATCTTCAAAGAGAC
AAACGCCACCTATCCTAGCAGCGACGTGCCCTGTGATGCCACACTGACCGAGAA
GTCCTTCGAGACAGACATGAACCTGAACTTCCAGAACCTGCTGGTCATCGTGCTG
AGAATCCTGCTGCTGAAGGTGGCCGGCTTCAACCTGCTGATGACCCTGAGACTGT
GGTCCAGCAGGGCCAAGAGAAGTGGAAGCGGCGCCACAAACTTCAGCCTGTTGA
AACAGGCCGGGGACGTCGAAGAGAATCCTGGACCAATGGCACTGCCCGTCACTG
CCTTGTTGCTGCCACTTGCACTGCTGCTCCATGCCGCCAGGCCAGAGGACCTGAG
AAACGTGACCCCTCCTAAGGTGTCCCTGTTCGAGCCTAGCAAGGCCGAGATCGCC
AACAAGCAGAAAGCCACACTCGTGTGCCTGGCCAGAGGCTTCTTTCCCGATCAC
GTGGAACTGTCTTGGTGGGTCAACGGCAAAGAGGTGCACAGCGGCGTCTGTACT
GATCCCCAGGCCTACAAAGAGAGCAACTACAGCTACTGCCTGAGCAGCAGACTG
AGAGTGTCCGCCACCTTCTGGCACAACCCCAGAAACCACTTCAGATGCCAGGTG
CAGTTTCACGGCCTGAGCGAAGAGGACAAGTGGCCTGAGGGAAGCCCCAAGCCA
GTGACACAGAATATCTCTGCCGAAGCCTGGGGCAGAGCCGATTGTGGAATTACC
AGCGCCAGCTACCAGCAAGGCGTGCTGTCTGCCACAATCCTGTACGAGATCCTGC
TGGGCAAAGCCACTCTGTACGCCGTGCTGGTGTCTACCCTGGTCGTGATGGCCAT
GGTCAAGCGGAAGAACAGCCGGGCCAAACGATCTGGCTCTGGCCAGTGTACCAA
CTATGCCCTGCTCAAGCTCGCTGGCGACGTCGAGTCTAACCCTGGACCAATGGGA
GCTGGCGCTACAGGCAGAGCTATGGATGGACCTAGACTGCTGCTGCTCCTGCTTC
TGGGAGTGTCTCTTGGCGGAGCCAAAGAGGCCTGTCCTACCGGCCTGTATACACA
CTCTGGCGAGTGCTGCAAGGCCTGCAATCTTGGAGAAGGCGTCGCACAGCCTTG
CGGCGCTAATCAGACAGTGTGCGAGCCTTGCCTGGACAGCGTGACCTTTAGCGA
CGTGGTGTCAGCCACCGAGCCATGCAAGCCATGTACCGAGTGTGTGGGCCTGCA
GAGCATGTCTGCCCCTTGCGTGGAAGCTGACGATGCCGTGTGTAGATGCGCCTAC
GGCTACTACCAGGACGAGACAACAGGCAGATGCGAGGCCTGTAGAGTGTGCGAA
GCTGGATCTGGCCTGGTGTTCAGCTGCCAAGACAAGCAGAACACCGTGTGCGAG
GAATGCCCCGATGGCACCTATAGCGACGAGGCCAACCATGTGGATCCCTGCCTG
CCTTGTACCGTGTGTGAAGATACCGAGCGGCAGCTGCGCGAGTGTACAAGATGG
GCTGATGCCGAGTGCGAAGAGATCCCTGGCAGATGGATCACCAGAAGCACACCT
CCAGAGGGCAGCGATAGCACAGCCCCTTCTACACAAGAGCCCGAGGCTCCTCCT
GAGCAGGACCTGATTGCTTCTACAGTGGCTGGCGTCGTGACCACCGTGATGGGAT
CTAGTCAGCCCGTGGTCACCAGAGGCACCACCGACAATCTGATCCCCGTGTACTG
TAGCATCCTGGCCGCCGTGGTTGTTGGACTGGTGGCCTATATCGCCTTCAAGCGG
TGGAACAGCTGATGATTCGAAGCGGCCGCTTCGAGCAGACATGATAAGATACAT
TGATGAGTTTGGACAAACCACAACTAGAATGCAGTGAAAAAAATGCTTTATTTGT
GAAATTTGTGATGCTATTGCTTTATTTGTAACCATTATAAGCTGCAATAAACAAG
TTACACGGCAGGGTCAGGGTTCTGGATATctgtgggacaagaggatcagggttaggacatgatctcatttc
cctctttgccccaacccaggctggagtccagatgccagtgatggacaagggcggggctctgtggggctggcaagtcacggtctcatg
ctttatacgggaaatagcatcttagaaaccagctgctcgtgatggactgggactcagggacaggcacaagctatcaatcttggccaag
aggccatgatttcagtgaacgttcacggccaggcctggcctgccactcaaggaaacCACTGGCCGTCGTTTTACAA
CGTCGTGACTgggaaaaccctggcgttacccaacttaatcgccttgcagcacatccccctttcgccagctggcgtaatagcga
agaggcccgcaccgatcgcccttcccaacagttgcgcagcctgaatggcgaatggcgcctgatgcggtattttctccttacgcatctgt
gcggtatttcacaccgcatatggtgcactctcagtacaatctgctctgatgccgcatagttaagccagccccgacacccgccaacaccc
gctgacgcgccctgacgggcttgtctgctcccggcatccgcttacagacaagctgtgaccgtctccgggagctgcatgtgtcagaggt
tttcaccgtcatcaccgaaacgcgcga
-H2-VHVL-Cα-2A-Cβ (amino acid sequence)
SEQ ID NO: 24
MALPVTALLLPLALLLHAARPEVQLVESGGGLVQPGGSLRLSCAASGFNVYASGMH
WVRQAPGKGLEWVAKIYPDSDYTYYADSVKGRFTISADTSKNTAYLQMNSLRAED
TAVYYCSRDSSFYYVYAMDYWGQGTLVTVSSRTGGGSGGGGSGGGASDIQMTQSP
SSLSASVGDRVTITCRASQDVNTAVAWYQQKPGKAPKLLIYSAYFLYSGVPSRFSGS
RSGTDFTLTISSLQPEDFATYYCQQYSRYSPVTFGQGTKVEIKDIQNPEPAVYQLKDP
RSQDSTLCLFTDFDSQINVPKTMESGTFITDKCVLDMKAMDSKSNGAIAWSNQTSFT
CQDIFKETNATYPSSDVPCDATLTEKSFETDMNLNFQNLLVIVLRILLLKVAGFNLLM
TLRLWSSRAKRSGSGATNFSLLKQAGDVEENPGPMALPVTALLLPLALLLHAARPED
LRNVTPPKVSLFEPSKAELANKQKATLVCLARGFFPDHVELSWWVNGKEVHSGVCT
DPQAYKESNYSYCLSSRLRVSATFWHNPRNHFRCQVQFHGLSEEDKWPEGSPKPVT
QNISAEAWGRADCGITSASYQQGVLSATILYEILLGKATLYAVLVSTLVVMAMVKR
KNSRAKRSGSGQCTNYALLKLAGDVESNPGPMGAGATGRAMDGPRLLLLLLLGVS
LGGAKEACPTGLYTHSGECCKACNLGEGVAQPCGANQTVCEPCLDSVTFSDVVSAT
EPCKPCTECVGLQSMSAPCVEADDAVCRCAYGYYQDETTGRCEACRVCEAGSGLVF
SCQDKQNTVCEECPDGTYSDEANHVDPCLPCTVCEDTERQLRECTRWADAECEEIP
GRWITRSTPPEGSDSTAPSTQEPEAPPEQDLIASTVAGVVTTVMGSSQPVVTRGTTDN
LIPVYCSILAAVVVGLVAYIAFKRWNS
-H2-VHVL-Cβ-2A-Cα (plasmid sequence)
SEQ ID NO: 25
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
CTCTGCCTGTGACAGCTCTGCTGCTGCCTCTTGCTCTGCTTCTGCATGCCGCCAGA
CCTGAAGTGCAGCTGGTTGAAAGTGGCGGCGGACTGGTTCAACCTGGCGGATCT
CTGAGACTGAGCTGTGCCGCCAGCGGCTTCAATGTGTATGCCTCTGGCATGCACT
GGGTCCGACAGGCCCCTGGAAAAGGCCTTGAGTGGGTCGCCAAGATCTACCCCG
ACAGCGACTACACCTACTATGCCGACAGCGTGAAGGGCAGATTCACCATCAGCG
CCGACACCAGCAAGAACACCGCCTACCTGCAGATGAACAGCCTGAGAGCCGAGG
ACACCGCCGTGTACTACTGCTCCAGAGACAGCAGCTTCTACTACGTGTACGCCAT
GGACTACTGGGGCCAGGGAACACTGGTCACCGTGTCCAGCAGAACAGGCGGAGG
CTCTGGCGGAGGTGGAAGCGGAGGCGGAGCTTCTGACATCCAGATGACACAGAG
CCCTAGCAGCCTGTCTGCCAGCGTGGGAGACAGAGTGACCATCACCTGTAGAGC
CAGCCAGGACGTGAACACAGCCGTGGCTTGGTATCAGCAGAAGCCTGGCAAGGC
CCCTAAGCTGCTGATCTACAGCGCCTACTTCCTGTACAGCGGCGTGCCCAGCAGA
TTCAGCGGCTCTAGAAGCGGCACCGACTTCACCCTGACCATAAGCAGTCTGCAGC
CCGAGGACTTCGCCACCTACTACTGTCAGCAGTACAGCCGGTACAGCCCCGTGAC
ATTTGGCCAGGGCACCAAGGTGGAAATCAAGGAGGACCTGAGAAACGTGACCCC
TCCTAAGGTGTCCCTGTTCGAGCCTAGCAAGGCCGAGATCGCCAACAAGCAGAA
AGCCACACTCGTGTGCCTGGCCAGAGGCTTCTTTCCCGATCACGTGGAACTGTCT
TGGTGGGTCAACGGCAAAGAGGTGCACAGCGGCGTCTGTACTGATCCCCAGGCC
TACAAAGAGAGCAACTACAGCTACTGCCTGAGCAGCAGACTGAGAGTGTCCGCC
ACCTTCTGGCACAACCCCAGAAACCACTTCAGATGCCAGGTGCAGTTTCACGGCC
TGAGCGAAGAGGACAAGTGGCCTGAGGGAAGCCCCAAGCCAGTGACACAGAAT
ATCTCTGCCGAAGCCTGGGGCAGAGCCGATTGTGGAATTACCAGCGCCAGCTAC
CAGCAAGGCGTGCTGTCTGCCACAATCCTGTACGAGATCCTGCTGGGCAAAGCC
ACTCTGTACGCCGTGCTGGTGTCTACCCTGGTCGTGATGGCCATGGTCAAGCGGA
AGAACAGCAGGGCCAAGAGAAGTGGAAGCGGCGCCACAAACTTCAGCCTGTTG
AAACAGGCCGGGGACGTCGAAGAGAATCCTGGACCAATGGCACTGCCCGTCACT
GCCTTGTTGCTGCCACTTGCACTGCTGCTCCATGCCGCCAGGCCAGACATTCAGA
ATCCTGAGCCTGCCGTGTACCAGCTGAAGGACCCTAGAAGCCAGGACAGCACCC
TGTGCCTGTTCACCGACTTCGACAGCCAGATCAACGTGCCCAAGACCATGGAAA
GCGGCACCTTCATCACCGATAAGTGCGTGCTGGACATGAAGGCCATGGACAGCA
AGAGCAACGGCGCCATTGCCTGGTCCAACCAGACCAGCTTCACGTGCCAGGACA
TCTTCAAAGAGACAAACGCCACCTATCCTAGCAGCGACGTGCCCTGTGATGCCAC
ACTGACCGAGAAGTCCTTCGAGACAGACATGAACCTGAACTTCCAGAACCTGCT
GGTCATCGTGCTGAGAATCCTGCTGCTGAAGGTGGCCGGCTTCAACCTGCTGATG
ACCCTGAGACTGTGGTCCAGCCGGGCCAAACGATCTGGCTCTGGCCAGTGTACC
AACTATGCCCTGCTCAAGCTCGCTGGCGACGTCGAGTCTAACCCTGGACCAATGG
GAGCTGGCGCTACAGGCAGAGCTATGGATGGACCTAGACTGCTGCTGCTCCTGCT
TCTGGGAGTGTCTCTTGGCGGAGCCAAAGAGGCCTGTCCTACCGGCCTGTATACA
CACTCTGGCGAGTGCTGCAAGGCCTGCAATCTTGGAGAAGGCGTCGCACAGCCT
TGCGGCGCTAATCAGACAGTGTGCGAGCCTTGCCTGGACAGCGTGACCTTTAGCG
ACGTGGTGTCAGCCACCGAGCCATGCAAGCCATGTACCGAGTGTGTGGGCCTGC
AGAGCATGTCTGCCCCTTGCGTGGAAGCTGACGATGCCGTGTGTAGATGCGCCTA
CGGCTACTACCAGGACGAGACAACAGGCAGATGCGAGGCCTGTAGAGTGTGCGA
AGCTGGATCTGGCCTGGTGTTCAGCTGCCAAGACAAGCAGAACACCGTGTGCGA
GGAATGCCCCGATGGCACCTATAGCGACGAGGCCAACCATGTGGATCCCTGCCT
GCCTTGTACCGTGTGTGAAGATACCGAGCGGCAGCTGCGCGAGTGTACAAGATG
GGCTGATGCCGAGTGCGAAGAGATCCCTGGCAGATGGATCACCAGAAGCACACC
TCCAGAGGGCAGCGATAGCACAGCCCCTTCTACACAAGAGCCCGAGGCTCCTCC
TGAGCAGGACCTGATTGCTTCTACAGTGGCTGGCGTCGTGACCACCGTGATGGGA
TCTAGTCAGCCCGTGGTCACCAGAGGCACCACCGACAATCTGATCCCCGTGTACT
GTAGCATCCTGGCCGCCGTGGTTGTTGGACTGGTGGCCTATATCGCCTTCAAGCG
GTGGAACAGCTGATGATTCGAAGCGGCCGCTTCGAGCAGACATGATAAGATACA
TTGATGAGTTTGGACAAACCACAACTAGAATGCAGTGAAAAAAATGCTTTATTTG
TGAAATTTGTGATGCTATTGCTTTATTTGTAACCATTATAAGCTGCAATAAACAA
GTTACACGGCAGGGTCAGGGTTCTGGATATctgtgggacaagaggatcagggttaggacatgatctcattt
ccctctttgccccaacccaggctggagtccagatgccagtgatggacaagggcggggctctgtggggctggcaagtcacggtctcat
gctttatacgggaaatagcatcttagaaaccagctgctcgtgatggactgggactcagggacaggcacaagctatcaatcttggccaa
gaggccatgatttcagtgaacgttcacggccaggcctggcctgccactcaaggaaacCACTGGCCGTCGTTTTACA
ACGTCGTGACTgggaaaaccctggcgttacccaacttaatcgccttgcagcacatccccctttcgccagctggcgtaatagc
gaagaggcccgcaccgatcgcccttcccaacagttgcgcagcctgaatggcgaatggcgcctgatgcggtattttctccttacgcatct
gtgcggtatttcacaccgcatatggtgcactctcagtacaatctgctctgatgccgcatagttaagccagccccgacacccgccaacac
ccgctgacgcgccctgacgggcttgtctgctcccggcatccgcttacagacaagctgtgaccgtctccgggagctgcatgtgtcagag
gttttcaccgtcatcaccgaaacgcgcga
-H2-VHVL-Cβ-2A-Cα (amino acid sequence)
SEQ ID NO: 26
MALPVTALLLPLALLLHAARPEVQLVESGGGLVQPGGSLRLSCAASGFNVYASGMH
WVRQAPGKGLEWVAKIYPDSDYTYYADSVKGRFTISADTSKNTAYLQMNSLRAED
TAVYYCSRDSSFYYVYAMDYWGQGTLVTVSSRTGGGSGGGGSGGGASDIQMTQSP
SSLSASVGDRVTITCRASQDVNTAVAWYQQKPGKAPKLLIYSAYFLYSGVPSRFSGS
RSGTDFTLTISSLQPEDFATYYCQQYSRYSPVTFGQGTKVEIKEDLRNVTPPKVSLFEP
SKAEIANKQKATLVCLARGFFPDHVELSWWVNGKEVHSGVCTDPQAYKESNYSYC
LSSRLRVSATFWHNPRNHFRCQVQFHGLSEEDKWPEGSPKPVTQNISAEAWGRADC
GITSASYQQGVLSATILYEILLGKATLYAVLVSTLVVMAMVKRKNSRAKRSGSGATN
FSLLKQAGDVEENPGPMALPVTALLLPLALLLHAARPDIQNPEPAVYQLKDPRSQDS
TLCLFTDFDSQINVPKTMESGTFITDKCVLDMKAMDSKSNGAIAWSNQTSFTCQDIF
KETNATYPSSDVPCDATLTEKSFETDMNLNFQNLLVIVLRILLLKVAGFNLLMTLRL
WSSRAKRSGSGQCTNYALLKLAGDVESNPGPMGAGATGRAMDGPRLLLLLLLGVS
LGGAKEACPTGLYTHSGECCKACNLGEGVAQPCGANQTVCEPCLDSVTFSDVVSAT
EPCKPCTECVGLQSMSAPCVEADDAVCRCAYGYYQDETTGRCEACRVCEAGSGLVF
SCQDKQNTVCEECPDGTYSDEANHVDPCLPCTVCEDTERQLRECTRWADAECEEIP
GRWITRSTPPEGSDSTAPSTQEPEAPPEQDLIASTVAGVVTTVMGSSQPVVTRGTTDN
LIPVYCSILAAVVVGLVAYIAFKRWNS
-H2-VLVH-Cα-2A-Cβ (plasmid sequence)
SEQ ID NO: 27
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcggggggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
CTCTGCCTGTGACAGCTCTGCTGCTGCCTCTTGCTCTGCTTCTGCATGCCGCCAGA
CCTGACATCCAGATGACACAGAGCCCTAGCAGCCTGTCTGCCAGCGTGGGAGAC
AGAGTGACCATCACCTGTAGAGCCAGCCAGGACGTGAACACAGCCGTGGCTTGG
TATCAGCAGAAGCCTGGCAAGGCCCCTAAGCTGCTGATCTACAGCGCCTACTTCC
TGTACAGCGGCGTGCCCAGCAGATTCAGCGGCTCTAGAAGCGGCACCGACTTCA
CCCTGACCATAAGCAGTCTGCAGCCCGAGGACTTCGCCACCTACTACTGTCAGCA
GTACAGCCGGTACAGCCCCGTGACATTTGGCCAGGGCACCAAGGTGGAAATCAA
GAGAACAGGCGGAGGCTCTGGCGGAGGTGGAAGCGGAGGCGGAGCTTCTGAAG
TGCAGCTGGTTGAAAGTGGCGGCGGACTGGTTCAACCTGGCGGATCTCTGAGAC
TGAGCTGTGCCGCCAGCGGCTTCAATGTGTATGCCTCTGGCATGCACTGGGTCCG
ACAGGCCCCTGGAAAAGGCCTTGAGTGGGTCGCCAAGATCTACCCCGACAGCGA
CTACACCTACTATGCCGACAGCGTGAAGGGCAGATTCACCATCAGCGCCGACAC
CAGCAAGAACACCGCCTACCTGCAGATGAACAGCCTGAGAGCCGAGGACACCGC
CGTGTACTACTGCTCCAGAGACAGCAGCTTCTACTACGTGTACGCCATGGACTAC
TGGGGCCAGGGAACACTGGTCACCGTGTCCAGCGACATTCAGAATCCTGAGCCT
GCCGTGTACCAGCTGAAGGACCCTAGAAGCCAGGACAGCACCCTGTGCCTGTTC
ACCGACTTCGACAGCCAGATCAACGTGCCCAAGACCATGGAAAGCGGCACCTTC
ATCACCGATAAGTGCGTGCTGGACATGAAGGCCATGGACAGCAAGAGCAACGGC
GCCATTGCCTGGTCCAACCAGACCAGCTTCACGTGCCAGGACATCTTCAAAGAG
ACAAACGCCACCTATCCTAGCAGCGACGTGCCCTGTGATGCCACACTGACCGAG
AAGTCCTTCGAGACAGACATGAACCTGAACTTCCAGAACCTGCTGGTCATCGTGC
TGAGAATCCTGCTGCTGAAGGTGGCCGGCTTCAACCTGCTGATGACCCTGAGACT
GTGGTCCAGCAGGGCCAAGAGAAGTGGAAGCGGCGCCACAAACTTCAGCCTGTT
GAAACAGGCCGGGGACGTCGAAGAGAATCCTGGACCAATGGCACTGCCCGTCAC
TGCCTTGTTGCTGCCACTTGCACTGCTGCTCCATGCCGCCAGGCCAGAGGACCTG
AGAAACGTGACCCCTCCTAAGGTGTCCCTGTTCGAGCCTAGCAAGGCCGAGATC
GCCAACAAGCAGAAAGCCACACTCGTGTGCCTGGCCAGAGGCTTCTTTCCCGATC
ACGTGGAACTGTCTTGGTGGGTCAACGGCAAAGAGGTGCACAGCGGCGTCTGTA
CTGATCCCCAGGCCTACAAAGAGAGCAACTACAGCTACTGCCTGAGCAGCAGAC
TGAGAGTGTCCGCCACCTTCTGGCACAACCCCAGAAACCACTTCAGATGCCAGGT
GCAGTTTCACGGCCTGAGCGAAGAGGACAAGTGGCCTGAGGGAAGCCCCAAGCC
AGTGACACAGAATATCTCTGCCGAAGCCTGGGGCAGAGCCGATTGTGGAATTAC
CAGCGCCAGCTACCAGCAAGGCGTGCTGTCTGCCACAATCCTGTACGAGATCCTG
CTGGGCAAAGCCACTCTGTACGCCGTGCTGGTGTCTACCCTGGTCGTGATGGCCA
TGGTCAAGCGGAAGAACAGCCGGGCCAAACGATCTGGCTCTGGCCAGTGTACCA
ACTATGCCCTGCTCAAGCTCGCTGGCGACGTCGAGTCTAACCCTGGACCAATGGG
AGCTGGCGCTACAGGCAGAGCTATGGATGGACCTAGACTGCTGCTGCTCCTGCTT
CTGGGAGTGTCTCTTGGCGGAGCCAAAGAGGCCTGTCCTACCGGCCTGTATACAC
ACTCTGGCGAGTGCTGCAAGGCCTGCAATCTTGGAGAAGGCGTCGCACAGCCTT
GCGGCGCTAATCAGACAGTGTGCGAGCCTTGCCTGGACAGCGTGACCTTTAGCG
ACGTGGTGTCAGCCACCGAGCCATGCAAGCCATGTACCGAGTGTGTGGGCCTGC
AGAGCATGTCTGCCCCTTGCGTGGAAGCTGACGATGCCGTGTGTAGATGCGCCTA
CGGCTACTACCAGGACGAGACAACAGGCAGATGCGAGGCCTGTAGAGTGTGCGA
AGCTGGATCTGGCCTGGTGTTCAGCTGCCAAGACAAGCAGAACACCGTGTGCGA
GGAATGCCCCGATGGCACCTATAGCGACGAGGCCAACCATGTGGATCCCTGCCT
GCCTTGTACCGTGTGTGAAGATACCGAGCGGCAGCTGCGCGAGTGTACAAGATG
GGCTGATGCCGAGTGCGAAGAGATCCCTGGCAGATGGATCACCAGAAGCACACC
TCCAGAGGGCAGCGATAGCACAGCCCCTTCTACACAAGAGCCCGAGGCTCCTCC
TGAGCAGGACCTGATTGCTTCTACAGTGGCTGGCGTCGTGACCACCGTGATGGGA
TCTAGTCAGCCCGTGGTCACCAGAGGCACCACCGACAATCTGATCCCCGTGTACT
GTAGCATCCTGGCCGCCGTGGTTGTTGGACTGGTGGCCTATATCGCCTTCAAGCG
GTGGAACAGCTGATGATTCGAAGCGGCCGCTTCGAGCAGACATGATAAGATACA
TTGATGAGTTTGGACAAACCACAACTAGAATGCAGTGAAAAAAATGCTTTATTTG
TGAAATTTGTGATGCTATTGCTTTATTTGTAACCATTATAAGCTGCAATAAACAA
GTTACACGGCAGGGTCAGGGTTCTGGATATctgtgggacaagaggatcagggttaggacatgatctcattt
ccctctttgccccaacccaggctggagtccagatgccagtgatggacaagggcggggctctgtggggctggcaagtcacggtctcat
gctttatacgggaaatagcatcttagaaaccagctgctcgtgatggactgggactcagggacaggcacaagctatcaatcttggccaa
gaggccatgatttcagtgaacgttcacggccaggcctggcctgccactcaaggaaacCACTGGCCGTCGTTTTACA
ACGTCGTGACTgggaaaaccctggcgttacccaacttaatcgccttgcagcacatccccctttcgccagctggcgtaatagc
gaagaggcccgcaccgatcgcccttcccaacagttgcgcagcctgaatggcgaatggcgcctgatgcggtattttctccttacgcatct
gtgcggtatttcacaccgcatatggtgcactctcagtacaatctgctctgatgccgcatagttaagccagccccgacacccgccaacac
ccgctgacgcgccctgacgggcttgtctgctcccggcatccgcttacagacaagctgtgaccgtctccgggagctgcatgtgtcagag
gttttcaccgtcatcaccgaaacgcgcga
-H2-VLVH-Cα-2A-Cβ (amino acid sequence)
SEQ ID NO: 28
MALPVTALLLPLALLLHAARPDIQMTQSPSSLSASVGDRVTITCRASQDVNTAVAWY
QQKPGKAPKLLIYSAYFLYSGVPSRFSGSRSGTDFTLTISSLQPEDFATYYCQQYSRYS
PVTFGQGTKVEIKRTGGGSGGGGSGGGASEVQLVESGGGLVQPGGSLRLSCAASGF
NVYASGMHWVRQAPGKGLEWVAKIYPDSDYTYYADSVKGRFTISADTSKNTAYLQ
MNSLRAEDTAVYYCSRDSSFYYVYAMDYWGQGTLVTVSSDIQNPEPAVYQLKDPR
SQDSTLCLFTDFDSQINVPKTMESGTFITDKCVLDMKAMDSKSNGAIAWSNQTSFTC
QDIFKETNATYPSSDVPCDATLTEKSFETDMNLNFQNLLVIVLRILLLKVAGFNLLMT
LRLWSSRAKRSGSGATNFSLLKQAGDVEENPGPMALPVTALLLPLALLLHAARPEDL
RNVTPPKVSLFEPSKAEIANKQKATLVCLARGFFPDHVELSWWVNGKEVHSGVCTD
PQAYKESNYSYCLSSRLRVSATFWHNPRNHFRCQVQFHGLSEEDKWPEGSPKPVTQ
NISAEAWGRADCGITSASYQQGVLSATILYEILLGKATLYAVLVSTLVVMAMVKRK
NSRAKRSGSGQCTNYALLKLAGDVESNPGPMGAGATGRAMDGPRLLLLLLLGVSL
GGAKEACPTGLYTHSGECCKACNLGEGVAQPCGANQTVCEPCLDSVTFSDVVSATE
PCKPCTECVGLQSMSAPCVEADDAVCRCAYGYYQDETTGRCEACRVCEAGSGLVFS
CQDKQNTVCEECPDGTYSDEANHVDPCLPCTVCEDTERQLRECTRWADAECEEIPG
RWITRSTPPEGSDSTAPSTQEPEAPPEQDLIASTVAGVVTTVMGSSQPVVTRGTTDNLI
PVYCSILAAVVVGLVAYIAFKRWNS
-H2-VLVH-Cβ-2A-Cα (plasmid sequence)
SEQ ID NO: 29
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcggggggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
CTCTGCCTGTGACAGCTCTGCTGCTGCCTCTTGCTCTGCTTCTGCATGCCGCCAGA
CCTGACATCCAGATGACACAGAGCCCTAGCAGCCTGTCTGCCAGCGTGGGAGAC
AGAGTGACCATCACCTGTAGAGCCAGCCAGGACGTGAACACAGCCGTGGCTTGG
TATCAGCAGAAGCCTGGCAAGGCCCCTAAGCTGCTGATCTACAGCGCCTACTTCC
TGTACAGCGGCGTGCCCAGCAGATTCAGCGGCTCTAGAAGCGGCACCGACTTCA
CCCTGACCATAAGCAGTCTGCAGCCCGAGGACTTCGCCACCTACTACTGTCAGCA
GTACAGCCGGTACAGCCCCGTGACATTTGGCCAGGGCACCAAGGTGGAAATCAA
GAGAACAGGCGGAGGCTCTGGCGGAGGTGGAAGCGGAGGCGGAGCTTCTGAAG
TGCAGCTGGTTGAAAGTGGCGGCGGACTGGTTCAACCTGGCGGATCTCTGAGAC
TGAGCTGTGCCGCCAGCGGCTTCAATGTGTATGCCTCTGGCATGCACTGGGTCCG
ACAGGCCCCTGGAAAAGGCCTTGAGTGGGTCGCCAAGATCTACCCCGACAGCGA
CTACACCTACTATGCCGACAGCGTGAAGGGCAGATTCACCATCAGCGCCGACAC
CAGCAAGAACACCGCCTACCTGCAGATGAACAGCCTGAGAGCCGAGGACACCGC
CGTGTACTACTGCTCCAGAGACAGCAGCTTCTACTACGTGTACGCCATGGACTAC
TGGGGCCAGGGAACACTGGTCACCGTGTCCAGCGAGGACCTGAGAAACGTGACC
CCTCCTAAGGTGTCCCTGTTCGAGCCTAGCAAGGCCGAGATCGCCAACAAGCAG
AAAGCCACACTCGTGTGCCTGGCCAGAGGCTTCTTTCCCGATCACGTGGAACTGT
CTTGGTGGGTCAACGGCAAAGAGGTGCACAGCGGCGTCTGTACTGATCCCCAGG
CCTACAAAGAGAGCAACTACAGCTACTGCCTGAGCAGCAGACTGAGAGTGTCCG
CCACCTTCTGGCACAACCCCAGAAACCACTTCAGATGCCAGGTGCAGTTTCACGG
CCTGAGCGAAGAGGACAAGTGGCCTGAGGGAAGCCCCAAGCCAGTGACACAGA
ATATCTCTGCCGAAGCCTGGGGCAGAGCCGATTGTGGAATTACCAGCGCCAGCT
ACCAGCAAGGCGTGCTGTCTGCCACAATCCTGTACGAGATCCTGCTGGGCAAAG
CCACTCTGTACGCCGTGCTGGTGTCTACCCTGGTCGTGATGGCCATGGTCAAGCG
GAAGAACAGCAGGGCCAAGAGAAGTGGAAGCGGCGCCACAAACTTCAGCCTGT
TGAAACAGGCCGGGGACGTCGAAGAGAATCCTGGACCAATGGCACTGCCCGTCA
CTGCCTTGTTGCTGCCACTTGCACTGCTGCTCCATGCCGCCAGGCCAGACATTCA
GAATCCTGAGCCTGCCGTGTACCAGCTGAAGGACCCTAGAAGCCAGGACAGCAC
CCTGTGCCTGTTCACCGACTTCGACAGCCAGATCAACGTGCCCAAGACCATGGAA
AGCGGCACCTTCATCACCGATAAGTGCGTGCTGGACATGAAGGCCATGGACAGC
AAGAGCAACGGCGCCATTGCCTGGTCCAACCAGACCAGCTTCACGTGCCAGGAC
ATCTTCAAAGAGACAAACGCCACCTATCCTAGCAGCGACGTGCCCTGTGATGCC
ACACTGACCGAGAAGTCCTTCGAGACAGACATGAACCTGAACTTCCAGAACCTG
CTGGTCATCGTGCTGAGAATCCTGCTGCTGAAGGTGGCCGGCTTCAACCTGCTGA
TGACCCTGAGACTGTGGTCCAGCCGGGCCAAACGATCTGGCTCTGGCCAGTGTAC
CAACTATGCCCTGCTCAAGCTCGCTGGCGACGTCGAGTCTAACCCTGGACCAATG
GGAGCTGGCGCTACAGGCAGAGCTATGGATGGACCTAGACTGCTGCTGCTCCTG
CTTCTGGGAGTGTCTCTTGGCGGAGCCAAAGAGGCCTGTCCTACCGGCCTGTATA
CACACTCTGGCGAGTGCTGCAAGGCCTGCAATCTTGGAGAAGGCGTCGCACAGC
CTTGCGGCGCTAATCAGACAGTGTGCGAGCCTTGCCTGGACAGCGTGACCTTTAG
CGACGTGGTGTCAGCCACCGAGCCATGCAAGCCATGTACCGAGTGTGTGGGCCT
GCAGAGCATGTCTGCCCCTTGCGTGGAAGCTGACGATGCCGTGTGTAGATGCGCC
TACGGCTACTACCAGGACGAGACAACAGGCAGATGCGAGGCCTGTAGAGTGTGC
GAAGCTGGATCTGGCCTGGTGTTCAGCTGCCAAGACAAGCAGAACACCGTGTGC
GAGGAATGCCCCGATGGCACCTATAGCGACGAGGCCAACCATGTGGATCCCTGC
CTGCCTTGTACCGTGTGTGAAGATACCGAGCGGCAGCTGCGCGAGTGTACAAGA
TGGGCTGATGCCGAGTGCGAAGAGATCCCTGGCAGATGGATCACCAGAAGCACA
CCTCCAGAGGGCAGCGATAGCACAGCCCCTTCTACACAAGAGCCCGAGGCTCCT
CCTGAGCAGGACCTGATTGCTTCTACAGTGGCTGGCGTCGTGACCACCGTGATGG
GATCTAGTCAGCCCGTGGTCACCAGAGGCACCACCGACAATCTGATCCCCGTGTA
CTGTAGCATCCTGGCCGCCGTGGTTGTTGGACTGGTGGCCTATATCGCCTTCAAG
CGGTGGAACAGCTGATGATTCGAAGCGGCCGCTTCGAGCAGACATGATAAGATA
CATTGATGAGTTTGGACAAACCACAACTAGAATGCAGTGAAAAAAATGCTTTATT
TGTGAAATTTGTGATGCTATTGCTTTATTTGTAACCATTATAAGCTGCAATAAACA
AGTTACACGGCAGGGTCAGGGTTCTGGATATctgtgggacaagaggatcagggttaggacatgatctca
tttccctctttgccccaacccaggctggagtccagatgccagtgatggacaagggcggggctctgtggggctggcaagtcacggtctc
atgctttatacgggaaatagcatcttagaaaccagctgctcgtgatggactgggactcagggacaggcacaagctatcaatcttggcca
agaggccatgatttcagtgaacgttcacggccaggcctggcctgccactcaaggaaacCACTGGCCGTCGTTTTAC
AACGTCGTGACTgggaaaaccctggcgttacccaacttaatcgccttgcagcacatccccctttcgccagctggcgtaatag
cgaagaggcccgcaccgatcgcccttcccaacagttgcgcagcctgaatggcgaatggcgcctgatgcggtattttctccttacgcat
ctgtgcggtatttcacaccgcatatggtgcactctcagtacaatctgctctgatgccgcatagttaagccagccccgacacccgccaac
acccgctgacgcgccctgacgggcttgtctgctcccggcatccgcttacagacaagctgtgaccgtctccgggagctgcatgtgtcag
aggttttcaccgtcatcaccgaaacgcgcga
-H2-VLVH-Cβ-2A-Cα (amino acid sequence)
SEQ ID NO: 30
MALPVTALLLPLALLLHAARPDIQMTQSPSSLSASVGDRVTITCRASQDVNTAVAWY
QQKPGKAPKLLIYSAYFLYSGVPSRFSGSRSGTDFTLTISSLQPEDFATYYCQQYSRYS
PVTFGQGTKVEIKRTGGGSGGGGSGGGASEVQLVESGGGLVQPGGSLRLSCAASGF
NVYASGMHWVRQAPGKGLEWVAKIYPDSDYTYYADSVKGRFTISADTSKNTAYLQ
MNSLRAEDTAVYYCSRDSSFYYVYAMDYWGQGTLVTVSSEDLRNVTPPKVSLFEPS
KAEIANKQKATLVCLARGFFPDHVELSWWVNGKEVHSGVCTDPQAYKESNYSYCL
SSRLRVSATFWHNPRNHFRCQVQFHGLSEEDKWPEGSPKPVTQNISAEAWGRADCG
ITSASYQQGVLSATILYEILLGKATLYAVLVSTLVVMAMVKRKNSRAKRSGSGATNF
SLLKQAGDVEENPGPMALPVTALLLPLALLLHAARPDIQNPEPAVYQLKDPRSQDST
LCLFTDFDSQINVPKTMESGTFITDKCVLDMKAMDSKSNGAIAWSNQTSFTCQDIFK
ETNATYPSSDVPCDATLTEKSFETDMNLNFQNLLVIVLRILLLKVAGFNLLMTLRLW
SSRAKRSGSGQCTNYALLKLAGDVESNPGPMGAGATGRAMDGPRLLLLLLLGVSLG
GAKEACPTGLYTHSGECCKACNLGEGVAQPCGANQTVCEPCLDSVTFSDVVSATEP
CKPCTECVGLQSMSAPCVEADDAVCRCAYGYYQDETTGRCEACRVCEAGSGLVFS
CQDKQNTVCEECPDGTYSDEANHVDPCLPCTVCEDTERQLRECTRWADAECEEIPG
RWITRSTPPEGSDSTAPSTQEPEAPPEQDLIASTVAGVVTTVMGSSQPVVTRGTTDNLI
PVYCSILAAVVVGLVAYIAFKRWNS
-H2-VHVL-A6-TCRα-2A-A6-TCRβ (plasmid sequence)
SEQ ID NO: 31
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGT
CTATCGGCCTGCTGTGTTGTGCCGCTCTGTCTCTGCTTTGGGCCGGACCTGTTAAT
GCCGGCGTGACCCAGACACCTAAGTTCCAGGTGCTGAAAACCGGCCAGAGCATG
ACCCTGCAGTGCGCCCAGGATATGAACCACGAGTACATGAGCTGGTACAGACAG
GACCCTGGCATGGGCCTGAGACTGATCCACTATTCTGTCGGAGCCGGCATCACCG
ACCAGGGCGAAGTTCCTAATGGCTACAACGTGTCCAGAAGCACCACCGAGGACT
TCCCACTGAGACTGCTGTCTGCCGCTCCTAGCCAGACCAGCGTGTACTTCTGTGC
CTCTAGACCTGGACTGGCTGGCGGCAGACCTGAGCAGTATTTTGGCCCTGGCACC
AGACTGACCGTGACCGAGGACCTGAGAAACGTGACCCCTCCTAAGGTGTCCCTG
TTCGAGCCTAGCAAGGCCGAGATCGCCAACAAGCAGAAAGCCACACTCGTGTGC
CTGGCCAGAGGCTTCTTTCCCGATCACGTGGAACTGTCTTGGTGGGTCAACGGCA
AAGAGGTGCACAGCGGCGTCTGTACTGATCCCCAGGCCTACAAAGAGAGCAACT
ACAGCTACTGCCTGAGCAGCAGACTGAGAGTGTCCGCCACCTTCTGGCACAACC
CCAGAAACCACTTCAGATGCCAGGTGCAGTTTCACGGCCTGAGCGAAGAGGACA
AGTGGCCTGAGGGAAGCCCCAAGCCAGTGACACAGAATATCTCTGCCGAAGCCT
GGGGCAGAGCCGATTGTGGAATTACCAGCGCCAGCTACCAGCAAGGCGTGCTGT
CTGCCACAATCCTGTACGAGATCCTGCTGGGCAAAGCCACTCTGTACGCCGTGCT
GGTGTCTACCCTGGTCGTGATGGCCATGGTCAAGCGGAAGAACAGCAGGGCCAA
GAGAAGTGGAAGCGGCGCCACAAACTTCAGCCTGTTGAAACAGGCCGGGGACGT
CGAAGAGAATCCTGGACCAATGGCTCTGCCTGTGACAGCTCTGCTGCTGCCTCTT
GCTCTGCTTCTGCATGCCGCCAGACCTGAAGTGCAGCTGGTTGAAAGTGGCGGCG
GACTGGTTCAACCTGGCGGATCTCTGAGACTGAGCTGTGCCGCCAGCGGCTTCAA
TGTGTATGCCTCTGGCATGCACTGGGTCCGACAGGCCCCTGGAAAAGGCCTTGAG
TGGGTCGCCAAGATCTACCCCGACAGCGACTACACCTACTATGCCGACAGCGTG
AAGGGCAGATTCACCATCAGCGCCGACACCAGCAAGAACACCGCCTACCTGCAG
ATGAACAGCCTGAGAGCCGAGGACACCGCCGTGTACTACTGCTCCAGAGACAGC
AGCTTCTACTACGTGTACGCCATGGACTACTGGGGCCAGGGAACACTGGTCACC
GTGTCCAGCAGAACAGGCGGAGGCTCTGGCGGAGGTGGAAGCGGAGGCGGAGC
TTCTGACATCCAGATGACACAGAGCCCTAGCAGCCTGTCTGCCAGCGTGGGAGA
CAGAGTGACCATCACCTGTAGAGCCAGCCAGGACGTGAACACAGCCGTGGCTTG
GTATCAGCAGAAGCCTGGCAAGGCCCCTAAGCTGCTGATCTACAGCGCCTACTTC
CTGTACAGCGGCGTGCCCAGCAGATTCAGCGGCTCTAGAAGCGGCACCGACTTC
ACCCTGACCATAAGCAGTCTGCAGCCCGAGGACTTCGCCACCTACTACTGTCAGC
AGTACAGCCGGTACAGCCCCGTGACATTTGGCCAGGGCACCAAGGTGGAAATCA
AGCAGAAAGAGGTGGAACAGAACAGCGGCCCTCTGTCTGTTCCTGAAGGCGCTA
TCGCCAGCCTGAACTGCACCTACAGCGATAGAGGCAGCCAGAGCTTCTTCTGGTA
CAGACAGTACAGCGGCAAGAGCCCCGAGCTGATCATGAGCATCTACAGCAACGG
CGACAAAGAGGACGGCCGGTTTACAGCCCAGCTGAACAAGGCCAGCCAGTACGT
GTCCCTGCTGATCAGAGATAGCCAGCCTAGCGACAGCGCCACCTATCTGTGTGCC
GTGACCACAGATAGCTGGGGCAAGCTGCAGTTTGGCGCCGGAACACAGGTGGTG
GTTACACCTGACATTCAGAATCCTGAGCCTGCCGTGTACCAGCTGAAGGACCCTA
GAAGCCAGGACAGCACCCTGTGCCTGTTCACCGACTTCGACAGCCAGATCAACG
TGCCCAAGACCATGGAAAGCGGCACCTTCATCACCGATAAGTGCGTGCTGGACA
TGAAGGCCATGGACAGCAAGAGCAACGGCGCCATTGCCTGGTCCAACCAGACCA
GCTTCACGTGCCAGGACATCTTCAAAGAGACAAACGCCACCTATCCTAGCAGCG
ACGTGCCCTGTGATGCCACACTGACCGAGAAGTCCTTCGAGACAGACATGAACC
TGAACTTCCAGAACCTGCTGGTCATCGTGCTGAGAATCCTGCTGCTGAAGGTGGC
CGGCTTCAACCTGCTGATGACCCTGAGACTGTGGTCCAGCCGGGCCAAACGATCT
GGCTCTGGCCAGTGTACCAACTATGCCCTGCTCAAGCTCGCTGGCGACGTCGAGT
CTAACCCTGGACCAATGGGAGCTGGCGCTACAGGCAGAGCTATGGATGGACCTA
GACTGCTGCTGCTCCTGCTTCTGGGAGTGTCTCTTGGCGGAGCCAAAGAGGCCTG
TCCTACCGGCCTGTATACACACTCTGGCGAGTGCTGCAAGGCCTGCAATCTTGGA
GAAGGCGTCGCACAGCCTTGCGGCGCTAATCAGACAGTGTGCGAGCCTTGCCTG
GACAGCGTGACCTTTAGCGACGTGGTGTCAGCCACCGAGCCATGCAAGCCATGT
ACCGAGTGTGTGGGCCTGCAGAGCATGTCTGCCCCTTGCGTGGAAGCTGACGAT
GCCGTGTGTAGATGCGCCTACGGCTACTACCAGGACGAGACAACAGGCAGATGC
GAGGCCTGTAGAGTGTGCGAAGCTGGATCTGGCCTGGTGTTCAGCTGCCAAGAC
AAGCAGAACACCGTGTGCGAGGAATGCCCCGATGGCACCTATAGCGACGAGGCC
AACCATGTGGATCCCTGCCTGCCTTGTACCGTGTGTGAAGATACCGAGCGGCAGC
TGCGCGAGTGTACAAGATGGGCTGATGCCGAGTGCGAAGAGATCCCTGGCAGAT
GGATCACCAGAAGCACACCTCCAGAGGGCAGCGATAGCACAGCCCCTTCTACAC
AAGAGCCCGAGGCTCCTCCTGAGCAGGACCTGATTGCTTCTACAGTGGCTGGCGT
CGTGACCACCGTGATGGGATCTAGTCAGCCCGTGGTCACCAGAGGCACCACCGA
CAATCTGATCCCCGTGTACTGTAGCATCCTGGCCGCCGTGGTTGTTGGACTGGTG
GCCTATATCGCCTTCAAGCGGTGGAACAGCTGATGATTCGAAGCGGCCGCTTCGA
GCAGACATGATAAGATACATTGATGAGTTTGGACAAACCACAACTAGAATGCAG
TGAAAAAAATGCTTTATTTGTGAAATTTGTGATGCTATTGCTTTATTTGTAACCAT
TATAAGCTGCAATAAACAAGTTACACGGCAGGGTCAGGGTTCTGGATATctgtgggac
aagaggatcagggttaggacatgatctcatttccctctttgccccaacccaggctggagtccagatgccagtgatggacaagggcggg
gctctgtggggctggcaagtcacggtctcatgctttatacgggaaatagcatcttagaaaccagctgctcgtgatggactgggactcag
ggacaggcacaagctatcaatcttggccaagaggccatgatttcagtgaacgttcacggccaggcctggcctgccactcaaggaaac
CACTGGCCGTCGTTTTACAACGTCGTGACTgggaaaaccctggcgttacccaacttaatcgccttgcagca
catccccctttcgccagctggcgtaatagcgaagaggcccgcaccgatcgcccttcccaacagttgcgcagcctgaatggcgaatgg
cgcctgatgcggtattttctccttacgcatctgtgcggtatttcacaccgcatatggtgcactctcagtacaatctgctctgatgccgcatag
ttaagccagccccgacacccgccaacacccgctgacgcgccctgacgggcttgtctgctcccggcatccgcttacagacaagctgtg
accgtctccgggagctgcatgtgtcagaggttttcaccgtcatcaccgaaacgcgcga
-H2-VHVL-A6-TCRα-2A-A6-TCRβ (amino acid sequence)
SEQ ID NO: 32
MSIGLLCCAALSLLWAGPVNAGVTQTPKFQVLKTGQSMTLQCAQDMNHEYMSWY
RQDPGMGLRLIHYSVGAGITDQGEVPNGYNVSRSTTEDFPLRLLSAAPSQTSVYFCA
SRPGLAGGRPEQYFGPGTRLTVTEDLRNVTPPKVSLFEPSKAEIANKQKATLVCLAR
GFFPDHVELSWWVNGKEVHSGVCTDPQAYKESNYSYCLSSRLRVSATFWHNPRNH
FRCQVQFHGLSEEDKWPEGSPKPVTQNISAEAWGRADCGITSASYQQGVLSATILYEI
LLGKATLYAVLVSTLVVMAMVKRKNSRAKRSGSGATNFSLLKQAGDVEENPGPMA
LPVTALLLPLALLLHAARPEVQLVESGGGLVQPGGSLRLSCAASGFNVYASGMHWV
RQAPGKGLEWVAKIYPDSDYTYYADSVKGRFTISADTSKNTAYLQMNSLRAEDTAV
YYCSRDSSFYYVYAMDYWGQGTLVTVSSRTGGGSGGGGSGGGASDIQMTQSPSSLS
ASVGDRVTITCRASQDVNTAVAWYQQKPGKAPKLLIYSAYFLYSGVPSRFSGSRSGT
DFTLTISSLQPEDFATYYCQQYSRYSPVTFGQGTKVEIKQKEVEQNSGPLSVPEGAIAS
LNCTYSDRGSQSFFWYRQYSGKSPELIMSIYSNGDKEDGRFTAQLNKASQYVSLLIR
DSQPSDSATYLCAVTTDSWGKLQFGAGTQVVVTPDIQNPEPAVYQLKDPRSQDSTL
CLFTDFDSQINVPKTMESGTFITDKCVLDMKAMDSKSNGAIAWSNQTSFTCQDIFKE
TNATYPSSDVPCDATLTEKSFETDMNLNFQNLLVIVLRILLLKVAGFNLLMTLRLWS
SRAKRSGSGQCTNYALLKLAGDVESNPGPMGAGATGRAMDGPRLLLLLLLGVSLG
GAKEACPTGLYTHSGECCKACNLGEGVAQPCGANQTVCEPCLDSVTFSDVVSATEP
CKPCTECVGLQSMSAPCVEADDAVCRCAYGYYQDETTGRCEACRVCEAGSGLVFS
CQDKQNTVCEECPDGTYSDEANHVDPCLPCTVCEDTERQLRECTRWADAECEEIPG
RWITRSTPPEGSDSTAPSTQEPEAPPEQDLIASTVAGVVTTVMGSSQPVVTRGTTDNLI
PVYCSILAAVVVGLVAYIAFKRWNS
-H2-VHVL-A6-TCRβ-2A-A6-TCRα (plasmid sequence)
SEQ ID NO: 33
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccccccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcggggggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
CTCTGCCTGTGACAGCTCTGCTGCTGCCTCTTGCTCTGCTTCTGCATGCCGCCAGA
CCTGAAGTGCAGCTGGTTGAAAGTGGCGGCGGACTGGTTCAACCTGGCGGATCT
CTGAGACTGAGCTGTGCCGCCAGCGGCTTCAATGTGTATGCCTCTGGCATGCACT
GGGTCCGACAGGCCCCTGGAAAAGGCCTTGAGTGGGTCGCCAAGATCTACCCCG
ACAGCGACTACACCTACTATGCCGACAGCGTGAAGGGCAGATTCACCATCAGCG
CCGACACCAGCAAGAACACCGCCTACCTGCAGATGAACAGCCTGAGAGCCGAGG
ACACCGCCGTGTACTACTGCTCCAGAGACAGCAGCTTCTACTACGTGTACGCCAT
GGACTACTGGGGCCAGGGAACACTGGTCACCGTGTCCAGCAGAACAGGCGGAGG
CTCTGGCGGAGGTGGAAGCGGAGGCGGAGCTTCTGACATCCAGATGACACAGAG
CCCTAGCAGCCTGTCTGCCAGCGTGGGAGACAGAGTGACCATCACCTGTAGAGC
CAGCCAGGACGTGAACACAGCCGTGGCTTGGTATCAGCAGAAGCCTGGCAAGGC
CCCTAAGCTGCTGATCTACAGCGCCTACTTCCTGTACAGCGGCGTGCCCAGCAGA
TTCAGCGGCTCTAGAAGCGGCACCGACTTCACCCTGACCATAAGCAGTCTGCAGC
CCGAGGACTTCGCCACCTACTACTGTCAGCAGTACAGCCGGTACAGCCCCGTGAC
ATTTGGCCAGGGCACCAAGGTGGAAATCAAGAATGCCGGCGTGACCCAGACACC
TAAGTTCCAGGTGCTGAAAACCGGCCAGAGCATGACCCTGCAGTGCGCCCAGGA
TATGAACCACGAGTACATGAGCTGGTACAGACAGGACCCTGGCATGGGCCTGAG
ACTGATCCACTATTCTGTCGGAGCCGGCATCACCGACCAGGGCGAAGTTCCTAAT
GGCTACAACGTGTCCAGAAGCACCACCGAGGACTTCCCACTGAGACTGCTGTCT
GCCGCTCCTAGCCAGACCAGCGTGTACTTCTGTGCCTCTAGACCTGGACTGGCTG
GCGGCAGACCTGAGCAGTATTTTGGCCCTGGCACCAGACTGACCGTGACCGAGG
ACCTGAGAAACGTGACCCCTCCTAAGGTGTCCCTGTTCGAGCCTAGCAAGGCCG
AGATCGCCAACAAGCAGAAAGCCACACTCGTGTGCCTGGCCAGAGGCTTCTTTC
CCGATCACGTGGAACTGTCTTGGTGGGTCAACGGCAAAGAGGTGCACAGCGGCG
TCTGTACTGATCCCCAGGCCTACAAAGAGAGCAACTACAGCTACTGCCTGAGCA
GCAGACTGAGAGTGTCCGCCACCTTCTGGCACAACCCCAGAAACCACTTCAGAT
GCCAGGTGCAGTTTCACGGCCTGAGCGAAGAGGACAAGTGGCCTGAGGGAAGCC
CCAAGCCAGTGACACAGAATATCTCTGCCGAAGCCTGGGGCAGAGCCGATTGTG
GAATTACCAGCGCCAGCTACCAGCAAGGCGTGCTGTCTGCCACAATCCTGTACG
AGATCCTGCTGGGCAAAGCCACTCTGTACGCCGTGCTGGTGTCTACCCTGGTCGT
GATGGCCATGGTCAAGCGGAAGAACAGCAGGGCCAAGAGAAGTGGAAGCGGCG
CCACAAACTTCAGCCTGTTGAAACAGGCCGGGGACGTCGAAGAGAATCCTGGAC
CAATGAAGTCCCTGAGAGTGCTGCTGGTCATCCTGTGGCTGCAGCTGTCTTGGGT
CTGGTCCCAGCAGAAAGAGGTGGAACAGAACAGCGGCCCTCTGTCTGTTCCTGA
AGGCGCTATCGCCAGCCTGAACTGCACCTACAGCGATAGAGGCAGCCAGAGCTT
CTTCTGGTACAGACAGTACAGCGGCAAGAGCCCCGAGCTGATCATGAGCATCTA
CAGCAACGGCGACAAAGAGGACGGCCGGTTTACAGCCCAGCTGAACAAGGCCA
GCCAGTACGTGTCCCTGCTGATCAGAGATAGCCAGCCTAGCGACAGCGCCACCT
ATCTGTGTGCCGTGACCACAGATAGCTGGGGCAAGCTGCAGTTTGGCGCCGGAA
CACAGGTGGTGGTTACACCTGACATTCAGAATCCTGAGCCTGCCGTGTACCAGCT
GAAGGACCCTAGAAGCCAGGACAGCACCCTGTGCCTGTTCACCGACTTCGACAG
CCAGATCAACGTGCCCAAGACCATGGAAAGCGGCACCTTCATCACCGATAAGTG
CGTGCTGGACATGAAGGCCATGGACAGCAAGAGCAACGGCGCCATTGCCTGGTC
CAACCAGACCAGCTTCACGTGCCAGGACATCTTCAAAGAGACAAACGCCACCTA
TCCTAGCAGCGACGTGCCCTGTGATGCCACACTGACCGAGAAGTCCTTCGAGAC
AGACATGAACCTGAACTTCCAGAACCTGCTGGTCATCGTGCTGAGAATCCTGCTG
CTGAAGGTGGCCGGCTTCAACCTGCTGATGACCCTGAGACTGTGGTCCAGCCGG
GCCAAACGATCTGGCTCTGGCCAGTGTACCAACTATGCCCTGCTCAAGCTCGCTG
GCGACGTCGAGTCTAACCCTGGACCAATGGGAGCTGGCGCTACAGGCAGAGCTA
TGGATGGACCTAGACTGCTGCTGCTCCTGCTTCTGGGAGTGTCTCTTGGCGGAGC
CAAAGAGGCCTGTCCTACCGGCCTGTATACACACTCTGGCGAGTGCTGCAAGGC
CTGCAATCTTGGAGAAGGCGTCGCACAGCCTTGCGGCGCTAATCAGACAGTGTG
CGAGCCTTGCCTGGACAGCGTGACCTTTAGCGACGTGGTGTCAGCCACCGAGCC
ATGCAAGCCATGTACCGAGTGTGTGGGCCTGCAGAGCATGTCTGCCCCTTGCGTG
GAAGCTGACGATGCCGTGTGTAGATGCGCCTACGGCTACTACCAGGACGAGACA
ACAGGCAGATGCGAGGCCTGTAGAGTGTGCGAAGCTGGATCTGGCCTGGTGTTC
AGCTGCCAAGACAAGCAGAACACCGTGTGCGAGGAATGCCCCGATGGCACCTAT
AGCGACGAGGCCAACCATGTGGATCCCTGCCTGCCTTGTACCGTGTGTGAAGATA
CCGAGCGGCAGCTGCGCGAGTGTACAAGATGGGCTGATGCCGAGTGCGAAGAGA
TCCCTGGCAGATGGATCACCAGAAGCACACCTCCAGAGGGCAGCGATAGCACAG
CCCCTTCTACACAAGAGCCCGAGGCTCCTCCTGAGCAGGACCTGATTGCTTCTAC
AGTGGCTGGCGTCGTGACCACCGTGATGGGATCTAGTCAGCCCGTGGTCACCAG
AGGCACCACCGACAATCTGATCCCCGTGTACTGTAGCATCCTGGCCGCCGTGGTT
GTTGGACTGGTGGCCTATATCGCCTTCAAGCGGTGGAACAGCTGATGATTCGAAG
CGGCCGCTTCGAGCAGACATGATAAGATACATTGATGAGTTTGGACAAACCACA
ACTAGAATGCAGTGAAAAAAATGCTTTATTTGTGAAATTTGTGATGCTATTGCTT
TATTTGTAACCATTATAAGCTGCAATAAACAAGTTACACGGCAGGGTCAGGGTTC
TGGATATctgtgggacaagaggatcagggttaggacatgatctcatttccctctttgccccaacccaggctggagtccagatgcc
agtgatggacaagggcggggctctgtggggctggcaagtcacggtctcatgctttatacgggaaatagcatcttagaaaccagctgct
cgtgatggactgggactcagggacaggcacaagctatcaatcttggccaagaggccatgatttcagtgaacgttcacggccaggcct
ggcctgccactcaaggaaacCACTGGCCGTCGTTTTACAACGTCGTGACTgggaaaaccctggcgttac
ccaacttaatcgccttgcagcacatccccctttcgccagctggcgtaatagcgaagaggcccgcaccgatcgcccttcccaacagttg
cgcagcctgaatggcgaatggcgcctgatgcggtattttctccttacgcatctgtgcggtatttcacaccgcatatggtgcactctcagta
caatctgctctgatgccgcatagttaagccagccccgacacccgccaacacccgctgacgcgccctgacgggcttgtctgctcccgg
catccgcttacagacaagctgtgaccgtctccgggagctgcatgtgtcagaggttttcaccgtcatcaccgaaacgcgcga
-H2-VHVL-A6-TCRβ-2A-A6-TCRα (amino acid sequence)
SEQ ID NO: 34
MALPVTALLLPLALLLHAARPEVQLVESGGGLVQPGGSLRLSCAASGFNVYASGMH
WVRQAPGKGLEWVAKIYPDSDYTYYADSVKGRFTISADTSKNTAYLQMNSLRAED
TAVYYCSRDSSFYYVYAMDYWGQGTLVTVSSRTGGGSGGGGSGGGASDIQMTQSP
SSLSASVGDRVTITCRASQDVNTAVAWYQQKPGKAPKLLIYSAYFLYSGVPSRFSGS
RSGTDFTLTISSLQPEDFATYYCQQYSRYSPVTFGQGTKVEIKNAGVTQTPKFQVLKT
GQSMTLQCAQDMNHEYMSWYRQDPGMGLRLIHYSVGAGITDQGEVPNGYNVSRST
TEDFPLRLLSAAPSQTSVYFCASRPGLAGGRPEQYFGPGTRLTVTEDLRNVTPPKVSL
FEPSKAEIANKQKATLVCLARGFFPDHVELSWWVNGKEVHSGVCTDPQAYKESNYS
YCLSSRLRVSATFWHNPRNHFRCQVQFHGLSEEDKWPEGSPKPVTQNISAEAWGRA
DCGITSASYQQGVLSATILYEILLGKATLYAVLVSTLVVMAMVKRKNSRAKRSGSG
ATNFSLLKQAGDVEENPGPMKSLRVLLVILWLQLSWVWSQQKEVEQNSGPLSVPEG
AIASLNCTYSDRGSQSFFWYRQYSGKSPELIMSIYSNGDKEDGRFTAQLNKASQYVS
LLIRDSQPSDSATYLCAVTTDSWGKLQFGAGTQVVVTPDIQNPEPAVYQLKDPRSQD
STLCLFTDFDSQINVPKTMESGTFITDKCVLDMKAMDSKSNGAIAWSNQTSFTCQDIF
KETNATYPSSDVPCDATLTEKSFETDMNLNFQNLLVIVLRILLLKVAGENLLMTLRL
WSSRAKRSGSGQCTNYALLKLAGDVESNPGPMGAGATGRAMDGPRLLLLLLLGVS
LGGAKEACPTGLYTHSGECCKACNLGEGVAQPCGANQTVCEPCLDSVTFSDVVSAT
EPCKPCTECVGLQSMSAPCVEADDAVCRCAYGYYQDETTGRCEACRVCEAGSGLVF
SCQDKQNTVCEECPDGTYSDEANHVDPCLPCTVCEDTERQLRECTRWADAECEEIP
GRWITRSTPPEGSDSTAPSTQEPEAPPEQDLIASTVAGVVTTVMGSSQPVVTRGTTDN
LIPVYCSILAAVVVGLVAYIAFKRWNS
-H2-VLVH-A6-TCRα-2A-A6-TCRβ (plasmid sequence)
SEQ ID NO: 35
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgagggcgggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcggggggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGT
CTATCGGCCTGCTGTGTTGTGCCGCTCTGTCTCTGCTTTGGGCCGGACCTGTTAAT
GCCGGCGTGACCCAGACACCTAAGTTCCAGGTGCTGAAAACCGGCCAGAGCATG
ACCCTGCAGTGCGCCCAGGATATGAACCACGAGTACATGAGCTGGTACAGACAG
GACCCTGGCATGGGCCTGAGACTGATCCACTATTCTGTCGGAGCCGGCATCACCG
ACCAGGGCGAAGTTCCTAATGGCTACAACGTGTCCAGAAGCACCACCGAGGACT
TCCCACTGAGACTGCTGTCTGCCGCTCCTAGCCAGACCAGCGTGTACTTCTGTGC
CTCTAGACCTGGACTGGCTGGCGGCAGACCTGAGCAGTATTTTGGCCCTGGCACC
AGACTGACCGTGACCGAGGACCTGAGAAACGTGACCCCTCCTAAGGTGTCCCTG
TTCGAGCCTAGCAAGGCCGAGATCGCCAACAAGCAGAAAGCCACACTCGTGTGC
CTGGCCAGAGGCTTCTTTCCCGATCACGTGGAACTGTCTTGGTGGGTCAACGGCA
AAGAGGTGCACAGCGGCGTCTGTACTGATCCCCAGGCCTACAAAGAGAGCAACT
ACAGCTACTGCCTGAGCAGCAGACTGAGAGTGTCCGCCACCTTCTGGCACAACC
CCAGAAACCACTTCAGATGCCAGGTGCAGTTTCACGGCCTGAGCGAAGAGGACA
AGTGGCCTGAGGGAAGCCCCAAGCCAGTGACACAGAATATCTCTGCCGAAGCCT
GGGGCAGAGCCGATTGTGGAATTACCAGCGCCAGCTACCAGCAAGGCGTGCTGT
CTGCCACAATCCTGTACGAGATCCTGCTGGGCAAAGCCACTCTGTACGCCGTGCT
GGTGTCTACCCTGGTCGTGATGGCCATGGTCAAGCGGAAGAACAGCAGGGCCAA
GAGAAGTGGAAGCGGCGCCACAAACTTCAGCCTGTTGAAACAGGCCGGGGACGT
CGAAGAGAATCCTGGACCAATGGCTCTGCCTGTGACAGCTCTGCTGCTGCCTCTT
GCTCTGCTTCTGCATGCCGCCAGACCTGACATCCAGATGACACAGAGCCCTAGCA
GCCTGTCTGCCAGCGTGGGAGACAGAGTGACCATCACCTGTAGAGCCAGCCAGG
ACGTGAACACAGCCGTGGCTTGGTATCAGCAGAAGCCTGGCAAGGCCCCTAAGC
TGCTGATCTACAGCGCCTACTTCCTGTACAGCGGCGTGCCCAGCAGATTCAGCGG
CTCTAGAAGCGGCACCGACTTCACCCTGACCATAAGCAGTCTGCAGCCCGAGGA
CTTCGCCACCTACTACTGTCAGCAGTACAGCCGGTACAGCCCCGTGACATTTGGC
CAGGGCACCAAGGTGGAAATCAAGAGAACAGGCGGAGGCTCTGGCGGAGGTGG
AAGCGGAGGCGGAGCTTCTGAAGTGCAGCTGGTTGAAAGTGGCGGCGGACTGGT
TCAACCTGGCGGATCTCTGAGACTGAGCTGTGCCGCCAGCGGCTTCAATGTGTAT
GCCTCTGGCATGCACTGGGTCCGACAGGCCCCTGGAAAAGGCCTTGAGTGGGTC
GCCAAGATCTACCCCGACAGCGACTACACCTACTATGCCGACAGCGTGAAGGGC
AGATTCACCATCAGCGCCGACACCAGCAAGAACACCGCCTACCTGCAGATGAAC
AGCCTGAGAGCCGAGGACACCGCCGTGTACTACTGCTCCAGAGACAGCAGCTTC
TACTACGTGTACGCCATGGACTACTGGGGCCAGGGAACACTGGTCACCGTGTCC
AGCCAGAAAGAGGTGGAACAGAACAGCGGCCCTCTGTCTGTTCCTGAAGGCGCT
ATCGCCAGCCTGAACTGCACCTACAGCGATAGAGGCAGCCAGAGCTTCTTCTGGT
ACAGACAGTACAGCGGCAAGAGCCCCGAGCTGATCATGAGCATCTACAGCAACG
GCGACAAAGAGGACGGCCGGTTTACAGCCCAGCTGAACAAGGCCAGCCAGTACG
TGTCCCTGCTGATCAGAGATAGCCAGCCTAGCGACAGCGCCACCTATCTGTGTGC
CGTGACCACAGATAGCTGGGGCAAGCTGCAGTTTGGCGCCGGAACACAGGTGGT
GGTTACACCTGACATTCAGAATCCTGAGCCTGCCGTGTACCAGCTGAAGGACCCT
AGAAGCCAGGACAGCACCCTGTGCCTGTTCACCGACTTCGACAGCCAGATCAAC
GTGCCCAAGACCATGGAAAGCGGCACCTTCATCACCGATAAGTGCGTGCTGGAC
ATGAAGGCCATGGACAGCAAGAGCAACGGCGCCATTGCCTGGTCCAACCAGACC
AGCTTCACGTGCCAGGACATCTTCAAAGAGACAAACGCCACCTATCCTAGCAGC
GACGTGCCCTGTGATGCCACACTGACCGAGAAGTCCTTCGAGACAGACATGAAC
CTGAACTTCCAGAACCTGCTGGTCATCGTGCTGAGAATCCTGCTGCTGAAGGTGG
CCGGCTTCAACCTGCTGATGACCCTGAGACTGTGGTCCAGCCGGGCCAAACGATC
TGGCTCTGGCCAGTGTACCAACTATGCCCTGCTCAAGCTCGCTGGCGACGTCGAG
TCTAACCCTGGACCAATGGGAGCTGGCGCTACAGGCAGAGCTATGGATGGACCT
AGACTGCTGCTGCTCCTGCTTCTGGGAGTGTCTCTTGGCGGAGCCAAAGAGGCCT
GTCCTACCGGCCTGTATACACACTCTGGCGAGTGCTGCAAGGCCTGCAATCTTGG
AGAAGGCGTCGCACAGCCTTGCGGCGCTAATCAGACAGTGTGCGAGCCTTGCCT
GGACAGCGTGACCTTTAGCGACGTGGTGTCAGCCACCGAGCCATGCAAGCCATG
TACCGAGTGTGTGGGCCTGCAGAGCATGTCTGCCCCTTGCGTGGAAGCTGACGAT
GCCGTGTGTAGATGCGCCTACGGCTACTACCAGGACGAGACAACAGGCAGATGC
GAGGCCTGTAGAGTGTGCGAAGCTGGATCTGGCCTGGTGTTCAGCTGCCAAGAC
AAGCAGAACACCGTGTGCGAGGAATGCCCCGATGGCACCTATAGCGACGAGGCC
AACCATGTGGATCCCTGCCTGCCTTGTACCGTGTGTGAAGATACCGAGCGGCAGC
TGCGCGAGTGTACAAGATGGGCTGATGCCGAGTGCGAAGAGATCCCTGGCAGAT
GGATCACCAGAAGCACACCTCCAGAGGGCAGCGATAGCACAGCCCCTTCTACAC
AAGAGCCCGAGGCTCCTCCTGAGCAGGACCTGATTGCTTCTACAGTGGCTGGCGT
CGTGACCACCGTGATGGGATCTAGTCAGCCCGTGGTCACCAGAGGCACCACCGA
CAATCTGATCCCCGTGTACTGTAGCATCCTGGCCGCCGTGGTTGTTGGACTGGTG
GCCTATATCGCCTTCAAGCGGTGGAACAGCTGATGATTCGAAGCGGCCGCTTCGA
GCAGACATGATAAGATACATTGATGAGTTTGGACAAACCACAACTAGAATGCAG
TGAAAAAAATGCTTTATTTGTGAAATTTGTGATGCTATTGCTTTATTTGTAACCAT
TATAAGCTGCAATAAACAAGTTACACGGCAGGGTCAGGGTTCTGGATATctgtgggac
aagaggatcagggttaggacatgatctcatttccctctttgccccaacccaggctggagtccagatgccagtgatggacaagggcggg
gctctgtggggctggcaagtcacggtctcatgctttatacgggaaatagcatcttagaaaccagctgctcgtgatggactgggactcag
ggacaggcacaagctatcaatcttggccaagaggccatgatttcagtgaacgttcacggccaggcctggcctgccactcaaggaaac
CACTGGCCGTCGTTTTACAACGTCGTGACTgggaaaaccctggcgttacccaacttaatcgccttgcagca
catccccctttcgccagctggcgtaatagcgaagaggcccgcaccgatcgcccttcccaacagttgcgcagcctgaatggcgaatgg
cgcctgatgcggtattttctccttacgcatctgtgcggtatttcacaccgcatatggtgcactctcagtacaatctgctctgatgccgcatag
ttaagccagccccgacacccgccaacacccgctgacgcgccctgacgggcttgtctgctcccggcatccgcttacagacaagctgtg
accgtctccgggagctgcatgtgtcagaggttttcaccgtcatcaccgaaacgcgcga
-H2-VLVH-A6-TCRα-2A-A6-TCRβ (amino acid sequence)
SEQ ID NO: 36
MSIGLLCCAALSLLWAGPVNAGVTQTPKFQVLKTGQSMTLQCAQDMNHEYMSWY
RQDPGMGLRLIHYSVGAGITDQGEVPNGYNVSRSTTEDFPLRLLSAAPSQTSVYFCA
SRPGLAGGRPEQYFGPGTRLTVTEDLRNVTPPKVSLFEPSKAEIANKQKATLVCLAR
GFFPDHVELSWWVNGKEVHSGVCTDPQAYKESNYSYCLSSRLRVSATFWHNPRNH
FRCQVQFHGLSEEDKWPEGSPKPVTQNISAEAWGRADCGITSASYQQGVLSATILYEI
LLGKATLYAVLVSTLVVMAMVKRKNSRAKRSGSGATNFSLLKQAGDVEENPGPMA
LPVTALLLPLALLLHAARPDIQMTQSPSSLSASVGDRVTITCRASQDVNTAVAWYQQ
KPGKAPKLLIYSAYFLYSGVPSRFSGSRSGTDFTLTISSLQPEDFATYYCQQYSRYSPV
TFGQGTKVEIKRTGGGSGGGGSGGGASEVQLVESGGGLVQPGGSLRLSCAASGFNV
YASGMHWVRQAPGKGLEWVAKIYPDSDYTYYADSVKGRFTISADTSKNTAYLQMN
SLRAEDTAVYYCSRDSSFYYVYAMDYWGQGTLVTVSSQKEVEQNSGPLSVPEGAIA
SLNCTYSDRGSQSFFWYRQYSGKSPELIMSIYSNGDKEDGRFTAQLNKASQYVSLLIR
DSQPSDSATYLCAVTTDSWGKLQFGAGTQVVVTPDIQNPEPAVYQLKDPRSQDSTL
CLFTDFDSQINVPKTMESGTFITDKCVLDMKAMDSKSNGAIAWSNQTSFTCQDIFKE
TNATYPSSDVPCDATLTEKSFETDMNLNFQNLLVIVLRILLLKVAGFNLLMTLRLWS
SRAKRSGSGQCTNYALLKLAGDVESNPGPMGAGATGRAMDGPRLLLLLLLGVSLG
GAKEACPTGLYTHSGECCKACNLGEGVAQPCGANQTVCEPCLDSVTFSDVVSATEP
CKPCTECVGLQSMSAPCVEADDAVCRCAYGYYQDETTGRCEACRVCEAGSGLVFS
CQDKQNTVCEECPDGTYSDEANHVDPCLPCTVCEDTERQLRECTRWADAECEEIPG
RWITRSTPPEGSDSTAPSTQEPEAPPEQDLIASTVAGVVTTVMGSSQPVVTRGTTDNLI
PVYCSILAAVVVGLVAYIAFKRWNS
-H2-VLVH-A6-TCRβ-2A-A6-TCRα (plasmid sequence)
SEQ ID NO: 37
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcggggggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
CTCTGCCTGTGACAGCTCTGCTGCTGCCTCTTGCTCTGCTTCTGCATGCCGCCAGA
CCTGACATCCAGATGACACAGAGCCCTAGCAGCCTGTCTGCCAGCGTGGGAGAC
AGAGTGACCATCACCTGTAGAGCCAGCCAGGACGTGAACACAGCCGTGGCTTGG
TATCAGCAGAAGCCTGGCAAGGCCCCTAAGCTGCTGATCTACAGCGCCTACTTCC
TGTACAGCGGCGTGCCCAGCAGATTCAGCGGCTCTAGAAGCGGCACCGACTTCA
CCCTGACCATAAGCAGTCTGCAGCCCGAGGACTTCGCCACCTACTACTGTCAGCA
GTACAGCCGGTACAGCCCCGTGACATTTGGCCAGGGCACCAAGGTGGAAATCAA
GAGAACAGGCGGAGGCTCTGGCGGAGGTGGAAGCGGAGGCGGAGCTTCTGAAG
TGCAGCTGGTTGAAAGTGGCGGCGGACTGGTTCAACCTGGCGGATCTCTGAGAC
TGAGCTGTGCCGCCAGCGGCTTCAATGTGTATGCCTCTGGCATGCACTGGGTCCG
ACAGGCCCCTGGAAAAGGCCTTGAGTGGGTCGCCAAGATCTACCCCGACAGCGA
CTACACCTACTATGCCGACAGCGTGAAGGGCAGATTCACCATCAGCGCCGACAC
CAGCAAGAACACCGCCTACCTGCAGATGAACAGCCTGAGAGCCGAGGACACCGC
CGTGTACTACTGCTCCAGAGACAGCAGCTTCTACTACGTGTACGCCATGGACTAC
TGGGGCCAGGGAACACTGGTCACCGTGTCCAGCAATGCCGGCGTGACCCAGACA
CCTAAGTTCCAGGTGCTGAAAACCGGCCAGAGCATGACCCTGCAGTGCGCCCAG
GATATGAACCACGAGTACATGAGCTGGTACAGACAGGACCCTGGCATGGGCCTG
AGACTGATCCACTATTCTGTCGGAGCCGGCATCACCGACCAGGGCGAAGTTCCTA
ATGGCTACAACGTGTCCAGAAGCACCACCGAGGACTTCCCACTGAGACTGCTGT
CTGCCGCTCCTAGCCAGACCAGCGTGTACTTCTGTGCCTCTAGACCTGGACTGGC
TGGCGGCAGACCTGAGCAGTATTTTGGCCCTGGCACCAGACTGACCGTGACCGA
GGACCTGAGAAACGTGACCCCTCCTAAGGTGTCCCTGTTCGAGCCTAGCAAGGC
CGAGATCGCCAACAAGCAGAAAGCCACACTCGTGTGCCTGGCCAGAGGCTTCTT
TCCCGATCACGTGGAACTGTCTTGGTGGGTCAACGGCAAAGAGGTGCACAGCGG
CGTCTGTACTGATCCCCAGGCCTACAAAGAGAGCAACTACAGCTACTGCCTGAG
CAGCAGACTGAGAGTGTCCGCCACCTTCTGGCACAACCCCAGAAACCACTTCAG
ATGCCAGGTGCAGTTTCACGGCCTGAGCGAAGAGGACAAGTGGCCTGAGGGAAG
CCCCAAGCCAGTGACACAGAATATCTCTGCCGAAGCCTGGGGCAGAGCCGATTG
TGGAATTACCAGCGCCAGCTACCAGCAAGGCGTGCTGTCTGCCACAATCCTGTAC
GAGATCCTGCTGGGCAAAGCCACTCTGTACGCCGTGCTGGTGTCTACCCTGGTCG
TGATGGCCATGGTCAAGCGGAAGAACAGCAGGGCCAAGAGAAGTGGAAGCGGC
GCCACAAACTTCAGCCTGTTGAAACAGGCCGGGGACGTCGAAGAGAATCCTGGA
CCAATGAAGTCCCTGAGAGTGCTGCTGGTCATCCTGTGGCTGCAGCTGTCTTGGG
TCTGGTCCCAGCAGAAAGAGGTGGAACAGAACAGCGGCCCTCTGTCTGTTCCTG
AAGGCGCTATCGCCAGCCTGAACTGCACCTACAGCGATAGAGGCAGCCAGAGCT
TCTTCTGGTACAGACAGTACAGCGGCAAGAGCCCCGAGCTGATCATGAGCATCT
ACAGCAACGGCGACAAAGAGGACGGCCGGTTTACAGCCCAGCTGAACAAGGCC
AGCCAGTACGTGTCCCTGCTGATCAGAGATAGCCAGCCTAGCGACAGCGCCACC
TATCTGTGTGCCGTGACCACAGATAGCTGGGGCAAGCTGCAGTTTGGCGCCGGA
ACACAGGTGGTGGTTACACCTGACATTCAGAATCCTGAGCCTGCCGTGTACCAGC
TGAAGGACCCTAGAAGCCAGGACAGCACCCTGTGCCTGTTCACCGACTTCGACA
GCCAGATCAACGTGCCCAAGACCATGGAAAGCGGCACCTTCATCACCGATAAGT
GCGTGCTGGACATGAAGGCCATGGACAGCAAGAGCAACGGCGCCATTGCCTGGT
CCAACCAGACCAGCTTCACGTGCCAGGACATCTTCAAAGAGACAAACGCCACCT
ATCCTAGCAGCGACGTGCCCTGTGATGCCACACTGACCGAGAAGTCCTTCGAGA
CAGACATGAACCTGAACTTCCAGAACCTGCTGGTCATCGTGCTGAGAATCCTGCT
GCTGAAGGTGGCCGGCTTCAACCTGCTGATGACCCTGAGACTGTGGTCCAGCCG
GGCCAAACGATCTGGCTCTGGCCAGTGTACCAACTATGCCCTGCTCAAGCTCGCT
GGCGACGTCGAGTCTAACCCTGGACCAATGGGAGCTGGCGCTACAGGCAGAGCT
ATGGATGGACCTAGACTGCTGCTGCTCCTGCTTCTGGGAGTGTCTCTTGGCGGAG
CCAAAGAGGCCTGTCCTACCGGCCTGTATACACACTCTGGCGAGTGCTGCAAGG
CCTGCAATCTTGGAGAAGGCGTCGCACAGCCTTGCGGCGCTAATCAGACAGTGT
GCGAGCCTTGCCTGGACAGCGTGACCTTTAGCGACGTGGTGTCAGCCACCGAGC
CATGCAAGCCATGTACCGAGTGTGTGGGCCTGCAGAGCATGTCTGCCCCTTGCGT
GGAAGCTGACGATGCCGTGTGTAGATGCGCCTACGGCTACTACCAGGACGAGAC
AACAGGCAGATGCGAGGCCTGTAGAGTGTGCGAAGCTGGATCTGGCCTGGTGTT
CAGCTGCCAAGACAAGCAGAACACCGTGTGCGAGGAATGCCCCGATGGCACCTA
TAGCGACGAGGCCAACCATGTGGATCCCTGCCTGCCTTGTACCGTGTGTGAAGAT
ACCGAGCGGCAGCTGCGCGAGTGTACAAGATGGGCTGATGCCGAGTGCGAAGAG
ATCCCTGGCAGATGGATCACCAGAAGCACACCTCCAGAGGGCAGCGATAGCACA
GCCCCTTCTACACAAGAGCCCGAGGCTCCTCCTGAGCAGGACCTGATTGCTTCTA
CAGTGGCTGGCGTCGTGACCACCGTGATGGGATCTAGTCAGCCCGTGGTCACCA
GAGGCACCACCGACAATCTGATCCCCGTGTACTGTAGCATCCTGGCCGCCGTGGT
TGTTGGACTGGTGGCCTATATCGCCTTCAAGCGGTGGAACAGCTGATGATTCGAA
GCGGCCGCTTCGAGCAGACATGATAAGATACATTGATGAGTTTGGACAAACCAC
AACTAGAATGCAGTGAAAAAAATGCTTTATTTGTGAAATTTGTGATGCTATTGCT
TTATTTGTAACCATTATAAGCTGCAATAAACAAGTTACACGGCAGGGTCAGGGTT
CTGGATATctgtgggacaagaggatcagggttaggacatgatctcatttccctctttgccccaacccaggctggagtccagatg
ccagtgatggacaagggcggggctctgtggggctggcaagtcacggtctcatgctttatacgggaaatagcatcttagaaaccagctg
ctcgtgatggactgggactcagggacaggcacaagctatcaatcttggccaagaggccatgatttcagtgaacgttcacggccaggc
ctggcctgccactcaaggaaacCACTGGCCGTCGTTTTACAACGTCGTGACTgggaaaaccctggcgtta
cccaacttaatcgccttgcagcacatccccctttcgccagctggcgtaatagcgaagaggcccgcaccgatcgcccttcccaacagtt
gcgcagcctgaatggcgaatggcgcctgatgcggtattttctccttacgcatctgtgcggtatttcacaccgcatatggtgcactctcagt
acaatctgctctgatgccgcatagttaagccagccccgacacccgccaacacccgctgacgcgccctgacgggcttgtctgctcccg
gcatccgcttacagacaagctgtgaccgtctccgggagctgcatgtgtcagaggttttcaccgtcatcaccgaaacgcgcga
-H2-VLVH-A6-TCRβ-2A-A6-TCRα (amino acid sequence)
SEQ ID NO: 38
MALPVTALLLPLALLLHAARPDIQMTQSPSSLSASVGDRVTITCRASQDVNTAVAWY
QQKPGKAPKLLIYSAYFLYSGVPSRFSGSRSGTDFTLTISSLQPEDFATYYCQQYSRYS
PVTFGQGTKVEIKRTGGGSGGGGSGGGASEVQLVESGGGLVQPGGSLRLSCAASGF
NVYASGMHWVRQAPGKGLEWVAKIYPDSDYTYYADSVKGRFTISADTSKNTAYLQ
MNSLRAEDTAVYYCSRDSSFYYVYAMDYWGQGTLVTVSSNAGVTQTPKFQVLKTG
QSMTLQCAQDMNHEYMSWYRQDPGMGLRLIHYSVGAGITDQGEVPNGYNVSRSTT
EDFPLRLLSAAPSQTSVYFCASRPGLAGGRPEQYFGPGTRLTVTEDLRNVTPPKVSLF
EPSKAEIANKQKATLVCLARGFFPDHVELSWWVNGKEVHSGVCTDPQAYKESNYSY
CLSSRLRVSATFWHNPRNHFRCQVQFHGLSEEDKWPEGSPKPVTQNISAEAWGRAD
CGITSASYQQGVLSATILYEILLGKATLYAVLVSTLVVMAMVKRKNSRAKRSGSGAT
NFSLLKQAGDVEENPGPMKSLRVLLVILWLQLSWVWSQQKEVEQNSGPLSVPEGAI
ASLNCTYSDRGSQSFFWYRQYSGKSPELIMSIYSNGDKEDGRFTAQLNKASQYVSLLI
RDSQPSDSATYLCAVTTDSWGKLQFGAGTQVVVTPDIQNPEPAVYQLKDPRSQDST
LCLFTDFDSQINVPKTMESGTFITDKCVLDMKAMDSKSNGAIAWSNQTSFTCQDIFK
ETNATYPSSDVPCDATLTEKSFETDMNLNFQNLLVIVLRILLLKVAGFNLLMTLRLW
SSRAKRSGSGQCTNYALLKLAGDVESNPGPMGAGATGRAMDGPRLLLLLLLGVSLG
GAKEACPTGLYTHSGECCKACNLGEGVAQPCGANQTVCEPCLDSVTFSDVVSATEP
CKPCTECVGLQSMSAPCVEADDAVCRCAYGYYQDETTGRCEACRVCEAGSGLVFS
CQDKQNTVCEECPDGTYSDEANHVDPCLPCTVCEDTERQLRECTRWADAECEEIPG
RWITRSTPPEGSDSTAPSTQEPEAPPEQDLIASTVAGVVTTVMGSSQPVVTRGTTDNLI
PVYCSILAAVVVGLVAYIAFKRWNS
-H2-VL-Cβ-2A-VH-Cα (plasmid sequence)
SEQ ID NO: 39
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcggggggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
CTCTGCCTGTGACAGCTCTGCTGCTGCCTCTTGCTCTGCTTCTGCATGCCGCCAGA
CCTGACATCCAGATGACACAGAGCCCTAGCAGCCTGTCTGCCAGCGTGGGAGAC
AGAGTGACCATCACCTGTAGAGCCAGCCAGGACGTGAACACAGCCGTGGCTTGG
TATCAGCAGAAGCCTGGCAAGGCCCCTAAGCTGCTGATCTACAGCGCCTACTTCC
TGTACAGCGGCGTGCCCAGCAGATTCAGCGGCTCTAGAAGCGGCACCGACTTCA
CCCTGACCATAAGCAGTCTGCAGCCCGAGGACTTCGCCACCTACTACTGTCAGCA
GTACAGCCGGTACAGCCCCGTGACATTTGGCCAGGGCACCAAGGTGGAAATCAA
GGAGGACCTGAGAAACGTGACCCCTCCTAAGGTGTCCCTGTTCGAGCCTAGCAA
GGCCGAGATCGCCAACAAGCAGAAAGCCACACTCGTGTGCCTGGCCAGAGGCTT
CTTTCCCGATCACGTGGAACTGTCTTGGTGGGTCAACGGCAAAGAGGTGCACAG
CGGCGTCTGTACTGATCCCCAGGCCTACAAAGAGAGCAACTACAGCTACTGCCT
GAGCAGCAGACTGAGAGTGTCCGCCACCTTCTGGCACAACCCCAGAAACCACTT
CAGATGCCAGGTGCAGTTTCACGGCCTGAGCGAAGAGGACAAGTGGCCTGAGGG
AAGCCCCAAGCCAGTGACACAGAATATCTCTGCCGAAGCCTGGGGCAGAGCCGA
TTGTGGAATTACCAGCGCCAGCTACCAGCAAGGCGTGCTGTCTGCCACAATCCTG
TACGAGATCCTGCTGGGCAAAGCCACTCTGTACGCCGTGCTGGTGTCTACCCTGG
TCGTGATGGCCATGGTCAAGCGGAAGAACAGCAGGGCCAAGAGAAGTGGAAGC
GGCGCCACAAACTTCAGCCTGTTGAAACAGGCCGGGGACGTCGAAGAGAATCCT
GGACCAATGGCACTGCCCGTCACTGCCTTGTTGCTGCCACTTGCACTGCTGCTCC
ATGCCGCCAGGCCAGAAGTGCAGCTGGTTGAAAGTGGCGGCGGACTGGTTCAAC
CTGGCGGATCTCTGAGACTGAGCTGTGCCGCCAGCGGCTTCAATGTGTATGCCTC
TGGCATGCACTGGGTCCGACAGGCCCCTGGAAAAGGCCTTGAGTGGGTCGCCAA
GATCTACCCCGACAGCGACTACACCTACTATGCCGACAGCGTGAAGGGCAGATT
CACCATCAGCGCCGACACCAGCAAGAACACCGCCTACCTGCAGATGAACAGCCT
GAGAGCCGAGGACACCGCCGTGTACTACTGCTCCAGAGACAGCAGCTTCTACTA
CGTGTACGCCATGGACTACTGGGGCCAGGGAACACTGGTCACCGTGTCCAGCGA
CATTCAGAATCCTGAGCCTGCCGTGTACCAGCTGAAGGACCCTAGAAGCCAGGA
CAGCACCCTGTGCCTGTTCACCGACTTCGACAGCCAGATCAACGTGCCCAAGACC
ATGGAAAGCGGCACCTTCATCACCGATAAGTGCGTGCTGGACATGAAGGCCATG
GACAGCAAGAGCAACGGCGCCATTGCCTGGTCCAACCAGACCAGCTTCACGTGC
CAGGACATCTTCAAAGAGACAAACGCCACCTATCCTAGCAGCGACGTGCCCTGT
GATGCCACACTGACCGAGAAGTCCTTCGAGACAGACATGAACCTGAACTTCCAG
AACCTGCTGGTCATCGTGCTGAGAATCCTGCTGCTGAAGGTGGCCGGCTTCAACC
TGCTGATGACCCTGAGACTGTGGTCCAGCCGGGCCAAACGATCTGGCTCTGGCCA
GTGTACCAACTATGCCCTGCTCAAGCTCGCTGGCGACGTCGAGTCTAACCCTGGA
CCAATGGGAGCTGGCGCTACAGGCAGAGCTATGGATGGACCTAGACTGCTGCTG
CTCCTGCTTCTGGGAGTGTCTCTTGGCGGAGCCAAAGAGGCCTGTCCTACCGGCC
TGTATACACACTCTGGCGAGTGCTGCAAGGCCTGCAATCTTGGAGAAGGCGTCG
CACAGCCTTGCGGCGCTAATCAGACAGTGTGCGAGCCTTGCCTGGACAGCGTGA
CCTTTAGCGACGTGGTGTCAGCCACCGAGCCATGCAAGCCATGTACCGAGTGTGT
GGGCCTGCAGAGCATGTCTGCCCCTTGCGTGGAAGCTGACGATGCCGTGTGTAG
ATGCGCCTACGGCTACTACCAGGACGAGACAACAGGCAGATGCGAGGCCTGTAG
AGTGTGCGAAGCTGGATCTGGCCTGGTGTTCAGCTGCCAAGACAAGCAGAACAC
CGTGTGCGAGGAATGCCCCGATGGCACCTATAGCGACGAGGCCAACCATGTGGA
TCCCTGCCTGCCTTGTACCGTGTGTGAAGATACCGAGCGGCAGCTGCGCGAGTGT
ACAAGATGGGCTGATGCCGAGTGCGAAGAGATCCCTGGCAGATGGATCACCAGA
AGCACACCTCCAGAGGGCAGCGATAGCACAGCCCCTTCTACACAAGAGCCCGAG
GCTCCTCCTGAGCAGGACCTGATTGCTTCTACAGTGGCTGGCGTCGTGACCACCG
TGATGGGATCTAGTCAGCCCGTGGTCACCAGAGGCACCACCGACAATCTGATCC
CCGTGTACTGTAGCATCCTGGCCGCCGTGGTTGTTGGACTGGTGGCCTATATCGC
CTTCAAGCGGTGGAACAGCTGATGATTCGAAGCGGCCGCTTCGAGCAGACATGA
TAAGATACATTGATGAGTTTGGACAAACCACAACTAGAATGCAGTGAAAAAAAT
GCTTTATTTGTGAAATTTGTGATGCTATTGCTTTATTTGTAACCATTATAAGCTGC
AATAAACAAGTTACACGGCAGGGTCAGGGTTCTGGATATctgtgggacaagaggatcagggtt
aggacatgatctcatttccctctttgccccaacccaggctggagtccagatgccagtgatggacaagggcggggctctgtggggctgg
caagtcacggtctcatgctttatacgggaaatagcatcttagaaaccagctgctcgtgatggactgggactcagggacaggcacaagc
tatcaatcttggccaagaggccatgatttcagtgaacgttcacggccaggcctggcctgccactcaaggaaacCACTGGCCG
TCGTTTTACAACGTCGTGACTgggaaaaccctggcgttacccaacttaatcgccttgcagcacatccccctttcgcc
agctggcgtaatagcgaagaggcccgcaccgatcgcccttcccaacagttgcgcagcctgaatggcgaatggcgcctgatgcggta
ttttctccttacgcatctgtgcggtatttcacaccgcatatggtgcactctcagtacaatctgctctgatgccgcatagttaagccagcccc
gacacccgccaacacccgctgacgcgccctgacgggcttgtctgctcccggcatccgcttacagacaagctgtgaccgtctccggga
gctgcatgtgtcagaggttttcaccgtcatcaccgaaacgcgcga
-H2-VL-Cβ-2A-VH-Cα (amino acid sequence)
SEQ ID NO: 40
MALPVTALLLPLALLLHAARPDIQMTQSPSSLSASVGDRVTITCRASQDVNTAVAWY
QQKPGKAPKLLIYSAYFLYSGVPSRFSGSRSGTDFTLTISSLQPEDFATYYCQQYSRYS
PVTFGQGTKVEIKEDLRNVTPPKVSLFEPSKAEIANKQKATLVCLARGFFPDHVELS
WWVNGKEVHSGVCTDPQAYKESNYSYCLSSRLRVSATFWHNPRNHFRCQVQFHGL
SEEDKWPEGSPKPVTQNISAEAWGRADCGITSASYQQGVLSATILYEILLGKATLYA
VLVSTLVVMAMVKRKNSRAKRSGSGATNFSLLKQAGDVEENPGPMALPVTALLLPL
ALLLHAARPEVQLVESGGGLVQPGGSLRLSCAASGFNVYASGMHWVRQAPGKGLE
WVAKIYPDSDYTYYADSVKGRFTISADTSKNTAYLQMNSLRAEDTAVYYCSRDSSF
YYVYAMDYWGQGTLVTVSSDIQNPEPAVYQLKDPRSQDSTLCLFTDFDSQINVPKT
MESGTFITDKCVLDMKAMDSKSNGAIAWSNQTSFTCQDIFKETNATYPSSDVPCDAT
LTEKSFETDMNLNFQNLLVIVLRILLLKVAGFNLLMTLRLWSSRAKRSGSGQCTNYA
LLKLAGDVESNPGPMGAGATGRAMDGPRLLLLLLLGVSLGGAKEACPTGLYTHSGE
CCKACNLGEGVAQPCGANQTVCEPCLDSVTFSDVVSATEPCKPCTECVGLQSMSAP
CVEADDAVCRCAYGYYQDETTGRCEACRVCEAGSGLVFSCQDKQNTVCEECPDGT
YSDEANHVDPCLPCTVCEDTERQLRECTRWADAECEEIPGRWITRSTPPEGSDSTAPS
TQEPEAPPEQDLIASTVAGVVTTVMGSSQPVVTRGTTDNLIPVYCSILAAVVVGLVA
YIAFKRWNS
-H2-VH-Cβ-2A-VL-Cα (plasmid sequence)
SEQ ID NO: 41
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
CTCTGCCTGTGACAGCTCTGCTGCTGCCTCTTGCTCTGCTTCTGCATGCCGCCAGA
CCTGAAGTGCAGCTGGTTGAAAGTGGCGGCGGACTGGTTCAACCTGGCGGATCT
CTGAGACTGAGCTGTGCCGCCAGCGGCTTCAATGTGTATGCCTCTGGCATGCACT
GGGTCCGACAGGCCCCTGGAAAAGGCCTTGAGTGGGTCGCCAAGATCTACCCCG
ACAGCGACTACACCTACTATGCCGACAGCGTGAAGGGCAGATTCACCATCAGCG
CCGACACCAGCAAGAACACCGCCTACCTGCAGATGAACAGCCTGAGAGCCGAGG
ACACCGCCGTGTACTACTGCTCCAGAGACAGCAGCTTCTACTACGTGTACGCCAT
GGACTACTGGGGCCAGGGAACACTGGTCACCGTGTCCAGCGAGGACCTGAGAAA
CGTGACCCCTCCTAAGGTGTCCCTGTTCGAGCCTAGCAAGGCCGAGATCGCCAAC
AAGCAGAAAGCCACACTCGTGTGCCTGGCCAGAGGCTTCTTTCCCGATCACGTGG
AACTGTCTTGGTGGGTCAACGGCAAAGAGGTGCACAGCGGCGTCTGTACTGATC
CCCAGGCCTACAAAGAGAGCAACTACAGCTACTGCCTGAGCAGCAGACTGAGAG
TGTCCGCCACCTTCTGGCACAACCCCAGAAACCACTTCAGATGCCAGGTGCAGTT
TCACGGCCTGAGCGAAGAGGACAAGTGGCCTGAGGGAAGCCCCAAGCCAGTGA
CACAGAATATCTCTGCCGAAGCCTGGGGCAGAGCCGATTGTGGAATTACCAGCG
CCAGCTACCAGCAAGGCGTGCTGTCTGCCACAATCCTGTACGAGATCCTGCTGGG
CAAAGCCACTCTGTACGCCGTGCTGGTGTCTACCCTGGTCGTGATGGCCATGGTC
AAGCGGAAGAACAGCAGGGCCAAGAGAAGTGGAAGCGGCGCCACAAACTTCAG
CCTGTTGAAACAGGCCGGGGACGTCGAAGAGAATCCTGGACCAATGGCACTGCC
CGTCACTGCCTTGTTGCTGCCACTTGCACTGCTGCTCCATGCCGCCAGGCCAGAC
ATCCAGATGACACAGAGCCCTAGCAGCCTGTCTGCCAGCGTGGGAGACAGAGTG
ACCATCACCTGTAGAGCCAGCCAGGACGTGAACACAGCCGTGGCTTGGTATCAG
CAGAAGCCTGGCAAGGCCCCTAAGCTGCTGATCTACAGCGCCTACTTCCTGTACA
GCGGCGTGCCCAGCAGATTCAGCGGCTCTAGAAGCGGCACCGACTTCACCCTGA
CCATAAGCAGTCTGCAGCCCGAGGACTTCGCCACCTACTACTGTCAGCAGTACAG
CCGGTACAGCCCCGTGACATTTGGCCAGGGCACCAAGGTGGAAATCAAGGACAT
TCAGAATCCTGAGCCTGCCGTGTACCAGCTGAAGGACCCTAGAAGCCAGGACAG
CACCCTGTGCCTGTTCACCGACTTCGACAGCCAGATCAACGTGCCCAAGACCATG
GAAAGCGGCACCTTCATCACCGATAAGTGCGTGCTGGACATGAAGGCCATGGAC
AGCAAGAGCAACGGCGCCATTGCCTGGTCCAACCAGACCAGCTTCACGTGCCAG
GACATCTTCAAAGAGACAAACGCCACCTATCCTAGCAGCGACGTGCCCTGTGAT
GCCACACTGACCGAGAAGTCCTTCGAGACAGACATGAACCTGAACTTCCAGAAC
CTGCTGGTCATCGTGCTGAGAATCCTGCTGCTGAAGGTGGCCGGCTTCAACCTGC
TGATGACCCTGAGACTGTGGTCCAGCCGGGCCAAACGATCTGGCTCTGGCCAGT
GTACCAACTATGCCCTGCTCAAGCTCGCTGGCGACGTCGAGTCTAACCCTGGACC
AATGGGAGCTGGCGCTACAGGCAGAGCTATGGATGGACCTAGACTGCTGCTGCT
CCTGCTTCTGGGAGTGTCTCTTGGCGGAGCCAAAGAGGCCTGTCCTACCGGCCTG
TATACACACTCTGGCGAGTGCTGCAAGGCCTGCAATCTTGGAGAAGGCGTCGCA
CAGCCTTGCGGCGCTAATCAGACAGTGTGCGAGCCTTGCCTGGACAGCGTGACCT
TTAGCGACGTGGTGTCAGCCACCGAGCCATGCAAGCCATGTACCGAGTGTGTGG
GCCTGCAGAGCATGTCTGCCCCTTGCGTGGAAGCTGACGATGCCGTGTGTAGATG
CGCCTACGGCTACTACCAGGACGAGACAACAGGCAGATGCGAGGCCTGTAGAGT
GTGCGAAGCTGGATCTGGCCTGGTGTTCAGCTGCCAAGACAAGCAGAACACCGT
GTGCGAGGAATGCCCCGATGGCACCTATAGCGACGAGGCCAACCATGTGGATCC
CTGCCTGCCTTGTACCGTGTGTGAAGATACCGAGCGGCAGCTGCGCGAGTGTACA
AGATGGGCTGATGCCGAGTGCGAAGAGATCCCTGGCAGATGGATCACCAGAAGC
ACACCTCCAGAGGGCAGCGATAGCACAGCCCCTTCTACACAAGAGCCCGAGGCT
CCTCCTGAGCAGGACCTGATTGCTTCTACAGTGGCTGGCGTCGTGACCACCGTGA
TGGGATCTAGTCAGCCCGTGGTCACCAGAGGCACCACCGACAATCTGATCCCCGT
GTACTGTAGCATCCTGGCCGCCGTGGTTGTTGGACTGGTGGCCTATATCGCCTTC
AAGCGGTGGAACAGCTGATGATTCGAAGCGGCCGCTTCGAGCAGACATGATAAG
ATACATTGATGAGTTTGGACAAACCACAACTAGAATGCAGTGAAAAAAATGCTT
TATTTGTGAAATTTGTGATGCTATTGCTTTATTTGTAACCATTATAAGCTGCAATA
AACAAGTTACACGGCAGGGTCAGGGTTCTGGATATctgtgggacaagaggatcagggttaggacat
gatctcatttccctctttgccccaacccaggctggagtccagatgccagtgatggacaagggcggggctctgtggggctggcaagtca
cggtctcatgctttatacgggaaatagcatcttagaaaccagctgctcgtgatggactgggactcagggacaggcacaagctatcaatc
ttggccaagaggccatgatttcagtgaacgttcacggccaggcctggcctgccactcaaggaaacCACTGGCCGTCGTT
TTACAACGTCGTGACTgggaaaaccctggcgttacccaacttaatcgccttgcagcacatccccctttcgccagctggc
gtaatagcgaagaggcccgcaccgatcgcccttcccaacagttgcgcagcctgaatggcgaatggcgcctgatgcggtattttctcctt
acgcatctgtgcggtatttcacaccgcatatggtgcactctcagtacaatctgctctgatgccgcatagttaagccagccccgacacccg
ccaacacccgctgacgcgccctgacgggcttgtctgctcccggcatccgcttacagacaagctgtgaccgtctccgggagctgcatgt
gtcagaggttttcaccgtcatcaccgaaacgcgcga
-H2-VH-Cβ-2A-VL-Cα (amino acid sequence)
SEQ ID NO: 42
MALPVTALLLPLALLLHAARPEVQLVESGGGLVQPGGSLRLSCAASGFNVYASGMH
WVRQAPGKGLEWVAKIYPDSDYTYYADSVKGRFTISADTSKNTAYLQMNSLRAED
TAVYYCSRDSSFYYVYAMDYWGQGTLVTVSSEDLRNVTPPKVSLFEPSKAEIANKQ
KATLVCLARGFFPDHVELSWWVNGKEVHSGVCTDPQAYKESNYSYCLSSRLRVSAT
FWHNPRNHFRCQVQFHGLSEEDKWPEGSPKPVTQNISAEAWGRADCGITSASYQQG
VLSATILYEILLGKATLYAVLVSTLVVMAMVKRKNSRAKRSGSGATNFSLLKQAGD
VEENPGPMALPVTALLLPLALLLHAARPDIQMTQSPSSLSASVGDRVTITCRASQDVN
TAVAWYQQKPGKAPKLLIYSAYFLYSGVPSRFSGSRSGTDFTLTISSLQPEDFATYYC
QQYSRYSPVTFGQGTKVEIKDIQNPEPAVYQLKDPRSQDSTLCLFTDFDSQINVPKTM
ESGTFITDKCVLDMKAMDSKSNGAIAWSNQTSFTCQDIFKETNATYPSSDVPCDATL
TEKSFETDMNLNFQNLLVIVLRILLLKVAGFNLLMTLRLWSSRAKRSGSGQCTNYAL
LKLAGDVESNPGPMGAGATGRAMDGPRLLLLLLLGVSLGGAKEACPTGLYTHSGEC
CKACNLGEGVAQPCGANQTVCEPCLDSVTFSDVVSATEPCKPCTECVGLQSMSAPC
VEADDAVCRCAYGYYQDETTGRCEACRVCEAGSGLVFSCQDKQNTVCEECPDGTY
SDEANHVDPCLPCTVCEDTERQLRECTRWADAECEEIPGRWITRSTPPEGSDSTAPST
QEPEAPPEQDLIASTVAGVVTTVMGSSQPVVTRGTTDNLIPVYCSILAAVVVGLVAYI
AFKRWNS
-H2-VL-EAAAK-Cβ-2A-VH-EAAAK-Ca (plasmid sequence)
SEQ ID NO: 43
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattacccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccccccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
CTCTGCCTGTGACAGCTCTGCTGCTGCCTCTTGCTCTGCTTCTGCATGCCGCCAGA
CCTGACATCCAGATGACACAGAGCCCTAGCAGCCTGTCTGCCAGCGTGGGAGAC
AGAGTGACCATCACCTGTAGAGCCAGCCAGGACGTGAACACAGCCGTGGCTTGG
TATCAGCAGAAGCCTGGCAAGGCCCCTAAGCTGCTGATCTACAGCGCCTACTTCC
TGTACAGCGGCGTGCCCAGCAGATTCAGCGGCTCTAGAAGCGGCACCGACTTCA
CCCTGACCATAAGCAGTCTGCAGCCCGAGGACTTCGCCACCTACTACTGTCAGCA
GTACAGCCGGTACAGCCCCGTGACATTTGGCCAGGGCACCAAGGTGGAAATCAA
GGAAGCCGCCGCTAAAGAGGACCTGAGAAACGTGACCCCTCCTAAGGTGTCCCT
GTTCGAGCCTAGCAAGGCCGAGATCGCCAACAAGCAGAAAGCCACACTCGTGTG
CCTGGCCAGAGGCTTCTTTCCCGATCACGTGGAACTGTCTTGGTGGGTCAACGGC
AAAGAGGTGCACAGCGGCGTCTGTACTGATCCCCAGGCCTACAAAGAGAGCAAC
TACAGCTACTGCCTGAGCAGCAGACTGAGAGTGTCCGCCACCTTCTGGCACAACC
CCAGAAACCACTTCAGATGCCAGGTGCAGTTTCACGGCCTGAGCGAAGAGGACA
AGTGGCCTGAGGGAAGCCCCAAGCCAGTGACACAGAATATCTCTGCCGAAGCCT
GGGGCAGAGCCGATTGTGGAATTACCAGCGCCAGCTACCAGCAAGGCGTGCTGT
CTGCCACAATCCTGTACGAGATCCTGCTGGGCAAAGCCACTCTGTACGCCGTGCT
GGTGTCTACCCTGGTCGTGATGGCCATGGTCAAGCGGAAGAACAGCAGGGCCAA
GAGAAGTGGAAGCGGCGCCACAAACTTCAGCCTGTTGAAACAGGCCGGGGACGT
CGAAGAGAATCCTGGACCAATGGCACTGCCCGTCACTGCCTTGTTGCTGCCACTT
GCACTGCTGCTCCATGCCGCCAGGCCAGAAGTGCAGCTGGTTGAAAGTGGCGGC
GGACTGGTTCAACCTGGCGGATCTCTGAGACTGAGCTGTGCCGCCAGCGGCTTCA
ATGTGTATGCCTCTGGCATGCACTGGGTCCGACAGGCCCCTGGAAAAGGCCTTGA
GTGGGTCGCCAAGATCTACCCCGACAGCGACTACACCTACTATGCCGACAGCGT
GAAGGGCAGATTCACCATCAGCGCCGACACCAGCAAGAACACCGCCTACCTGCA
GATGAACAGCCTGAGAGCCGAGGACACCGCCGTGTACTACTGCTCCAGAGACAG
CAGCTTCTACTACGTGTACGCCATGGACTACTGGGGCCAGGGAACACTGGTCACC
GTGTCCAGCGAAGCCGCCGCTAAAGACATTCAGAATCCTGAGCCTGCCGTGTAC
CAGCTGAAGGACCCTAGAAGCCAGGACAGCACCCTGTGCCTGTTCACCGACTTC
GACAGCCAGATCAACGTGCCCAAGACCATGGAAAGCGGCACCTTCATCACCGAT
AAGTGCGTGCTGGACATGAAGGCCATGGACAGCAAGAGCAACGGCGCCATTGCC
TGGTCCAACCAGACCAGCTTCACGTGCCAGGACATCTTCAAAGAGACAAACGCC
ACCTATCCTAGCAGCGACGTGCCCTGTGATGCCACACTGACCGAGAAGTCCTTCG
AGACAGACATGAACCTGAACTTCCAGAACCTGCTGGTCATCGTGCTGAGAATCCT
GCTGCTGAAGGTGGCCGGCTTCAACCTGCTGATGACCCTGAGACTGTGGTCCAGC
CGGGCCAAACGATCTGGCTCTGGCCAGTGTACCAACTATGCCCTGCTCAAGCTCG
CTGGCGACGTCGAGTCTAACCCTGGACCAATGGGAGCTGGCGCTACAGGCAGAG
CTATGGATGGACCTAGACTGCTGCTGCTCCTGCTTCTGGGAGTGTCTCTTGGCGG
AGCCAAAGAGGCCTGTCCTACCGGCCTGTATACACACTCTGGCGAGTGCTGCAA
GGCCTGCAATCTTGGAGAAGGCGTCGCACAGCCTTGCGGCGCTAATCAGACAGT
GTGCGAGCCTTGCCTGGACAGCGTGACCTTTAGCGACGTGGTGTCAGCCACCGA
GCCATGCAAGCCATGTACCGAGTGTGTGGGCCTGCAGAGCATGTCTGCCCCTTGC
GTGGAAGCTGACGATGCCGTGTGTAGATGCGCCTACGGCTACTACCAGGACGAG
ACAACAGGCAGATGCGAGGCCTGTAGAGTGTGCGAAGCTGGATCTGGCCTGGTG
TTCAGCTGCCAAGACAAGCAGAACACCGTGTGCGAGGAATGCCCCGATGGCACC
TATAGCGACGAGGCCAACCATGTGGATCCCTGCCTGCCTTGTACCGTGTGTGAAG
ATACCGAGCGGCAGCTGCGCGAGTGTACAAGATGGGCTGATGCCGAGTGCGAAG
AGATCCCTGGCAGATGGATCACCAGAAGCACACCTCCAGAGGGCAGCGATAGCA
CAGCCCCTTCTACACAAGAGCCCGAGGCTCCTCCTGAGCAGGACCTGATTGCTTC
TACAGTGGCTGGCGTCGTGACCACCGTGATGGGATCTAGTCAGCCCGTGGTCACC
AGAGGCACCACCGACAATCTGATCCCCGTGTACTGTAGCATCCTGGCCGCCGTGG
TTGTTGGACTGGTGGCCTATATCGCCTTCAAGCGGTGGAACAGCTGATGATTCGA
AGCGGCCGCTTCGAGCAGACATGATAAGATACATTGATGAGTTTGGACAAACCA
CAACTAGAATGCAGTGAAAAAAATGCTTTATTTGTGAAATTTGTGATGCTATTGC
TTTATTTGTAACCATTATAAGCTGCAATAAACAAGTTACACGGCAGGGTCAGGGT
TCTGGATATctgtgggacaagaggatcagggttaggacatgatctcatttccctctttgccccaacccaggctggagtccagat
gccagtgatggacaagggcggggctctgtggggctggcaagtcacggtctcatgctttatacgggaaatagcatcttagaaaccagct
gctcgtgatggactgggactcagggacaggcacaagctatcaatcttggccaagaggccatgatttcagtgaacgttcacggccagg
cctggcctgccactcaaggaaacCACTGGCCGTCGTTTTACAACGTCGTGACTgggaaaaccctggcgtt
acccaacttaatcgccttgcagcacatccccctttcgccagctggcgtaatagcgaagaggcccgcaccgatcgcccttcccaacagt
tgcgcagcctgaatggcgaatggcgcctgatgcggtattttctccttacgcatctgtgcggtatttcacaccgcatatggtgcactctcag
tacaatctgctctgatgccgcatagttaagccagccccgacacccgccaacacccgctgacgcgccctgacgggcttgtctgctcccg
gcatccgcttacagacaagctgtgaccgtctccgggagctgcatgtgtcagaggttttcaccgtcatcaccgaaacgcgcga
-H2-VL-EAAAK-Cβ-2A-VH-EAAAK-Cα (amino acid sequence)
SEQ ID NO: 44
MALPVTALLLPLALLLHAARPDIQMTQSPSSLSASVGDRVTITCRASQDVNTAVAWY
QQKPGKAPKLLIYSAYFLYSGVPSRFSGSRSGTDFTLTISSLQPEDFATYYCQQYSRYS
PVTFGQGTKVEIKEAAAKEDLRNVTPPKVSLFEPSKAEIANKQKATLVCLARGFFPD
HVELSWWVNGKEVHSGVCTDPQAYKESNYSYCLSSRLRVSATFWHNPRNHFRCQV
QFHGLSEEDKWPEGSPKPVTQNISAEAWGRADCGITSASYQQGVLSATILYEILLGKA
TLYAVLVSTLVVMAMVKRKNSRAKRSGSGATNFSLLKQAGDVEENPGPMALPVTA
LLLPLALLLHAARPEVQLVESGGGLVQPGGSLRLSCAASGFNVYASGMHWVRQAPG
KGLEWVAKIYPDSDYTYYADSVKGRFTISADTSKNTAYLQMNSLRAEDTAVYYCSR
DSSFYYVYAMDYWGQGTLVTVSSEAAAKDIQNPEPAVYQLKDPRSQDSTLCLFTDF
DSQINVPKTMESGTFITDKCVLDMKAMDSKSNGAIAWSNQTSFTCQDIFKETNATYP
SSDVPCDATLTEKSFETDMNLNFQNLLVIVLRILLLKVAGFNLLMTLRLWSSRAKRS
GSGQCTNYALLKLAGDVESNPGPMGAGATGRAMDGPRLLLLLLLGVSLGGAKEAC
PTGLYTHSGECCKACNLGEGVAQPCGANQTVCEPCLDSVTFSDVVSATEPCKPCTEC
VGLQSMSAPCVEADDAVCRCAYGYYQDETTGRCEACRVCEAGSGLVFSCQDKQNT
VCEECPDGTYSDEANHVDPCLPCTVCEDTERQLRECTRWADAECEEIPGRWITRSTP
PEGSDSTAPSTQEPEAPPEQDLIASTVAGVVTTVMGSSQPVVTRGTTDNLIPVYCSILA
AVVVGLVAYIAFKRWNS
-H2-VH-EAAAK-Cβ-2A-VL-EAAAK-Cα (plasmid sequence)
SEQ ID NO: 45
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccccccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
CTCTGCCTGTGACAGCTCTGCTGCTGCCTCTTGCTCTGCTTCTGCATGCCGCCAGA
CCTGAAGTGCAGCTGGTTGAAAGTGGCGGCGGACTGGTTCAACCTGGCGGATCT
CTGAGACTGAGCTGTGCCGCCAGCGGCTTCAATGTGTATGCCTCTGGCATGCACT
GGGTCCGACAGGCCCCTGGAAAAGGCCTTGAGTGGGTCGCCAAGATCTACCCCG
ACAGCGACTACACCTACTATGCCGACAGCGTGAAGGGCAGATTCACCATCAGCG
CCGACACCAGCAAGAACACCGCCTACCTGCAGATGAACAGCCTGAGAGCCGAGG
ACACCGCCGTGTACTACTGCTCCAGAGACAGCAGCTTCTACTACGTGTACGCCAT
GGACTACTGGGGCCAGGGAACACTGGTCACCGTGTCCAGCGAAGCCGCCGCTAA
AGAGGACCTGAGAAACGTGACCCCTCCTAAGGTGTCCCTGTTCGAGCCTAGCAA
GGCCGAGATCGCCAACAAGCAGAAAGCCACACTCGTGTGCCTGGCCAGAGGCTT
CTTTCCCGATCACGTGGAACTGTCTTGGTGGGTCAACGGCAAAGAGGTGCACAG
CGGCGTCTGTACTGATCCCCAGGCCTACAAAGAGAGCAACTACAGCTACTGCCT
GAGCAGCAGACTGAGAGTGTCCGCCACCTTCTGGCACAACCCCAGAAACCACTT
CAGATGCCAGGTGCAGTTTCACGGCCTGAGCGAAGAGGACAAGTGGCCTGAGGG
AAGCCCCAAGCCAGTGACACAGAATATCTCTGCCGAAGCCTGGGGCAGAGCCGA
TTGTGGAATTACCAGCGCCAGCTACCAGCAAGGCGTGCTGTCTGCCACAATCCTG
TACGAGATCCTGCTGGGCAAAGCCACTCTGTACGCCGTGCTGGTGTCTACCCTGG
TCGTGATGGCCATGGTCAAGCGGAAGAACAGCAGGGCCAAGAGAAGTGGAAGC
GGCGCCACAAACTTCAGCCTGTTGAAACAGGCCGGGGACGTCGAAGAGAATCCT
GGACCAATGGCACTGCCCGTCACTGCCTTGTTGCTGCCACTTGCACTGCTGCTCC
ATGCCGCCAGGCCAGACATCCAGATGACACAGAGCCCTAGCAGCCTGTCTGCCA
GCGTGGGAGACAGAGTGACCATCACCTGTAGAGCCAGCCAGGACGTGAACACAG
CCGTGGCTTGGTATCAGCAGAAGCCTGGCAAGGCCCCTAAGCTGCTGATCTACA
GCGCCTACTTCCTGTACAGCGGCGTGCCCAGCAGATTCAGCGGCTCTAGAAGCG
GCACCGACTTCACCCTGACCATAAGCAGTCTGCAGCCCGAGGACTTCGCCACCTA
CTACTGTCAGCAGTACAGCCGGTACAGCCCCGTGACATTTGGCCAGGGCACCAA
GGTGGAAATCAAGGAAGCCGCCGCTAAAGACATTCAGAATCCTGAGCCTGCCGT
GTACCAGCTGAAGGACCCTAGAAGCCAGGACAGCACCCTGTGCCTGTTCACCGA
CTTCGACAGCCAGATCAACGTGCCCAAGACCATGGAAAGCGGCACCTTCATCAC
CGATAAGTGCGTGCTGGACATGAAGGCCATGGACAGCAAGAGCAACGGCGCCAT
TGCCTGGTCCAACCAGACCAGCTTCACGTGCCAGGACATCTTCAAAGAGACAAA
CGCCACCTATCCTAGCAGCGACGTGCCCTGTGATGCCACACTGACCGAGAAGTCC
TTCGAGACAGACATGAACCTGAACTTCCAGAACCTGCTGGTCATCGTGCTGAGA
ATCCTGCTGCTGAAGGTGGCCGGCTTCAACCTGCTGATGACCCTGAGACTGTGGT
CCAGCCGGGCCAAACGATCTGGCTCTGGCCAGTGTACCAACTATGCCCTGCTCAA
GCTCGCTGGCGACGTCGAGTCTAACCCTGGACCAATGGGAGCTGGCGCTACAGG
CAGAGCTATGGATGGACCTAGACTGCTGCTGCTCCTGCTTCTGGGAGTGTCTCTT
GGCGGAGCCAAAGAGGCCTGTCCTACCGGCCTGTATACACACTCTGGCGAGTGC
TGCAAGGCCTGCAATCTTGGAGAAGGCGTCGCACAGCCTTGCGGCGCTAATCAG
ACAGTGTGCGAGCCTTGCCTGGACAGCGTGACCTTTAGCGACGTGGTGTCAGCCA
CCGAGCCATGCAAGCCATGTACCGAGTGTGTGGGCCTGCAGAGCATGTCTGCCC
CTTGCGTGGAAGCTGACGATGCCGTGTGTAGATGCGCCTACGGCTACTACCAGG
ACGAGACAACAGGCAGATGCGAGGCCTGTAGAGTGTGCGAAGCTGGATCTGGCC
TGGTGTTCAGCTGCCAAGACAAGCAGAACACCGTGTGCGAGGAATGCCCCGATG
GCACCTATAGCGACGAGGCCAACCATGTGGATCCCTGCCTGCCTTGTACCGTGTG
TGAAGATACCGAGCGGCAGCTGCGCGAGTGTACAAGATGGGCTGATGCCGAGTG
CGAAGAGATCCCTGGCAGATGGATCACCAGAAGCACACCTCCAGAGGGCAGCGA
TAGCACAGCCCCTTCTACACAAGAGCCCGAGGCTCCTCCTGAGCAGGACCTGATT
GCTTCTACAGTGGCTGGCGTCGTGACCACCGTGATGGGATCTAGTCAGCCCGTGG
TCACCAGAGGCACCACCGACAATCTGATCCCCGTGTACTGTAGCATCCTGGCCGC
CGTGGTTGTTGGACTGGTGGCCTATATCGCCTTCAAGCGGTGGAACAGCTGATGA
TTCGAAGCGGCCGCTTCGAGCAGACATGATAAGATACATTGATGAGTTTGGACA
AACCACAACTAGAATGCAGTGAAAAAAATGCTTTATTTGTGAAATTTGTGATGCT
ATTGCTTTATTTGTAACCATTATAAGCTGCAATAAACAAGTTACACGGCAGGGTC
AGGGTTCTGGATATctgtgggacaagaggatcagggttaggacatgatctcatttccctctttgccccaacccaggctgga
gtccagatgccagtgatggacaagggcggggctctgtggggctggcaagtcacggtctcatgctttatacgggaaatagcatcttaga
aaccagctgctcgtgatggactgggactcagggacaggcacaagctatcaatcttggccaagaggccatgatttcagtgaacgttcac
ggccaggcctggcctgccactcaaggaaacCACTGGCCGTCGTTTTACAACGTCGTGACTgggaaaac
cctggcgttacccaacttaatcgccttgcagcacatccccctttcgccagctggcgtaatagcgaagaggcccgcaccgatcgcccttc
ccaacagttgcgcagcctgaatggcgaatggcgcctgatgcggtattttctccttacgcatctgtgcggtatttcacaccgcatatggtgc
actctcagtacaatctgctctgatgccgcatagttaagccagccccgacacccgccaacacccgctgacgcgccctgacgggcttgtc
tgctcccggcatccgcttacagacaagctgtgaccgtctccgggagctgcatgtgtcagaggttttcaccgtcatcaccgaaacgcgc
ga
-H2-VH-EAAAK-Cβ-2A-VL-EAAAK-Cα (amino acid sequence)
SEQ ID NO: 46
MALPVTALLLPLALLLHAARPEVQLVESGGGLVQPGGSLRLSCAASGFNVYASGMH
WVRQAPGKGLEWVAKIYPDSDYTYYADSVKGRFTISADTSKNTAYLQMNSLRAED
TAVYYCSRDSSFYYVYAMDYWGQGTLVTVSSEAAAKEDLRNVTPPKVSLFEPSKAE
IANKQKATLVCLARGFFPDHVELSWWVNGKEVHSGVCTDPQAYKESNYSYCLSSRL
RVSATFWHNPRNHFRCQVQFHGLSEEDKWPEGSPKPVTQNISAEAWGRADCGITSA
SYQQGVLSATILYEILLGKATLYAVLVSTLVVMAMVKRKNSRAKRSGSGATNFSLL
KQAGDVEENPGPMALPVTALLLPLALLLHAARPDIQMTQSPSSLSASVGDRVTITCR
ASQDVNTAVAWYQQKPGKAPKLLIYSAYFLYSGVPSRFSGSRSGTDFTLTISSLQPED
FATYYCQQYSRYSPVTFGQGTKVEIKEAAAKDIQNPEPAVYQLKDPRSQDSTLCLFT
DFDSQINVPKTMESGTFITDKCVLDMKAMDSKSNGAIAWSNQTSFTCQDIFKETNAT
YPSSDVPCDATLTEKSFETDMNLNFQNLLVIVLRILLLKVAGFNLLMTLRLWSSRAK
RSGSGQCTNYALLKLAGDVESNPGPMGAGATGRAMDGPRLLLLLLLGVSLGGAKE
ACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDKQ
NTVCEECPDGTYSDEANHVDPCLPCTVCEDTERQLRECTRWADAECEEIPGRWITRS
TPPEGSDSTAPSTQEPEAPPEQDLIASTVAGVVTTVMGSSQPVVTRGTTDNLIPVYCSI
LAAVVVGLVAYIAFKRWNS
-H2-VL-A6-TCRβ-2A-VH-A6-TCRα (plasmid sequence)
SEQ ID NO: 47
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcggggggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
CTCTGCCTGTGACAGCTCTGCTGCTGCCTCTTGCTCTGCTTCTGCATGCCGCCAGA
CCTGACATCCAGATGACACAGAGCCCTAGCAGCCTGTCTGCCAGCGTGGGAGAC
AGAGTGACCATCACCTGTAGAGCCAGCCAGGACGTGAACACAGCCGTGGCTTGG
TATCAGCAGAAGCCTGGCAAGGCCCCTAAGCTGCTGATCTACAGCGCCTACTTCC
TGTACAGCGGCGTGCCCAGCAGATTCAGCGGCTCTAGAAGCGGCACCGACTTCA
CCCTGACCATAAGCAGTCTGCAGCCCGAGGACTTCGCCACCTACTACTGTCAGCA
GTACAGCCGGTACAGCCCCGTGACATTTGGCCAGGGCACCAAGGTGGAAATCAA
GGGCGGTGGCGGTTCTAATGCCGGCGTGACCCAGACACCTAAGTTCCAGGTGCT
GAAAACCGGCCAGAGCATGACCCTGCAGTGCGCCCAGGATATGAACCACGAGTA
CATGAGCTGGTACAGACAGGACCCTGGCATGGGCCTGAGACTGATCCACTATTCT
GTCGGAGCCGGCATCACCGACCAGGGCGAAGTTCCTAATGGCTACAACGTGTCC
AGAAGCACCACCGAGGACTTCCCACTGAGACTGCTGTCTGCCGCTCCTAGCCAG
ACCAGCGTGTACTTCTGTGCCTCTAGACCTGGACTGGCTGGCGGCAGACCTGAGC
AGTATTTTGGCCCTGGCACCAGACTGACCGTGACCGAGGACCTGAGAAACGTGA
CCCCTCCTAAGGTGTCCCTGTTCGAGCCTAGCAAGGCCGAGATCGCCAACAAGC
AGAAAGCCACACTCGTGTGCCTGGCCAGAGGCTTCTTTCCCGATCACGTGGAACT
GTCTTGGTGGGTCAACGGCAAAGAGGTGCACAGCGGCGTCTGTACTGATCCCCA
GGCCTACAAAGAGAGCAACTACAGCTACTGCCTGAGCAGCAGACTGAGAGTGTC
CGCCACCTTCTGGCACAACCCCAGAAACCACTTCAGATGCCAGGTGCAGTTTCAC
GGCCTGAGCGAAGAGGACAAGTGGCCTGAGGGAAGCCCCAAGCCAGTGACACA
GAATATCTCTGCCGAAGCCTGGGGCAGAGCCGATTGTGGAATTACCAGCGCCAG
CTACCAGCAAGGCGTGCTGTCTGCCACAATCCTGTACGAGATCCTGCTGGGCAAA
GCCACTCTGTACGCCGTGCTGGTGTCTACCCTGGTCGTGATGGCCATGGTCAAGC
GGAAGAACAGCAGGGCCAAGAGAAGTGGAAGCGGCGCCACAAACTTCAGCCTG
TTGAAACAGGCCGGGGACGTCGAAGAGAATCCTGGACCAATGGCACTGCCCGTC
ACTGCCTTGTTGCTGCCACTTGCACTGCTGCTCCATGCCGCCAGGCCAGAAGTGC
AGCTGGTTGAAAGTGGCGGCGGACTGGTTCAACCTGGCGGATCTCTGAGACTGA
GCTGTGCCGCCAGCGGCTTCAATGTGTATGCCTCTGGCATGCACTGGGTCCGACA
GGCCCCTGGAAAAGGCCTTGAGTGGGTCGCCAAGATCTACCCCGACAGCGACTA
CACCTACTATGCCGACAGCGTGAAGGGCAGATTCACCATCAGCGCCGACACCAG
CAAGAACACCGCCTACCTGCAGATGAACAGCCTGAGAGCCGAGGACACCGCCGT
GTACTACTGCTCCAGAGACAGCAGCTTCTACTACGTGTACGCCATGGACTACTGG
GGCCAGGGAACACTGGTCACCGTGTCCAGCGGCGGTGGCGGTTCTCAGAAAGAG
GTGGAACAGAACAGCGGCCCTCTGTCTGTTCCTGAAGGCGCTATCGCCAGCCTGA
ACTGCACCTACAGCGATAGAGGCAGCCAGAGCTTCTTCTGGTACAGACAGTACA
GCGGCAAGAGCCCCGAGCTGATCATGAGCATCTACAGCAACGGCGACAAAGAG
GACGGCCGGTTTACAGCCCAGCTGAACAAGGCCAGCCAGTACGTGTCCCTGCTG
ATCAGAGATAGCCAGCCTAGCGACAGCGCCACCTATCTGTGTGCCGTGACCACA
GATAGCTGGGGCAAGCTGCAGTTTGGCGCCGGAACACAGGTGGTGGTTACACCT
GACATTCAGAATCCTGAGCCTGCCGTGTACCAGCTGAAGGACCCTAGAAGCCAG
GACAGCACCCTGTGCCTGTTCACCGACTTCGACAGCCAGATCAACGTGCCCAAG
ACCATGGAAAGCGGCACCTTCATCACCGATAAGTGCGTGCTGGACATGAAGGCC
ATGGACAGCAAGAGCAACGGCGCCATTGCCTGGTCCAACCAGACCAGCTTCACG
TGCCAGGACATCTTCAAAGAGACAAACGCCACCTATCCTAGCAGCGACGTGCCC
TGTGATGCCACACTGACCGAGAAGTCCTTCGAGACAGACATGAACCTGAACTTC
CAGAACCTGCTGGTCATCGTGCTGAGAATCCTGCTGCTGAAGGTGGCCGGCTTCA
ACCTGCTGATGACCCTGAGACTGTGGTCCAGCCGGGCCAAACGATCTGGCTCTGG
CCAGTGTACCAACTATGCCCTGCTCAAGCTCGCTGGCGACGTCGAGTCTAACCCT
GGACCAATGGGAGCTGGCGCTACAGGCAGAGCTATGGATGGACCTAGACTGCTG
CTGCTCCTGCTTCTGGGAGTGTCTCTTGGCGGAGCCAAAGAGGCCTGTCCTACCG
GCCTGTATACACACTCTGGCGAGTGCTGCAAGGCCTGCAATCTTGGAGAAGGCG
TCGCACAGCCTTGCGGCGCTAATCAGACAGTGTGCGAGCCTTGCCTGGACAGCGT
GACCTTTAGCGACGTGGTGTCAGCCACCGAGCCATGCAAGCCATGTACCGAGTG
TGTGGGCCTGCAGAGCATGTCTGCCCCTTGCGTGGAAGCTGACGATGCCGTGTGT
AGATGCGCCTACGGCTACTACCAGGACGAGACAACAGGCAGATGCGAGGCCTGT
AGAGTGTGCGAAGCTGGATCTGGCCTGGTGTTCAGCTGCCAAGACAAGCAGAAC
ACCGTGTGCGAGGAATGCCCCGATGGCACCTATAGCGACGAGGCCAACCATGTG
GATCCCTGCCTGCCTTGTACCGTGTGTGAAGATACCGAGCGGCAGCTGCGCGAGT
GTACAAGATGGGCTGATGCCGAGTGCGAAGAGATCCCTGGCAGATGGATCACCA
GAAGCACACCTCCAGAGGGCAGCGATAGCACAGCCCCTTCTACACAAGAGCCCG
AGGCTCCTCCTGAGCAGGACCTGATTGCTTCTACAGTGGCTGGCGTCGTGACCAC
CGTGATGGGATCTAGTCAGCCCGTGGTCACCAGAGGCACCACCGACAATCTGAT
CCCCGTGTACTGTAGCATCCTGGCCGCCGTGGTTGTTGGACTGGTGGCCTATATC
GCCTTCAAGCGGTGGAACAGCTGATGATTCGAAGCGGCCGCTTCGAGCAGACAT
GATAAGATACATTGATGAGTTTGGACAAACCACAACTAGAATGCAGTGAAAAAA
ATGCTTTATTTGTGAAATTTGTGATGCTATTGCTTTATTTGTAACCATTATAAGCT
GCAATAAACAAGTTACACGGCAGGGTCAGGGTTCTGGATATctgtgggacaagaggatcagg
gttaggacatgatctcatttccctctttgccccaacccaggctggagtccagatgccagtgatggacaagggcggggctctgtggggct
ggcaagtcacggtctcatgctttatacgggaaatagcatcttagaaaccagctgctcgtgatggactgggactcagggacaggcacaa
gctatcaatcttggccaagaggccatgatttcagtgaacgttcacggccaggcctggcctgccactcaaggaaacCACTGGCC
GTCGTTTTACAACGTCGTGACTgggaaaaccctggcgttacccaacttaatcgccttgcagcacatccccctttcg
ccagctggcgtaatagcgaagaggcccgcaccgatcgcccttcccaacagttgcgcagcctgaatggcgaatggcgcctgatgcgg
tattttctccttacgcatctgtgcggtatttcacaccgcatatggtgcactctcagtacaatctgctctgatgccgcatagttaagccagccc
cgacacccgccaacacccgctgacgcgccctgacgggcttgtctgctcccggcatccgcttacagacaagctgtgaccgtctccggg
agctgcatgtgtcagaggttttcaccgtcatcaccgaaacgcgcga
-H2-VL-A6-TCRβ-2A-VH-A6-TCRα (amino acid sequence)
SEQ ID NO: 48
MALPVTALLLPLALLLHAARPDIQMTQSPSSLSASVGDRVTITCRASQDVNTAVAWY
QQKPGKAPKLLIYSAYFLYSGVPSRFSGSRSGTDFTLTISSLQPEDFATYYCQQYSRYS
PVTFGQGTKVEIKGGGGSNAGVTQTPKFQVLKTGQSMTLQCAQDMNHEYMSWYR
QDPGMGLRLIHYSVGAGITDQGEVPNGYNVSRSTTEDFPLRLLSAAPSQTSVYFCAS
RPGLAGGRPEQYFGPGTRLTVTEDLRNVTPPKVSLFEPSKAEIANKQKATLVCLARG
FFPDHVELSWWVNGKEVHSGVCTDPQAYKESNYSYCLSSRLRVSATFWHNPRNHFR
CQVQFHGLSEEDKWPEGSPKPVTQNISAEAWGRADCGITSASYQQGVLSATILYEILL
GKATLYAVLVSTLVVMAMVKRKNSRAKRSGSGATNFSLLKQAGDVEENPGPMALP
VTALLLPLALLLHAARPEVQLVESGGGLVQPGGSLRLSCAASGFNVYASGMHWVRQ
APGKGLEWVAKIYPDSDYTYYADSVKGRFTISADTSKNTAYLQMNSLRAEDTAVYY
CSRDSSFYYVYAMDYWGQGTLVTVSSGGGGSQKEVEQNSGPLSVPEGAIASLNCTY
SDRGSQSFFWYRQYSGKSPELIMSIYSNGDKEDGRFTAQLNKASQYVSLLIRDSQPSD
SATYLCAVTTDSWGKLQFGAGTQVVVTPDIQNPEPAVYQLKDPRSQDSTLCLFTDFD
SQINVPKTMESGTFITDKCVLDMKAMDSKSNGAIAWSNQTSFTCQDIFKETNATYPS
SDVPCDATLTEKSFETDMNLNFQNLLVIVLRILLLKVAGFNLLMTLRLWSSRAKRSG
SGQCTNYALLKLAGDVESNPGPMGAGATGRAMDGPRLLLLLLLGVSLGGAKEACP
TGLYTHSGECCKACNLGEGVAQPCGANQTVCEPCLDSVTFSDVVSATEPCKPCTEC
VGLQSMSAPCVEADDAVCRCAYGYYQDETTGRCEACRVCEAGSGLVFSCQDKQNT
VCEECPDGTYSDEANHVDPCLPCTVCEDTERQLRECTRWADAECEEIPGRWITRSTP
PEGSDSTAPSTQEPEAPPEQDLIASTVAGVVTTVMGSSQPVVTRGTTDNLIPVYCSILA
AVVVGLVAYIAFKRWNS
-H2-VH-A6-TCRβ-2A-VL-A6-TCRα (plasmid sequence)
SEQ ID NO: 49
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
CTCTGCCTGTGACAGCTCTGCTGCTGCCTCTTGCTCTGCTTCTGCATGCCGCCAGA
CCTGAAGTGCAGCTGGTTGAAAGTGGCGGCGGACTGGTTCAACCTGGCGGATCT
CTGAGACTGAGCTGTGCCGCCAGCGGCTTCAATGTGTATGCCTCTGGCATGCACT
GGGTCCGACAGGCCCCTGGAAAAGGCCTTGAGTGGGTCGCCAAGATCTACCCCG
ACAGCGACTACACCTACTATGCCGACAGCGTGAAGGGCAGATTCACCATCAGCG
CCGACACCAGCAAGAACACCGCCTACCTGCAGATGAACAGCCTGAGAGCCGAGG
ACACCGCCGTGTACTACTGCTCCAGAGACAGCAGCTTCTACTACGTGTACGCCAT
GGACTACTGGGGCCAGGGAACACTGGTCACCGTGTCCAGCGGCGGTGGCGGTTC
TAATGCCGGCGTGACCCAGACACCTAAGTTCCAGGTGCTGAAAACCGGCCAGAG
CATGACCCTGCAGTGCGCCCAGGATATGAACCACGAGTACATGAGCTGGTACAG
ACAGGACCCTGGCATGGGCCTGAGACTGATCCACTATTCTGTCGGAGCCGGCATC
ACCGACCAGGGCGAAGTTCCTAATGGCTACAACGTGTCCAGAAGCACCACCGAG
GACTTCCCACTGAGACTGCTGTCTGCCGCTCCTAGCCAGACCAGCGTGTACTTCT
GTGCCTCTAGACCTGGACTGGCTGGCGGCAGACCTGAGCAGTATTTTGGCCCTGG
CACCAGACTGACCGTGACCGAGGACCTGAGAAACGTGACCCCTCCTAAGGTGTC
CCTGTTCGAGCCTAGCAAGGCCGAGATCGCCAACAAGCAGAAAGCCACACTCGT
GTGCCTGGCCAGAGGCTTCTTTCCCGATCACGTGGAACTGTCTTGGTGGGTCAAC
GGCAAAGAGGTGCACAGCGGCGTCTGTACTGATCCCCAGGCCTACAAAGAGAGC
AACTACAGCTACTGCCTGAGCAGCAGACTGAGAGTGTCCGCCACCTTCTGGCAC
AACCCCAGAAACCACTTCAGATGCCAGGTGCAGTTTCACGGCCTGAGCGAAGAG
GACAAGTGGCCTGAGGGAAGCCCCAAGCCAGTGACACAGAATATCTCTGCCGAA
GCCTGGGGCAGAGCCGATTGTGGAATTACCAGCGCCAGCTACCAGCAAGGCGTG
CTGTCTGCCACAATCCTGTACGAGATCCTGCTGGGCAAAGCCACTCTGTACGCCG
TGCTGGTGTCTACCCTGGTCGTGATGGCCATGGTCAAGCGGAAGAACAGCAGGG
CCAAGAGAAGTGGAAGCGGCGCCACAAACTTCAGCCTGTTGAAACAGGCCGGGG
ACGTCGAAGAGAATCCTGGACCAATGGCACTGCCCGTCACTGCCTTGTTGCTGCC
ACTTGCACTGCTGCTCCATGCCGCCAGGCCAGACATCCAGATGACACAGAGCCCT
AGCAGCCTGTCTGCCAGCGTGGGAGACAGAGTGACCATCACCTGTAGAGCCAGC
CAGGACGTGAACACAGCCGTGGCTTGGTATCAGCAGAAGCCTGGCAAGGCCCCT
AAGCTGCTGATCTACAGCGCCTACTTCCTGTACAGCGGCGTGCCCAGCAGATTCA
GCGGCTCTAGAAGCGGCACCGACTTCACCCTGACCATAAGCAGTCTGCAGCCCG
AGGACTTCGCCACCTACTACTGTCAGCAGTACAGCCGGTACAGCCCCGTGACATT
TGGCCAGGGCACCAAGGTGGAAATCAAGGGCGGTGGCGGTTCTCAGAAAGAGGT
GGAACAGAACAGCGGCCCTCTGTCTGTTCCTGAAGGCGCTATCGCCAGCCTGAA
CTGCACCTACAGCGATAGAGGCAGCCAGAGCTTCTTCTGGTACAGACAGTACAG
CGGCAAGAGCCCCGAGCTGATCATGAGCATCTACAGCAACGGCGACAAAGAGG
ACGGCCGGTTTACAGCCCAGCTGAACAAGGCCAGCCAGTACGTGTCCCTGCTGA
TCAGAGATAGCCAGCCTAGCGACAGCGCCACCTATCTGTGTGCCGTGACCACAG
ATAGCTGGGGCAAGCTGCAGTTTGGCGCCGGAACACAGGTGGTGGTTACACCTG
ACATTCAGAATCCTGAGCCTGCCGTGTACCAGCTGAAGGACCCTAGAAGCCAGG
ACAGCACCCTGTGCCTGTTCACCGACTTCGACAGCCAGATCAACGTGCCCAAGAC
CATGGAAAGCGGCACCTTCATCACCGATAAGTGCGTGCTGGACATGAAGGCCAT
GGACAGCAAGAGCAACGGCGCCATTGCCTGGTCCAACCAGACCAGCTTCACGTG
CCAGGACATCTTCAAAGAGACAAACGCCACCTATCCTAGCAGCGACGTGCCCTG
TGATGCCACACTGACCGAGAAGTCCTTCGAGACAGACATGAACCTGAACTTCCA
GAACCTGCTGGTCATCGTGCTGAGAATCCTGCTGCTGAAGGTGGCCGGCTTCAAC
CTGCTGATGACCCTGAGACTGTGGTCCAGCCGGGCCAAACGATCTGGCTCTGGCC
AGTGTACCAACTATGCCCTGCTCAAGCTCGCTGGCGACGTCGAGTCTAACCCTGG
ACCAATGGGAGCTGGCGCTACAGGCAGAGCTATGGATGGACCTAGACTGCTGCT
GCTCCTGCTTCTGGGAGTGTCTCTTGGCGGAGCCAAAGAGGCCTGTCCTACCGGC
CTGTATACACACTCTGGCGAGTGCTGCAAGGCCTGCAATCTTGGAGAAGGCGTC
GCACAGCCTTGCGGCGCTAATCAGACAGTGTGCGAGCCTTGCCTGGACAGCGTG
ACCTTTAGCGACGTGGTGTCAGCCACCGAGCCATGCAAGCCATGTACCGAGTGT
GTGGGCCTGCAGAGCATGTCTGCCCCTTGCGTGGAAGCTGACGATGCCGTGTGTA
GATGCGCCTACGGCTACTACCAGGACGAGACAACAGGCAGATGCGAGGCCTGTA
GAGTGTGCGAAGCTGGATCTGGCCTGGTGTTCAGCTGCCAAGACAAGCAGAACA
CCGTGTGCGAGGAATGCCCCGATGGCACCTATAGCGACGAGGCCAACCATGTGG
ATCCCTGCCTGCCTTGTACCGTGTGTGAAGATACCGAGCGGCAGCTGCGCGAGTG
TACAAGATGGGCTGATGCCGAGTGCGAAGAGATCCCTGGCAGATGGATCACCAG
AAGCACACCTCCAGAGGGCAGCGATAGCACAGCCCCTTCTACACAAGAGCCCGA
GGCTCCTCCTGAGCAGGACCTGATTGCTTCTACAGTGGCTGGCGTCGTGACCACC
GTGATGGGATCTAGTCAGCCCGTGGTCACCAGAGGCACCACCGACAATCTGATC
CCCGTGTACTGTAGCATCCTGGCCGCCGTGGTTGTTGGACTGGTGGCCTATATCG
CCTTCAAGCGGTGGAACAGCTGATGATTCGAAGCGGCCGCTTCGAGCAGACATG
ATAAGATACATTGATGAGTTTGGACAAACCACAACTAGAATGCAGTGAAAAAAA
TGCTTTATTTGTGAAATTTGTGATGCTATTGCTTTATTTGTAACCATTATAAGCTG
CAATAAACAAGTTACACGGCAGGGTCAGGGTTCTGGATATctgtgggacaagaggatcagggt
taggacatgatctcatttccctctttgccccaacccaggctggagtccagatgccagtgatggacaagggcggggctctgtggggctg
gcaagtcacggtctcatgctttatacgggaaatagcatcttagaaaccagctgctcgtgatggactgggactcagggacaggcacaag
ctatcaatcttggccaagaggccatgatttcagtgaacgttcacggccaggcctggcctgccactcaaggaaacCACTGGCCG
TCGTTTTACAACGTCGTGACTgggaaaaccctggcgttacccaacttaatcgccttgcagcacatccccctttcgcc
agctggcgtaatagcgaagaggcccgcaccgatcgcccttcccaacagttgcgcagcctgaatggcgaatggcgcctgatgcggta
ttttctccttacgcatctgtgcggtatttcacaccgcatatggtgcactctcagtacaatctgctctgatgccgcatagttaagccagcccc
gacacccgccaacacccgctgacgcgccctgacgggcttgtctgctcccggcatccgcttacagacaagctgtgaccgtctccggga
gctgcatgtgtcagaggttttcaccgtcatcaccgaaacgcgcga
-H2-VH-A6-TCRβ-2A-VL-A6-TCRα (amino acid sequence)
SEQ ID NO: 50
MALPVTALLLPLALLLHAARPEVQLVESGGGLVQPGGSLRLSCAASGFNVYASGMH
WVRQAPGKGLEWVAKIYPDSDYTYYADSVKGRFTISADTSKNTAYLQMNSLRAED
TAVYYCSRDSSFYYVYAMDYWGQGTLVTVSSGGGGSNAGVTQTPKFQVLKTGQS
MTLQCAQDMNHEYMSWYRQDPGMGLRLIHYSVGAGITDQGEVPNGYNVSRSTTED
FPLRLLSAAPSQTSVYFCASRPGLAGGRPEQYFGPGTRLTVTEDLRNVTPPKVSLFEP
SKAEIANKQKATLVCLARGFFPDHVELSWWVNGKEVHSGVCTDPQAYKESNYSYC
LSSRLRVSATFWHNPRNHFRCQVQFHGLSEEDKWPEGSPKPVTQNISAEAWGRADC
GITSASYQQGVLSATILYEILLGKATLYAVLVSTLVVMAMVKRKNSRAKRSGSGATN
FSLLKQAGDVEENPGPMALPVTALLLPLALLLHAARPDIQMTQSPSSLSASVGDRVTI
TCRASQDVNTAVAWYQQKPGKAPKLLIYSAYFLYSGVPSRFSGSRSGTDFTLTISSLQ
PEDFATYYCQQYSRYSPVTFGQGTKVEIKGGGGSQKEVEQNSGPLSVPEGAIASLNC
TYSDRGSQSFFWYRQYSGKSPELIMSIYSNGDKEDGRFTAQLNKASQYVSLLIRDSQP
SDSATYLCAVTTDSWGKLQFGAGTQVVVTPDIQNPEPAVYQLKDPRSQDSTLCLFTD
FDSQINVPKTMESGTFITDKCVLDMKAMDSKSNGAIAWSNQTSFTCQDIFKETNATY
PSSDVPCDATLTEKSFETDMNLNFQNLLVIVLRILLLKVAGFNLLMTLRLWSSRAKRS
GSGQCTNYALLKLAGDVESNPGPMGAGATGRAMDGPRLLLLLLLGVSLGGAKEAC
PTGLYTHSGECCKACNLGEGVAQPCGANQTVCEPCLDSVTFSDVVSATEPCKPCTEC
VGLQSMSAPCVEADDAVCRCAYGYYQDETTGRCEACRVCEAGSGLVFSCQDKQNT
VCEECPDGTYSDEANHVDPCLPCTVCEDTERQLRECTRWADAECEEIPGRWITRSTP
PEGSDSTAPSTQEPEAPPEQDLIASTVAGVVTTVMGSSQPVVTRGTTDNLIPVYCSILA
AVVVGLVAYIAFKRWNS
-TCR-1 + Cβ-MC (plasmid sequence)
SEQ ID NO: 51
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggggcaaggctggc
ccggtcggcaccagttgcgtgagcggaaagatggccgcttcccggccctgctgcagggagctcaaaatggaggacgcggcgctcg
ggagagcggggggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
GCACCAGACTGCTGTGTTGGGCTGCTCTGTGTCTGCTGGGAGCCGAACTTACTGA
AGCTGGCGTGGCCCAGTCTCCTCGGTACAAGATCATCGAGAAGCGGCAGAGCGT
GGCCTTCTGGTGCAATCCTATCAGCGGACACGCCACACTGTACTGGTATCAGCAG
ATCCTCGGACAGGGCCCCAAGCTGCTGATTCAGTTCCAGAACAACGGCGTGGTG
GACGACAGCCAGCTGCCTAAGGATAGATTCAGCGCCGAGCGGCTGAAAGGCGTG
GACAGCACACTGAAGATCCAGCCTGCCAAGCTGGAAGATAGCGCCGTGTACCTG
TGTGCCAGCTCTCTGGATCCTGGCGATACCGGCGAGCTGTTCTTTGGCGAGGGCA
GCAGACTGACCGTGCTCGAGGACCTGAAGAACGTGTTCCCACCTGAGGTGGCCG
TGTTCGAGCCTTCTGAGGCCGAGATCAGCCACACACAGAAAGCCACACTCGTGT
GTCTGGCCACCGGCTTCTATCCCGATCACGTGGAACTGTCTTGGTGGGTCAACGG
CAAAGAGGTGCACAGCGGCGTCAGCACAGATCCCCAGCCTCTGAAAGAGCAGCC
AGCTCTGAACGACAGCCGGTACTGTCTGAGCAGCAGGCTGAGAGTGTCCGCCAC
CTTCTGGCAGAACCCCAGAAACCACTTCAGATGCCAGGTGCAGTTCTACGGCCTG
AGCGAGAACGATGAGTGGACACAGGATAGAGCCAAGCCTGTGACACAGATCGT
GTCTGCCGAAGCCTGGGGCAGAGCCGATTGTGGCTTTACCAGCGAGAGCTACCA
GCAGGGCGTGCTGTCTGCCACAATCCTGTACGAGATCCTGCTGGGCAAAGCCACT
CTGTACGCCGTGCTGGTGTCTGCCCTGGTGCTGATGGCCATGATGGCTGCTGGTG
GACCTGGTGCTGGATCTGCTGCCCCTGTGTCTAGCACATCTAGCCTGCCTCTGGC
CGCTCTGAACATGAGAGTCAGAAGAAGGCTGAGCCTGTTCCTGAACGTGCGGAC
TCAGGTGGCCGCTGATTGGACAGCTCTGGCCGAGGAAATGGACTTCGAGTACCT
GGAAATCCGGCAGCTGGAAACCCAGGCCGATCCTACAGGCAGACTGCTGGATGC
TTGGCAAGGCAGACCTGGCGCTTCTGTGGGGAGACTGCTTGAGCTGCTGACAAA
GCTGGGCAGAGATGACGTGCTGCTGGAACTGGGCCCTAGCATCGAGGAAGATTG
CCAGAAGTACATCCTGAAGCAGCAGCAAGAGGAAGCCGAGAAGCCTCTGCAAGT
GGCCGCCGTGGATAGCAGCGTTCCAAGAACAGCTGAGCTGGCCGGCATCACCAC
ACTGGATGATCCTCTGGGAAAGAAGGTGGCCAAGAAGCCCACCAACAAGGCCCC
TCATCCTAAGCAAGAGCCCCAAGAGATCAACTTCCCCGACGATCTGCCCGGCAG
CAATACTGCTGCTCCCGTGCAAGAAACCCTGCACGGTTGTCAGCCCGTGACACAA
GAGGACGGCAAAGAAAGCCGGATCAGCGTGCAAGAGAGACAGAGAGCCAAGAG
ATCTGGCAGCGGCGCCACAAACTTTAGCCTGCTGAAACAGGCCGGCGACGTGGA
AGAGAACCCCGGACCTATGGAATCCTTTCTCGGTGGCGTGCTGCTCATCCTGTGG
CTGCAAGTGGATTGGGTCAAGAGCCAGAAGATCGAGCAGAACAGCGAGGCCCTG
AACATCCAAGAGGGCAAGACCGCCACTCTGACCTGCAACTACACCAACTACAGC
CCCGCCTACCTGCAGTGGTACAGACAGGACCCTGGAAGAGGCCCTGTGTTCCTGC
TGCTGATCCGCGAGAATGAGAAAGAGAAGCGCAAAGAGCGCCTGAAAGTCACCT
TCGACACAACCCTGAAGCAGAGCCTGTTCCACATCACCGCCTCTCAGCCAGCCGA
TAGCGCCACATATCTGTGCGCCCTGGACATCTACCCTCACGACATGAGATTCGGA
GCCGGCACCAGGCTGACAGTGAAGCCCGATATTCAGAACCCCGATCCTGCCGTC
TATCAGCTGAGAGACAGCAAGAGCAGCGACAAGAGCGTGTGCCTGTTCACCGAC
TTCGACTCCCAGACCAACGTGTCCCAGAGCAAGGACAGCGACGTGTACATCACC
GACAAGACCGTGCTGGACATGCGGAGCATGGACTTCAAGAGCAACAGCGCCGTG
GCCTGGTCCAACAAGAGCGATTTCGCCTGCGCCAACGCCTTCAACAACAGCATTA
TCCCCGAGGACACATTCTTCCCAAGTCCTGAGAGCAGCTGCGACGTGAAGCTGGT
GGAAAAGAGCTTCGAGACAGACACCAACCTGAACTTCCAGAACCTGAGCGTGAT
CGGCTTCCGGATCCTGCTCCTGAAAGTGGCCGGCTTCAACCTGCTGATGACCCTG
AGACTGTGGTCCAGCCGGGCCAAACGATCTGGCTCTGGCCAGTGTACCAACTAT
GCCCTGCTCAAGCTCGCTGGCGACGTCGAGTCTAACCCTGGACCAATGGGAGCT
GGCGCTACAGGCAGAGCTATGGATGGACCTAGACTGCTGCTGCTCCTGCTTCTGG
GAGTGTCTCTTGGCGGAGCCAAAGAGGCCTGTCCTACCGGCCTGTATACACACTC
TGGCGAGTGCTGCAAGGCCTGCAATCTTGGAGAAGGCGTCGCACAGCCTTGCGG
CGCTAATCAGACAGTGTGCGAGCCTTGCCTGGACAGCGTGACCTTTAGCGACGTG
GTGTCAGCCACCGAGCCATGCAAGCCATGTACCGAGTGTGTGGGCCTGCAGAGC
ATGTCTGCCCCTTGCGTGGAAGCTGACGATGCCGTGTGTAGATGCGCCTACGGCT
ACTACCAGGACGAGACAACAGGCAGATGCGAGGCCTGTAGAGTGTGCGAAGCTG
GATCTGGCCTGGTGTTCAGCTGCCAAGACAAGCAGAACACCGTGTGCGAGGAAT
GCCCCGATGGCACCTATAGCGACGAGGCCAACCATGTGGATCCCTGCCTGCCTTG
TACCGTGTGTGAAGATACCGAGCGGCAGCTGCGCGAGTGTACAAGATGGGCTGA
TGCCGAGTGCGAAGAGATCCCTGGCAGATGGATCACCAGAAGCACACCTCCAGA
GGGCAGCGATAGCACAGCCCCTTCTACACAAGAGCCCGAGGCTCCTCCTGAGCA
GGACCTGATTGCTTCTACAGTGGCTGGCGTCGTGACCACCGTGATGGGATCTAGT
CAGCCCGTGGTCACCAGAGGCACCACCGACAATCTGATCCCCGTGTACTGTAGC
ATCCTGGCCGCCGTGGTTGTTGGACTGGTGGCCTATATCGCCTTCAAGCGGTGGA
ACAGCTGATGATTCGAAGCGGCCGCTTCGAGCAGACATGATAAGATACATTGAT
GAGTTTGGACAAACCACAACTAGAATGCAGTGAAAAAAATGCTTTATTTGTGAA
ATTTGTGATGCTATTGCTTTATTTGTAACCATTATAAGCTGCAATAAACAAGTTAC
ACGGCAGGGTCAGGGTTCTGGATATctgtgggacaagaggatcagggttaggacatgatctcatttccctcttt
gccccaacccaggctggagtccagatgccagtgatggacaagggcggggctctgtggggctggcaagtcacggtctcatgctttata
cgggaaatagcatcttagaaaccagctgctcgtgatggactgggactcagggacaggcacaagctatcaatcttggccaagaggcca
tgatttcagtgaacgttcacggccaggcctggcctgccactcaaggaaacCACTGGCCGTCGTTTTACAACGTC
GTGACTgggaaaaccctggcgttacccaacttaatcgccttgcagcacatccccctttcgccagctggcgtaatagcgaagagg
cccgcaccgatcgcccttcccaacagttgcgcagcctgaatggcgaatggcgcctgatgcggtattttctccttacgcatctgtgcggta
tttcacaccgcatatggtgcactctcagtacaatctgctctgatgccgcatagttaagccagccccgacacccgccaacacccgctgac
gcgccctgacgggcttgtctgctcccggcatccgcttacagacaagctgtgaccgtctccgggagctgcatgtgtcagaggttttcacc
gtcatcaccgaaacgcgcga
-TCR-1 + Cβ-MC (amino acid sequence)
SEQ ID NO: 52
MGTRLLCWAALCLLGAELTEAGVAQSPRYKIIEKRQSVAFWCNPISGHATLYWYQQ
ILGQGPKLLIQFQNNGVVDDSQLPKDRFSAERLKGVDSTLKIQPAKLEDSAVYLCASS
LDPGDTGELFFGEGSRLTVLEDLKNVFPPEVAVFEPSEAEISHTQKATLVCLATGFYP
DHVELSWWVNGKEVHSGVSTDPQPLKEQPALNDSRYCLSSRLRVSATFWQNPRNHF
RCQVQFYGLSENDEWTQDRAKPVTQIVSAEAWGRADCGFTSESYQQGVLSATILYEI
LLGKATLYAVLVSALVLMAMMAAGGPGAGSAAPVSSTSSLPLAALNMRVRRRLSL
FLNVRTQVAADWTALAEEMDFEYLEIRQLETQADPTGRLLDAWQGRPGASVGRLLE
LLTKLGRDDVLLELGPSIEEDCQKYILKQQQEEAEKPLQVAAVDSSVPRTAELAGITT
LDDPLGKKVAKKPTNKAPHPKQEPQEINFPDDLPGSNTAAPVQETLHGCQPVTQEDG
KESRISVQERQRAKRSGSGATNFSLLKQAGDVEENPGPMESFLGGVLLILWLQVDW
VKSQKIEQNSEALNIQEGKTATLTCNYTNYSPAYLQWYRQDPGRGPVFLLLIRENEK
EKRKERLKVTFDTTLKQSLFHITASQPADSATYLCALDIYPHDMRFGAGTRLTVKPDI
QNPDPAVYQLRDSKSSDKSVCLFTDFDSQTNVSQSKDSDVYITDKTVLDMRSMDFK
SNSAVAWSNKSDFACANAFNNSIIPEDTFFPSPESSCDVKLVEKSFETDTNLNFQNLS
VIGFRILLLKVAGFNLLMTLRLWSSRAKRSGSGQCTNYALLKLAGDVESNPGPMGA
GATGRAMDGPRLLLLLLLGVSLGGAKEACPTGLYTHSGECCKACNLGEGVAQPCG
ANQTVCEPCLDSVTFSDVVSATEPCKPCTECVGLQSMSAPCVEADDAVCRCAYGYY
QDETTGRCEACRVCEAGSGLVFSCQDKQNTVCEECPDGTYSDEANHVDPCLPCTVC
EDTERQLRECTRWADAECEEIPGRWITRSTPPEGSDSTAPSTQEPEAPPEQDLIASTVA
GVVTTVMGSSQPVVTRGTTDNLIPVYCSILAAVVVGLVAYIAFKRWNS
-TCR-1 + Fas-MC (plasmid sequence)
SEQ ID NO: 53
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
gaagataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcggggggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
GCACCAGACTGCTGTGTTGGGCTGCTCTGTGTCTGCTGGGAGCCGAACTTACTGA
AGCTGGCGTGGCCCAGTCTCCTCGGTACAAGATCATCGAGAAGCGGCAGAGCGT
GGCCTTCTGGTGCAATCCTATCAGCGGACACGCCACACTGTACTGGTATCAGCAG
ATCCTCGGACAGGGCCCCAAGCTGCTGATTCAGTTCCAGAACAACGGCGTGGTG
GACGACAGCCAGCTGCCTAAGGATAGATTCAGCGCCGAGCGGCTGAAAGGCGTG
GACAGCACACTGAAGATCCAGCCTGCCAAGCTGGAAGATAGCGCCGTGTACCTG
TGTGCCAGCTCTCTGGATCCTGGCGATACCGGCGAGCTGTTCTTTGGCGAGGGCA
GCAGACTGACCGTGCTCGAGGACCTGAAGAACGTGTTCCCACCTGAGGTGGCCG
TGTTCGAGCCTTCTGAGGCCGAGATCAGCCACACACAGAAAGCCACACTCGTGT
GTCTGGCCACCGGCTTCTATCCCGATCACGTGGAACTGTCTTGGTGGGTCAACGG
CAAAGAGGTGCACAGCGGCGTCAGCACAGATCCCCAGCCTCTGAAAGAGCAGCC
AGCTCTGAACGACAGCCGGTACTGTCTGAGCAGCAGGCTGAGAGTGTCCGCCAC
CTTCTGGCAGAACCCCAGAAACCACTTCAGATGCCAGGTGCAGTTCTACGGCCTG
AGCGAGAACGATGAGTGGACACAGGATAGAGCCAAGCCTGTGACACAGATCGT
GTCTGCCGAAGCCTGGGGCAGAGCCGATTGTGGCTTTACCAGCGAGAGCTACCA
GCAGGGCGTGCTGTCTGCCACAATCCTGTACGAGATCCTGCTGGGCAAAGCCACT
CTGTACGCCGTGCTGGTGTCTGCCCTGGTGCTGATGGCCATGGTCAAGCGGAAGG
ACTCTAGAGGCAGAGCCAAGAGATCTGGCAGCGGCGCCACAAACTTTAGCCTGC
TGAAACAGGCCGGCGACGTGGAAGAGAACCCCGGACCTATGGAATCCTTTCTCG
GTGGCGTGCTGCTCATCCTGTGGCTGCAAGTGGATTGGGTCAAGAGCCAGAAGA
TCGAGCAGAACAGCGAGGCCCTGAACATCCAAGAGGGCAAGACCGCCACTCTGA
CCTGCAACTACACCAACTACAGCCCCGCCTACCTGCAGTGGTACAGACAGGACC
CTGGAAGAGGCCCTGTGTTCCTGCTGCTGATCCGCGAGAATGAGAAAGAGAAGC
GCAAAGAGCGCCTGAAAGTCACCTTCGACACAACCCTGAAGCAGAGCCTGTTCC
ACATCACCGCCTCTCAGCCAGCCGATAGCGCCACATATCTGTGCGCCCTGGACAT
CTACCCTCACGACATGAGATTCGGAGCCGGCACCAGGCTGACAGTGAAGCCCGA
TATTCAGAACCCCGATCCTGCCGTCTATCAGCTGAGAGACAGCAAGAGCAGCGA
CAAGAGCGTGTGCCTGTTCACCGACTTCGACTCCCAGACCAACGTGTCCCAGAGC
AAGGACAGCGACGTGTACATCACCGACAAGACCGTGCTGGACATGCGGAGCATG
GACTTCAAGAGCAACAGCGCCGTGGCCTGGTCCAACAAGAGCGATTTCGCCTGC
GCCAACGCCTTCAACAACAGCATTATCCCCGAGGACACATTCTTCCCAAGTCCTG
AGAGCAGCTGCGACGTGAAGCTGGTGGAAAAGAGCTTCGAGACAGACACCAAC
CTGAACTTCCAGAACCTGAGCGTGATCGGCTTCCGGATCCTGCTCCTGAAAGTGG
CCGGCTTCAACCTGCTGATGACCCTGAGACTGTGGTCCAGCCGGGCCAAACGATC
TGGCTCTGGCCAGTGTACCAACTATGCCCTGCTCAAGCTCGCTGGCGACGTCGAG
TCTAACCCTGGACCAATGCTCGGCATCTGGACACTGCTGCCTCTGGTGCTGACAA
GCGTGGCCAGACTGAGCAGCAAGAGCGTGAACGCCCAAGTGACCGACATCAACA
GCAAAGGCCTGGAACTGAGAAAGACCGTGACCACCGTGGAAACCCAGAACCTG
GAAGGCCTGCACCACGACGGCCAGTTCTGTCACAAACCTTGTCCACCTGGCGAG
CGGAAGGCCAGAGATTGCACAGTGAATGGCGACGAGCCTGACTGCGTGCCCTGT
CAAGAGGGCAAAGAGTACACCGACAAGGCCCACTTCAGCAGCAAGTGCAGACG
GTGCAGACTGTGCGACGAAGGCCACGGACTGGAAGTGGAAATCAACTGCACCCG
GACACAGAACACCAAGTGCCGGTGCAAGCCCAACTTCTTCTGCAACAGCACCGT
GTGCGAGCACTGCGACCCTTGTACCAAGTGCGAACACGGCATCATCAAAGAGTG
CACCCTGACCTCCAACACGAAGTGCAAAGAGGAAGGCAGCAGAAGCAACCTCG
GCTGGCTGTGTCTGCTGCTGCTCCCCATTCCTCTGATCGTGTGGGTTATGGCTGCT
GGCGGACCTGGTGCTGGATCTGCTGCACCTGTGTCCAGCACATCTAGCCTGCCAC
TGGCCGCTCTGAACATGAGAGTCAGAAGAAGGCTGAGCCTGTTCCTGAACGTGC
GCACTCAGGTGGCCGCTGATTGGACAGCTCTGGCCGAGGAAATGGACTTCGAGT
ACCTGGAAATCCGGCAGCTGGAAACACAGGCCGATCCTACAGGCAGACTGCTGG
ATGCTTGGCAAGGCAGACCTGGCGCTTCTGTGGGGAGACTGCTTGAGCTGCTGAC
AAAGCTGGGCAGAGATGACGTGCTGCTGGAACTGGGACCCAGCATCGAGGAAG
ATTGCCAGAAGTACATCCTGAAGCAGCAACAAGAGGAAGCCGAGAAGCCTCTGC
AGGTTGCCGCCGTGGATAGCAGCGTTCCAAGAACAGCTGAGCTGGCCGGCATCA
CCACACTGGATGATCCTCTGGGAAAGAAGGTGGCCAAGAAGCCCACAAACAAGG
CCCCTCATCCTAAGCAAGAGCCCCAAGAGATCAACTTCCCCGACGATCTGCCCGG
CAGCAATACTGCAGCTCCCGTGCAAGAAACCCTGCACGGTTGTCAGCCCGTGAC
ACAAGAGGACGGCAAAGAAAGCCGGATCAGCGTGCAAGAGAGACAGTGATGAT
TCGAAGCGGCCGCTTCGAGCAGACATGATAAGATACATTGATGAGTTTGGACAA
ACCACAACTAGAATGCAGTGAAAAAAATGCTTTATTTGTGAAATTTGTGATGCTA
TTGCTTTATTTGTAACCATTATAAGCTGCAATAAACAAGTTACACGGCAGGGTCA
GGGTTCTGGATATctgtgggacaagaggatcagggttaggacatgatctcatttccctctttgccccaacccaggctggag
tccagatgccagtgatggacaagggcggggctctgtggggctggcaagtcacggtctcatgctttatacgggaaatagcatcttagaa
accagctgctcgtgatggactgggactcagggacaggcacaagctatcaatcttggccaagaggccatgatttcagtgaacgttcacg
gccaggcctggcctgccactcaaggaaacCACTGGCCGTCGTTTTACAACGTCGTGACTgggaaaaccc
tggcgttacccaacttaatcgccttgcagcacatccccctttcgccagctggcgtaatagcgaagaggcccgcaccgatcgcccttccc
aacagttgcgcagcctgaatggcgaatggcgcctgatgcggtattttctccttacgcatctgtgcggtatttcacaccgcatatggtgcac
tctcagtacaatctgctctgatgccgcatagttaagccagccccgacacccgccaacacccgctgacgcgccctgacgggcttgtctg
ctcccggcatccgcttacagacaagctgtgaccgtctccgggagctgcatgtgtcagaggttttcaccgtcatcaccgaaacgcgcga
-TCR-1 + Fas-MC (amino acid sequence)
SEQ ID NO: 54
MGTRLLCWAALCLLGAELTEAGVAQSPRYKIIEKRQSVAFWCNPISGHATLYWYQQ
ILGQGPKLLIQFQNNGVVDDSQLPKDRFSAERLKGVDSTLKIQPAKLEDSAVYLCASS
LDPGDTGELFFGEGSRLTVLEDLKNVFPPEVAVFEPSEAEISHTQKATLVCLATGFYP
DHVELSWWVNGKEVHSGVSTDPQPLKEQPALNDSRYCLSSRLRVSATFWQNPRNHF
RCQVQFYGLSENDEWTQDRAKPVTQIVSAEAWGRADCGFTSESYQQGVLSATILYEI
LLGKATLYAVLVSALVLMAMVKRKDSRGRAKRSGSGATNFSLLKQAGDVEENPGP
MESFLGGVLLILWLQVDWVKSQKIEQNSEALNIQEGKTATLTCNYTNYSPAYLQWY
RQDPGRGPVFLLLIRENEKEKRKERLKVTFDTTLKQSLFHITASQPADSATYLCALDI
YPHDMRFGAGTRLTVKPDIQNPDPAVYQLRDSKSSDKSVCLFTDFDSQTNVSQSKDS
DVYITDKTVLDMRSMDFKSNSAVAWSNKSDFACANAFNNSIIPEDTFFPSPESSCDV
KLVEKSFETDTNLNFQNLSVIGFRILLLKVAGFNLLMTLRLWSSRAKRSGSGQCTNY
ALLKLAGDVESNPGPMLGIWTLLPLVLTSVARLSSKSVNAQVTDINSKGLELRKTVT
TVETQNLEGLHHDGQFCHKPCPPGERKARDCTVNGDEPDCVPCQEGKEYTDKAHFS
SKCRRCRLCDEGHGLEVEINCTRTQNTKCRCKPNFFCNSTVCEHCDPCTKCEHGIIKE
CTLTSNTKCKEEGSRSNLGWLCLLLLPIPLIVWVMAAGGPGAGSAAPVSSTSSLPLAA
LNMRVRRRLSLFLNVRTQVAADWTALAEEMDFEYLEIRQLETQADPTGRLLDAWQ
GRPGASVGRLLELLTKLGRDDVLLELGPSIEEDCQKYILKQQQEEAEKPLQVAAVDS
SVPRTAELAGITTLDDPLGKKVAKKPTNKAPHPKQEPQEINFPDDLPGSNTAAPVQET
LHGCQPVTQEDGKESRISVQERQ
-TCR-1 + Fas-OX40 (plasmid sequence)
SEQ ID NO: 55
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
GCACCAGACTGCTGTGTTGGGCTGCTCTGTGTCTGCTGGGAGCCGAACTTACTGA
AGCTGGCGTGGCCCAGTCTCCTCGGTACAAGATCATCGAGAAGCGGCAGAGCGT
GGCCTTCTGGTGCAATCCTATCAGCGGACACGCCACACTGTACTGGTATCAGCAG
ATCCTCGGACAGGGCCCCAAGCTGCTGATTCAGTTCCAGAACAACGGCGTGGTG
GACGACAGCCAGCTGCCTAAGGATAGATTCAGCGCCGAGCGGCTGAAAGGCGTG
GACAGCACACTGAAGATCCAGCCTGCCAAGCTGGAAGATAGCGCCGTGTACCTG
TGTGCCAGCTCTCTGGATCCTGGCGATACCGGCGAGCTGTTCTTTGGCGAGGGCA
GCAGACTGACCGTGCTCGAGGACCTGAAGAACGTGTTCCCACCTGAGGTGGCCG
TGTTCGAGCCTTCTGAGGCCGAGATCAGCCACACACAGAAAGCCACACTCGTGT
GTCTGGCCACCGGCTTCTATCCCGATCACGTGGAACTGTCTTGGTGGGTCAACGG
CAAAGAGGTGCACAGCGGCGTCAGCACAGATCCCCAGCCTCTGAAAGAGCAGCC
AGCTCTGAACGACAGCCGGTACTGTCTGAGCAGCAGGCTGAGAGTGTCCGCCAC
CTTCTGGCAGAACCCCAGAAACCACTTCAGATGCCAGGTGCAGTTCTACGGCCTG
AGCGAGAACGATGAGTGGACACAGGATAGAGCCAAGCCTGTGACACAGATCGT
GTCTGCCGAAGCCTGGGGCAGAGCCGATTGTGGCTTTACCAGCGAGAGCTACCA
GCAGGGCGTGCTGTCTGCCACAATCCTGTACGAGATCCTGCTGGGCAAAGCCACT
CTGTACGCCGTGCTGGTGTCTGCCCTGGTGCTGATGGCCATGGTCAAGCGGAAGG
ACTCTAGAGGCAGAGCCAAGAGATCTGGCAGCGGCGCCACAAACTTTAGCCTGC
TGAAACAGGCCGGCGACGTGGAAGAGAACCCCGGACCTATGGAATCCTTTCTCG
GTGGCGTGCTGCTCATCCTGTGGCTGCAAGTGGATTGGGTCAAGAGCCAGAAGA
TCGAGCAGAACAGCGAGGCCCTGAACATCCAAGAGGGCAAGACCGCCACTCTGA
CCTGCAACTACACCAACTACAGCCCCGCCTACCTGCAGTGGTACAGACAGGACC
CTGGAAGAGGCCCTGTGTTCCTGCTGCTGATCCGCGAGAATGAGAAAGAGAAGC
GCAAAGAGCGCCTGAAAGTCACCTTCGACACAACCCTGAAGCAGAGCCTGTTCC
ACATCACCGCCTCTCAGCCAGCCGATAGCGCCACATATCTGTGCGCCCTGGACAT
CTACCCTCACGACATGAGATTCGGAGCCGGCACCAGGCTGACAGTGAAGCCCGA
TATTCAGAACCCCGATCCTGCCGTCTATCAGCTGAGAGACAGCAAGAGCAGCGA
CAAGAGCGTGTGCCTGTTCACCGACTTCGACTCCCAGACCAACGTGTCCCAGAGC
AAGGACAGCGACGTGTACATCACCGACAAGACCGTGCTGGACATGCGGAGCATG
GACTTCAAGAGCAACAGCGCCGTGGCCTGGTCCAACAAGAGCGATTTCGCCTGC
GCCAACGCCTTCAACAACAGCATTATCCCCGAGGACACATTCTTCCCAAGTCCTG
AGAGCAGCTGCGACGTGAAGCTGGTGGAAAAGAGCTTCGAGACAGACACCAAC
CTGAACTTCCAGAACCTGAGCGTGATCGGCTTCCGGATCCTGCTCCTGAAAGTGG
CCGGCTTCAACCTGCTGATGACCCTGAGACTGTGGTCCAGCCGGGCCAAACGATC
TGGCTCTGGCCAGTGTACCAACTATGCCCTGCTCAAGCTCGCTGGCGACGTCGAG
TCTAACCCTGGACCAATGCTCGGCATCTGGACACTGCTGCCTCTGGTGCTGACAA
GCGTGGCCAGACTGAGCAGCAAGAGCGTGAACGCCCAAGTGACCGACATCAACA
GCAAAGGCCTGGAACTGAGAAAGACCGTGACCACCGTGGAAACCCAGAACCTG
GAAGGCCTGCACCACGACGGCCAGTTCTGTCACAAACCTTGTCCACCTGGCGAG
CGGAAGGCCAGAGATTGCACAGTGAATGGCGACGAGCCTGACTGCGTGCCCTGT
CAAGAGGGCAAAGAGTACACCGACAAGGCCCACTTCAGCAGCAAGTGCAGACG
GTGCAGACTGTGCGACGAAGGCCACGGACTGGAAGTGGAAATCAACTGCACCCG
GACACAGAACACCAAGTGCCGGTGCAAGCCCAACTTCTTCTGCAACAGCACCGT
GTGCGAGCACTGCGACCCTTGTACCAAGTGCGAACACGGCATCATCAAAGAGTG
CACCCTGACCTCCAACACGAAGTGCAAAGAGGAAGGCAGCAGAAGCAACCTCG
GCTGGCTGTGTCTGCTGCTGCTCCCCATTCCTCTGATCGTGTGGGTCGCCCTGTAC
CTGCTGAGAAGGGATCAGAGACTGCCTCCTGACGCTCACAAACCACCAGGCGGA
GGCAGCTTCAGAACCCCTATCCAAGAGGAACAGGCTGACGCCCACAGCACCCTG
GCCAAAATTTGATGATTCGAAGCGGCCGCTTCGAGCAGACATGATAAGATACAT
TGATGAGTTTGGACAAACCACAACTAGAATGCAGTGAAAAAAATGCTTTATTTGT
GAAATTTGTGATGCTATTGCTTTATTTGTAACCATTATAAGCTGCAATAAACAAG
TTACACGGCAGGGTCAGGGTTCTGGATATctgtgggacaagaggatcagggttaggacatgatctcatttc
cctctttgccccaacccaggctggagtccagatgccagtgatggacaagggcggggctctgtggggctggcaagtcacggtctcatg
ctttatacgggaaatagcatcttagaaaccagctgctcgtgatggactgggactcagggacaggcacaagctatcaatcttggccaag
aggccatgatttcagtgaacgttcacggccaggcctggcctgccactcaaggaaacCACTGGCCGTCGTTTTACAA
CGTCGTGACTgggaaaaccctggcgttacccaacttaatcgccttgcagcacatccccctttcgccagctggcgtaatagcga
agaggcccgcaccgatcgcccttcccaacagttgcgcagcctgaatggcgaatggcgcctgatgcggtattttctccttacgcatctgt
gcggtatttcacaccgcatatgggcactctcagtacaatctgctctgatgccgcatagttaagccagccccgacacccgccaacaccc
gctgacgcgccctgacgggcttgtctgctcccggcatccgcttacagacaagctgtgaccgtctccgggagctgcatgtgtcagaggt
tttcaccgtcatcaccgaaacgcgcga
-TCR-1 + Fas-OX40 (amino acid sequence)
SEQ ID NO: 56
MGTRLLCWAALCLLGAELTEAGVAQSPRYKIIEKRQSVAFWCNPISGHATLYWYQQ
ILGQGPKLLIQFQNNGVVDDSQLPKDRFSAERLKGVDSTLKIQPAKLEDSAVYLCASS
LDPGDTGELFFGEGSRLTVLEDLKNVFPPEVAVFEPSEAEISHTQKATLVCLATGFYP
DHVELSWWVNGKEVHSGVSTDPQPLKEQPALNDSRYCLSSRLRVSATFWQNPRNHF
RCQVQFYGLSENDEWTQDRAKPVTQIVSAEAWGRADCGFTSESYQQGVLSATILYEI
LLGKATLYAVLVSALVLMAMVKRKDSRGRAKRSGSGATNFSLLKQAGDVEENPGP
MESFLGGVLLILWLQVDWVKSQKIEQNSEALNIQEGKTATLTCNYTNYSPAYLQWY
RQDPGRGPVFLLLIRENEKEKRKERLKVTFDTTLKQSLFHITASQPADSATYLCALDI
YPHDMRFGAGTRLTVKPDIQNPDPAVYQLRDSKSSDKSVCLFTDFDSQTNVSQSKDS
DVYITDKTVLDMRSMDFKSNSAVAWSNKSDFACANAFNNSIIPEDTFFPSPESSCDV
KLVEKSFETDTNLNFQNLSVIGFRILLLKVAGFNLLMTLRLWSSRAKRSGSGQCTNY
ALLKLAGDVESNPGPMLGIWTLLPLVLTSVARLSSKSVNAQVTDINSKGLELRKTVT
TVETQNLEGLHHDGQFCHKPCPPGERKARDCTVNGDEPDCVPCQEGKEYTDKAHFS
SKCRRCRLCDEGHGLEVEINCTRTQNTKCRCKPNFFCNSTVCEHCDPCTKCEHGIIKE
CTLTSNTKCKEEGSRSNLGWLCLLLLPIPLIVWVALYLLRRDQRLPPDAHKPPGGGSF
RTPIQEEQADAHSTLAKI
-TCR-1 + Fas-41BB (plasmid sequence)
SEQ ID NO: 57
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcggggggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
GCACCAGACTGCTGTGTTGGGCTGCTCTGTGTCTGCTGGGAGCCGAACTTACTGA
AGCTGGCGTGGCCCAGTCTCCTCGGTACAAGATCATCGAGAAGCGGCAGAGCGT
GGCCTTCTGGTGCAATCCTATCAGCGGACACGCCACACTGTACTGGTATCAGCAG
ATCCTCGGACAGGGCCCCAAGCTGCTGATTCAGTTCCAGAACAACGGCGTGGTG
GACGACAGCCAGCTGCCTAAGGATAGATTCAGCGCCGAGCGGCTGAAAGGCGTG
GACAGCACACTGAAGATCCAGCCTGCCAAGCTGGAAGATAGCGCCGTGTACCTG
TGTGCCAGCTCTCTGGATCCTGGCGATACCGGCGAGCTGTTCTTTGGCGAGGGCA
GCAGACTGACCGTGCTCGAGGACCTGAAGAACGTGTTCCCACCTGAGGTGGCCG
TGTTCGAGCCTTCTGAGGCCGAGATCAGCCACACACAGAAAGCCACACTCGTGT
GTCTGGCCACCGGCTTCTATCCCGATCACGTGGAACTGTCTTGGTGGGTCAACGG
CAAAGAGGTGCACAGCGGCGTCAGCACAGATCCCCAGCCTCTGAAAGAGCAGCC
AGCTCTGAACGACAGCCGGTACTGTCTGAGCAGCAGGCTGAGAGTGTCCGCCAC
CTTCTGGCAGAACCCCAGAAACCACTTCAGATGCCAGGTGCAGTTCTACGGCCTG
AGCGAGAACGATGAGTGGACACAGGATAGAGCCAAGCCTGTGACACAGATCGT
GTCTGCCGAAGCCTGGGGCAGAGCCGATTGTGGCTTTACCAGCGAGAGCTACCA
GCAGGGCGTGCTGTCTGCCACAATCCTGTACGAGATCCTGCTGGGCAAAGCCACT
CTGTACGCCGTGCTGGTGTCTGCCCTGGTGCTGATGGCCATGGTCAAGCGGAAGG
ACTCTAGAGGCAGAGCCAAGAGATCTGGCAGCGGCGCCACAAACTTTAGCCTGC
TGAAACAGGCCGGCGACGTGGAAGAGAACCCCGGACCTATGGAATCCTTTCTCG
GTGGCGTGCTGCTCATCCTGTGGCTGCAAGTGGATTGGGTCAAGAGCCAGAAGA
TCGAGCAGAACAGCGAGGCCCTGAACATCCAAGAGGGCAAGACCGCCACTCTGA
CCTGCAACTACACCAACTACAGCCCCGCCTACCTGCAGTGGTACAGACAGGACC
CTGGAAGAGGCCCTGTGTTCCTGCTGCTGATCCGCGAGAATGAGAAAGAGAAGC
GCAAAGAGCGCCTGAAAGTCACCTTCGACACAACCCTGAAGCAGAGCCTGTTCC
ACATCACCGCCTCTCAGCCAGCCGATAGCGCCACATATCTGTGCGCCCTGGACAT
CTACCCTCACGACATGAGATTCGGAGCCGGCACCAGGCTGACAGTGAAGCCCGA
TATTCAGAACCCCGATCCTGCCGTCTATCAGCTGAGAGACAGCAAGAGCAGCGA
CAAGAGCGTGTGCCTGTTCACCGACTTCGACTCCCAGACCAACGTGTCCCAGAGC
AAGGACAGCGACGTGTACATCACCGACAAGACCGTGCTGGACATGCGGAGCATG
GACTTCAAGAGCAACAGCGCCGTGGCCTGGTCCAACAAGAGCGATTTCGCCTGC
GCCAACGCCTTCAACAACAGCATTATCCCCGAGGACACATTCTTCCCAAGTCCTG
AGAGCAGCTGCGACGTGAAGCTGGTGGAAAAGAGCTTCGAGACAGACACCAAC
CTGAACTTCCAGAACCTGAGCGTGATCGGCTTCCGGATCCTGCTCCTGAAAGTGG
CCGGCTTCAACCTGCTGATGACCCTGAGACTGTGGTCCAGCCGGGCCAAACGATC
TGGCTCTGGCCAGTGTACCAACTATGCCCTGCTCAAGCTCGCTGGCGACGTCGAG
TCTAACCCTGGACCAATGCTCGGCATCTGGACACTGCTGCCTCTGGTGCTGACAA
GCGTGGCCAGACTGAGCAGCAAGAGCGTGAACGCCCAAGTGACCGACATCAACA
GCAAAGGCCTGGAACTGAGAAAGACCGTGACCACCGTGGAAACCCAGAACCTG
GAAGGCCTGCACCACGACGGCCAGTTCTGTCACAAACCTTGTCCACCTGGCGAG
CGGAAGGCCAGAGATTGCACAGTGAATGGCGACGAGCCTGACTGCGTGCCCTGT
CAAGAGGGCAAAGAGTACACCGACAAGGCCCACTTCAGCAGCAAGTGCAGACG
GTGCAGACTGTGCGACGAAGGCCACGGACTGGAAGTGGAAATCAACTGCACCCG
GACACAGAACACCAAGTGCCGGTGCAAGCCCAACTTCTTCTGCAACAGCACCGT
GTGCGAGCACTGCGACCCTTGTACCAAGTGCGAACACGGCATCATCAAAGAGTG
CACCCTGACCTCCAACACGAAGTGCAAAGAGGAAGGCAGCAGAAGCAACCTCG
GCTGGCTGTGTCTGCTGCTGCTCCCCATTCCTCTGATCGTGTGGGTCAAGCGGGG
CAGAAAGAAGCTGCTGTACATCTTCAAGCAGCCCTTCATGCGGCCCGTGCAGAC
CACACAAGAGGAAGATGGCTGCTCCTGCAGATTCCCCGAGGAAGAAGAAGGCG
GCTGCGAACTTTGATGATTCGAAGCGGCCGCTTCGAGCAGACATGATAAGATAC
ATTGATGAGTTTGGACAAACCACAACTAGAATGCAGTGAAAAAAATGCTTTATTT
GTGAAATTTGTGATGCTATTGCTTTATTTGTAACCATTATAAGCTGCAATAAACA
AGTTACACGGCAGGGTCAGGGTTCTGGATATctgtgggacaagaggatcagggttaggacatgatctca
tttccctctttgccccaacccaggctggagtccagatgccagtgatggacaagggcggggctctgtggggctggcaagtcacggtctc
atgctttatacgggaaatagcatcttagaaaccagctgctcgtgatggactgggactcagggacaggcacaagctatcaatcttggcca
agaggccatgatttcagtgaacgttcacggccaggcctggcctgccactcaaggaaacCACTGGCCGTCGTTTTAC
AACGTCGTGACTgggaaaaccctggcgttacccaacttaatcgccttgcagcacatccccctttcgccagctggcgtaatag
cgaagaggcccgcaccgatcgcccttcccaacagttgcgcagcctgaatggcgaatggcgcctgatgcggtattttctccttacgcat
ctgtgcggtatttcacaccgcatatggtgcactctcagtacaatctgctctgatgccgcatagttaagccagccccgacacccgccaac
acccgctgacgcgccctgacgggcttgtctgctcccggcatccgcttacagacaagctgtgaccgtctccgggagctgcatgtgtcag
aggttttcaccgtcatcaccgaaacgcgcga
-TCR-1 + Fas-41BB (amino acid sequence)
SEQ ID NO: 58
MGTRLLCWAALCLLGAELTEAGVAQSPRYKIIEKRQSVAFWCNPISGHATLYWYQQ
ILGQGPKLLIQFQNNGVVDDSQLPKDRFSAERLKGVDSTLKIQPAKLEDSAVYLCASS
LDPGDTGELFFGEGSRLTVLEDLKNVFPPEVAVFEPSEAEISHTQKATLVCLATGFYP
DHVELSWWVNGKEVHSGVSTDPQPLKEQPALNDSRYCLSSRLRVSATFWQNPRNHF
RCQVQFYGLSENDEWTQDRAKPVTQIVSAEAWGRADCGFTSESYQQGVLSATILYEI
LLGKATLYAVLVSALVLMAMVKRKDSRGRAKRSGSGATNFSLLKQAGDVEENPGP
MESFLGGVLLILWLQVDWVKSQKIEQNSEALNIQEGKTATLTCNYTNYSPAYLQWY
RQDPGRGPVFLLLIRENEKEKRKERLKVTFDTTLKQSLFHITASQPADSATYLCALDI
YPHDMRFGAGTRLTVKPDIQNPDPAVYQLRDSKSSDKSVCLFTDFDSQTNVSQSKDS
DVYITDKTVLDMRSMDFKSNSAVAWSNKSDFACANAFNNSIIPEDTFFPSPESSCDV
KLVEKSFETDTNLNFQNLSVIGFRILLLKVAGFNLLMTLRLWSSRAKRSGSGQCTNY
ALLKLAGDVESNPGPMLGIWTLLPLVLTSVARLSSKSVNAQVTDINSKGLELRKTVT
TVETQNLEGLHHDGQFCHKPCPPGERKARDCTVNGDEPDCVPCQEGKEYTDKAHFS
SKCRRCRLCDEGHGLEVEINCTRTQNTKCRCKPNFFCNSTVCEHCDPCTKCEHGIIKE
CTLTSNTKCKEEGSRSNLGWLCLLLLPIPLIVWVKRGRKKLLYIFKQPFMRPVQTTQE
EDGCSCRFPEEEEGGCEL
-TCR-1 + Fas-IL7Rα (plasmid sequence)
SEQ ID NO: 59
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcggggggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
GCACCAGACTGCTGTGTTGGGCTGCTCTGTGTCTGCTGGGAGCCGAACTTACTGA
AGCTGGCGTGGCCCAGTCTCCTCGGTACAAGATCATCGAGAAGCGGCAGAGCGT
GGCCTTCTGGTGCAATCCTATCAGCGGACACGCCACACTGTACTGGTATCAGCAG
ATCCTCGGACAGGGCCCCAAGCTGCTGATTCAGTTCCAGAACAACGGCGTGGTG
GACGACAGCCAGCTGCCTAAGGATAGATTCAGCGCCGAGCGGCTGAAAGGCGTG
GACAGCACACTGAAGATCCAGCCTGCCAAGCTGGAAGATAGCGCCGTGTACCTG
TGTGCCAGCTCTCTGGATCCTGGCGATACCGGCGAGCTGTTCTTTGGCGAGGGCA
GCAGACTGACCGTGCTCGAGGACCTGAAGAACGTGTTCCCACCTGAGGTGGCCG
TGTTCGAGCCTTCTGAGGCCGAGATCAGCCACACACAGAAAGCCACACTCGTGT
GTCTGGCCACCGGCTTCTATCCCGATCACGTGGAACTGTCTTGGTGGGTCAACGG
CAAAGAGGTGCACAGCGGCGTCAGCACAGATCCCCAGCCTCTGAAAGAGCAGCC
AGCTCTGAACGACAGCCGGTACTGTCTGAGCAGCAGGCTGAGAGTGTCCGCCAC
CTTCTGGCAGAACCCCAGAAACCACTTCAGATGCCAGGTGCAGTTCTACGGCCTG
AGCGAGAACGATGAGTGGACACAGGATAGAGCCAAGCCTGTGACACAGATCGT
GTCTGCCGAAGCCTGGGGCAGAGCCGATTGTGGCTTTACCAGCGAGAGCTACCA
GCAGGGCGTGCTGTCTGCCACAATCCTGTACGAGATCCTGCTGGGCAAAGCCACT
CTGTACGCCGTGCTGGTGTCTGCCCTGGTGCTGATGGCCATGGTCAAGCGGAAGG
ACTCTAGAGGCAGAGCCAAGAGATCTGGCAGCGGCGCCACAAACTTTAGCCTGC
TGAAACAGGCCGGCGACGTGGAAGAGAACCCCGGACCTATGGAATCCTTTCTCG
GTGGCGTGCTGCTCATCCTGTGGCTGCAAGTGGATTGGGTCAAGAGCCAGAAGA
TCGAGCAGAACAGCGAGGCCCTGAACATCCAAGAGGGCAAGACCGCCACTCTGA
CCTGCAACTACACCAACTACAGCCCCGCCTACCTGCAGTGGTACAGACAGGACC
CTGGAAGAGGCCCTGTGTTCCTGCTGCTGATCCGCGAGAATGAGAAAGAGAAGC
GCAAAGAGCGCCTGAAAGTCACCTTCGACACAACCCTGAAGCAGAGCCTGTTCC
ACATCACCGCCTCTCAGCCAGCCGATAGCGCCACATATCTGTGCGCCCTGGACAT
CTACCCTCACGACATGAGATTCGGAGCCGGCACCAGGCTGACAGTGAAGCCCGA
TATTCAGAACCCCGATCCTGCCGTCTATCAGCTGAGAGACAGCAAGAGCAGCGA
CAAGAGCGTGTGCCTGTTCACCGACTTCGACTCCCAGACCAACGTGTCCCAGAGC
AAGGACAGCGACGTGTACATCACCGACAAGACCGTGCTGGACATGCGGAGCATG
GACTTCAAGAGCAACAGCGCCGTGGCCTGGTCCAACAAGAGCGATTTCGCCTGC
GCCAACGCCTTCAACAACAGCATTATCCCCGAGGACACATTCTTCCCAAGTCCTG
AGAGCAGCTGCGACGTGAAGCTGGTGGAAAAGAGCTTCGAGACAGACACCAAC
CTGAACTTCCAGAACCTGAGCGTGATCGGCTTCCGGATCCTGCTCCTGAAAGTGG
CCGGCTTCAACCTGCTGATGACCCTGAGACTGTGGTCCAGCCGGGCCAAACGATC
TGGCTCTGGCCAGTGTACCAACTATGCCCTGCTCAAGCTCGCTGGCGACGTCGAG
TCTAACCCTGGACCAATGCTCGGCATCTGGACACTGCTGCCTCTGGTGCTGACAA
GCGTGGCCAGACTGAGCAGCAAGAGCGTGAACGCCCAAGTGACCGACATCAACA
GCAAAGGCCTGGAACTGAGAAAGACCGTGACCACCGTGGAAACCCAGAACCTG
GAAGGCCTGCACCACGACGGCCAGTTCTGTCACAAACCTTGTCCACCTGGCGAG
CGGAAGGCCAGAGATTGCACAGTGAATGGCGACGAGCCTGACTGCGTGCCCTGT
CAAGAGGGCAAAGAGTACACCGACAAGGCCCACTTCAGCAGCAAGTGCAGACG
GTGCAGACTGTGCGACGAAGGCCACGGACTGGAAGTGGAAATCAACTGCACCCG
GACACAGAACACCAAGTGCCGGTGCAAGCCCAACTTCTTCTGCAACAGCACCGT
GTGCGAGCACTGCGACCCTTGTACCAAGTGCGAACACGGCATCATCAAAGAGTG
CACCCTGACCTCCAACACGAAGTGCAAAGAGGAAGGCAGCAGAAGCAACCTCG
GCTGGCTGTGTCTGCTGCTGCTCCCCATTCCTCTGATCGTGTGGGTCAAGAAGCG
GATCAAGCCCATCGTGTGGCCCAGCCTGCCTGACCACAAGAAAACCCTGGAACA
CCTGTGCAAGAAGCCCCGGAAGAACCTGAACGTGTCCTTCAATCCCGAGAGCTT
CCTGGACTGCCAGATCCACAGAGTGGACGACATCCAGGCCAGGGATGAAGTCGA
GGGCTTTCTGCAGGACACCTTTCCACAGCAGCTGGAAGAGAGCGAGAAGCAGAG
ACTCGGCGGAGATGTGCAGAGCCCTAATTGCCCTAGCGAGGACGTGGTCATCAC
CCCTGAGAGCTTCGGCAGAGATAGCAGCCTGACATGTCTGGCCGGCAATGTGTC
CGCCTGTGATGCCCCTATCCTGAGCAGCTCCAGAAGCCTGGATTGCAGAGAGAG
CGGCAAGAACGGCCCTCACGTGTACCAGGATCTGCTCCTGTCTCTGGGCACCACC
AATAGCACACTGCCTCCACCATTCAGCCTGCAGAGCGGCATCCTGACACTGAACC
CTGTTGCTCAGGGCCAGCCAATCCTGACAAGCCTGGGCAGCAATCAAGAAGAGG
CCTACGTCACCATGAGCAGCTTCTACCAGAACCAGTGATGATTCGAAGCGGCCG
CTTCGAGCAGACATGATAAGATACATTGATGAGTTTGGACAAACCACAACTAGA
ATGCAGTGAAAAAAATGCTTTATTTGTGAAATTTGTGATGCTATTGCTTTATTTGT
AACCATTATAAGCTGCAATAAACAAGTTACACGGCAGGGTCAGGGTTCTGGATA
Tctgtgggacaagaggatcagggttaggacatgatctcatttccctctttgccccaacccaggctggagtccagatgccagtgatgga
caagggcggggctctgtggggctggcaagtcacggtctcatgctttatacgggaaatagcatcttagaaaccagctgctcgtgatgga
ctgggactcagggacaggcacaagctatcaatcttggccaagaggccatgatttcagtgaacgttcacggccaggcctggcctgcca
ctcaaggaaacCACTGGCCGTCGTTTTACAACGTCGTGACTgggaaaaccctggcgttacccaacttaat
cgccttgcagcacatccccctttcgccagctggcgtaatagcgaagaggcccgcaccgatcgcccttcccaacagttgcgcagcctg
aatggcgaatggcgcctgatgcggtattttctccttacgcatctgtgcggtatttcacaccgcatatggtgcactctcagtacaatctgctc
tgatgccgcatagttaagccagccccgacacccgccaacacccgctgacgcgccctgacgggcttgtctgctcccggcatccgctta
cagacaagctgtgaccgtctccgggagctgcatgtgtcagaggttttcaccgtcatcaccgaaacgcgcga
-TCR-1 + Fas-IL7Rα (amino acid sequence)
SEQ ID NO: 60
MGTRLLCWAALCLLGAELTEAGVAQSPRYKIIEKRQSVAFWCNPISGHATLYWYQQ
ILGQGPKLLIQFQNNGVVDDSQLPKDRFSAERLKGVDSTLKIQPAKLEDSAVYLCASS
LDPGDTGELFFGEGSRLTVLEDLKNVFPPEVAVFEPSEAEISHTQKATLVCLATGFYP
DHVELSWWVNGKEVHSGVSTDPQPLKEQPALNDSRYCLSSRLRVSATFWQNPRNHF
RCQVQFYGLSENDEWTQDRAKPVTQIVSAEAWGRADCGFTSESYQQGVLSATILYEI
LLGKATLYAVLVSALVLMAMVKRKDSRGRAKRSGSGATNFSLLKQAGDVEENPGP
MESFLGGVLLILWLQVDWVKSQKIEQNSEALNIQEGKTATLTCNYTNYSPAYLQWY
RQDPGRGPVFLLLIRENEKEKRKERLKVTFDTTLKQSLFHITASQPADSATYLCALDI
YPHDMRFGAGTRLTVKPDIQNPDPAVYQLRDSKSSDKSVCLFTDFDSQTNVSQSKDS
DVYITDKTVLDMRSMDFKSNSAVAWSNKSDFACANAFNNSIIPEDTFFPSPESSCDV
KLVEKSFETDTNLNFQNLSVIGFRILLLKVAGFNLLMTLRLWSSRAKRSGSGQCTNY
ALLKLAGDVESNPGPMLGIWTLLPLVLTSVARLSSKSVNAQVTDINSKGLELRKTVT
TVETQNLEGLHHDGQFCHKPCPPGERKARDCTVNGDEPDCVPCQEGKEYTDKAHFS
SKCRRCRLCDEGHGLEVEINCTRTQNTKCRCKPNFFCNSTVCEHCDPCTKCEHGIIKE
CTLTSNTKCKEEGSRSNLGWLCLLLLPIPLIVWVKKRIKPIVWPSLPDHKKTLEHLCK
KPRKNLNVSFNPESFLDCQIHRVDDIQARDEVEGFLQDTFPQQLEESEKQRLGGDVQ
SPNCPSEDVVITPESFGRDSSLTCLAGNVSACDAPILSSSRSLDCRESGKNGPHVYQDL
LLSLGTTNSTLPPPFSLQSGILTLNPVAQGQPILTSLGSNQEEAYVTMSSFYQNQ
-TCR-1 + CASTAT3 (plasmid sequence)
SEQ ID NO: 61
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
GCACCAGACTGCTGTGTTGGGCTGCTCTGTGTCTGCTGGGAGCCGAACTTACTGA
AGCTGGCGTGGCCCAGTCTCCTCGGTACAAGATCATCGAGAAGCGGCAGAGCGT
GGCCTTCTGGTGCAATCCTATCAGCGGACACGCCACACTGTACTGGTATCAGCAG
ATCCTCGGACAGGGCCCCAAGCTGCTGATTCAGTTCCAGAACAACGGCGTGGTG
GACGACAGCCAGCTGCCTAAGGATAGATTCAGCGCCGAGCGGCTGAAAGGCGTG
GACAGCACACTGAAGATCCAGCCTGCCAAGCTGGAAGATAGCGCCGTGTACCTG
TGTGCCAGCTCTCTGGATCCTGGCGATACCGGCGAGCTGTTCTTTGGCGAGGGCA
GCAGACTGACCGTGCTCGAGGACCTGAAGAACGTGTTCCCACCTGAGGTGGCCG
TGTTCGAGCCTTCTGAGGCCGAGATCAGCCACACACAGAAAGCCACACTCGTGT
GTCTGGCCACCGGCTTCTATCCCGATCACGTGGAACTGTCTTGGTGGGTCAACGG
CAAAGAGGTGCACAGCGGCGTCAGCACAGATCCCCAGCCTCTGAAAGAGCAGCC
AGCTCTGAACGACAGCCGGTACTGTCTGAGCAGCAGGCTGAGAGTGTCCGCCAC
CTTCTGGCAGAACCCCAGAAACCACTTCAGATGCCAGGTGCAGTTCTACGGCCTG
AGCGAGAACGATGAGTGGACACAGGATAGAGCCAAGCCTGTGACACAGATCGT
GTCTGCCGAAGCCTGGGGCAGAGCCGATTGTGGCTTTACCAGCGAGAGCTACCA
GCAGGGCGTGCTGTCTGCCACAATCCTGTACGAGATCCTGCTGGGCAAAGCCACT
CTGTACGCCGTGCTGGTGTCTGCCCTGGTGCTGATGGCCATGGTCAAGCGGAAGG
ACTCTAGAGGCAGAGCCAAGAGATCTGGCAGCGGCGCCACAAACTTTAGCCTGC
TGAAACAGGCCGGCGACGTGGAAGAGAACCCCGGACCTATGGAATCCTTTCTCG
GTGGCGTGCTGCTCATCCTGTGGCTGCAAGTGGATTGGGTCAAGAGCCAGAAGA
TCGAGCAGAACAGCGAGGCCCTGAACATCCAAGAGGGCAAGACCGCCACTCTGA
CCTGCAACTACACCAACTACAGCCCCGCCTACCTGCAGTGGTACAGACAGGACC
CTGGAAGAGGCCCTGTGTTCCTGCTGCTGATCCGCGAGAATGAGAAAGAGAAGC
GCAAAGAGCGCCTGAAAGTCACCTTCGACACAACCCTGAAGCAGAGCCTGTTCC
ACATCACCGCCTCTCAGCCAGCCGATAGCGCCACATATCTGTGCGCCCTGGACAT
CTACCCTCACGACATGAGATTCGGAGCCGGCACCAGGCTGACAGTGAAGCCCGA
TATTCAGAACCCCGATCCTGCCGTCTATCAGCTGAGAGACAGCAAGAGCAGCGA
CAAGAGCGTGTGCCTGTTCACCGACTTCGACTCCCAGACCAACGTGTCCCAGAGC
AAGGACAGCGACGTGTACATCACCGACAAGACCGTGCTGGACATGCGGAGCATG
GACTTCAAGAGCAACAGCGCCGTGGCCTGGTCCAACAAGAGCGATTTCGCCTGC
GCCAACGCCTTCAACAACAGCATTATCCCCGAGGACACATTCTTCCCAAGTCCTG
AGAGCAGCTGCGACGTGAAGCTGGTGGAAAAGAGCTTCGAGACAGACACCAAC
CTGAACTTCCAGAACCTGAGCGTGATCGGCTTCCGGATCCTGCTCCTGAAAGTGG
CCGGCTTCAACCTGCTGATGACCCTGAGACTGTGGTCCAGCCGGGCCAAACGATC
TGGCTCTGGCCAGTGTACCAACTATGCCCTGCTCAAGCTCGCTGGCGACGTCGAG
TCTAACCCTGGACCAATGGCCCAGTGGAATCAGCTGCAGCAGCTCGACACCAGA
TACCTCGAACAGCTCCACCAGCTGTACTCCGACAGCTTCCCTATGGAACTGCGGC
AGTTTCTGGCCCCTTGGATCGAGTCTCAGGATTGGGCCTACGCCGCCAGCAAAGA
GTCTCACGCCACACTGGTGTTCCACAACCTGCTGGGCGAGATCGACCAGCAGTAC
AGCCGGTTTCTGCAAGAGTCCAACGTGCTGTACCAGCACAACCTGAGGCGGATC
AAGCAGTTCCTGCAGAGCAGATACCTGGAAAAGCCCATGGAAATCGCCCGGATC
GTGGCCAGATGTCTGTGGGAAGAAAGCAGACTGCTGCAGACCGCCGCTACAGCT
GCTCAACAAGGCGGACAGGCCAACCATCCTACAGCCGCCGTGGTTACAGAGAAG
CAGCAGATGCTGGAACAGCATCTCCAGGACGTGCGGAAGAGAGTGCAGGACCTG
GAACAGAAAATGAAGGTGGTGGAAAACCTGCAGGACGACTTCGACTTCAACTAC
AAGACCCTGAAGTCCCAGGGCGACATGCAGGATCTGAACGGCAACAACCAGAGC
GTGACCCGGCAGAAGATGCAGCAACTCGAACAGATGCTGACAGCCCTGGACCAG
ATGAGGCGGAGCATTGTGTCTGAACTGGCCGGACTGCTGAGCGCCATGGAATAC
GTGCAGAAAACCCTGACCGACGAGGAACTGGCTGACTGGAAAAGACGGCAGCA
GATCGCCTGTATCGGCGGACCTCCTAACATCTGCCTGGACCGGCTGGAAAACTGG
ATCACAAGCCTGGCCGAAAGCCAGCTGCAGACAAGACAGCAGATCAAGAAGCT
GGAAGAACTGCAGCAGAAGGTGTCCTACAAGGGCGACCCCATCGTGCAGCACAG
ACCCATGCTCGAGGAACGCATCGTGGAACTGTTCCGGAATCTGATGAAGTCCGC
CTTCGTGGTGGAACGGCAGCCCTGTATGCCTATGCACCCCGATAGACCCCTGGTC
ATCAAGACCGGCGTGCAGTTCACCACCAAAGTGCGGCTGCTGGTCAAGTTCCCC
GAGCTGAACTACCAGCTGAAGATCAAAGTGTGCATCGACAAGGACAGCGGCGAC
GTGGCAGCTCTGAGAGGCAGCAGAAAGTTCAACATCCTGGGCACCAACACCAGG
GTCATGAACATGGAAGAGAGCAACAACGGCTCCCTGAGCGCCGAGTTCAAGCAC
CTGACACTGCGCGAGCAGAGATGCGGAAATGGCGGCAGAGCCAACTGTGACGCC
AGCCTGATTGTGACAGAGGAACTGCACCTGATCACCTTCGAGACAGAGGTGTAC
CACCAGGGCCTGAAGATCGACCTGGAAACCCACTCTCTGCCCGTGGTGGTCATCA
GCAACATCTGTCAGATGCCCAACGCCTGGGCCAGCATCCTGTGGTACAACATGCT
GACCAACAATCCCAAGAACGTGAACTTCTTCACCAAGCCTCCAATCGGCACCTG
GGACCAAGTGGCCGAAGTTCTGTCCTGGCAGTTCAGCAGCACCACCAAGAGAGG
CCTGAGCATCGAGCAGCTGACAACACTGGCCGAGAAGCTGCTTGGACCCGGCGT
GAACTATAGCGGCTGTCAGATCACATGGGCCAAGTTCTGCAAAGAAAACATGGC
CGGCAAGGGCTTCAGCTTCTGGGTCTGGCTGGACAACATCATCGACCTCGTCAAG
AAGTACATTCTGGCCCTGTGGAACGAGGGCTACATCATGGGCTTCATCTCCAAAG
AGAGAGAGCGGGCCATCCTGAGCACAAAGCCTCCAGGCACATTCCTGCTGCGGT
TCAGCGAGAGCAGCAAAGAAGGCGGCGTCACCTTTACCTGGGTCGAGAAGGATA
TCAGCGGCAAGACCCAGATCCAGTCCGTGGAACCCTACACCAAGCAGCAGCTGA
ACAATATGAGCTTCGCCGAGATCATCATGGGGTACAAGATCATGGACGCCACCA
ATATCCTGGTGTCCCCACTGGTGTACCTGTATCCTGACATCCCCAAAGAGGAAGC
CTTCGGCAAGTACTGCAGACCCGAGAGCCAAGAGCACCCTGAAGCCGATCCTGG
AAGCGCCGCTCCTTACCTGAAAACAAAGTTCATCTGCGTGACCCCTACCACCTGT
AGCAACACCATCGATCTGCCTATGAGCCCCAGAACACTGGACAGCCTGATGCAG
TTCGGCAACAATGGCGAAGGCGCCGAACCATCTGCTGGCGGACAGTTTGAGAGC
CTGACCTTCGACATGGAACTGACCTCCGAGTGCGCCACCTCTCCTATGTGATGAT
TCGAAGCGGCCGCTTCGAGCAGACATGATAAGATACATTGATGAGTTTGGACAA
ACCACAACTAGAATGCAGTGAAAAAAATGCTTTATTTGTGAAATTTGTGATGCTA
TTGCTTTATTTGTAACCATTATAAGCTGCAATAAACAAGTTACACGGCAGGGTCA
GGGTTCTGGATATctgtgggacaagaggatcagggttaggacatgatctcatttccctctttgccccaacccaggctggag
tccagatgccagtgatggacaagggcggggctctgtggggctggcaagtcacggtctcatgctttatacgggaaatagcatcttagaa
accagctgctcgtgatggactgggactcagggacaggcacaagctatcaatcttggccaagaggccatgatttcagtgaacgttcacg
gccaggcctggcctgccactcaaggaaacCACTGGCCGTCGTTTTACAACGTCGTGACTgggaaaaccc
tggcgttacccaacttaatcgccttgcagcacatccccctttcgccagctggcgtaatagcgaagaggcccgcaccgatcgcccttccc
aacagttgcgcagcctgaatggcgaatggcgcctgatgcggtattttctccttacgcatctgtgcggtatttcacaccgcatatggtgcac
tctcagtacaatctgctctgatgccgcatagttaagccagccccgacacccgccaacacccgctgacgcgccctgacgggcttgtctg
ctcccggcatccgcttacagacaagctgtgaccgtctccgggagctgcatgtgtcagaggttttcaccgtcatcaccgaaacgcgcga
-TCR-1 + CASTAT3 (amino acid sequence)
SEQ ID NO: 62
MGTRLLCWAALCLLGAELTEAGVAQSPRYKIIEKRQSVAFWCNPISGHATLYWYQQ
ILGQGPKLLIQFQNNGVVDDSQLPKDRFSAERLKGVDSTLKIQPAKLEDSAVYLCASS
LDPGDTGELFFGEGSRLTVLEDLKNVFPPEVAVFEPSEAEISHTQKATLVCLATGFYP
DHVELSWWVNGKEVHSGVSTDPQPLKEQPALNDSRYCLSSRLRVSATFWQNPRNHF
RCQVQFYGLSENDEWTQDRAKPVTQIVSAEAWGRADCGFTSESYQQGVLSATILYEI
LLGKATLYAVLVSALVLMAMVKRKDSRGRAKRSGSGATNFSLLKQAGDVEENPGP
MESFLGGVLLILWLQVDWVKSQKIEQNSEALNIQEGKTATLTCNYTNYSPAYLQWY
RQDPGRGPVFLLLIRENEKEKRKERLKVTFDTTLKQSLFHITASQPADSATYLCALDI
YPHDMRFGAGTRLTVKPDIQNPDPAVYQLRDSKSSDKSVCLFTDFDSQTNVSQSKDS
DVYITDKTVLDMRSMDFKSNSAVAWSNKSDFACANAFNNSIIPEDTFFPSPESSCDV
KLVEKSFETDTNLNFQNLSVIGFRILLLKVAGFNLLMTLRLWSSRAKRSGSGQCTNY
ALLKLAGDVESNPGPMAQWNQLQQLDTRYLEQLHQLYSDSFPMELRQFLAPWIESQ
DWAYAASKESHATLVFHNLLGEIDQQYSRFLQESNVLYQHNLRRIKQFLQSRYLEKP
MEIARIVARCLWEESRLLQTAATAAQQGGQANHPTAAVVTEKQQMLEQHLQDVRK
RVQDLEQKMKVVENLQDDFDFNYKTLKSQGDMQDLNGNNQSVTRQKMQQLEQM
LTALDQMRRSIVSELAGLLSAMEYVQKTLTDEELADWKRRQQIACIGGPPNICLDRL
ENWITSLAESQLQTRQQIKKLEELQQKVSYKGDPIVQHRPMLEERIVELFRNLMKSAF
VVERQPCMPMHPDRPLVIKTGVQFTTKVRLLVKFPELNYQLKIKVCIDKDSGDVAAL
RGSRKFNILGTNTRVMNMEESNNGSLSAEFKHLTLREQRCGNGGRANCDASLIVTEE
LHLITFETEVYHQGLKIDLETHSLPVVVISNICQMPNAWASILWYNMLTNNPKNVNF
FTKPPIGTWDQVAEVLSWQFSSTTKRGLSIEQLTTLAEKLLGPGVNYSGCQITWAKF
CKENMAGKGFSFWVWLDNIIDLVKKYILALWNEGYIMGFISKERERAILSTKPPGTFL
LRFSESSKEGGVTFTWVEKDISGKTQIQSVEPYTKQQLNNMSFAEIIMGYKIMDATNI
LVSPLVYLYPDIPKEEAFGKYCRPESQEHPEADPGSAAPYLKTKFICVTPTTCSNTIDL
PMSPRTLDSLMQFGNNGEGAEPSAGGQFESLTFDMELTSECATSPM
-TCR-1 + CASTAT5 (plasmid sequence)
SEQ ID NO: 63
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
GCACCAGACTGCTGTGTTGGGCTGCTCTGTGTCTGCTGGGAGCCGAACTTACTGA
AGCTGGCGTGGCCCAGTCTCCTCGGTACAAGATCATCGAGAAGCGGCAGAGCGT
GGCCTTCTGGTGCAATCCTATCAGCGGACACGCCACACTGTACTGGTATCAGCAG
ATCCTCGGACAGGGCCCCAAGCTGCTGATTCAGTTCCAGAACAACGGCGTGGTG
GACGACAGCCAGCTGCCTAAGGATAGATTCAGCGCCGAGCGGCTGAAAGGCGTG
GACAGCACACTGAAGATCCAGCCTGCCAAGCTGGAAGATAGCGCCGTGTACCTG
TGTGCCAGCTCTCTGGATCCTGGCGATACCGGCGAGCTGTTCTTTGGCGAGGGCA
GCAGACTGACCGTGCTCGAGGACCTGAAGAACGTGTTCCCACCTGAGGTGGCCG
TGTTCGAGCCTTCTGAGGCCGAGATCAGCCACACACAGAAAGCCACACTCGTGT
GTCTGGCCACCGGCTTCTATCCCGATCACGTGGAACTGTCTTGGTGGGTCAACGG
CAAAGAGGTGCACAGCGGCGTCAGCACAGATCCCCAGCCTCTGAAAGAGCAGCC
AGCTCTGAACGACAGCCGGTACTGTCTGAGCAGCAGGCTGAGAGTGTCCGCCAC
CTTCTGGCAGAACCCCAGAAACCACTTCAGATGCCAGGTGCAGTTCTACGGCCTG
AGCGAGAACGATGAGTGGACACAGGATAGAGCCAAGCCTGTGACACAGATCGT
GTCTGCCGAAGCCTGGGGCAGAGCCGATTGTGGCTTTACCAGCGAGAGCTACCA
GCAGGGCGTGCTGTCTGCCACAATCCTGTACGAGATCCTGCTGGGCAAAGCCACT
CTGTACGCCGTGCTGGTGTCTGCCCTGGTGCTGATGGCCATGGTCAAGCGGAAGG
ACTCTAGAGGCAGAGCCAAGAGATCTGGCAGCGGCGCCACAAACTTTAGCCTGC
TGAAACAGGCCGGCGACGTGGAAGAGAACCCCGGACCTATGGAATCCTTTCTCG
GTGGCGTGCTGCTCATCCTGTGGCTGCAAGTGGATTGGGTCAAGAGCCAGAAGA
TCGAGCAGAACAGCGAGGCCCTGAACATCCAAGAGGGCAAGACCGCCACTCTGA
CCTGCAACTACACCAACTACAGCCCCGCCTACCTGCAGTGGTACAGACAGGACC
CTGGAAGAGGCCCTGTGTTCCTGCTGCTGATCCGCGAGAATGAGAAAGAGAAGC
GCAAAGAGCGCCTGAAAGTCACCTTCGACACAACCCTGAAGCAGAGCCTGTTCC
ACATCACCGCCTCTCAGCCAGCCGATAGCGCCACATATCTGTGCGCCCTGGACAT
CTACCCTCACGACATGAGATTCGGAGCCGGCACCAGGCTGACAGTGAAGCCCGA
TATTCAGAACCCCGATCCTGCCGTCTATCAGCTGAGAGACAGCAAGAGCAGCGA
CAAGAGCGTGTGCCTGTTCACCGACTTCGACTCCCAGACCAACGTGTCCCAGAGC
AAGGACAGCGACGTGTACATCACCGACAAGACCGTGCTGGACATGCGGAGCATG
GACTTCAAGAGCAACAGCGCCGTGGCCTGGTCCAACAAGAGCGATTTCGCCTGC
GCCAACGCCTTCAACAACAGCATTATCCCCGAGGACACATTCTTCCCAAGTCCTG
AGAGCAGCTGCGACGTGAAGCTGGTGGAAAAGAGCTTCGAGACAGACACCAAC
CTGAACTTCCAGAACCTGAGCGTGATCGGCTTCCGGATCCTGCTCCTGAAAGTGG
CCGGCTTCAACCTGCTGATGACCCTGAGACTGTGGTCCAGCCGGGCCAAACGATC
TGGCTCTGGCCAGTGTACCAACTATGCCCTGCTCAAGCTCGCTGGCGACGTCGAG
TCTAACCCTGGACCAATGGCCGGATGGATTCAGGCTCAGCAGCTTCAAGGCGAC
GCCCTGAGACAGATGCAGGTCCTGTATGGCCAGCACTTCCCCATCGAAGTGCGG
CACTATCTGGCCCAGTGGATCGAGTCTCAGCCCTGGGATGCCATCGACCTGGACA
ACCCTCAGGATAGAGGACAGGCCACACAGCTGCTGGAAGGCCTGGTTCAAGAGC
TGCAGAAGAAAGCCGAGCACCAAGTGGGCGAAGATGGCTTCCTGCTGAAGATCA
AGCTGGGCCACTACGCCACTCAGCTCCAGAACACCTACGACAGATGCCCCATGG
AACTCGTGCGGTGCATCCGGCACATCCTGTACAACGAGCAGAGGCTCGTCAGAG
AGGCCAACAACTGTTCTTCTCCTGCCGGCGTGCTGGTGGATGCCATGAGCCAGAA
ACACCTCCAGATCAACCAGCGCTTCGAGGAACTGCGGCTGATCACCCAGGACAC
CGAGAACGAGCTGAAGAAGCTGCAGCAGACCCAAGAGTACTTCATCATCCAGTA
CCAAGAGAGCCTGCGCATTCAGGCCCAGTTTGCTCAGCTGGGACAGCTGAACCC
TCAAGAGCGGATGAGCAGAGAGACAGCCCTCCAGCAGAAACAGGTGTCCCTGGA
AACCTGGCTGCAGAGAGAGGCTCAGACCCTGCAGCAGTACAGAGTGGAACTGGC
CGAGAAGCACCAGAAAACACTGCAGCTGCTGCGGAAGCAGCAGACAATCATCCT
GGACGACGAGCTGATCCAGTGGAAGCGGAGACAGCAGCTGGCTGGAAATGGCG
GACCTCCTGAGGGATCTCTGGACGTGTTGCAGTCTTGGTGCGAGAAGCTGGCCGA
AATCATCTGGCAGAACCGGCAGCAGATTCGGAGAGCCGAGAGACTGTGTCAGCA
GCTCCCTATTCCTGGACCTGTGGAAGAGATGCTGGCTGAAGTGAACGCCACCATC
ACCGACATCATCAGCGCCCTGGTCACCAGCACCTTTATCATCGAGAAGCAGCCTC
CACAGGTGCTGAAAACCCAGACCAAGTTTGCCGCCACAGTGCGGTTGCTCGTTG
GCGGCAAGCTGAACGTGCACATGAACCCTCCACAAGTGAAGGCCACAATTATCA
GCGAGCAGCAGGCCAAGAGCCTGCTGAAAAACGAGAACACCCGGAACGAGTGC
AGCGGCGAGATCCTGAACAACTGCTGCGTGATGGAATACCACCAGGCCACCGGA
ACACTGAGCGCCCACTTCAGAAATATGTCCCTGAAGCGGATCAAGCGCGCCGAT
AGAAGAGGCGCCGAGTCTGTGACCGAAGAGAAGTTCACCGTGCTGTTCGAGAGC
CAGTTCAGCGTGGGCAGCAACGAGCTGGTGTTCCAAGTGAAAACCCTGAGCCTG
CCTGTGGTGGTCATCGTGCACGGAAGCCAGGATCACAATGCCACCGCTACAGTG
CTGTGGGACAACGCCTTTGCCGAGCCTGGCAGAGTGCCTTTTGCCGTGCCTGATA
AGGTCCTGTGGCCTCAGCTTTGCGAGGCCCTGAACATGAAGTTCAAGGCCGAGG
TGCAGAGCAACCGGGGCCTGACCAAAGAAAACCTGGTGTTTCTTGCCCAGAAGC
TGTTCAACATCAGCAGCAACCACCTCGAGGACTACAACAGCATGTCCGTGTCTTG
GTCCCAGTTCAACAGAGAGAACTTGCCCGGCTGGAACTACACCTTCTGGCAGTG
GTTCGACGGCGTCATGGAAGTCCTGAAGAAACATCACAAGCCCCACTGGAACGA
CGGCGCCATCCTGGGCTTTGTGAACAAACAGCAGGCCCACGATCTGCTGATCAA
CAAGCCCGATGGCACCTTTCTGCTGAGATTCAGCGACAGCGAGATCGGCGGCAT
CACAATCGCCTGGAAGTTCGACAGCCCCGACCGGAATCTGTGGAACCTGAAGCC
ATTCACCACCAGAGACTTCAGCATCCGCAGCCTGGCTGACAGACTGGGCGACCT
GAACTACCTGATCTACGTGTTCCCCGATCGGCCCAAGGATGAGGTGTTCGCCAAG
TACTACACCCCTGTGCTGGCTAAGGCCGTGGACGGCTATGTGAAGCCCCAGATCA
AACAGGTGGTGCCCGAGTTCGTGAACGCCTTCACTGATGCTGGCGCCTCCGCCAC
CTATATGGATCAGGCACCTTCTCCAGTCGTGTGCCCTCAGCCTCACTACAACATG
TACCCACCTAATCCTGATCCTGTGCTCGACCAGGACGGCGAGTTCGATCTGGACG
AGTCTATGGACGTGGCCCGGCACGTTGAAGAACTGCTCAGACGGCCTATGGACA
GCCTGGACGCTAGACTGTCTCCTCCAGCCGGCCTGTTTACAAGCGCCAGAAGCTC
TCTGAGCTGATGATTCGAAGCGGCCGCTTCGAGCAGACATGATAAGATACATTG
ATGAGTTTGGACAAACCACAACTAGAATGCAGTGAAAAAAATGCTTTATTTGTG
AAATTTGTGATGCTATTGCTTTATTTGTAACCATTATAAGCTGCAATAAACAAGTT
ACACGGCAGGGTCAGGGTTCTGGATATctgtgggacaagaggatcagggttaggacatgatctcatttccct
ctttgccccaacccaggctggagtccagatgccagtgatggacaagggcggggctctgtggggctggcaagtcacggtctcatgcttt
atacgggaaatagcatcttagaaaccagctgctcgtgatggactgggactcagggacaggcacaagctatcaatcttggccaagagg
ccatgatttcagtgaacgttcacggccaggcctggcctgccactcaaggaaacCACTGGCCGTCGTTTTACAACG
TCGTGACTgggaaaaccctggcgttacccaacttaatcgccttgcagcacatccccctttcgccagctggcgtaatagcgaaga
ggcccgcaccgatcgcccttcccaacagttgcgcagcctgaatggcgaatggcgcctgatgcggtattttctccttacgcatctgtgcg
gtatttcacaccgcatatggtgcactctcagtacaatctgctctgatgccgcatagttaagccagccccgacacccgccaacacccgct
gacgcgccctgacgggcttgtctgctcccggcatccgcttacagacaagctgtgaccgtctccgggagctgcatgtgtcagaggttttc
accgtcatcaccgaaacgcgcga
-TCR-1 + CASTAT5 (amino acid sequence)
SEQ ID NO: 64
MGTRLLCWAALCLLGAELTEAGVAQSPRYKIIEKRQSVAFWCNPISGHATLYWYQQ
ILGQGPKLLIQFQNNGVVDDSQLPKDRFSAERLKGVDSTLKIQPAKLEDSAVYLCASS
LDPGDTGELFFGEGSRLTVLEDLKNVFPPEVAVFEPSEAEISHTQKATLVCLATGFYP
DHVELSWWVNGKEVHSGVSTDPQPLKEQPALNDSRYCLSSRLRVSATFWQNPRNHF
RCQVQFYGLSENDEWTQDRAKPVTQIVSAEAWGRADCGFTSESYQQGVLSATILYEI
LLGKATLYAVLVSALVLMAMVKRKDSRGRAKRSGSGATNFSLLKQAGDVEENPGP
MESFLGGVLLILWLQVDWVKSQKIEQNSEALNIQEGKTATLTCNYTNYSPAYLQWY
RQDPGRGPVFLLLIRENEKEKRKERLKVTFDTTLKQSLFHITASQPADSATYLCALDI
YPHDMRFGAGTRLTVKPDIQNPDPAVYQLRDSKSSDKSVCLFTDFDSQTNVSQSKDS
DVYITDKTVLDMRSMDFKSNSAVAWSNKSDFACANAFNNSIIPEDTFFPSPESSCDV
KLVEKSFETDTNLNFQNLSVIGFRILLLKVAGFNLLMTLRLWSSRAKRSGSGQCTNY
ALLKLAGDVESNPGPMAGWIQAQQLQGDALRQMQVLYGQHFPIEVRHYLAQWIES
QPWDAIDLDNPQDRGQATQLLEGLVQELQKKAEHQVGEDGFLLKIKLGHYATQLQ
NTYDRCPMELVRCIRHILYNEQRLVREANNCSSPAGVLVDAMSQKHLQINQRFEELR
LITQDTENELKKLQQTQEYFIIQYQESLRIQAQFAQLGQLNPQERMSRETALQQKQVS
LETWLQREAQTLQQYRVELAEKHQKTLQLLRKQQTIILDDELIQWKRRQQLAGNGG
PPEGSLDVLQSWCEKLAEIIWQNRQQIRRAERLCQQLPIPGPVEEMLAEVNATITDIIS
ALVTSTFIIEKQPPQVLKTQTKFAATVRLLVGGKLNVHMNPPQVKATIISEQQAKSLL
KNENTRNECSGEILNNCCVMEYHQATGTLSAHFRNMSLKRIKRADRRGAESVTEEK
FTVLFESQFSVGSNELVFQVKTLSLPVVVIVHGSQDHNATATVLWDNAFAEPGRVPF
AVPDKVLWPQLCEALNMKFKAEVQSNRGLTKENLVFLAQKLFNISSNHLEDYNSMS
VSWSQFNRENLPGWNYTFWQWFDGVMEVLKKHHKPHWNDGAILGFVNKQQAHD
LLINKPDGTFLLRFSDSEIGGITIAWKFDSPDRNLWNLKPFTTRDFSIRSLADRLGDLN
YLIYVFPDRPKDEVFAKYYTPVLAKAVDGYVKPQIKQVVPEFVNAFTDAGASATYM
DQAPSPVVCPQPHYNMYPPNPDPVLDQDGEFDLDESMDVARHVEELLRRPMDSLDA
RLSPPAGLFTSARSSLS
-TCR-1 + C7R-1 (plasmid sequence)
SEQ ID NO: 65
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
GCACCAGACTGCTGTGTTGGGCTGCTCTGTGTCTGCTGGGAGCCGAACTTACTGA
AGCTGGCGTGGCCCAGTCTCCTCGGTACAAGATCATCGAGAAGCGGCAGAGCGT
GGCCTTCTGGTGCAATCCTATCAGCGGACACGCCACACTGTACTGGTATCAGCAG
ATCCTCGGACAGGGCCCCAAGCTGCTGATTCAGTTCCAGAACAACGGCGTGGTG
GACGACAGCCAGCTGCCTAAGGATAGATTCAGCGCCGAGCGGCTGAAAGGCGTG
GACAGCACACTGAAGATCCAGCCTGCCAAGCTGGAAGATAGCGCCGTGTACCTG
TGTGCCAGCTCTCTGGATCCTGGCGATACCGGCGAGCTGTTCTTTGGCGAGGGCA
GCAGACTGACCGTGCTCGAGGACCTGAAGAACGTGTTCCCACCTGAGGTGGCCG
TGTTCGAGCCTTCTGAGGCCGAGATCAGCCACACACAGAAAGCCACACTCGTGT
GTCTGGCCACCGGCTTCTATCCCGATCACGTGGAACTGTCTTGGTGGGTCAACGG
CAAAGAGGTGCACAGCGGCGTCAGCACAGATCCCCAGCCTCTGAAAGAGCAGCC
AGCTCTGAACGACAGCCGGTACTGTCTGAGCAGCAGGCTGAGAGTGTCCGCCAC
CTTCTGGCAGAACCCCAGAAACCACTTCAGATGCCAGGTGCAGTTCTACGGCCTG
AGCGAGAACGATGAGTGGACACAGGATAGAGCCAAGCCTGTGACACAGATCGT
GTCTGCCGAAGCCTGGGGCAGAGCCGATTGTGGCTTTACCAGCGAGAGCTACCA
GCAGGGCGTGCTGTCTGCCACAATCCTGTACGAGATCCTGCTGGGCAAAGCCACT
CTGTACGCCGTGCTGGTGTCTGCCCTGGTGCTGATGGCCATGGTCAAGCGGAAGG
ACTCTAGAGGCAGAGCCAAGAGATCTGGCAGCGGCGCCACAAACTTTAGCCTGC
TGAAACAGGCCGGCGACGTGGAAGAGAACCCCGGACCTATGGAATCCTTTCTCG
GTGGCGTGCTGCTCATCCTGTGGCTGCAAGTGGATTGGGTCAAGAGCCAGAAGA
TCGAGCAGAACAGCGAGGCCCTGAACATCCAAGAGGGCAAGACCGCCACTCTGA
CCTGCAACTACACCAACTACAGCCCCGCCTACCTGCAGTGGTACAGACAGGACC
CTGGAAGAGGCCCTGTGTTCCTGCTGCTGATCCGCGAGAATGAGAAAGAGAAGC
GCAAAGAGCGCCTGAAAGTCACCTTCGACACAACCCTGAAGCAGAGCCTGTTCC
ACATCACCGCCTCTCAGCCAGCCGATAGCGCCACATATCTGTGCGCCCTGGACAT
CTACCCTCACGACATGAGATTCGGAGCCGGCACCAGGCTGACAGTGAAGCCCGA
TATTCAGAACCCCGATCCTGCCGTCTATCAGCTGAGAGACAGCAAGAGCAGCGA
CAAGAGCGTGTGCCTGTTCACCGACTTCGACTCCCAGACCAACGTGTCCCAGAGC
AAGGACAGCGACGTGTACATCACCGACAAGACCGTGCTGGACATGCGGAGCATG
GACTTCAAGAGCAACAGCGCCGTGGCCTGGTCCAACAAGAGCGATTTCGCCTGC
GCCAACGCCTTCAACAACAGCATTATCCCCGAGGACACATTCTTCCCAAGTCCTG
AGAGCAGCTGCGACGTGAAGCTGGTGGAAAAGAGCTTCGAGACAGACACCAAC
CTGAACTTCCAGAACCTGAGCGTGATCGGCTTCCGGATCCTGCTCCTGAAAGTGG
CCGGCTTCAACCTGCTGATGACCCTGAGACTGTGGTCCAGCCGGGCCAAACGATC
TGGCTCTGGCCAGTGTACCAACTATGCCCTGCTCAAGCTCGCTGGCGACGTCGAG
TCTAACCCTGGACCAATGACCATCCTGGGCACCACCTTCGGCATGGTGTTTAGCC
TGCTGCAGGTCGTGTCTGGCAGCCTGGATAACAACGGCACAGCCACACCTGAGC
TGCCTACACAGGGCACCTTCAGCAACGTGTCCACCAATGTGTCCTACCAAGAGAC
AACCACACCTAGCACACTGGGCAGCACATCTCTGCACCCTGTGTCTCAGCACGGC
AATGAGGCCACCACCAACATCACCGAGACAACCGTGAAGTTCACCAGCACCAGC
GTGATCACCTCCGTGTACGGCAACACCAACAGCAGCGTGCAGAGCCAGACCTCC
GTGATCAGCACCGTGTTTACAACCCCTGCCAATGTCTCTACCCCTGAGACAACTC
TGAAGCCCAGCCTGTCTCCTGGAAACGTGTCCGATCTGAGCACCACCTCTACCAG
CCTGGCCACCTCTCCTACAAAGCCCTACACAAGCAGCAGCCCCATCCTGAGCGAT
ATCAAGGCCGAGATCAAGTGCAGCGGCATCCGGGAAGTGAAGCTGACCCAGGGC
ATCTGCCTGGAACAGAACAAGACCAGCAGCTGCGCCGAGTTCAAGAAGGACAGA
GGCGAAGGACTGGCCAGAGTGCTGTGTGGCGAAGAACAGGCCGATGCTGATGCT
GGCGCTCAAGTGTGCTCTCTGCTGCTGGCTCAGTCTGAAGTGCGGCCTCAGTGTC
TGCTTCTGGTCCTGGCCAACAGAACCGAGATCAGCAGCAAACTGCAGCTGATGA
AGAAGCACCAGAGCGACCTGAAGAAGCTGGGCATCCTGGACTTCACCGAGCAGG
ATGTGGCCAGCCACCAGAGCTACAGCCAGAAAACCCCTATCCTGCTGACATGCC
CCACAATCAGCATCCTGTCCTTTTTCAGCGTGGCCCTGCTCGTGATCCTGGCCTGT
GTGCTGTGGAAGAAGCGGATCAAGCCCATCGTGTGGCCTAGCCTGCCTGACCAC
AAGAAAACCCTGGAACACCTGTGCAAGAAGCCCCGGAAGAACCTGAACGTGTCC
TTCAATCCCGAGAGCTTCCTGGACTGCCAGATCCACAGAGTGGACGACATCCAG
GCCAGAGATGAGGTGGAAGGCTTTCTGCAGGACACTTTCCCACAGCAGCTGGAA
GAGAGCGAGAAGCAGAGACTCGGCGGAGATGTGCAGTCCCCTAATTGCCCTAGC
GAGGACGTGGTCATCACCCCAGAGAGCTTCGGCAGAGATAGCAGCCTGACATGT
CTGGCCGGCAATGTGTCTGCCTGTGACGCCCCTATTCTGAGCAGCTCCAGAAGCC
TGGATTGCAGAGAGAGCGGCAAGAACGGCCCTCACGTGTACCAGGATCTGCTCC
TGTCTCTGGGCACTACCAATAGCACCCTGCCTCCACCATTCAGCCTGCAGAGCGG
AATCCTGACACTGAACCCAGTGGCTCAGGGCCAGCCTATCCTGACAAGCCTGGG
CTCCAATCAAGAAGAGGCCTACGTCACCATGAGCAGCTTCTACCAGAACCAGTG
ATGATTCGAAGCGGCCGCTTCGAGCAGACATGATAAGATACATTGATGAGTTTG
GACAAACCACAACTAGAATGCAGTGAAAAAAATGCTTTATTTGTGAAATTTGTG
ATGCTATTGCTTTATTTGTAACCATTATAAGCTGCAATAAACAAGTTACACGGCA
GGGTCAGGGTTCTGGATATctgtgggacaagaggatcagggttaggacatgatctcatttccctctttgccccaaccc
aggctggagtccagatgccagtgatggacaagggcggggctctgtggggctggcaagtcacggtctcatgctttatacgggaaatag
catcttagaaaccagctgctcgtgatggactgggactcagggacaggcacaagctatcaatcttggccaagaggccatgatttcagtg
aacgttcacggccaggcctggcctgccactcaaggaaacCACTGGCCGTCGTTTTACAACGTCGTGACT
gggaaaaccctggcgttacccaacttaatcgccttgcagcacatccccctttcgccagctggcgtaatagcgaagaggcccgcaccg
atcgcccttcccaacagttgcgcagcctgaatggcgaatggcgcctgatgcggtattttctccttacgcatctgtgcggtatttcacaccg
catatggtgcactctcagtacaatctgctctgatgccgcatagttaagccagccccgacacccgccaacacccgctgacgcgccctga
cgggcttgtctgctcccggcatccgcttacagacaagctgtgaccgtctccgggagctgcatgtgtcagaggttttcaccgtcatcacc
gaaacgcgcga
-TCR-1 + C7R-1 (amino acid sequence)
SEQ ID NO: 66
MGTRLLCWAALCLLGAELTEAGVAQSPRYKIIEKRQSVAFWCNPISGHATLYWYQQ
ILGQGPKLLIQFQNNGVVDDSQLPKDRFSAERLKGVDSTLKIQPAKLEDSAVYLCASS
LDPGDTGELFFGEGSRLTVLEDLKNVFPPEVAVFEPSEAEISHTQKATLVCLATGFYP
DHVELSWWVNGKEVHSGVSTDPQPLKEQPALNDSRYCLSSRLRVSATFWQNPRNHF
RCQVQFYGLSENDEWTQDRAKPVTQIVSAEAWGRADCGFTSESYQQGVLSATILYEI
LLGKATLYAVLVSALVLMAMVKRKDSRGRAKRSGSGATNFSLLKQAGDVEENPGP
MESFLGGVLLILWLQVDWVKSQKIEQNSEALNIQEGKTATLTCNYTNYSPAYLQWY
RQDPGRGPVFLLLIRENEKEKRKERLKVTFDTTLKQSLFHITASQPADSATYLCALDI
YPHDMRFGAGTRLTVKPDIQNPDPAVYQLRDSKSSDKSVCLFTDFDSQTNVSQSKDS
DVYITDKTVLDMRSMDFKSNSAVAWSNKSDFACANAFNNSIIPEDTFFPSPESSCDV
KLVEKSFETDTNLNFQNLSVIGFRILLLKVAGFNLLMTLRLWSSRAKRSGSGQCTNY
ALLKLAGDVESNPGPMTILGTTFGMVFSLLQVVSGSLDNNGTATPELPTQGTFSNVS
TNVSYQETTTPSTLGSTSLHPVSQHGNEATTNITETTVKFTSTSVITSVYGNTNSSVQS
QTSVISTVFTTPANVSTPETTLKPSLSPGNVSDLSTTSTSLATSPTKPYTSSSPILSDIKA
EIKCSGIREVKLTQGICLEQNKTSSCAEFKKDRGEGLARVLCGEEQADADAGAQVCS
LLLAQSEVRPQCLLLVLANRTEISSKLQLMKKHQSDLKKLGILDFTEQDVASHQSYS
QKTPILLTCPTISILSFFSVALLVILACVLWKKRIKPIVWPSLPDHKKTLEHLCKKPRKN
LNVSFNPESFLDCQIHRVDDIQARDEVEGFLQDTFPQQLEESEKQRLGGDVQSPNCPS
EDVVITPESFGRDSSLTCLAGNVSACDAPILSSSRSLDCRESGKNGPHVYQDLLLSLG
TTNSTLPPPFSLQSGILTLNPVAQGQPILTSLGSNQEEAYVTMSSFYQNQ
-TCR-1 + C7R-2 (plasmid sequence)
SEQ ID NO: 67
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
GCACCAGACTGCTGTGTTGGGCTGCTCTGTGTCTGCTGGGAGCCGAACTTACTGA
AGCTGGCGTGGCCCAGTCTCCTCGGTACAAGATCATCGAGAAGCGGCAGAGCGT
GGCCTTCTGGTGCAATCCTATCAGCGGACACGCCACACTGTACTGGTATCAGCAG
ATCCTCGGACAGGGCCCCAAGCTGCTGATTCAGTTCCAGAACAACGGCGTGGTG
GACGACAGCCAGCTGCCTAAGGATAGATTCAGCGCCGAGCGGCTGAAAGGCGTG
GACAGCACACTGAAGATCCAGCCTGCCAAGCTGGAAGATAGCGCCGTGTACCTG
TGTGCCAGCTCTCTGGATCCTGGCGATACCGGCGAGCTGTTCTTTGGCGAGGGCA
GCAGACTGACCGTGCTCGAGGACCTGAAGAACGTGTTCCCACCTGAGGTGGCCG
TGTTCGAGCCTTCTGAGGCCGAGATCAGCCACACACAGAAAGCCACACTCGTGT
GTCTGGCCACCGGCTTCTATCCCGATCACGTGGAACTGTCTTGGTGGGTCAACGG
CAAAGAGGTGCACAGCGGCGTCAGCACAGATCCCCAGCCTCTGAAAGAGCAGCC
AGCTCTGAACGACAGCCGGTACTGTCTGAGCAGCAGGCTGAGAGTGTCCGCCAC
CTTCTGGCAGAACCCCAGAAACCACTTCAGATGCCAGGTGCAGTTCTACGGCCTG
AGCGAGAACGATGAGTGGACACAGGATAGAGCCAAGCCTGTGACACAGATCGT
GTCTGCCGAAGCCTGGGGCAGAGCCGATTGTGGCTTTACCAGCGAGAGCTACCA
GCAGGGCGTGCTGTCTGCCACAATCCTGTACGAGATCCTGCTGGGCAAAGCCACT
CTGTACGCCGTGCTGGTGTCTGCCCTGGTGCTGATGGCCATGGTCAAGCGGAAGG
ACTCTAGAGGCAGAGCCAAGAGATCTGGCAGCGGCGCCACAAACTTTAGCCTGC
TGAAACAGGCCGGCGACGTGGAAGAGAACCCCGGACCTATGGAATCCTTTCTCG
GTGGCGTGCTGCTCATCCTGTGGCTGCAAGTGGATTGGGTCAAGAGCCAGAAGA
TCGAGCAGAACAGCGAGGCCCTGAACATCCAAGAGGGCAAGACCGCCACTCTGA
CCTGCAACTACACCAACTACAGCCCCGCCTACCTGCAGTGGTACAGACAGGACC
CTGGAAGAGGCCCTGTGTTCCTGCTGCTGATCCGCGAGAATGAGAAAGAGAAGC
GCAAAGAGCGCCTGAAAGTCACCTTCGACACAACCCTGAAGCAGAGCCTGTTCC
ACATCACCGCCTCTCAGCCAGCCGATAGCGCCACATATCTGTGCGCCCTGGACAT
CTACCCTCACGACATGAGATTCGGAGCCGGCACCAGGCTGACAGTGAAGCCCGA
TATTCAGAACCCCGATCCTGCCGTCTATCAGCTGAGAGACAGCAAGAGCAGCGA
CAAGAGCGTGTGCCTGTTCACCGACTTCGACTCCCAGACCAACGTGTCCCAGAGC
AAGGACAGCGACGTGTACATCACCGACAAGACCGTGCTGGACATGCGGAGCATG
GACTTCAAGAGCAACAGCGCCGTGGCCTGGTCCAACAAGAGCGATTTCGCCTGC
GCCAACGCCTTCAACAACAGCATTATCCCCGAGGACACATTCTTCCCAAGTCCTG
AGAGCAGCTGCGACGTGAAGCTGGTGGAAAAGAGCTTCGAGACAGACACCAAC
CTGAACTTCCAGAACCTGAGCGTGATCGGCTTCCGGATCCTGCTCCTGAAAGTGG
CCGGCTTCAACCTGCTGATGACCCTGAGACTGTGGTCCAGCCGGGCCAAACGATC
TGGCTCTGGCCAGTGTACCAACTATGCCCTGCTCAAGCTCGCTGGCGACGTCGAG
TCTAACCCTGGACCAATGACCATCCTGGGCACCACCTTCGGCATGGTGTTTAGCC
TGCTGCAGGTCGTGTCTGGCGAGTCTGGCTATGCCCAGAATGGCGACCTGGAAG
ATGCCGAGCTGGACGACTACAGCTTCAGCTGCTACAGCCAGCTGGAAGTGAACG
GCAGCCAGCACTCTCTGACCTGCGCCTTCGAAGATCCCGACGTGAACATCACCAA
CCTGGAATTCGAGATCTGTGGCGCCCTGGTGGAAGTCAAGTGCCTGAACTTCAGA
AAGCTGCAAGAGATCTACTTTATCGAGACAAAGAAGTTCCTGCTGATCGGCAAG
AGCAACATCTGCGTGAAAGTGGGCGAGAAGTCCCTGACCTGCAAGAAGATCGAC
CTGACCACCATCGTGAAGCCCGAGGCTCCTTTCGATCTGAGCGTGGTGTATAGAG
AGGGCGCCAACGACTTCGTGGTCACCTTCAACACCAGCCACCTCCAAAAGAAAT
ACGTGAAGGTGCTGATGCACGACGTGGCCTACCGGCAAGAGAAGGACGAGAAC
AAGTGGACCCACGTGAACCTGAGCAGCACCAAGCTGACACTGCTGCAGAGGAAA
CTGCAGCCTGCCGCTATGTACGAGATCAAAGTGCGGAGCATCCCCGACCACTACT
TCAAAGGCTTTTGGAGCGAGTGGTCCCCGAGCTACTACTTTCGGACCCCTGAGAT
CAACAACAGCAGCGGCGAGATGGACCCCATCCTGCTGACCTGTCCTACAATCAG
CATCCTGAGCTTTTTCAGCGTGGCCCTGCTGGTCATCCTGGCCTGTGTGCTGTGGA
AGAAGCGGATCAAGCCCATCGTGTGGCCCAGCCTGCCTGACCACAAGAAAACCC
TGGAACACCTGTGCAAGAAGCCCCGGAAGAACCTGAACGTGTCCTTCAATCCCG
AGAGCTTCCTGGACTGCCAGATCCACAGAGTGGACGACATCCAGGCCAGGGACG
AAGTGGAAGGCTTTCTGCAGGACACATTCCCTCAGCAGCTCGAGGAAAGCGAGA
AGCAGAGACTCGGCGGAGATGTGCAGAGCCCTAATTGCCCTAGCGAGGACGTGG
TCATCACCCCAGAGAGCTTCGGCAGAGATAGCAGCCTGACATGTCTGGCCGGCA
ATGTGTCCGCCTGTGATGCCCCTATCCTGTCCAGCAGCAGAAGCCTGGATTGCAG
AGAGAGCGGCAAGAACGGCCCTCACGTGTACCAGGATCTGCTCCTGTCTCTGGG
AACCACCAACAGCACACTGCCTCCACCATTCAGCCTGCAGAGCGGCATCCTGAC
ACTGAACCCTGTTGCTCAGGGCCAGCCAATCCTGACAAGCCTGGGCAGCAATCA
AGAAGAGGCCTACGTCACCATGAGCAGCTTCTACCAGAACCAGTGATGATTCGA
AGCGGCCGCTTCGAGCAGACATGATAAGATACATTGATGAGTTTGGACAAACCA
CAACTAGAATGCAGTGAAAAAAATGCTTTATTTGTGAAATTTGTGATGCTATTGC
TTTATTTGTAACCATTATAAGCTGCAATAAACAAGTTACACGGCAGGGTCAGGGT
TCTGGATATctgtgggacaagaggatcagggttaggacatgatctcatttccctctttgccccaacccaggctggagtccagat
gccagtgatggacaagggcggggctctgtggggctggcaagtcacggtctcatgctttatacgggaaatagcatcttagaaaccagct
gctcgtgatggactgggactcagggacaggcacaagctatcaatcttggccaagaggccatgatttcagtgaacgttcacggccagg
cctggcctgccactcaaggaaacCACTGGCCGTCGTTTTACAACGTCGTGACTgggaaaaccctggcgtt
acccaacttaatcgccttgcagcacatccccctttcgccagctggcgtaatagcgaagaggcccgcaccgatcgcccttcccaacagt
tgcgcagcctgaatggcgaatggcgcctgatgcggtattttctccttacgcatctgtgcggtatttcacaccgcatatggtgcactctcag
tacaatctgctctgatgccgcatagttaagccagccccgacacccgccaacacccgctgacgcgccctgacgggcttgtctgctcccg
gcatccgcttacagacaagctgtgaccgtctccgggagctgcatgtgtcagaggttttcaccgtcatcaccgaaacgcgcga
-TCR-1 + C7R-2 (amino acid sequence)
SEQ ID NO: 68
MGTRLLCWAALCLLGAELTEAGVAQSPRYKIIEKRQSVAFWCNPISGHATLYWYQQ
ILGQGPKLLIQFQNNGVVDDSQLPKDRFSAERLKGVDSTLKIQPAKLEDSAVYLCASS
LDPGDTGELFFGEGSRLTVLEDLKNVFPPEVAVFEPSEAEISHTQKATLVCLATGFYP
DHVELSWWVNGKEVHSGVSTDPQPLKEQPALNDSRYCLSSRLRVSATFWQNPRNHF
RCQVQFYGLSENDEWTQDRAKPVTQIVSAEAWGRADCGFTSESYQQGVLSATILYEI
LLGKATLYAVLVSALVLMAMVKRKDSRGRAKRSGSGATNFSLLKQAGDVEENPGP
MESFLGGVLLILWLQVDWVKSQKIEQNSEALNIQEGKTATLTCNYTNYSPAYLQWY
RQDPGRGPVFLLLIRENEKEKRKERLKVTFDTTLKQSLFHITASQPADSATYLCALDI
YPHDMRFGAGTRLTVKPDIQNPDPAVYQLRDSKSSDKSVCLFTDFDSQTNVSQSKDS
DVYITDKTVLDMRSMDFKSNSAVAWSNKSDFACANAFNNSIIPEDTFFPSPESSCDV
KLVEKSFETDTNLNFQNLSVIGFRILLLKVAGFNLLMTLRLWSSRAKRSGSGQCTNY
ALLKLAGDVESNPGPMTILGTTFGMVFSLLQVVSGESGYAQNGDLEDAELDDYSFSC
YSQLEVNGSQHSLTCAFEDPDVNITNLEFEICGALVEVKCLNFRKLQEIYFIETKKFLL
IGKSNICVKVGEKSLTCKKIDLTTIVKPEAPFDLSVVYREGANDFVVTFNTSHLQKKY
VKVLMHDVAYRQEKDENKWTHVNLSSTKLTLLQRKLQPAAMYEIKVRSIPDHYFK
GFWSEWSPSYYFRTPEINNSSGEMDPILLTCPTISILSFFSVALLVILACVLWKKRIKPI
VWPSLPDHKKTLEHLCKKPRKNLNVSFNPESFLDCQIHRVDDIQARDEVEGFLQDTF
PQQLEESEKQRLGGDVQSPNCPSEDVVITPESFGRDSSLTCLAGNVSACDAPILSSSRS
LDCRESGKNGPHVYQDLLLSLGTTNSTLPPPFSLQSGILTLNPVAQGQPILTSLGSNQE
EAYVTMSSFYQNQ
-TCR-1 + Cβ-MC (plasmid sequence)
SEQ ID NO: 69
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
GCACTCGATTGCTCTGTTGGGCCGCGCTGTGCTTGCTGGGTGCCGAATTGACAGA
AGCGGGTGTAGCTCAAAGTCCTCGGTACAAGATTATTGAGAAACGCCAGTCTGTT
GCCTTCTGGTGTAACCCTATCTCTGGTCACGCAACTCTCTATTGGTATCAGCAAAT
CCTGGGTCAAGGTCCTAAACTTCTGATTCAATTTCAGAATAATGGCGTTGTGGAC
GACTCTCAACTGCCTAAAGACAGATTCAGTGCGGAAAGGTTGAAAGGCGTCGAT
AGCACACTCAAAATTCAGCCGGCAAAGCTTGAAGACTCCGCCGTTTATCTGTGCG
CGAGTTCTCTTGATCCTGGGGACACAGGGGAACTGTTCTTTGGCGAGGGATCACG
ATTGACCGTCCTGGAGGACCTGAGAAACGTGACCCCTCCTAAGGTGTCCCTGTTC
GAGCCTAGCAAGGCCGAGATCGCCAACAAGCAGAAAGCCACACTCGTGTGCCTG
GCCAGAGGCTTCTTTCCCGATCACGTGGAACTGTCTTGGTGGGTCAACGGCAAAG
AGGTGCACAGCGGCGTCTGTACTGATCCCCAGGCCTACAAAGAGAGCAACTACA
GCTACTGCCTGAGCAGCAGACTGAGAGTGTCCGCCACCTTCTGGCACAACCCCA
GAAACCACTTCAGATGCCAGGTGCAGTTTCACGGCCTGAGCGAAGAGGACAAGT
GGCCTGAGGGAAGCCCCAAGCCAGTGACACAGAATATCTCTGCCGAAGCCTGGG
GCAGAGCCGATTGTGGAATTACCAGCGCCAGCTACCAGCAAGGCGTGCTGTCTG
CCACAATCCTGTACGAGATCCTGCTGGGCAAAGCCACTCTGTACGCCGTGCTGGT
GTCTACCCTGGTCGTGATGGCCATGGTCATGGCTGCTGGTGGACCTGGTGCTGGA
TCTGCTGCCCCTGTGTCTAGCACATCTAGCCTGCCTCTGGCCGCTCTGAACATGA
GAGTCAGAAGAAGGCTGAGCCTGTTCCTGAACGTGCGGACTCAGGTGGCCGCTG
ATTGGACAGCTCTGGCCGAGGAAATGGACTTCGAGTACCTGGAAATCCGGCAGC
TGGAAACCCAGGCCGATCCTACAGGCAGACTGCTGGATGCTTGGCAAGGCAGAC
CTGGCGCTTCTGTGGGGAGACTGCTTGAGCTGCTGACAAAGCTGGGCAGAGATG
ACGTGCTGCTGGAACTGGGCCCTAGCATCGAGGAAGATTGCCAGAAGTACATCC
TGAAGCAGCAGCAAGAGGAAGCCGAGAAGCCTCTGCAAGTGGCCGCCGTGGAT
AGCAGCGTTCCAAGAACAGCTGAGCTGGCCGGCATCACCACACTGGATGATCCT
CTGGGAAAGAAGGTGGCCAAGAAGCCCACCAACAAGGCCCCTCATCCTAAGCAA
GAGCCCCAAGAGATCAACTTCCCCGACGATCTGCCCGGCAGCAATACTGCTGCTC
CCGTGCAAGAAACCCTGCACGGTTGTCAGCCCGTGACACAAGAGGACGGCAAAG
AAAGCCGGATCAGCGTGCAAGAGAGACAGAGGGCCAAGAGAAGTGGAAGCGGC
GCCACAAACTTCAGCCTGTTGAAACAGGCCGGGGACGTCGAAGAGAATCCTGGA
CCAATGGAATCCTTTCTCGGAGGTGTACTGCTCATACTCTGGCTGCAAGTGGATT
GGGTAAAGAGTCAAAAAATTGAACAAAACAGCGAGGCTTTGAATATCCAAGAG
GGCAAGACTGCCACGCTGACCTGTAACTATACAAACTATAGTCCGGCATACCTCC
AGTGGTACCGGCAAGATCCTGGTCGCGGGCCGGTTTTCCTGTTGCTTATTAGAGA
GAATGAAAAGGAAAAAAGAAAAGAACGCTTGAAGGTCACTTTCGACACGACGC
TTAAACAGAGTCTTTTCCACATTACCGCGTCACAGCCTGCGGACTCAGCTACATA
CCTGTGTGCTCTCGACATCTATCCCCACGACATGAGGTTTGGCGCAGGCACTCGA
CTCACGGTAAAACCCGACATTCAGAATCCTGAGCCTGCCGTGTACCAGCTGAAG
GACCCTAGAAGCCAGGACAGCACCCTGTGCCTGTTCACCGACTTCGACAGCCAG
ATCAACGTGCCCAAGACCATGGAAAGCGGCACCTTCATCACCGATAAGTGCGTG
CTGGACATGAAGGCCATGGACAGCAAGAGCAACGGCGCCATTGCCTGGTCCAAC
CAGACCAGCTTCACGTGCCAGGACATCTTCAAAGAGACAAACGCCACCTATCCT
AGCAGCGACGTGCCCTGTGATGCCACACTGACCGAGAAGTCCTTCGAGACAGAC
ATGAACCTGAACTTCCAGAACCTGCTGGTCATCGTGCTGAGAATCCTGCTGCTGA
AGGTGGCCGGCTTCAACCTGCTGATGACCCTGAGACTGTGGTCCAGCCGGGCCA
AACGATCTGGCTCTGGCCAGTGTACCAACTATGCCCTGCTCAAGCTCGCTGGCGA
CGTCGAGTCTAACCCTGGACCAATGGGAGCTGGCGCTACAGGCAGAGCTATGGA
TGGACCTAGACTGCTGCTGCTCCTGCTTCTGGGAGTGTCTCTTGGCGGAGCCAAA
GAGGCCTGTCCTACCGGCCTGTATACACACTCTGGCGAGTGCTGCAAGGCCTGCA
ATCTTGGAGAAGGCGTCGCACAGCCTTGCGGCGCTAATCAGACAGTGTGCGAGC
CTTGCCTGGACAGCGTGACCTTTAGCGACGTGGTGTCAGCCACCGAGCCATGCAA
GCCATGTACCGAGTGTGTGGGCCTGCAGAGCATGTCTGCCCCTTGCGTGGAAGCT
GACGATGCCGTGTGTAGATGCGCCTACGGCTACTACCAGGACGAGACAACAGGC
AGATGCGAGGCCTGTAGAGTGTGCGAAGCTGGATCTGGCCTGGTGTTCAGCTGC
CAAGACAAGCAGAACACCGTGTGCGAGGAATGCCCCGATGGCACCTATAGCGAC
GAGGCCAACCATGTGGATCCCTGCCTGCCTTGTACCGTGTGTGAAGATACCGAGC
GGCAGCTGCGCGAGTGTACAAGATGGGCTGATGCCGAGTGCGAAGAGATCCCTG
GCAGATGGATCACCAGAAGCACACCTCCAGAGGGCAGCGATAGCACAGCCCCTT
CTACACAAGAGCCCGAGGCTCCTCCTGAGCAGGACCTGATTGCTTCTACAGTGGC
TGGCGTCGTGACCACCGTGATGGGATCTAGTCAGCCCGTGGTCACCAGAGGCAC
CACCGACAATCTGATCCCCGTGTACTGTAGCATCCTGGCCGCCGTGGTTGTTGGA
CTGGTGGCCTATATCGCCTTCAAGCGGTGGAACAGCTGATGATTCGAAGCGGCC
GCTTCGAGCAGACATGATAAGATACATTGATGAGTTTGGACAAACCACAACTAG
AATGCAGTGAAAAAAATGCTTTATTTGTGAAATTTGTGATGCTATTGCTTTATTTG
TAACCATTATAAGCTGCAATAAACAAGTTACACGGCAGGGTCAGGGTTCTGGAT
ATctgtgggacaagaggatcagggttaggacatgatctcatttccctctttgccccaacccaggctggagtccagatgccagtgatgg
acaagggcggggctctgtggggctggcaagtcacggtctcatgctttatacgggaaatagcatcttagaaaccagctgctcgtgatgg
actgggactcagggacaggcacaagctatcaatcttggccaagaggccatgatttcagtgaacgttcacggccaggcctggcctgcc
actcaaggaaacCACTGGCCGTCGTTTTACAACGTCGTGACTgggaaaaccctggcgttacccaacttaa
tcgccttgcagcacatccccctttcgccagctggcgtaatagcgaagaggcccgcaccgatcgcccttcccaacagttgcgcagcctg
aatggcgaatggcgcctgatgcggtattttctccttacgcatctgtgcggtatttcacaccgcatatggtgcactctcagtacaatctgctc
tgatgccgcatagttaagccagccccgacacccgccaacacccgctgacgcgccctgacgggcttgtctgctcccggcatccgctta
cagacaagctgtgaccgtctccgggagctgcatgtgtcagaggttttcaccgtcatcaccgaaacgcgcga
-TCR-1 + Cβ-MC (amino acid sequence)
SEQ ID NO: 70
MGTRLLCWAALCLLGAELTEAGVAQSPRYKIIEKRQSVAFWCNPISGHATLYWYQQ
ILGQGPKLLIQFQNNGVVDDSQLPKDRFSAERLKGVDSTLKIQPAKLEDSAVYLCASS
LDPGDTGELFFGEGSRLTVLEDLRNVTPPKVSLFEPSKAEIANKQKATLVCLARGFFP
DHVELSWWVNGKEVHSGVCTDPQAYKESNYSYCLSSRLRVSATFWHNPRNHFRCQ
VQFHGLSEEDKWPEGSPKPVTQNISAEAWGRADCGITSASYQQGVLSATILYEILLGK
ATLYAVLVSTLVVMAMVMAAGGPGAGSAAPVSSTSSLPLAALNMRVRRRLSLFLN
VRTQVAADWTALAEEMDFEYLEIRQLETQADPTGRLLDAWQGRPGASVGRLLELLT
KLGRDDVLLELGPSIEEDCQKYILKQQQEEAEKPLQVAAVDSSVPRTAELAGITTLDD
PLGKKVAKKPTNKAPHPKQEPQEINFPDDLPGSNTAAPVQETLHGCQPVTQEDGKES
RISVQERQRAKRSGSGATNFSLLKQAGDVEENPGPMESFLGGVLLILWLQVDWVKS
QKIEQNSEALNIQEGKTATLTCNYTNYSPAYLQWYRQDPGRGPVFLLLIRENEKEKR
KERLKVTFDTTLKQSLFHITASQPADSATYLCALDIYPHDMRFGAGTRLTVKPDIQNP
EPAVYQLKDPRSQDSTLCLFTDFDSQINVPKTMESGTFITDKCVLDMKAMDSKSNGA
IAWSNQTSFTCQDIFKETNATYPSSDVPCDATLTEKSFETDMNLNFQNLLVIVLRILLL
KVAGFNLLMTLRLWSSRAKRSGSGQCTNYALLKLAGDVESNPGPMGAGATGRAM
DGPRLLLLLLLGVSLGGAKEACPTGLYTHSGECCKACNLGEGVAQPCGANQTVCEP
CLDSVTFSDVVSATEPCKPCTECVGLQSMSAPCVEADDAVCRCAYGYYQDETTGRC
EACRVCEAGSGLVFSCQDKQNTVCEECPDGTYSDEANHVDPCLPCTVCEDTERQLR
ECTRWADAECEEIPGRWITRSTPPEGSDSTAPSTQEPEAPPEQDLIASTVAGVVTTVM
GSSQPVVTRGTTDNLIPVYCSILAAVVVGLVAYIAFKRWNS
-TCR-1 + Fas-MC (plasmid sequence)
SEQ ID NO: 71
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
GCACTCGATTGCTCTGTTGGGCCGCGCTGTGCTTGCTGGGTGCCGAATTGACAGA
AGCGGGTGTAGCTCAAAGTCCTCGGTACAAGATTATTGAGAAACGCCAGTCTGTT
GCCTTCTGGTGTAACCCTATCTCTGGTCACGCAACTCTCTATTGGTATCAGCAAAT
CCTGGGTCAAGGTCCTAAACTTCTGATTCAATTTCAGAATAATGGCGTTGTGGAC
GACTCTCAACTGCCTAAAGACAGATTCAGTGCGGAAAGGTTGAAAGGCGTCGAT
AGCACACTCAAAATTCAGCCGGCAAAGCTTGAAGACTCCGCCGTTTATCTGTGCG
CGAGTTCTCTTGATCCTGGGGACACAGGGGAACTGTTCTTTGGCGAGGGATCACG
ATTGACCGTCCTGGAGGACCTGAGAAACGTGACCCCTCCTAAGGTGTCCCTGTTC
GAGCCTAGCAAGGCCGAGATCGCCAACAAGCAGAAAGCCACACTCGTGTGCCTG
GCCAGAGGCTTCTTTCCCGATCACGTGGAACTGTCTTGGTGGGTCAACGGCAAAG
AGGTGCACAGCGGCGTCTGTACTGATCCCCAGGCCTACAAAGAGAGCAACTACA
GCTACTGCCTGAGCAGCAGACTGAGAGTGTCCGCCACCTTCTGGCACAACCCCA
GAAACCACTTCAGATGCCAGGTGCAGTTTCACGGCCTGAGCGAAGAGGACAAGT
GGCCTGAGGGAAGCCCCAAGCCAGTGACACAGAATATCTCTGCCGAAGCCTGGG
GCAGAGCCGATTGTGGAATTACCAGCGCCAGCTACCAGCAAGGCGTGCTGTCTG
CCACAATCCTGTACGAGATCCTGCTGGGCAAAGCCACTCTGTACGCCGTGCTGGT
GTCTACCCTGGTCGTGATGGCCATGGTCAAGCGGAAGAACAGCAGGGCCAAGAG
AAGTGGAAGCGGCGCCACAAACTTCAGCCTGTTGAAACAGGCCGGGGACGTCGA
AGAGAATCCTGGACCAATGGAATCCTTTCTCGGAGGTGTACTGCTCATACTCTGG
CTGCAAGTGGATTGGGTAAAGAGTCAAAAAATTGAACAAAACAGCGAGGCTTTG
AATATCCAAGAGGGCAAGACTGCCACGCTGACCTGTAACTATACAAACTATAGT
CCGGCATACCTCCAGTGGTACCGGCAAGATCCTGGTCGCGGGCCGGTTTTCCTGT
TGCTTATTAGAGAGAATGAAAAGGAAAAAAGAAAAGAACGCTTGAAGGTCACTT
TCGACACGACGCTTAAACAGAGTCTTTTCCACATTACCGCGTCACAGCCTGCGGA
CTCAGCTACATACCTGTGTGCTCTCGACATCTATCCCCACGACATGAGGTTTGGC
GCAGGCACTCGACTCACGGTAAAACCCGACATTCAGAATCCTGAGCCTGCCGTG
TACCAGCTGAAGGACCCTAGAAGCCAGGACAGCACCCTGTGCCTGTTCACCGAC
TTCGACAGCCAGATCAACGTGCCCAAGACCATGGAAAGCGGCACCTTCATCACC
GATAAGTGCGTGCTGGACATGAAGGCCATGGACAGCAAGAGCAACGGCGCCATT
GCCTGGTCCAACCAGACCAGCTTCACGTGCCAGGACATCTTCAAAGAGACAAAC
GCCACCTATCCTAGCAGCGACGTGCCCTGTGATGCCACACTGACCGAGAAGTCCT
TCGAGACAGACATGAACCTGAACTTCCAGAACCTGCTGGTCATCGTGCTGAGAA
TCCTGCTGCTGAAGGTGGCCGGCTTCAACCTGCTGATGACCCTGAGACTGTGGTC
CAGCCGGGCCAAACGATCTGGCTCTGGCCAGTGTACCAACTATGCCCTGCTCAAG
CTCGCTGGCGACGTCGAGTCTAACCCTGGACCAATGCTCGGCATCTGGACACTGC
TGCCTCTGGTGCTGACAAGCGTGGCCAGACTGAGCAGCAAGAGCGTGAACGCCC
AAGTGACCGACATCAACAGCAAAGGCCTGGAACTGAGAAAGACCGTGACCACC
GTGGAAACCCAGAACCTGGAAGGCCTGCACCACGACGGCCAGTTCTGTCACAAA
CCTTGTCCACCTGGCGAGCGGAAGGCCAGAGATTGCACAGTGAATGGCGACGAG
CCTGACTGCGTGCCCTGTCAAGAGGGCAAAGAGTACACCGACAAGGCCCACTTC
AGCAGCAAGTGCAGACGGTGCAGACTGTGCGACGAAGGCCACGGACTGGAAGT
GGAAATCAACTGCACCCGGACACAGAACACCAAGTGCCGGTGCAAGCCCAACTT
CTTCTGCAACAGCACCGTGTGCGAGCACTGCGACCCTTGTACCAAGTGCGAACAC
GGCATCATCAAAGAGTGCACCCTGACCTCCAACACGAAGTGCAAAGAGGAAGGC
AGCAGAAGCAACCTCGGCTGGCTGTGTCTGCTGCTGCTCCCCATTCCTCTGATCG
TGTGGGTTATGGCTGCTGGCGGACCTGGTGCTGGATCTGCTGCACCTGTGTCCAG
CACATCTAGCCTGCCACTGGCCGCTCTGAACATGAGAGTCAGAAGAAGGCTGAG
CCTGTTCCTGAACGTGCGCACTCAGGTGGCCGCTGATTGGACAGCTCTGGCCGAG
GAAATGGACTTCGAGTACCTGGAAATCCGGCAGCTGGAAACACAGGCCGATCCT
ACAGGCAGACTGCTGGATGCTTGGCAAGGCAGACCTGGCGCTTCTGTGGGGAGA
CTGCTTGAGCTGCTGACAAAGCTGGGCAGAGATGACGTGCTGCTGGAACTGGGA
CCCAGCATCGAGGAAGATTGCCAGAAGTACATCCTGAAGCAGCAACAAGAGGA
AGCCGAGAAGCCTCTGCAGGTTGCCGCCGTGGATAGCAGCGTTCCAAGAACAGC
TGAGCTGGCCGGCATCACCACACTGGATGATCCTCTGGGAAAGAAGGTGGCCAA
GAAGCCCACAAACAAGGCCCCTCATCCTAAGCAAGAGCCCCAAGAGATCAACTT
CCCCGACGATCTGCCCGGCAGCAATACTGCAGCTCCCGTGCAAGAAACCCTGCA
CGGTTGTCAGCCCGTGACACAAGAGGACGGCAAAGAAAGCCGGATCAGCGTGCA
AGAGAGACAGTGATGATTCGAAGCGGCCGCTTCGAGCAGACATGATAAGATACA
TTGATGAGTTTGGACAAACCACAACTAGAATGCAGTGAAAAAAATGCTTTATTTG
TGAAATTTGTGATGCTATTGCTTTATTTGTAACCATTATAAGCTGCAATAAACAA
GTTACACGGCAGGGTCAGGGTTCTGGATATctgtgggacaagaggatcagggttaggacatgatctcattt
ccctctttgccccaacccaggctggagtccagatgccagtgatggacaagggcggggctctgtggggctggcaagtcacggtctcat
gctttatacgggaaatagcatcttagaaaccagctgctcgtgatggactgggactcagggacaggcacaagctatcaatcttggccaa
gaggccatgatttcagtgaacgttcacggccaggcctggcctgccactcaaggaaacCACTGGCCGTCGTTTTACA
ACGTCGTGACTgggaaaaccctggcgttacccaacttaatcgccttgcagcacatccccctttcgccagctggcgtaatagc
gaagaggcccgcaccgatcgcccttcccaacagttgcgcagcctgaatggcgaatggcgcctgatgcggtattttctccttacgcatct
gtgcggtatttcacaccgcatatggtgcactctcagtacaatctgctctgatgccgcatagttaagccagccccgacacccgccaacac
ccgctgacgcgccctgacgggcttgtctgctcccggcatccgcttacagacaagctgtgaccgtctccgggagctgcatgtgtcagag
gttttcaccgtcatcaccgaaacgcgcga
-TCR-1 + Fas-MC (amino acid sequence)
SEQ ID NO: 72
MGTRLLCWAALCLLGAELTEAGVAQSPRYKIIEKRQSVAFWCNPISGHATLYWYQQ
ILGQGPKLLIQFQNNGVVDDSQLPKDRFSAERLKGVDSTLKIQPAKLEDSAVYLCASS
LDPGDTGELFFGEGSRLTVLEDLRNVTPPKVSLFEPSKAEIANKQKATLVCLARGFFP
DHVELSWWVNGKEVHSGVCTDPQAYKESNYSYCLSSRLRVSATFWHNPRNHFRCQ
VQFHGLSEEDKWPEGSPKPVTQNISAEAWGRADCGITSASYQQGVLSATILYEILLGK
ATLYAVLVSTLVVMAMVKRKNSRAKRSGSGATNFSLLKQAGDVEENPGPMESFLG
GVLLILWLQVDWVKSQKIEQNSEALNIQEGKTATLTCNYTNYSPAYLQWYRQDPGR
GPVFLLLIRENEKEKRKERLKVTFDTTLKQSLFHITASQPADSATYLCALDIYPHDMR
FGAGTRLTVKPDIQNPEPAVYQLKDPRSQDSTLCLFTDFDSQINVPKTMESGTFITDK
CVLDMKAMDSKSNGAIAWSNQTSFTCQDIFKETNATYPSSDVPCDATLTEKSFETD
MNLNFQNLLVIVLRILLLKVAGENLLMTLRLWSSRAKRSGSGQCTNYALLKLAGDV
ESNPGPMLGIWTLLPLVLTSVARLSSKSVNAQVTDINSKGLELRKTVTTVETQNLEGL
HHDGQFCHKPCPPGERKARDCTVNGDEPDCVPCQEGKEYTDKAHFSSKCRRCRLCD
EGHGLEVEINCTRTQNTKCRCKPNFFCNSTVCEHCDPCTKCEHGIIKECTLTSNTKCK
EEGSRSNLGWLCLLLLPIPLIVWVMAAGGPGAGSAAPVSSTSSLPLAALNMRVRRRL
SLFLNVRTQVAADWTALAEEMDFEYLEIRQLETQADPTGRLLDAWQGRPGASVGRL
LELLTKLGRDDVLLELGPSIEEDCQKYILKQQQEEAEKPLQVAAVDSSVPRTAELAGI
TTLDDPLGKKVAKKPTNKAPHPKQEPQEINFPDDLPGSNTAAPVQETLHGCQPVTQE
DGKESRISVQERQ
-Split/CAR-3 + Cβ-MC (plasmid sequence)
SEQ ID NO: 73
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggttgggggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcggggggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
CTCTGCCTGTGACAGCTCTGCTGCTGCCTCTTGCTCTGCTTCTGCATGCCGCCAGA
CCTGACATCCAGATGACACAGAGCCCTAGCAGCCTGTCTGCCAGCGTGGGAGAC
AGAGTGACCATCACCTGTAGAGCCAGCCAGGACGTGAACACAGCCGTGGCTTGG
TATCAGCAGAAGCCTGGCAAGGCCCCTAAGCTGCTGATCTACAGCGCCTACTTCC
TGTACAGCGGCGTGCCCAGCAGATTCAGCGGCTCTAGAAGCGGCACCGACTTCA
CCCTGACCATAAGCAGTCTGCAGCCCGAGGACTTCGCCACCTACTACTGTCAGCA
GTACAGCCGGTACAGCCCCGTGACATTTGGCCAGGGCACCAAGGTGGAAATCAA
GGAAGCCGCCGCTAAAGAGGACCTGAGAAACGTGACCCCTCCTAAGGTGTCCCT
GTTCGAGCCTAGCAAGGCCGAGATCGCCAACAAGCAGAAAGCCACACTCGTGTG
CCTGGCCAGAGGCTTCTTTCCCGATCACGTGGAACTGTCTTGGTGGGTCAACGGC
AAAGAGGTGCACAGCGGCGTCTGTACTGATCCCCAGGCCTACAAAGAGAGCAAC
TACAGCTACTGCCTGAGCAGCAGACTGAGAGTGTCCGCCACCTTCTGGCACAACC
CCAGAAACCACTTCAGATGCCAGGTGCAGTTTCACGGCCTGAGCGAAGAGGACA
AGTGGCCTGAGGGAAGCCCCAAGCCAGTGACACAGAATATCTCTGCCGAAGCCT
GGGGCAGAGCCGATTGTGGAATTACCAGCGCCAGCTACCAGCAAGGCGTGCTGT
CTGCCACAATCCTGTACGAGATCCTGCTGGGCAAAGCCACTCTGTACGCCGTGCT
GGTGTCTACCCTGGTCGTGATGGCCATGGTCATGGCTGCTGGTGGACCTGGTGCT
GGATCTGCTGCCCCTGTGTCTAGCACATCTAGCCTGCCTCTGGCCGCTCTGAACA
TGAGAGTCAGAAGAAGGCTGAGCCTGTTCCTGAACGTGCGGACTCAGGTGGCCG
CTGATTGGACAGCTCTGGCCGAGGAAATGGACTTCGAGTACCTGGAAATCCGGC
AGCTGGAAACCCAGGCCGATCCTACAGGCAGACTGCTGGATGCTTGGCAAGGCA
GACCTGGCGCTTCTGTGGGGAGACTGCTTGAGCTGCTGACAAAGCTGGGCAGAG
ATGACGTGCTGCTGGAACTGGGCCCTAGCATCGAGGAAGATTGCCAGAAGTACA
TCCTGAAGCAGCAGCAAGAGGAAGCCGAGAAGCCTCTGCAAGTGGCCGCCGTGG
ATAGCAGCGTTCCAAGAACAGCTGAGCTGGCCGGCATCACCACACTGGATGATC
CTCTGGGAAAGAAGGTGGCCAAGAAGCCCACCAACAAGGCCCCTCATCCTAAGC
AAGAGCCCCAAGAGATCAACTTCCCCGACGATCTGCCCGGCAGCAATACTGCTG
CTCCCGTGCAAGAAACCCTGCACGGTTGTCAGCCCGTGACACAAGAGGACGGCA
AAGAAAGCCGGATCAGCGTGCAAGAGAGACAGAGGGCCAAGAGAAGTGGAAGC
GGCGCCACAAACTTCAGCCTGTTGAAACAGGCCGGGGACGTCGAAGAGAATCCT
GGACCAATGGCACTGCCCGTCACTGCCTTGTTGCTGCCACTTGCACTGCTGCTCC
ATGCCGCCAGGCCAGAAGTGCAGCTGGTTGAAAGTGGCGGCGGACTGGTTCAAC
CTGGCGGATCTCTGAGACTGAGCTGTGCCGCCAGCGGCTTCAATGTGTATGCCTC
TGGCATGCACTGGGTCCGACAGGCCCCTGGAAAAGGCCTTGAGTGGGTCGCCAA
GATCTACCCCGACAGCGACTACACCTACTATGCCGACAGCGTGAAGGGCAGATT
CACCATCAGCGCCGACACCAGCAAGAACACCGCCTACCTGCAGATGAACAGCCT
GAGAGCCGAGGACACCGCCGTGTACTACTGCTCCAGAGACAGCAGCTTCTACTA
CGTGTACGCCATGGACTACTGGGGCCAGGGAACACTGGTCACCGTGTCCAGCGA
AGCCGCCGCTAAAGACATTCAGAATCCTGAGCCTGCCGTGTACCAGCTGAAGGA
CCCTAGAAGCCAGGACAGCACCCTGTGCCTGTTCACCGACTTCGACAGCCAGATC
AACGTGCCCAAGACCATGGAAAGCGGCACCTTCATCACCGATAAGTGCGTGCTG
GACATGAAGGCCATGGACAGCAAGAGCAACGGCGCCATTGCCTGGTCCAACCAG
ACCAGCTTCACGTGCCAGGACATCTTCAAAGAGACAAACGCCACCTATCCTAGC
AGCGACGTGCCCTGTGATGCCACACTGACCGAGAAGTCCTTCGAGACAGACATG
AACCTGAACTTCCAGAACCTGCTGGTCATCGTGCTGAGAATCCTGCTGCTGAAGG
TGGCCGGCTTCAACCTGCTGATGACCCTGAGACTGTGGTCCAGCCGGGCCAAAC
GATCTGGCTCTGGCCAGTGTACCAACTATGCCCTGCTCAAGCTCGCTGGCGACGT
CGAGTCTAACCCTGGACCAATGGGAGCTGGCGCTACAGGCAGAGCTATGGATGG
ACCTAGACTGCTGCTGCTCCTGCTTCTGGGAGTGTCTCTTGGCGGAGCCAAAGAG
GCCTGTCCTACCGGCCTGTATACACACTCTGGCGAGTGCTGCAAGGCCTGCAATC
TTGGAGAAGGCGTCGCACAGCCTTGCGGCGCTAATCAGACAGTGTGCGAGCCTT
GCCTGGACAGCGTGACCTTTAGCGACGTGGTGTCAGCCACCGAGCCATGCAAGC
CATGTACCGAGTGTGTGGGCCTGCAGAGCATGTCTGCCCCTTGCGTGGAAGCTGA
CGATGCCGTGTGTAGATGCGCCTACGGCTACTACCAGGACGAGACAACAGGCAG
ATGCGAGGCCTGTAGAGTGTGCGAAGCTGGATCTGGCCTGGTGTTCAGCTGCCA
AGACAAGCAGAACACCGTGTGCGAGGAATGCCCCGATGGCACCTATAGCGACGA
GGCCAACCATGTGGATCCCTGCCTGCCTTGTACCGTGTGTGAAGATACCGAGCGG
CAGCTGCGCGAGTGTACAAGATGGGCTGATGCCGAGTGCGAAGAGATCCCTGGC
AGATGGATCACCAGAAGCACACCTCCAGAGGGCAGCGATAGCACAGCCCCTTCT
ACACAAGAGCCCGAGGCTCCTCCTGAGCAGGACCTGATTGCTTCTACAGTGGCTG
GCGTCGTGACCACCGTGATGGGATCTAGTCAGCCCGTGGTCACCAGAGGCACCA
CCGACAATCTGATCCCCGTGTACTGTAGCATCCTGGCCGCCGTGGTTGTTGGACT
GGTGGCCTATATCGCCTTCAAGCGGTGGAACAGCTGATGATTCGAAGCGGCCGC
TTCGAGCAGACATGATAAGATACATTGATGAGTTTGGACAAACCACAACTAGAA
TGCAGTGAAAAAAATGCTTTATTTGTGAAATTTGTGATGCTATTGCTTTATTTGTA
ACCATTATAAGCTGCAATAAACAAGTTACACGGCAGGGTCAGGGTTCTGGATATc
tgtgggacaagaggatcagggttaggacatgatctcatttccctctttgccccaacccaggctggagtccagatgccagtgatggacaa
gggcggggctctgtggggctggcaagtcacggtctcatgctttatacgggaaatagcatcttagaaaccagctgctcgtgatggactg
ggactcagggacaggcacaagctatcaatcttggccaagaggccatgatttcagtgaacgttcacggccaggcctggcctgccactc
aaggaaacCACTGGCCGTCGTTTTACAACGTCGTGACTgggaaaaccctggcgttacccaacttaatcgc
cttgcagcacatccccctttcgccagctggcgtaatagcgaagaggcccgcaccgatcgcccttcccaacagttgcgcagcctgaatg
gcgaatggcgcctgatgcggtattttctccttacgcatctgtgcggtatttcacaccgcatatggtgcactctcagtacaatctgctctgat
gccgcatagttaagccagccccgacacccgccaacacccgctgacgcgccctgacgggcttgtctgctcccggcatccgcttacag
acaagctgtgaccgtctccgggagctgcatgtgtcagaggttttcaccgtcatcaccgaaacgcgcga
-Split/CAR-3 + Cβ-MC (amino acid sequence)
SEQ ID NO: 74
MALPVTALLLPLALLLHAARPDIQMTQSPSSLSASVGDRVTITCRASQDVNTAVAWY
QQKPGKAPKLLIYSAYFLYSGVPSRFSGSRSGTDFTLTISSLQPEDFATYYCQQYSRYS
PVTFGQGTKVEIKEAAAKEDLRNVTPPKVSLFEPSKAEIANKQKATLVCLARGFFPD
HVELSWWVNGKEVHSGVCTDPQAYKESNYSYCLSSRLRVSATFWHNPRNHFRCQV
QFHGLSEEDKWPEGSPKPVTQNISAEAWGRADCGITSASYQQGVLSATILYEILLGKA
TLYAVLVSTLVVMAMVMAAGGPGAGSAAPVSSTSSLPLAALNMRVRRRLSLFLNV
RTQVAADWTALAEEMDFEYLEIRQLETQADPTGRLLDAWQGRPGASVGRLLELLTK
LGRDDVLLELGPSIEEDCQKYILKQQQEEAEKPLQVAAVDSSVPRTAELAGITTLDDP
LGKKVAKKPTNKAPHPKQEPQEINFPDDLPGSNTAAPVQETLHGCQPVTQEDGKESR
ISVQERQRAKRSGSGATNFSLLKQAGDVEENPGPMALPVTALLLPLALLLHAARPEV
QLVESGGGLVQPGGSLRLSCAASGFNVYASGMHWVRQAPGKGLEWVAKIYPDSDY
TYYADSVKGRFTISADTSKNTAYLQMNSLRAEDTAVYYCSRDSSFYYVYAMDYWG
QGTLVTVSSEAAAKDIQNPEPAVYQLKDPRSQDSTLCLFTDFDSQINVPKTMESGTFI
TDKCVLDMKAMDSKSNGAIAWSNQTSFTCQDIFKETNATYPSSDVPCDATLTEKSFE
TDMNLNFQNLLVIVLRILLLKVAGFNLLMTLRLWSSRAKRSGSGQCTNYALLKLAG
DVESNPGPMGAGATGRAMDGPRLLLLLLLGVSLGGAKEACPTGLYTHSGECCKAC
NLGEGVAQPCGANQTVCEPCLDSVTFSDVVSATEPCKPCTECVGLQSMSAPCVEAD
DAVCRCAYGYYQDETTGRCEACRVCEAGSGLVFSCQDKQNTVCEECPDGTYSDEA
NHVDPCLPCTVCEDTERQLRECTRWADAECEEIPGRWITRSTPPEGSDSTAPSTQEPE
APPEQDLIASTVAGVVTTVMGSSQPVVTRGTTDNLIPVYCSILAAVVVGLVAYIAFK
RWNS
-Split/CAR-3 + Fas-MC (plasmid sequence)
SEQ ID NO: 75
gacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacg
ctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagt
aagagaattatgcagtgctgccataaccatgagtgataacactgcggccaacttacttctgacaacgatcggaggaccgaaggagcta
accgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaa
tagactggatggaggcggataaagttgcaggaccacttctgcgctcggcccttccggctggctggtttattgctgataaatctggagcc
ggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgc
agataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagg
gagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtc
agtgagcgaggaagcggaagagcgcccaatacgcaaaccgcctctccccgcgcgttggccgattcattaatgcagctggcacgaca
ggtttcccgactggaaagcgggcagtgagcgcaacgcaattaatgtgagttagctcactcattaggcaccccaggctttacactttatgc
ttccggctcgtatgttgtgtggaattgtgagcggataacaatttcacaCAGGAAACAGCTATGACCATGATTAC
GCCAtggccattcctgaagcaaggaaacagcctgcgaaggcaccaaagctgcccttacCTGGGCTGGGGAAGAA
GGTGTCTTCTGGAATAATGCTGTTGTTGAAGGCGTTTGCACATGCAAAGTCAGAT
TTGTTGCTCCAGGCCACAGCACTGTTGCTCTTGAAGTCCATAGACCTCATGTCTA
GCACAGTTTTGTCTGTGATATACACATCAGAATCCTTACTTTGTGACACATTTGTT
TGAGAATCAAAATCGGTGAATAGGCAGACAGACTTGTCACTGGATTTAGAGTCT
CTCAGCTGGTGGCTCCGGTGCCCGTCagtgggcagagcgcacatcgcccacagtccccgagaagttgggg
ggaggggtcggcaattgaaccggtgcctagagaaggtggcgcggggtaaactgggaaagtgatgtcgtgtactggctccgccttttt
cccgagggtgggtggagaaccgtatataagtgcagtagtcgccgtgaacgttctttttcgcaacgggtttgccgccagaacacaggtaa
gtgccgtgtgtggttcccgcgggcctggcctctttacgggttatggcccttgcgtgccttgaattacttccacctggctgcagtacgtgatt
cttgatcccgagcttcgggttggaagtgggtgggagagttcgaggccttgcgcttaaggagccccttcgcctcgtgcttgagttgaggc
ctggcctgggcgctggggccgccgcgtgcgaatctggtggcaccttcgcgcctgtctcgctgctttcgataagtctctagccatttaaa
atttttgatgacctgctgcgacgctttttttctggcaagatagtcttgtaaatgcgggccaagatctgcacactggtatttcggtttttggggc
cgcgggcggcgacggggcccgtgcgtcccagcgcacatgttcggcgaggcggggcctgcgagcgcggccaccgagaatcgga
cgggggtagtctcaagctggccggcctgctctggtgcctggcctcgcgccgccgtgtatcgccccgccctgggcggcaaggctggc
ccggtcggcaccagttgcgtgagcggaaagatggccgcttcccggccctgctgcagggagctcaaaatggaggacgcggcgctcg
ggagagcgggcgggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccc
cacactgagtgggtggagactgaagttaggccagcttggcacttgatgtaattctccttggaatttgccctttttgagtttggatcttggttc
attctcaagcctcagacagtggttcaaagtttttttCTTCCATTTCAGGTGTCGTGAtctagaGCCACCATGG
CTCTGCCTGTGACAGCTCTGCTGCTGCCTCTTGCTCTGCTTCTGCATGCCGCCAGA
CCTGACATCCAGATGACACAGAGCCCTAGCAGCCTGTCTGCCAGCGTGGGAGAC
AGAGTGACCATCACCTGTAGAGCCAGCCAGGACGTGAACACAGCCGTGGCTTGG
TATCAGCAGAAGCCTGGCAAGGCCCCTAAGCTGCTGATCTACAGCGCCTACTTCC
TGTACAGCGGCGTGCCCAGCAGATTCAGCGGCTCTAGAAGCGGCACCGACTTCA
CCCTGACCATAAGCAGTCTGCAGCCCGAGGACTTCGCCACCTACTACTGTCAGCA
GTACAGCCGGTACAGCCCCGTGACATTTGGCCAGGGCACCAAGGTGGAAATCAA
GGAAGCCGCCGCTAAAGAGGACCTGAGAAACGTGACCCCTCCTAAGGTGTCCCT
GTTCGAGCCTAGCAAGGCCGAGATCGCCAACAAGCAGAAAGCCACACTCGTGTG
CCTGGCCAGAGGCTTCTTTCCCGATCACGTGGAACTGTCTTGGTGGGTCAACGGC
AAAGAGGTGCACAGCGGCGTCTGTACTGATCCCCAGGCCTACAAAGAGAGCAAC
TACAGCTACTGCCTGAGCAGCAGACTGAGAGTGTCCGCCACCTTCTGGCACAACC
CCAGAAACCACTTCAGATGCCAGGTGCAGTTTCACGGCCTGAGCGAAGAGGACA
AGTGGCCTGAGGGAAGCCCCAAGCCAGTGACACAGAATATCTCTGCCGAAGCCT
GGGGCAGAGCCGATTGTGGAATTACCAGCGCCAGCTACCAGCAAGGCGTGCTGT
CTGCCACAATCCTGTACGAGATCCTGCTGGGCAAAGCCACTCTGTACGCCGTGCT
GGTGTCTACCCTGGTCGTGATGGCCATGGTCAAGCGGAAGAACAGCAGGGCCAA
GAGAAGTGGAAGCGGCGCCACAAACTTCAGCCTGTTGAAACAGGCCGGGGACGT
CGAAGAGAATCCTGGACCAATGGCACTGCCCGTCACTGCCTTGTTGCTGCCACTT
GCACTGCTGCTCCATGCCGCCAGGCCAGAAGTGCAGCTGGTTGAAAGTGGCGGC
GGACTGGTTCAACCTGGCGGATCTCTGAGACTGAGCTGTGCCGCCAGCGGCTTCA
ATGTGTATGCCTCTGGCATGCACTGGGTCCGACAGGCCCCTGGAAAAGGCCTTGA
GTGGGTCGCCAAGATCTACCCCGACAGCGACTACACCTACTATGCCGACAGCGT
GAAGGGCAGATTCACCATCAGCGCCGACACCAGCAAGAACACCGCCTACCTGCA
GATGAACAGCCTGAGAGCCGAGGACACCGCCGTGTACTACTGCTCCAGAGACAG
CAGCTTCTACTACGTGTACGCCATGGACTACTGGGGCCAGGGAACACTGGTCACC
GTGTCCAGCGAAGCCGCCGCTAAAGACATTCAGAATCCTGAGCCTGCCGTGTAC
CAGCTGAAGGACCCTAGAAGCCAGGACAGCACCCTGTGCCTGTTCACCGACTTC
GACAGCCAGATCAACGTGCCCAAGACCATGGAAAGCGGCACCTTCATCACCGAT
AAGTGCGTGCTGGACATGAAGGCCATGGACAGCAAGAGCAACGGCGCCATTGCC
TGGTCCAACCAGACCAGCTTCACGTGCCAGGACATCTTCAAAGAGACAAACGCC
ACCTATCCTAGCAGCGACGTGCCCTGTGATGCCACACTGACCGAGAAGTCCTTCG
AGACAGACATGAACCTGAACTTCCAGAACCTGCTGGTCATCGTGCTGAGAATCCT
GCTGCTGAAGGTGGCCGGCTTCAACCTGCTGATGACCCTGAGACTGTGGTCCAGC
CGGGCCAAACGATCTGGCTCTGGCCAGTGTACCAACTATGCCCTGCTCAAGCTCG
CTGGCGACGTCGAGTCTAACCCTGGACCAATGCTCGGCATCTGGACACTGCTGCC
TCTGGTGCTGACAAGCGTGGCCAGACTGAGCAGCAAGAGCGTGAACGCCCAAGT
GACCGACATCAACAGCAAAGGCCTGGAACTGAGAAAGACCGTGACCACCGTGG
AAACCCAGAACCTGGAAGGCCTGCACCACGACGGCCAGTTCTGTCACAAACCTT
GTCCACCTGGCGAGCGGAAGGCCAGAGATTGCACAGTGAATGGCGACGAGCCTG
ACTGCGTGCCCTGTCAAGAGGGCAAAGAGTACACCGACAAGGCCCACTTCAGCA
GCAAGTGCAGACGGTGCAGACTGTGCGACGAAGGCCACGGACTGGAAGTGGAA
ATCAACTGCACCCGGACACAGAACACCAAGTGCCGGTGCAAGCCCAACTTCTTC
TGCAACAGCACCGTGTGCGAGCACTGCGACCCTTGTACCAAGTGCGAACACGGC
ATCATCAAAGAGTGCACCCTGACCTCCAACACGAAGTGCAAAGAGGAAGGCAGC
AGAAGCAACCTCGGCTGGCTGTGTCTGCTGCTGCTCCCCATTCCTCTGATCGTGT
GGGTTATGGCTGCTGGCGGACCTGGTGCTGGATCTGCTGCACCTGTGTCCAGCAC
ATCTAGCCTGCCACTGGCCGCTCTGAACATGAGAGTCAGAAGAAGGCTGAGCCT
GTTCCTGAACGTGCGCACTCAGGTGGCCGCTGATTGGACAGCTCTGGCCGAGGA
AATGGACTTCGAGTACCTGGAAATCCGGCAGCTGGAAACACAGGCCGATCCTAC
AGGCAGACTGCTGGATGCTTGGCAAGGCAGACCTGGCGCTTCTGTGGGGAGACT
GCTTGAGCTGCTGACAAAGCTGGGCAGAGATGACGTGCTGCTGGAACTGGGACC
CAGCATCGAGGAAGATTGCCAGAAGTACATCCTGAAGCAGCAACAAGAGGAAG
CCGAGAAGCCTCTGCAGGTTGCCGCCGTGGATAGCAGCGTTCCAAGAACAGCTG
AGCTGGCCGGCATCACCACACTGGATGATCCTCTGGGAAAGAAGGTGGCCAAGA
AGCCCACAAACAAGGCCCCTCATCCTAAGCAAGAGCCCCAAGAGATCAACTTCC
CCGACGATCTGCCCGGCAGCAATACTGCAGCTCCCGTGCAAGAAACCCTGCACG
GTTGTCAGCCCGTGACACAAGAGGACGGCAAAGAAAGCCGGATCAGCGTGCAA
GAGAGACAGTGATGATTCGAAGCGGCCGCTTCGAGCAGACATGATAAGATACAT
TGATGAGTTTGGACAAACCACAACTAGAATGCAGTGAAAAAAATGCTTTATTTGT
GAAATTTGTGATGCTATTGCTTTATTTGTAACCATTATAAGCTGCAATAAACAAG
TTACACGGCAGGGTCAGGGTTCTGGATATctgtgggacaagaggatcagggttaggacatgatctcatttc
cctctttgccccaacccaggctggagtccagatgccagtgatggacaagggcggggctctgtggggctggcaagtcacggtctcatg
ctttatacgggaaatagcatcttagaaaccagctgctcgtgatggactgggactcagggacaggcacaagctatcaatcttggccaag
aggccatgatttcagtgaacgttcacggccaggcctggcctgccactcaaggaaacCACTGGCCGTCGTTTTACAA
CGTCGTGACTgggaaaaccctggcgttacccaacttaatcgccttgcagcacatccccctttcgccagctggcgtaatagcga
agaggcccgcaccgatcgcccttcccaacagttgcgcagcctgaatggcgaatggcgcctgatgcggtattttctccttacgcatctgt
gcggtatttcacaccgcatatggtgcactctcagtacaatctgctctgatgccgcatagttaagccagccccgacacccgccaacaccc
gctgacgcgccctgacgggcttgtctgctcccggcatccgcttacagacaagctgtgaccgtctccgggagctgcatgtgtcagaggt
tttcaccgtcatcaccgaaacgcgcga
-Split/CAR-3 + Fas-MC (amino acid sequence)
SEQ ID NO: 76
MALPVTALLLPLALLLHAARPDIQMTQSPSSLSASVGDRVTITCRASQDVNTAVAWY
QQKPGKAPKLLIYSAYFLYSGVPSRFSGSRSGTDFTLTISSLQPEDFATYYCQQYSRYS
PVTFGQGTKVEIKEAAAKEDLRNVTPPKVSLFEPSKAEIANKQKATLVCLARGFFPD
HVELSWWVNGKEVHSGVCTDPQAYKESNYSYCLSSRLRVSATFWHNPRNHFRCQV
QFHGLSEEDKWPEGSPKPVTQNISAEAWGRADCGITSASYQQGVLSATILYEILLGKA
TLYAVLVSTLVVMAMVKRKNSRAKRSGSGATNFSLLKQAGDVEENPGPMALPVTA
LLLPLALLLHAARPEVQLVESGGGLVQPGGSLRLSCAASGFNVYASGMHWVRQAPG
KGLEWVAKIYPDSDYTYYADSVKGRFTISADTSKNTAYLQMNSLRAEDTAVYYCSR
DSSFYYVYAMDYWGQGTLVTVSSEAAAKDIQNPEPAVYQLKDPRSQDSTLCLFTDF
DSQINVPKTMESGTFITDKCVLDMKAMDSKSNGAIAWSNQTSFTCQDIFKETNATYP
SSDVPCDATLTEKSFETDMNLNFQNLLVIVLRILLLKVAGFNLLMTLRLWSSRAKRS
GSGQCTNYALLKLAGDVESNPGPMLGIWTLLPLVLTSVARLSSKSVNAQVTDINSKG
LELRKTVTTVETQNLEGLHHDGQFCHKPCPPGERKARDCTVNGDEPDCVPCQEGKE
YTDKAHFSSKCRRCRLCDEGHGLEVEINCTRTQNTKCRCKPNFFCNSTVCEHCDPCT
KCEHGIIKECTLTSNTKCKEEGSRSNLGWLCLLLLPIPLIVWVMAAGGPGAGSAAPVS
STSSLPLAALNMRVRRRLSLFLNVRTQVAADWTALAEEMDFEYLEIRQLETQADPTG
RLLDAWQGRPGASVGRLLELLTKLGRDDVLLELGPSIEEDCQKYILKQQQEEAEKPL
QVAAVDSSVPRTAELAGITTLDDPLGKKVAKKPTNKAPHPKQEPQEINFPDDLPGSN
TAAPVQETLHGCQPVTQEDGKESRISVQERQ

### CRISPR Editing of Primary Human T Cells

Peripheral blood mononuclear cells (PBMCs) were isolated by Ficoll-Paque PLUS (Cytiva, 17144002) gradient centrifugation from healthy donor leukopaks (StemCell and Charles River). CD3+ T cells were isolated by negative selection (StemCell Technologies, 17951) and activated with Dynabeads Human T-Activator CD3/CD28 (Thermo Fisher Scientific, 11132D) at a 1:1 bead-to-cell ratio in T cell media, which consisted of RPMI-1640 (ATCC, 30-2001) supplemented with 10% FBS (Cytiva, SH30070.03), 1% penicillin-streptomycin (Thermo Fisher Scientific, 15140163), 100 IU/mL recombinant human IL-2 (Proleukin, Prometheus Laboratories), and 5 ng/ml recombinant human IL-7 (BioLegend, 581908). After 48-56 hours of activation, T cells were separated from CD3/CD28 beads by two passes over a magnet and allowed to rest while CRISPR reagents were prepared. For each electroporation condition of simultaneous TRAC knock-in and TRBC knock-out, 50 pmols of each crRNA were independently mixed with 25 pmols of Alt-R A.s. Cas12a (Cpf1) Ultra (IDT, 10001273) and 37.5 pmols of Alt-R Cpf1 Electroporation Enhancer (IDT, 1076301) in a total volume of 1.27 μl of Nuclease Free Duplex Buffer (IDT, Nov. 1, 2003-01) for at least 15 minutes before combining the two RNPs at a 1:1 volume ratio. The TRAC and TRBC RNPs were then mixed with 0.5 μg HDRT diluted in 2.4 μL OptiMEM for a final volume of 5 μL per electroporation condition. Immediately prior to electroporation, T cells were centrifuged at 90 g for 10 minutes, then resuspended at 0.75-1.25×10⁶ T cells in 20 μl P3 buffer (Lonza, V4XP-3032) and combined with the 5 μL Cpf1 RNP and HDRT mixture. Cells were nucleofected in 16-well cuvettes (Lonza, V4XP-3032) with a 4D Nucleofector X-Unit (Lonza, AAF-1003X) using pulse code EH115. After nucleofection, 80 μl pre-warmed cytokine free T cell media was added to cells and the cuvette strip was placed in a 37° C. incubator for 20 min. The cells were then diluted in 1 mL of T cell media and transferred to a 24-well plate. T cell media was changed every 3 days until functional assays on days 11 or greater after initial activation.

When noted in figure legends, the CRISPR editing protocol was modified to target other genetic loci (e.g. CD3G instead of TRAC and TRBC) or to utilize Cas9 nuclease (IDT, 1081059) and Alt-R Cas9 Electroporation Enhancer (IDT, 1075916) instead of Cpf1 reagents. All gRNA sequences used are listed in Table 1.

### Flow Cytometry

Flow cytometry was performed with an IntelliCyt iQue Screener PLUS (Sartorius). Cells were labeled with a viability dye, either LIVE/DEAD Fixable Near-IR Dead Cell Stain (Thermo Fisher Scientific, L34975) or LIVE/DEAD Fixable Violet Dead Cell Stain (Thermo Fisher Scientific, L34955). To assess HLA-A*02:01 expression, NALM6 isogenics were stained with Brilliant Violet (BV)-785 anti-human HLA-A*02 (BB7.2, BioLegend, 343328) or a mouse isotype IgG2b, κ (HI100, BioLegend, 304140). T cells were stained with combinations of the following anti-human antibodies: APC-CD3 (SK7, BioLegend, 344812), BV421-CD3 (SK7, BioLegend, 344834), BV785-CD4 (RPA-T4, BioLegend, 300554), PE-Vio770-CD4 (M-T466, Miltenyi, 130-113-255), APC-CD2 (RPA-2.10, BioLegend, 300214), BV421-CD2 (RPA-2.10, BioLegend, 300230), APC-NGFR (ME20.4, BioLegend, 345108), BV785-PD-1 (EH12.1, BD, 563789), BV605-LAG-3 (11C3C65, BioLegend, 369316), or PE-dazzle/594-TIM-3 (F38-2E2, BioLegend, 345034). CD8+ T cell populations were defined as T cells that were not stained with CD4-targeted antibodies when included. The expression of TCR, CAR, or CAR-derivatives was assessed with PE, APC, or BV421 labeled p53RH tetramer (FHCRC Immune Monitoring Core facility), anti-mouse TRBC (H57-597, BioLegend, 109230), or anti-human Vβ21.3 (REA894, Miltenyi, 130-114-839). When staining T cells in peripheral blood of mice, a BV605 labeled anti-murine CD45 antibody (30-F11, BioLegend, 103140) and TruStain FcX (anti-mouse CD16/32) antibody (93, BioLegend, 101320) were also used. Flow cytometry data was analyzed with either the iQue Forecyt Software (Sartorius) or FlowJo v. 10.1 software (BD).

### Peptide Pulsing

T2 cells were washed with serum-free RPMI-1640 media, then incubated at 1-2×10⁶ cells/mL with 10 μg/mL beta-2 microglobulin (ProSpec, PRO-337) and specified concentrations of the p53RH peptide (HMTEVVRHC, Peptide 2.0). Pulsed cells were incubated on low attachment plates (Corning, 3471) in a 37° C. incubator for at least 4 hrs. Prior to use in functional assays, pulsed cells were washed 2 times with serum containing media.

### In Vitro Short-Term Co-Culture Assays

T cell knock-in (KI) frequency was normalized to the lowest frequency by TCR-Control T cells. Then a specified number of KI+ T cells were mixed with a specified number of cancer cells (generally 1×10⁴ KI+ T cells with 5×10⁴ cancer cells unless otherwise specified in the figure legend) in 200 μl of RPMI-1640 media supplemented with 10% FBS, 1% PS, and 100 IU/mL IL-2. Co-cultures were incubated from 18-24 hrs in a 37° C. incubator, as specified in figure legends. At the termination of the assay, 100 μl conditioned supernatant was used for the Human IFN-γ Quantikine Kit (R&D Systems, PDIF50C) or the MILLIPLEX Luminex assay (Millipore-Sigma, HCD8MAG-15K) per the manufacturer's instructions. A bioluminescence cytotoxicity assay (Promega, E2510) was performed per the manufacturer's instructions on the remaining 100 μl containing the cells. Cytotoxicity was calculated by normalizing to the TCR-Control luminescence signal: [1−(experimental well)/(TCR-Control well)]×100.

### Multiple Stimulation Assays

T cell knock-in (KI) frequency was normalized to the lowest frequency by TCR-Control T cells. Then 1×10⁴ KI+ T cells were mixed with 5×10⁴ NALM6-MUT cells in 200 μl of cytokine-free RPMI-1640 media supplemented with 10% FBS and 1% PS. Live cell imaging with an Incucyte SX5 (Sartorius) was used to quantify the number of GFP+ NALM6-MUT cells every 3-4 hrs. After approximately 48 hrs, 100 μl of conditioned supernatant was removed and 5×10⁴ NALM6-MUT cells in 100 μl of fresh media were added to the co-culture. This process was repeated for 24 days. At the end of the assay, the number of living KI+ T cells and NALM6-MUT cells were quantified by flow cytometry.

### Mouse Xenograft Models

Female NOD.Cg-Prkdc^{scid}I12rg^{tm1Wjl}/SzJ (NSG) mice at 6-24 weeks of age were obtained from Jackson Laboratory (005557) or the Johns Hopkins Sidney Kimmel Comprehensive Cancer Center Animal Resources facility and were maintained and treated in compliance with an JHU Animal Care and Use Committee-approved research protocol. Cancer cells and human T cells were injected individually via tail vein in 200 μL RPMI-1640 media. The timing of injections is noted in the figure legends. Cancer cell burden was measured by quantifying bioluminescence signal. Mice were intraperitoneally injected with 150 μl RediJect d-Luciferin Ultra Bioluminescent Substrate (PerkinElmer, 770505), anesthetized with inhaled isoflurane, and then were imaged with an IVIS system (PerkinElmer) 6 minutes after initial substrate injection. For survival studies, mice were euthanized when hind limb paralysis was noted. For the quantification of T cells in the peripheral blood of mice, blood from the submandibular veins of mice was collected in EDTA coated microvettes (Sarstedt, NC9299309). Then, 100 μl of blood was treated with ACK lysis buffer (Quality Biological, 118-156-721) and then washed with PBS. Flow cytometry staining was conducted as described herein.

### Statistical Analysis

Mean±standard deviation or standard error was used to summarize the data, as specified in figure legends. Statistical analyses were carried out as indicated in figure legends. Unless otherwise specified, a P value of <0.05 was used to denote statistical significance. Statistical analyses were performed using Prism version 9.4.1 (GraphPad).

### Bulk RNAseg and Analysis

Split/CAR-3 and TESLA-1 T cells were generated using CRISPR-HDR. On days 8-9 after activation, knock-in+ T cells were isolated using the EasySep™ Release Human PSC-Derived Neural Crest Cell Positive Selection Kit (StemCell, 100-0047). On days 13-14, T cells were cocultured with KMS26-MUT, KMS26-NULL, or no target cells at an E:T ratio of 1:5 for 18 hours followed by flow sorting of CD4+ and CD8+ T cells into RNAprotect Tissue Reagent (Qiagen, 76104). Total RNA was isolated with the RNeasy Mini Kit (Qiagen, 74104) according to the manufacturer's instructions and quantified by RNA ScreenTape (Agilent, 5067-5576). The SMART-Seq mRNA LP (with UMIs) kit (Takara, 634765) and the Unique Dual Index Kit (1-96) (Takara, 634752) were used to generate cDNA and prepare sequencing libraries from two independent batches of T cells, with 3 technical replicates per condition. Libraries were sequenced on a NovaSeq 6000 (Illumina) using the 100 cycle single-end configuration at approximately 25 million reads per technical replicate.

FASTQ files were demultiplexed and only reads containing the 5′ unique molecular identifiers (UMIs) were retained after trimming adapters and extracting the UMI. Reads were aligned to hg38 with RNA STAR followed by UMI deduplication using UMI-tools. Gene-counts of unique reads were generated with HTseq. Differentially expressed genes were identified with DEseq2 and defined as genes with adjusted p-value less than 0.05, base-mean expression greater than 4, and absolute value of fold change greater than 1.2. Heatmaps were Z-score scaled by row on variance stabilizing transformations of the normalized count matrix produced by DEseq2. Pathway analysis was performed using the STRING tool on differentially expressed genes and Reactome gene sets were reported.

### Example 1—Tier 1: Comparing Conventional CARs and TCRs at Low Antigen Densities

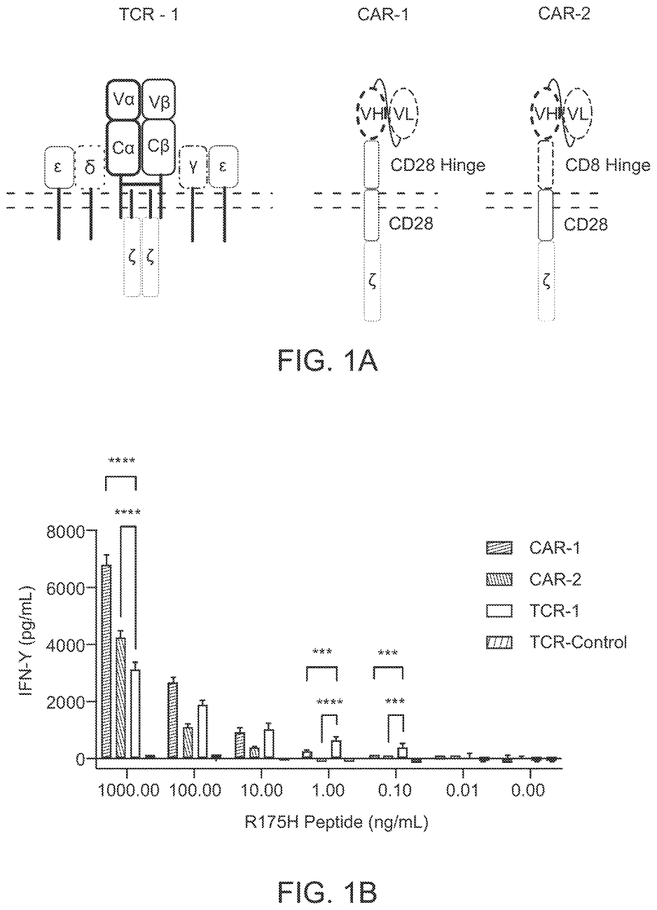

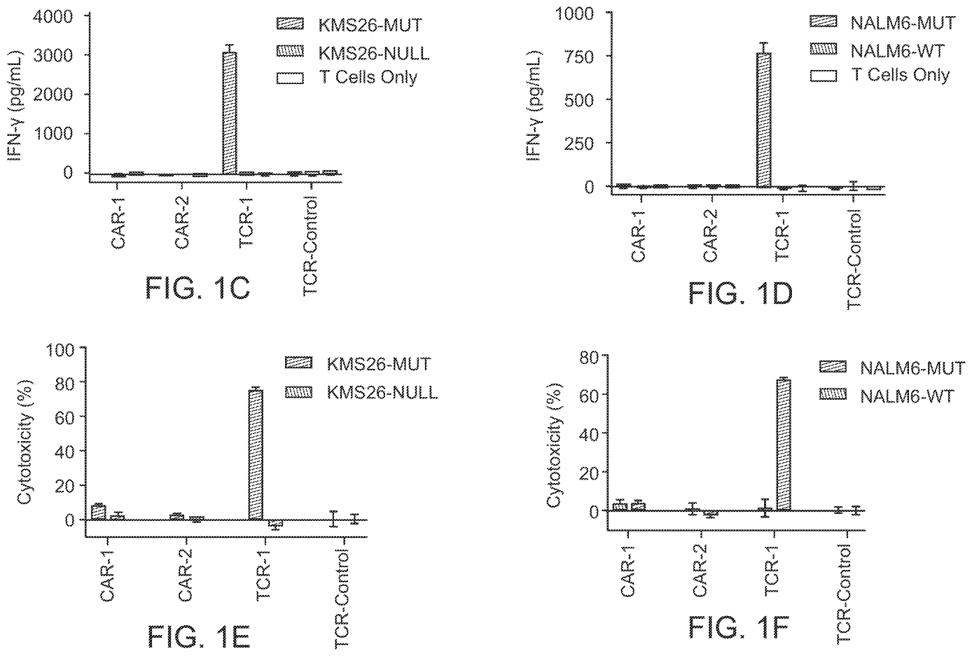

H2 is an antibody-based, single-chain variable fragment (scFv) directed against the p53RH antigen with an affinity of 86 nM when measured in a bispecific antibody format. The H2-scFv was grafted onto a conventional second-generation CAR containing a CD28 hinge and co-stimulatory domain linked to a CD32 intracellular domain to create CAR-1 (FIG. 1A). H2 was also grafted onto another second-generation CAR format that used a CD8α hinge domain instead of a CD28 hinge to create CAR-2 (FIG. 1A). A TCR construct targeting the p53RH antigen was also constructed (TCR-1 in FIG. 1A). TCR-1 was based on the patient-derived AV6/BV11 TCR demonstrated to have pre-clinical as well as clinical utility, with an affinity of 3.5 μM.

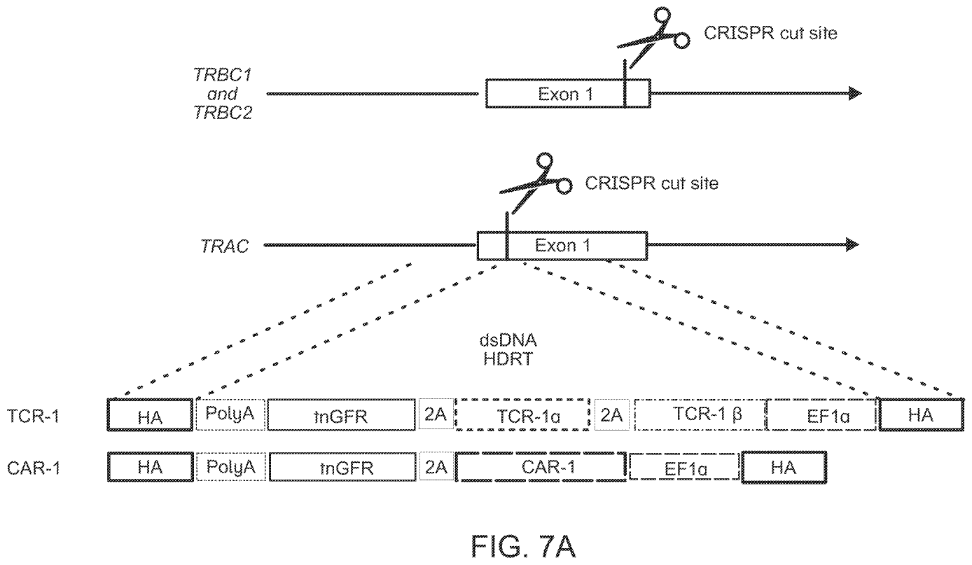

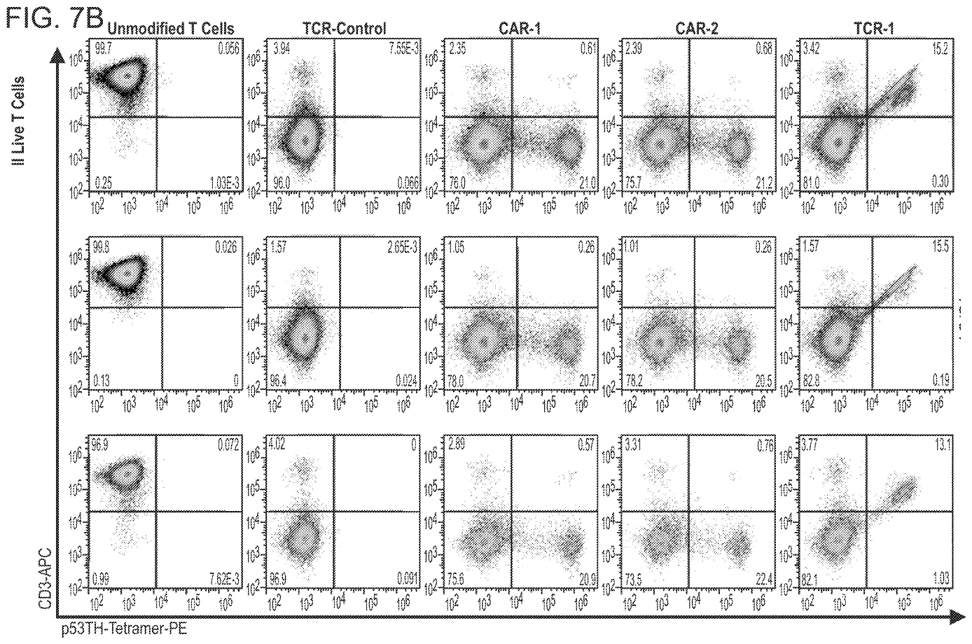

To compare different constructs in a way that minimized confounding factors that could impact the results, CRISPR-based homology-directed repair (HDR) strategies were used to introduce all constructs into the T cell receptor a constant (TRAC) locus of primary human T cells (FIG. 7A). In all cases, the expression was driven by an EF1α promoter based on the previous discovery that this promoter increases the level of expression of a transgenic TCR over that of the endogenous TRAC promoter. Concomitantly, the TRBC loci were inactivated to avoid competition from endogenous TCR chains in the engineered cells (FIG. 7A). Thus, the endogenous TCRs were not expressed in the engineered cells.

As expected from the knock-in strategy employed, primary human T cells engineered with CAR-1, CAR-2, or TCR-1 expressed similar levels of the receptors for the p53RH antigen in both the CD4 and CD8 T cell subsets when measured by flow cytometry (FIG. 7B). Control T cells (TCR-Control), in which the TRAC and TRBC loci were inactivated but no CAR or TCR components were introduced, were included in all experiments (FIG. 7B). To assess their signaling capacity, the cells were co-cultured with T2 cells that express low amounts of peptide-bound HLA-A*02:01 on the cell surface due to a deficiency of the transporter associated with antigen processing (TAP) gene, and therefore preferentially present exogenously provided peptides. T2 cells were first incubated with various concentrations of a synthetic nine amino acid peptide containing the TP53 R175H mutation to generate different p53RH antigen densities, and were then co-cultured with the modified T cells described above (FIG. 1A). At very high concentrations of antigen, CAR-1 and CAR-2 T cells were activated as much or more than TCR-1 T cells, as assessed by secretion of the cytokine interferon-γ (IFN-γ) (FIG. 1B). However, at lower levels of antigen presentation, TCR-1 cells were activated more than CAR-1 or CAR-2 cells (FIG. 1B). These data confirm previous observations that the TCRs can signal much more effectively than CARs against low-density antigens.

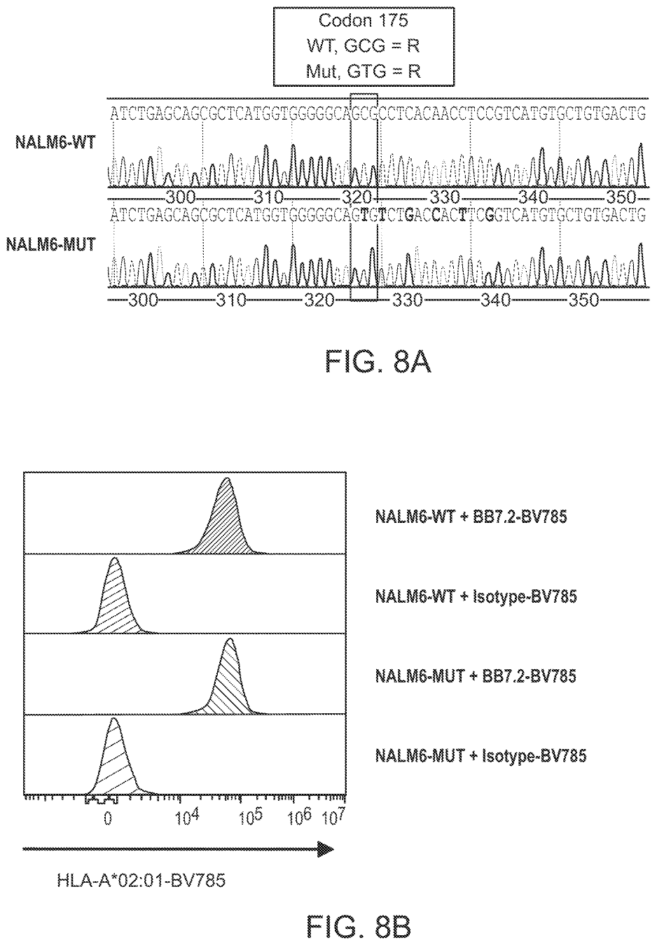

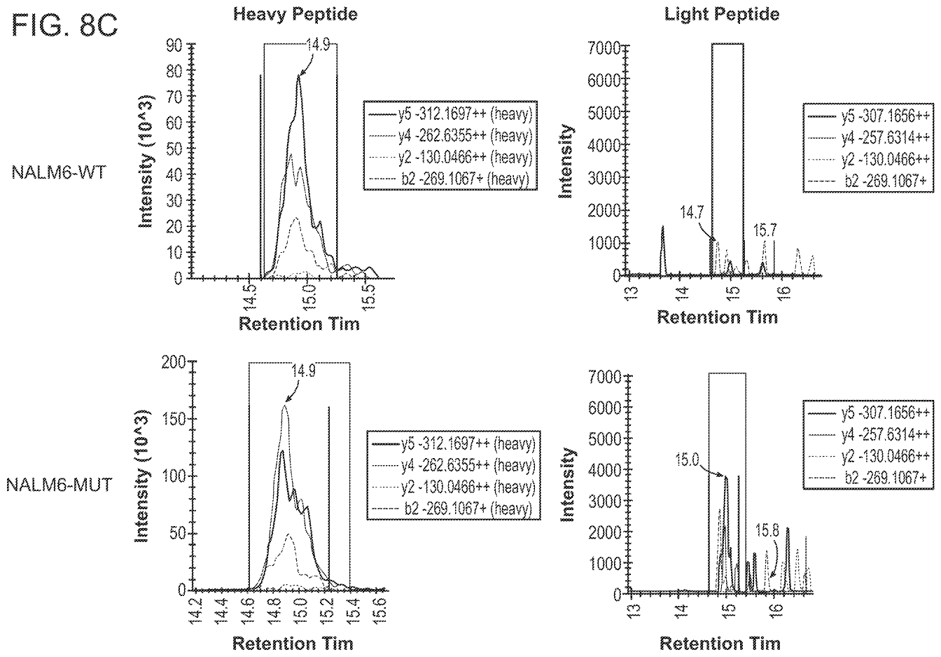

The potency of these cells were next determined when co-cultured with target cells that endogenously process and present the p53RH antigen. KMS26-MUT cells endogenously express HLA-A*02:01 and a naturally-occurring mutant TP53 R175H allele. CRISPR was used to create a paired line, KMS26-NULL, in which both alleles of the endogenous TP53 gene were disrupted. NALM6-WT cells express HLA-A*02:01 and wildtype (WT) TP53 alleles. CRISPR was used to create NALM6-MUT cells in which the WT TP53 allele was replaced with a mutant TP53 R175H allele (FIGS. 8A-8C). The average numbers of the p53RH antigen on the cell surface were 2.4 and 1.3 molecules per cell on KMS26-MUT and NALM6-MUT cells, respectively, as measured by mass spectrometry (Table 3). After co-culture with KMS26-MUT cells, TCR-1 T cells, but not CAR-1 or CAR-2 T cells, secreted IFN-γ (FIG. 1C). The same pattern of activation was observed after co-culture of TCR-1, CAR-1, and CAR-2 T cells with NALM6-MUT cells (FIG. 1D). The KMS26-NULL and NALM6-WT cancer cells served as specificity controls in these experiments (FIGS. 1C-1D).

TABLE 3

| | HLA and | No. cells (millions) | Abundance | Copy | |
|---|---|---|---|---|---|
| Cell Line | p53 status | used for analysis | (femtomole) | no/cell* | Reference |
| NALM6-MUT | Endogenous HLA-A*02:01/p53^{R175H} | 800 | 0.0124 | 1.26 | This manuscript |
| NALM6-WT | Endogenous HLA-A*02:01/p53^{WT} | 800 | Not detectable | Not detectable | This manuscript |
| KMS26-MUT | Endogenous HLA-A*02:01/p53^{R175H} | 157 | 0.48 | 2.4 | (35) |

To determine whether the cytokine activation was accompanied by killing of target cells, the T cells were co-cultured for 20 hours with the same p53RH-expressing cancer cells. Substantial cytotoxicity was observed with TCR-1 cells, but not with CAR-1, CAR-2, or TCR-Control cells (FIGS. 1E-1F). Specificity for the p53RH antigen was documented using isogenic KMS26-NULL and NALM6-WT cells (FIGS. 1E-1F). These results confirm the superior capacity of TCRs over conventional CARs to activate T cells in response low-density antigens as measured by IFN-γ secretion and cytotoxicity.

### Example 2—Tier 2: Hybrid TCR/CAR T Cells have Improved Antigen Sensitivity

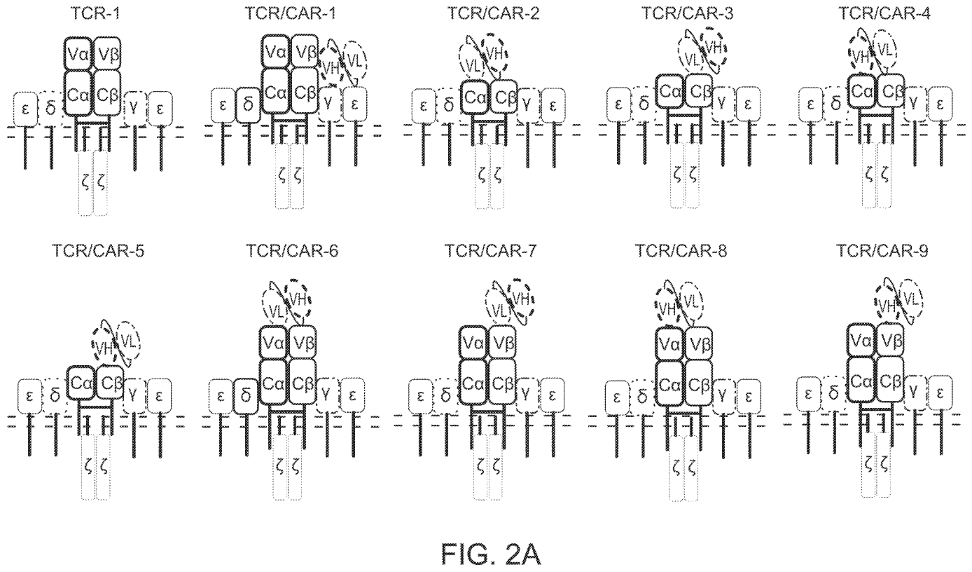

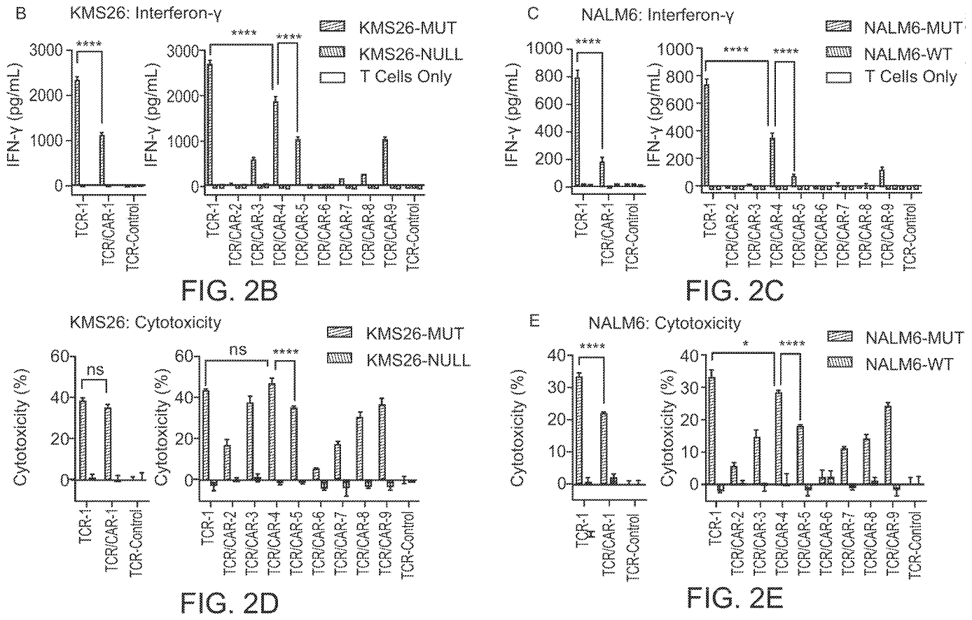

TCRs react to target cells with low antigen densities better than CARs, wherein neither antigen binding capacity as measured by flow cytometry (FIG. 7B) nor reactivity at higher antigen densities can explain this superiority (FIG. 1B). Aside from potential effects of the differences in affinity for the p53RH antigen, a likely explanation lies in the downstream signaling that is different in TCR-1 compared to CAR-1 T cells (FIG. 1A). In particular, in TCR-1 cells, the signaling complex includes multiple subunits encoded by distinct genes, while in CAR-1 T cells, the signaling comes only from the intracellular domain of the CAR protein. To evaluate this possibility, the scFv of the H2-CARs was appended to the N-terminus of the CD3γ subunit of the TCR complex and used CRISPR HDR to introduce this construct into primary human T cells, creating TCR/CAR-1 (FIG. 2A). Similarly, the scFv was appended to the N-terminus of the TCRα or TCRβ subunits in various ways, creating eight different T cell types named TCR/CAR-2 to TCR/CAR-9 (FIG. 2A).

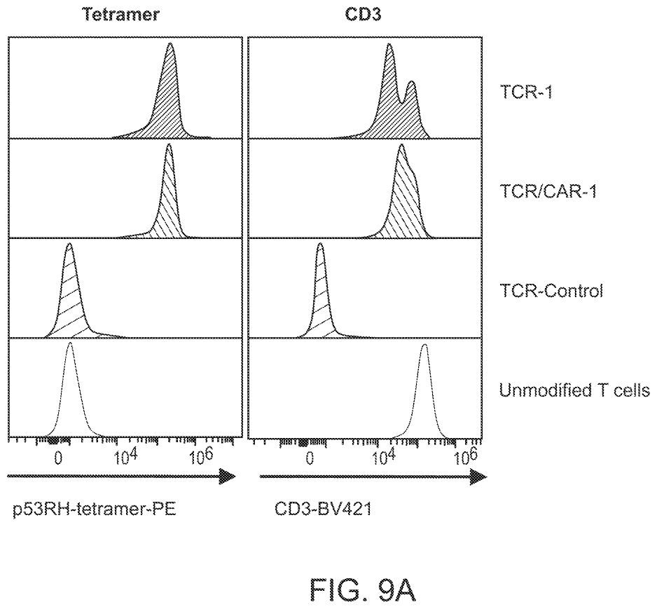

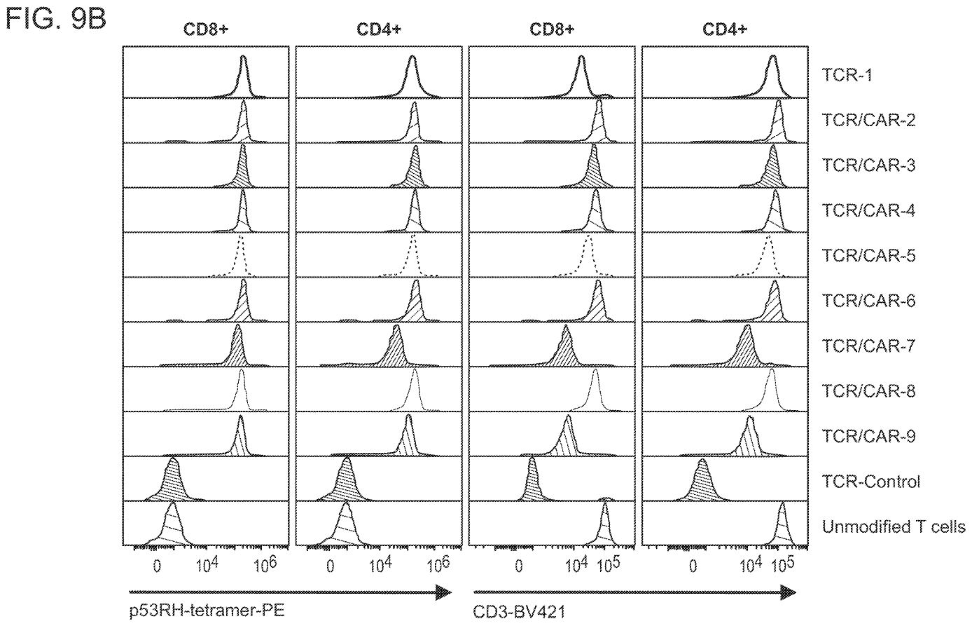

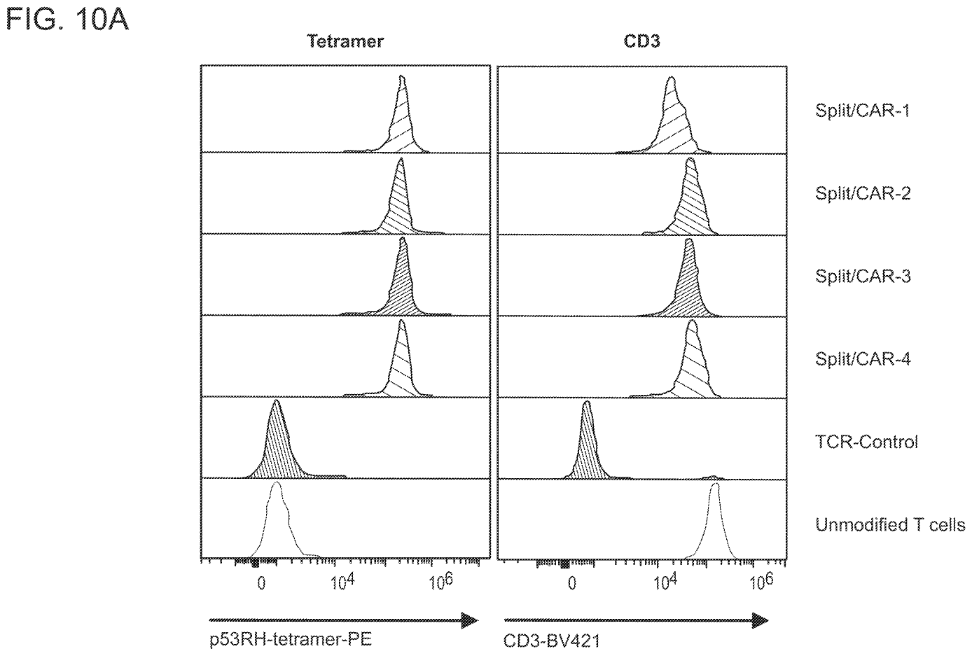

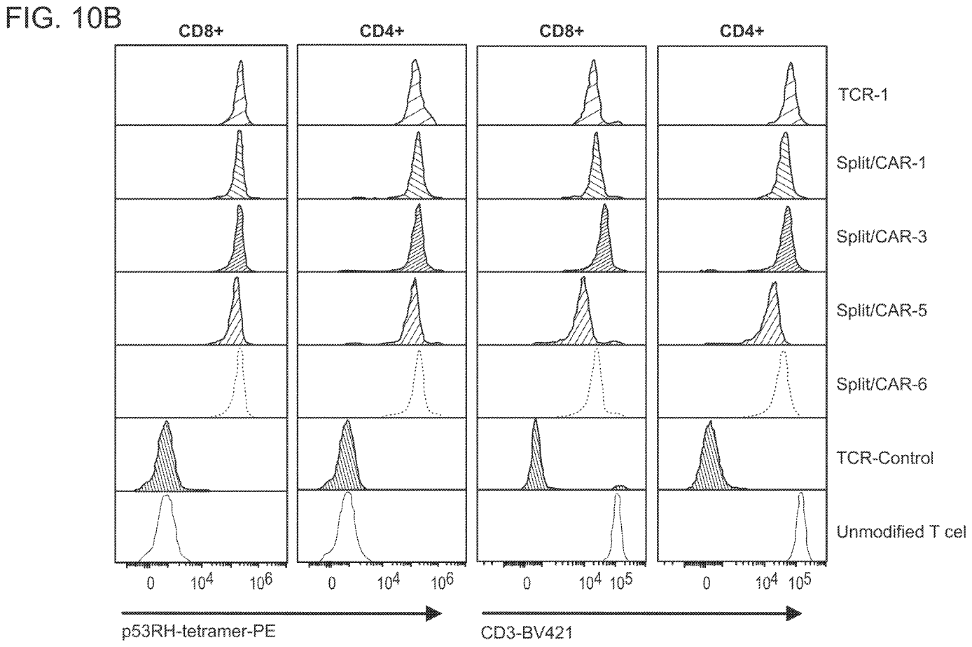

Cell surface expression of most of the hybrid TCR/CAR constructs was similar to the expression levels of TCR-1 in primary human T cells as assessed by binding to the p53RH antigen (FIGS. 9A-9B). Some but not all types of Tier 2 T cells could be activated by cancer cells expressing low levels of the p53RH antigen (FIGS. 2B-2C). Subtle differences in TCR/CAR structure had a major impact on reactivity and function. For example, orientation of the variable heavy (VH) and variable light chains (VL) of the H2-scFv with respect to the N-terminus of the receptor (VHVL or VLVH) was critical (e.g., compare TCR/CAR-2 with TCR/CAR-4 in FIGS. 2B-2C). Many of the TCR/CAR T cells were also able to kill target cells bearing low levels of p53RH antigen (FIGS. 2D-2E), unlike CAR T cells (FIGS. 1E-1F). Notably, cytotoxicity was observed with some TCR/CAR types even when they did not secrete IFN-γ (e.g., TCR/CAR-2). The specificity of all Tier 2 T cells was documented by performing identical assays with KMS26 or NALM6 cells devoid of the p53RH antigen (FIGS. 2B-2E).

### Example 3—Tier 3: Split/CAR T Cells Match Conventional TCR T Cell Antigen Sensitivity

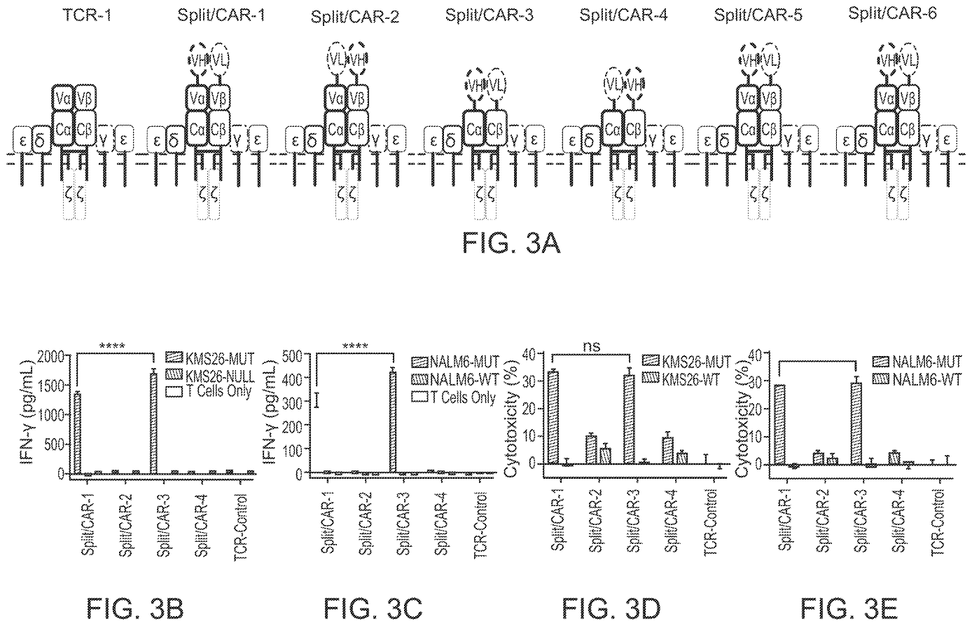

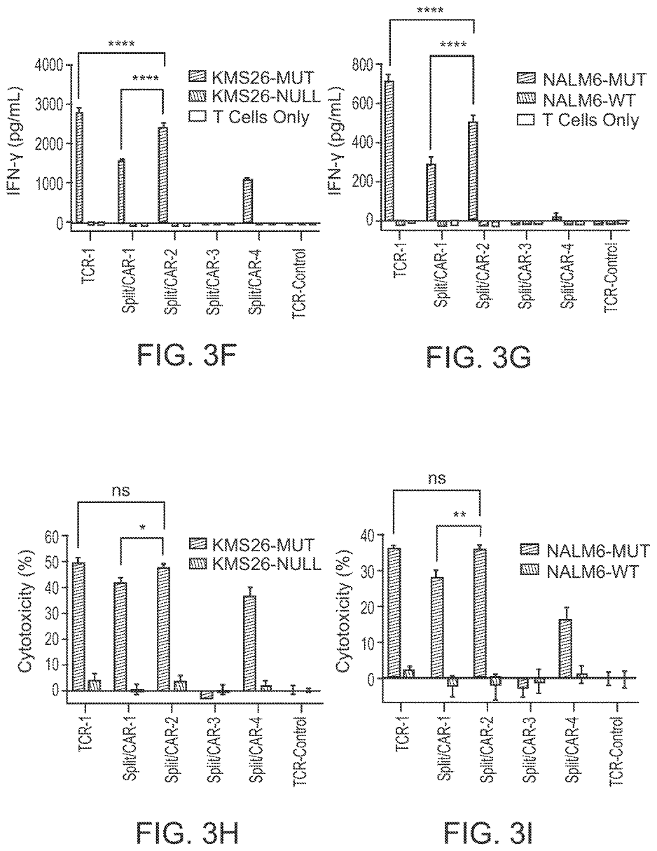

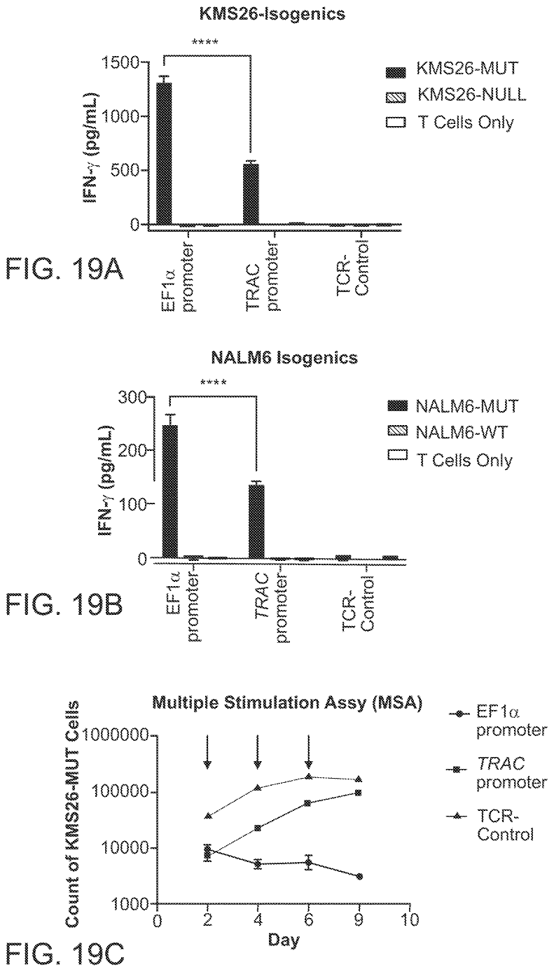

Although the Tier 2 T cells could react with target cells bearing low antigen densities, their reactivity was inferior to a conventional TCR (FIGS. 2B-2E). In an effort to further emulate naturally occurring TCRs, the VH and VL domains of the H2-scFv were split across two independent protein chains by replacing the normal recognition domains of the TCRα and β chains with the VH or VL domains to create Tier 3 T cells (Split/CAR-1 and -2, FIG. 3A). The VH and VL domains from the Cα and Cβ domains were also separated by insertion of a rigid five amino acid “EAAAK” linker (Split/CAR-3 and -4, FIG. 3A), which might allow for improved approximation of the H2 variable domains while maintaining structural rigidity. Finally, the individual VH and VL chains were appended to the N-termini of the Vα and Vβ domains of the intact TCR through a flexible five amino acid G4S linker (Split/CAR-5 and -6, FIG. 3A). A more flexible G4S linker was chosen to accommodate the wider distance between Vα and Vβ N-termini compared to the Cα and Cβ N-termini of the TCR. In contrast with the lentiviral-overexpression used in producing a synthetic TCR and antigen receptor (e.g., mutSTAR approach), CRISPR-based engineering of primary T cells eliminated competition from the endogenous TCR chains, generating truly monospecific T cells. While in some embodiments, T cells expressing an HLA-independent TCR (e.g., HIT approach) utilize CRISPR to insert the HLA-independent receptor under control of the endogenous TRAC promoter, it has been found that EF1α-driven rather than endogenous TRAC-driven expression of transgenic constructs integrated within the TRAC locus produces greater functionality of T cells against low density pHLAs (FIGS. 19A-19C).

All of these modifications were made by CRISPR-based engineering of primary T cells at the same locus. All Split/CARs and TCR-1 had similar capacity to bind to the p53RH antigen, as measured by flow cytometry (FIGS. 10A-10B). Tier 3 T cells were co-cultured with KMS26-MUT or NALM6-MUT target cells and evaluated for cytokine secretion (FIGS. 3B-3C, 3F-3G) and cytotoxicity (FIGS. 3D-3E, 3H-31) as described above. Interestingly, there was a dramatic reduction in the reactivity as shown by both IFN-γ secretion and cytotoxicity when the Vα domain of the TCR is replaced by VL of the H2-scFv and Vβ replaced by VH with or without the linker (Split/CAR-1 vs. Split/CAR-2 and Split/CAR-3 vs. Split/CAR-4, FIGS. 3B-3E). This arrangement also showed increased nonspecific cytotoxicity (FIGS. 3D-3E). Overall, the Split/CAR receptor design with the optimum performance was Split/CAR-3, in which the TCR Va and VB were substituted with the H2-scFv VH and VL chains, respectively, with the addition of the EAAAK linker (FIG. 3A). In fact, both cytokine secretion and cytotoxicity of the Split/CAR-3 T cells rivaled that of T cells containing their natural receptors (compare Split/CAR-3 with TCR-1 T cells in FIGS. 3F-3I).

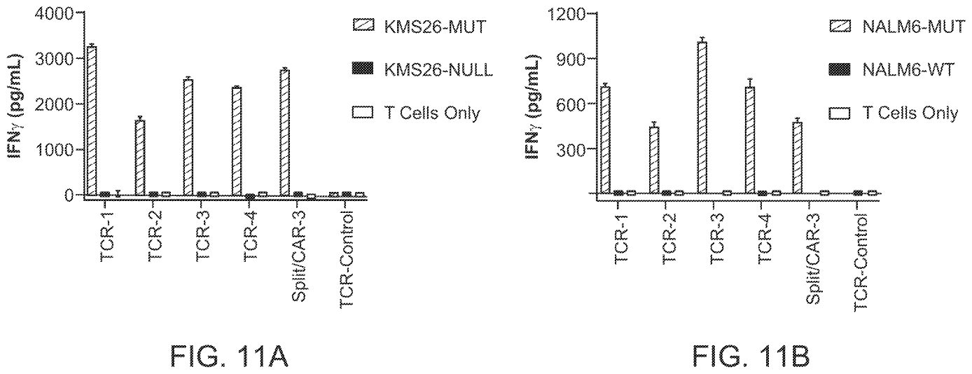

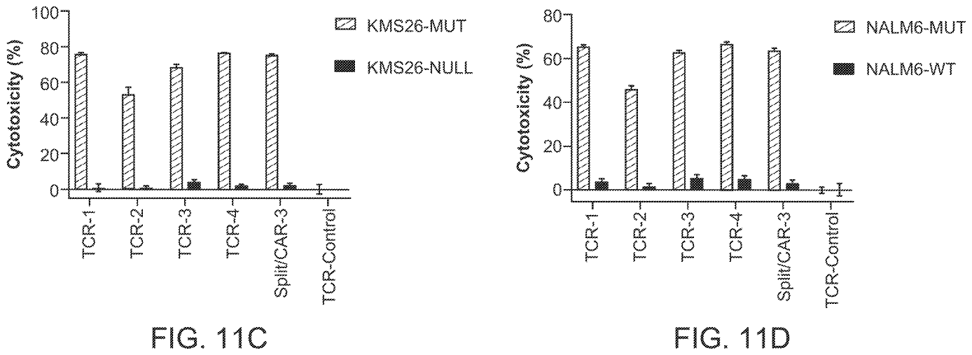

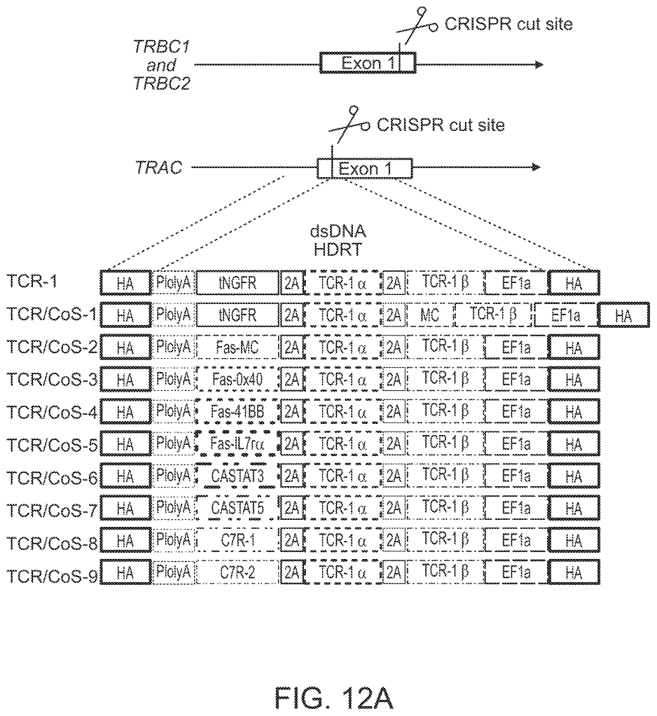

To further substantiate the equivalence of the Split/CAR-3 T cells with T cells expressing naturally occurring TCRs, three TCRs directed against the p53RH antigen were evaluated in addition to the TCR already tested in patients (i.e., TCR-1). The TCRs had affinities ranging from 1.1 to 39.9 μM, and each was inserted into the TRAC locus of primary T cells using CRISPR-based technologies (FIG. 7A) to create TCR-2, 3, and 4. Split/CAR-3 T cells performed comparably to all four naturally occurring TCRs, for both cytokine secretion (FIGS. 11A-11B) and cytotoxicity (FIGS. 11C-11D), after exposure to cancer cells expressing the p53RH antigen at low densities.

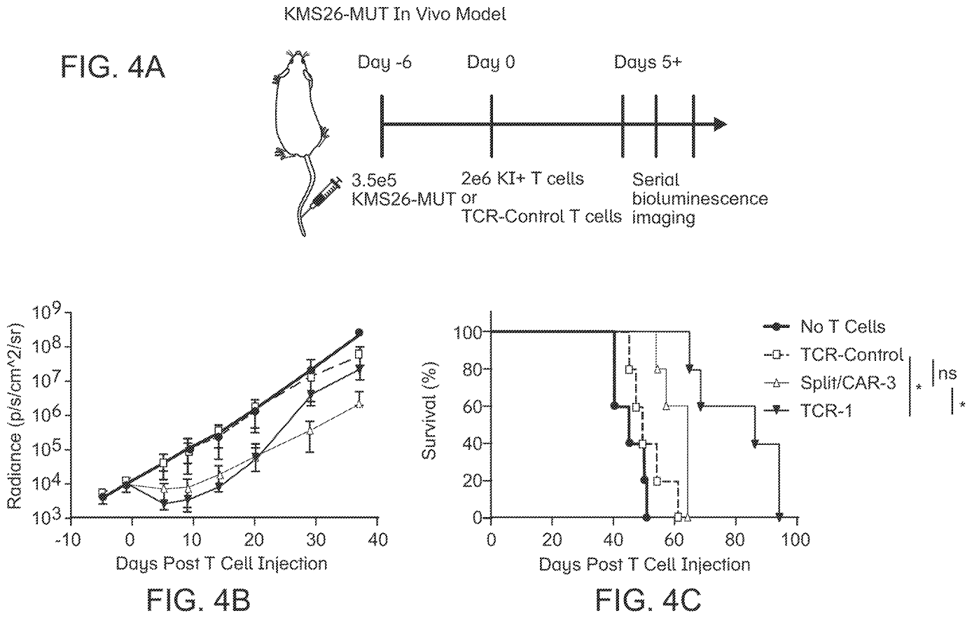

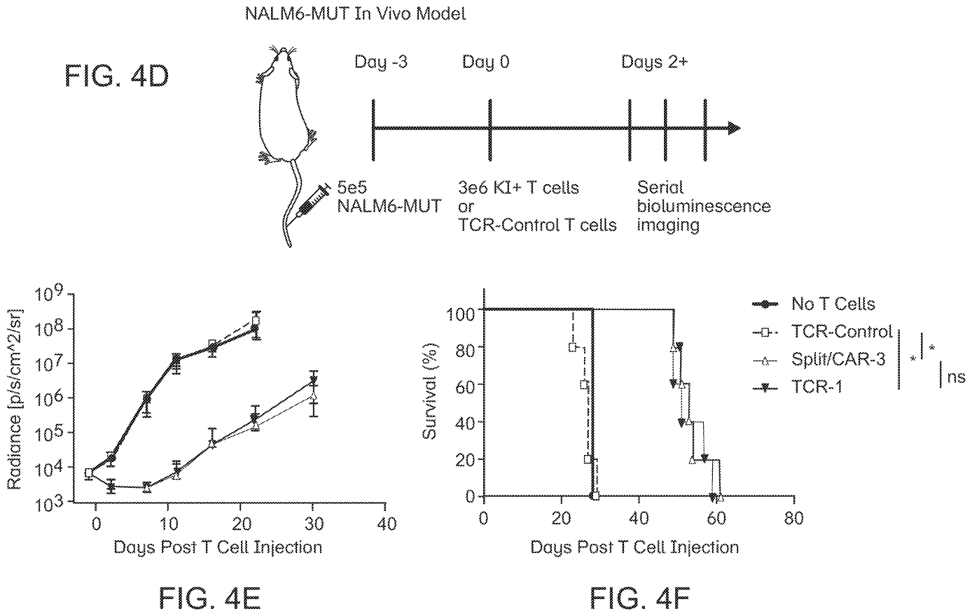

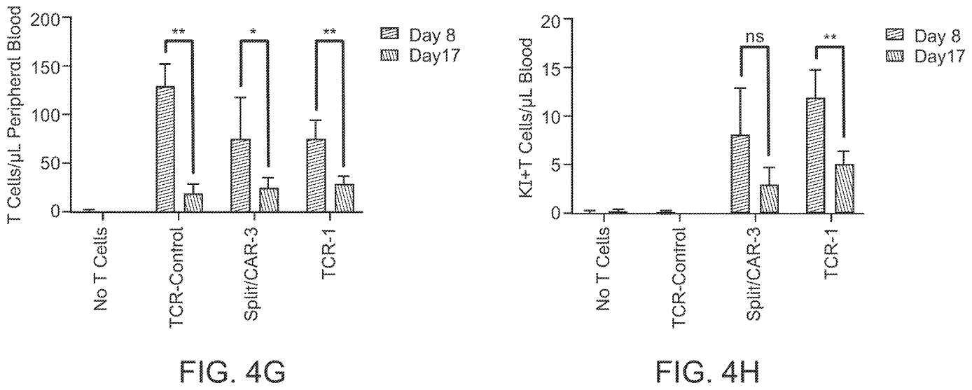

To determine whether the improved function of Split/CAR-3 T cells in vitro could also be observed in vivo, KMS26-MUT cells were injected into NSG mice. After documentation of tumor engraftment six days after implantation, the mice were treated with a single dose of Split/CAR-3, TCR-1, or TCR-Control cells, or no T cells (FIG. 4A). Regressions of the established tumors were observed after treatment with either Split/CAR-3 or TCR-1 cells (FIG. 4B). To validate these results in a different in vivo model, a similar experiment was conducted with NALM6-MUT cancer cells instead of KMS26-MUT cells (FIG. 4D). Equivalent tumor regressions were observed after treatment with Split/CAR-3 or TCR-1 cells (FIG. 4E). Importantly, however, both the KMS26-MUT and NALM6-MUT cancers eventually relapsed and the mice died of their cancers between 1.5 and 3 months after the initiation of T-cell therapy (FIGS. 4C and 4F). On days 8 and 17 after T cell injection in the NALM6-MUT model, peripheral blood was analyzed for the persistence of the engineered T cells. Neither TCR-1 nor Split/CAR-3 cells had expanded at the Day 17 time point compared to Day 8 (FIGS. 4G and 4H) even though the tumors were continuing to expand (FIG. 4E). This result suggested that the tumors recurred in these model systems because the T cell expansion was not sustained.

### Example 4—Tier 4: Co-Stimulation Boosts Activity of Conventional TCRs and Split/CAR T Cells

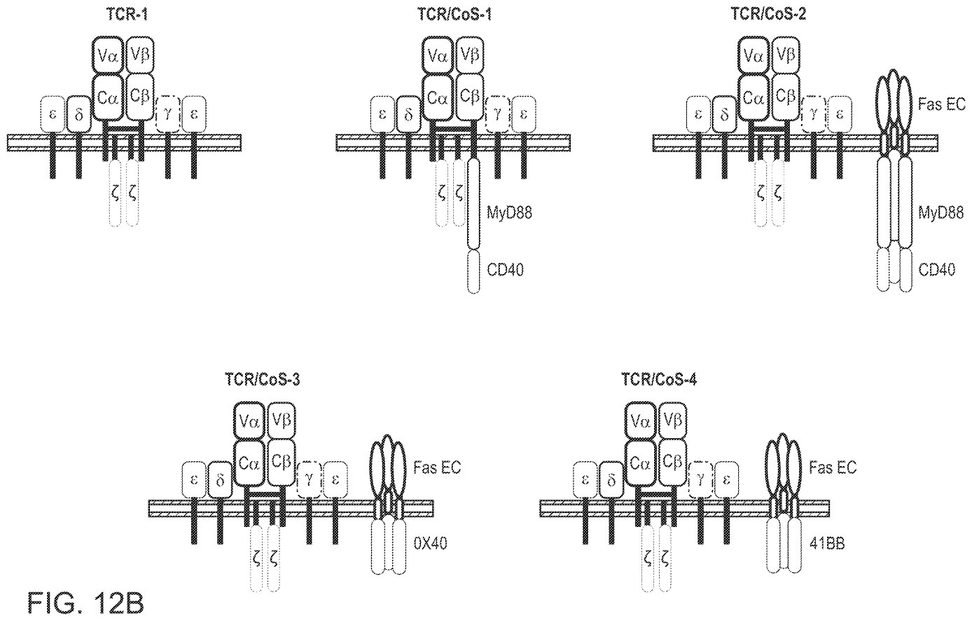

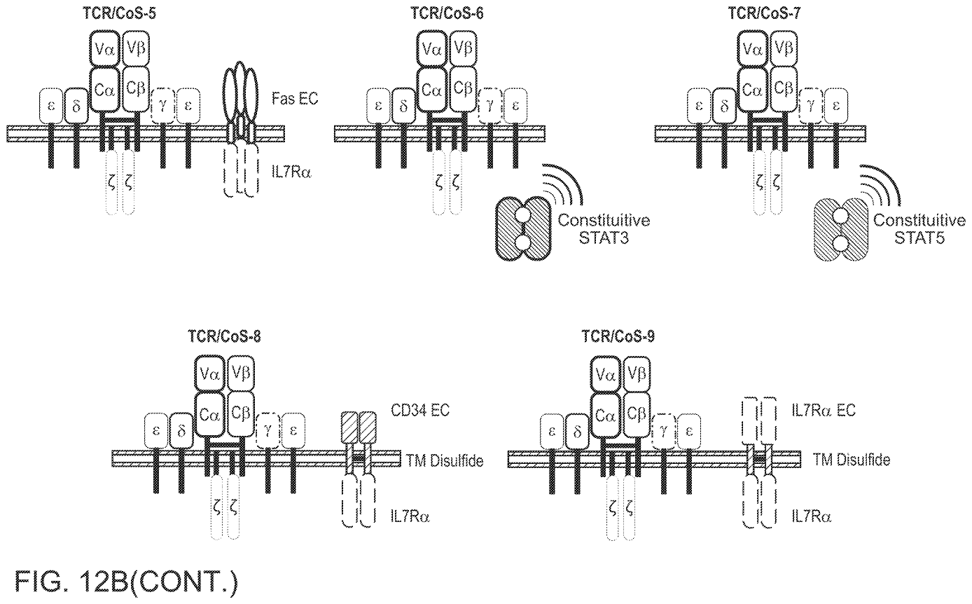

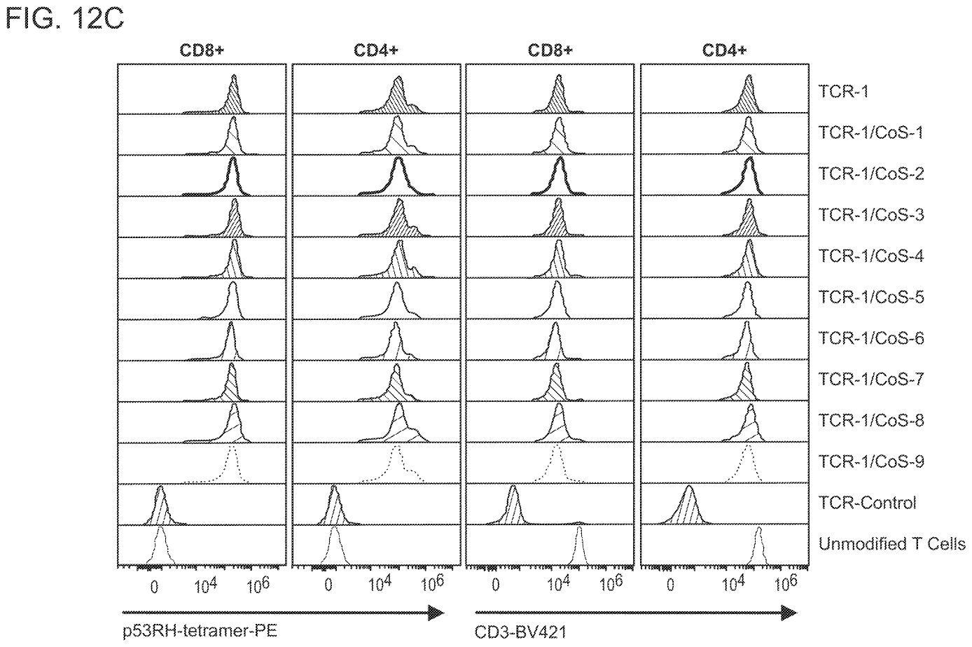

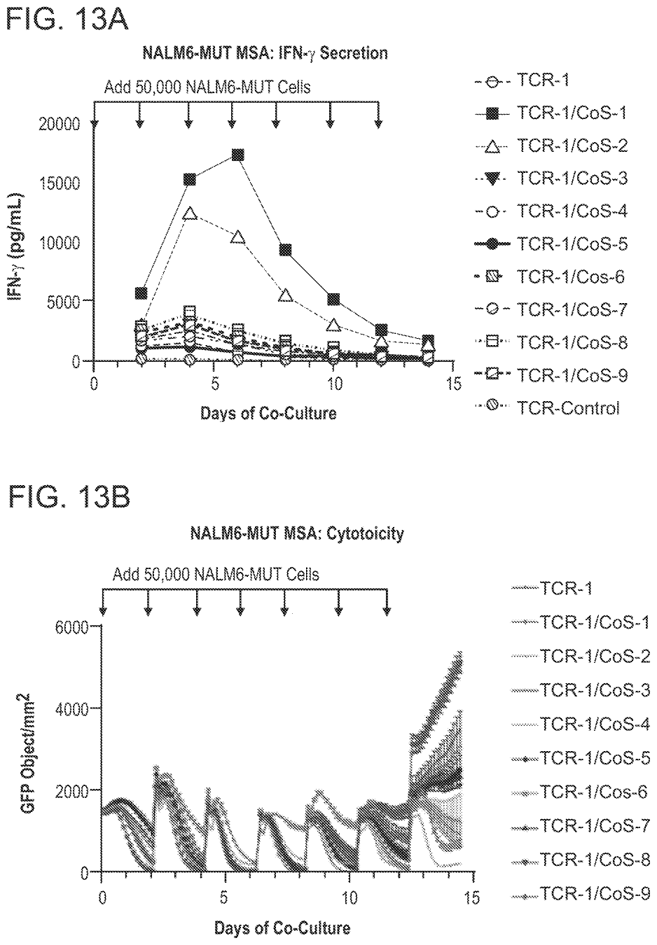

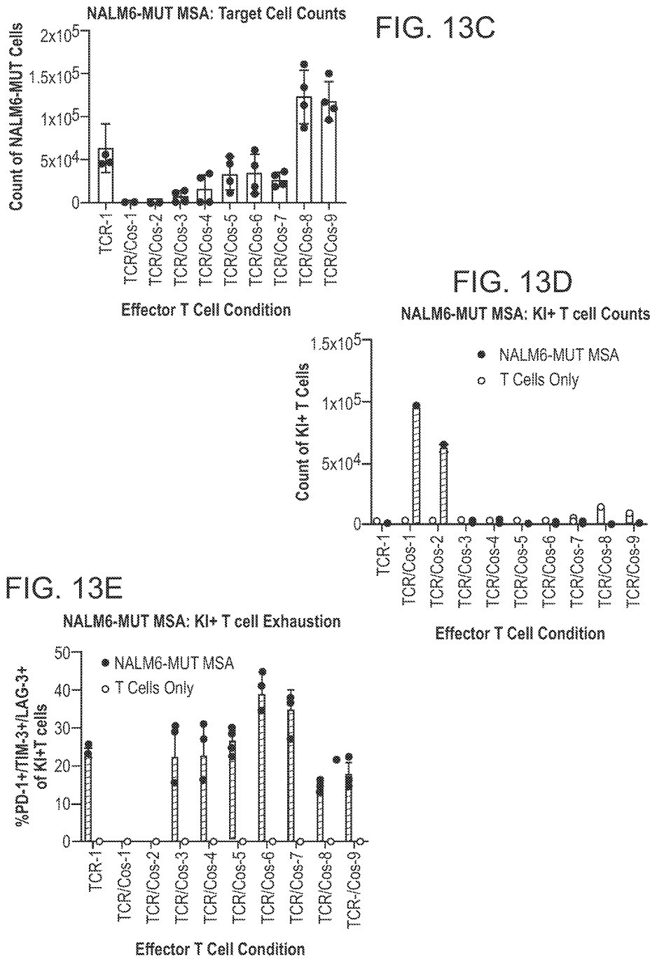

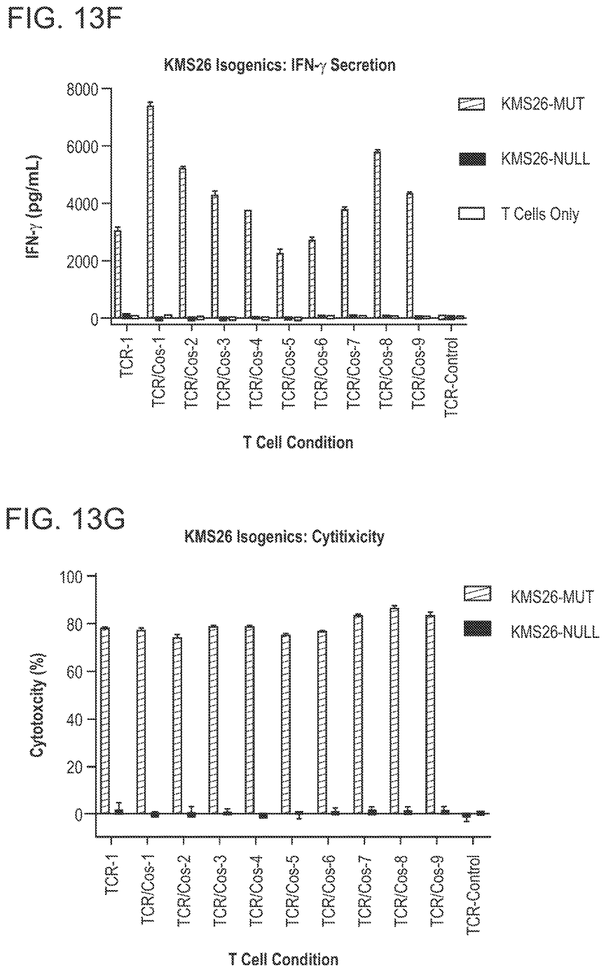

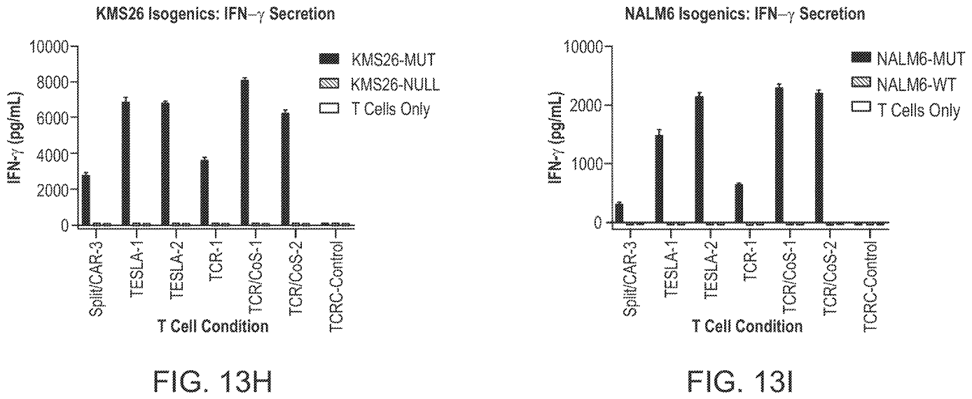

In light of the transient tumor control and un-sustained T cell proliferation achieved with TCR-1 or Split/CAR-3 T cells, it was sought to identify additional co-stimulatory components that might augment and prolong T-cell activity against cancer cells expressing low levels of surface antigen. It began by engineering TCR-1 T cells to express components of several different co-stimulatory proteins. The components included those from the 4-1BB receptor, interleukin-7 receptor alpha (IL7Rα), Fas receptor, STAT3, or STAT5 pathways and were based on studies of T cells activated by exposure to target cells with high antigen densities. Novel combinations of surface receptor domains with intracellular signaling domains were also explored. In each case, the co-stimulatory modules were introduced into the TRAC locus through CRISPR-based technologies along with TCR-1 (FIG. 12A), generating TCR/CoS (Co-Stimulated) T cells of nine types (FIG. 12B). The p53RH binding capacity was similar between TCR-1 and all TCR/CoS T cells as measured by flow cytometry regardless of the co-stimulatory domains included (FIG. 12C). When functionally tested against NALM6-MUT cells expressing low-density antigens, two of the TCR/CoS cell types (TCR/CoS-1 and -2) produced much more IFN-γ than the others (FIG. 13A). TCR/CoS-1 and -2 were also more cytotoxic than the other TCR/CoS T cell types (FIGS. 13B-13C). Moreover, these two T cell types proliferated longer and expressed lower levels of co-inhibitory receptors than the other TCR/CoS cell types tested (FIGS. 13D-13E). The specificity of all TCR/CoS T cell types was documented by the absence of cytokine secretion and cytotoxicity after co-culture with isogenic cancer cells devoid of the p53RH antigen (FIGS. 13F-13G). Interestingly, the TCR/CoS-1 and -2 T cells were the only ones tested with co-stimulation provided by a combination of MyD88 with CD40 (an MC domain), both of which signal through the NFκB pathway.

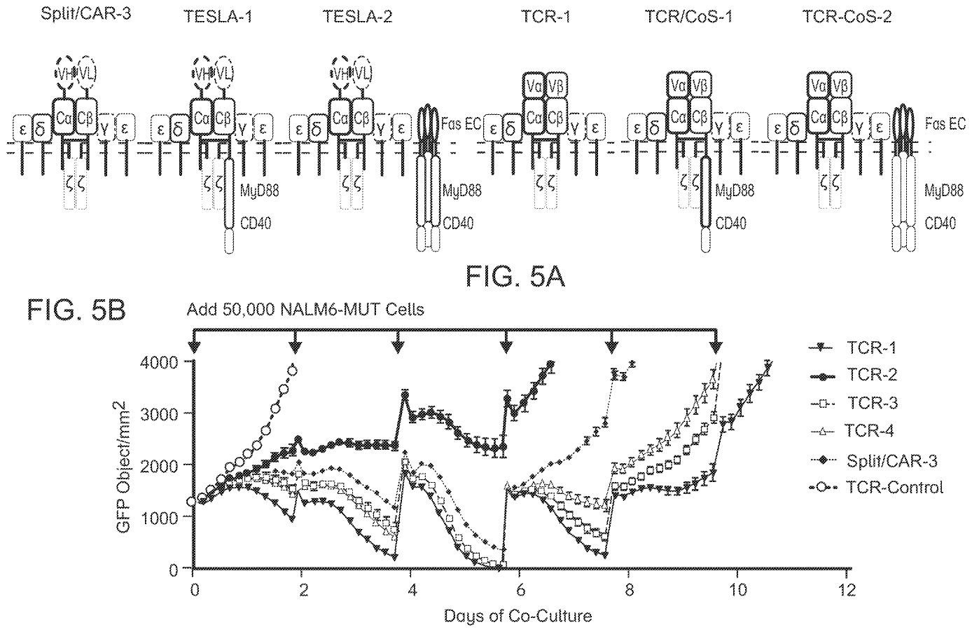

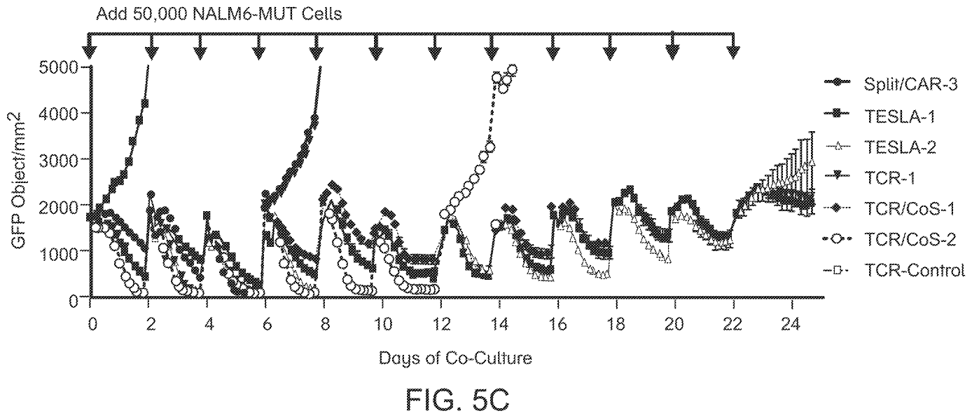

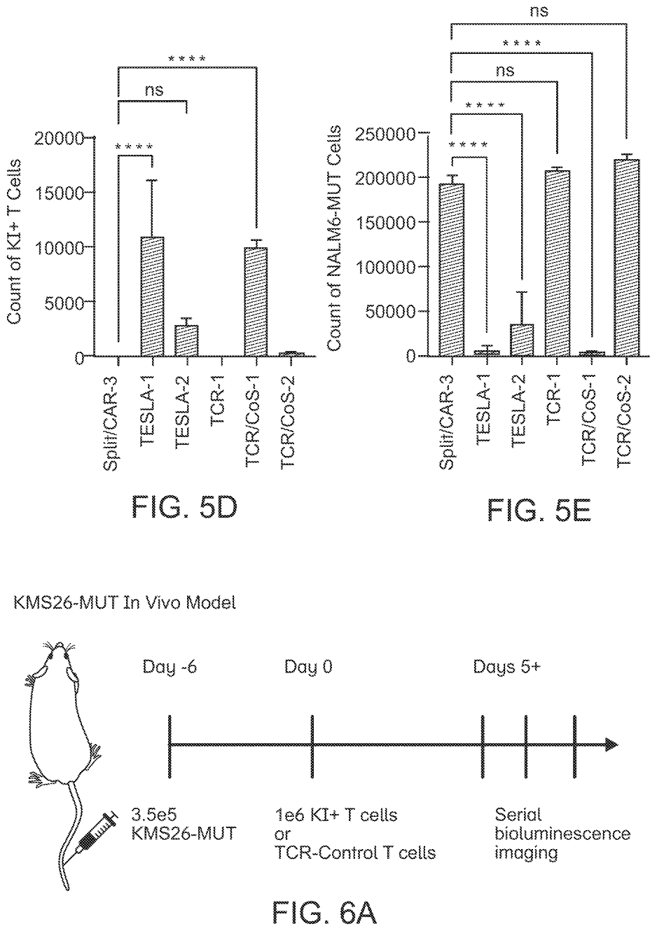

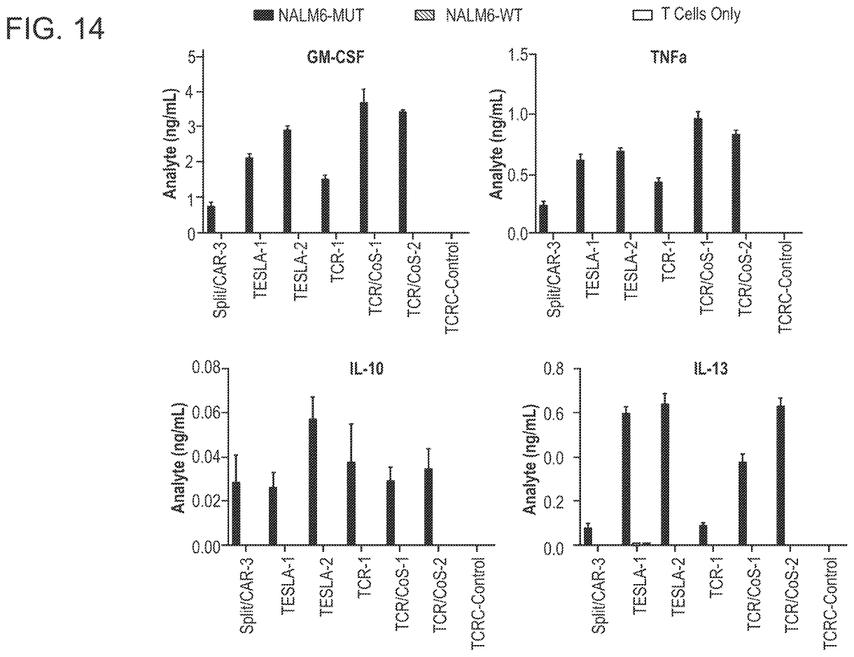

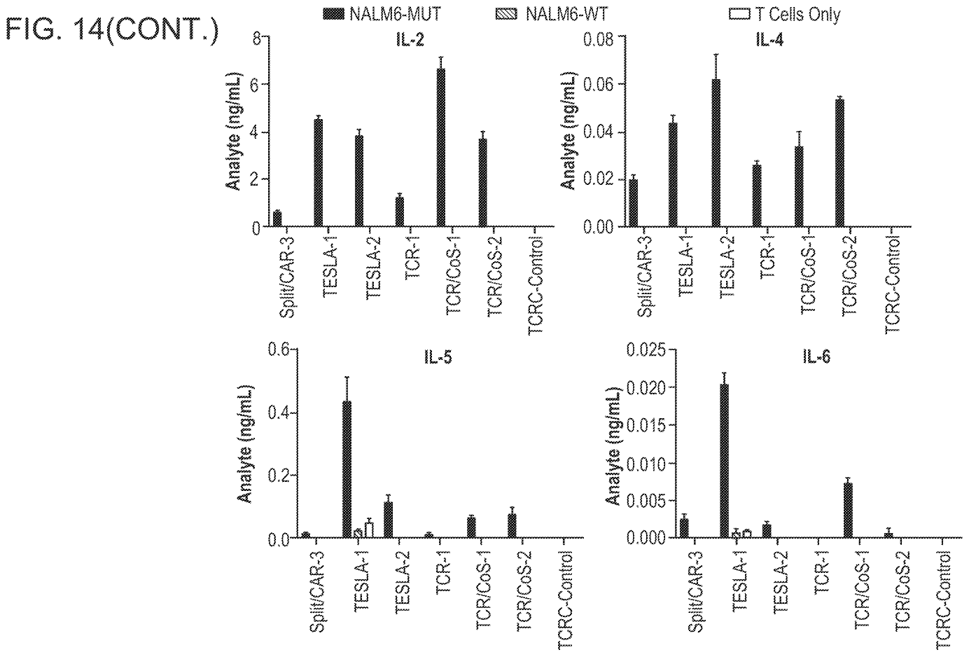

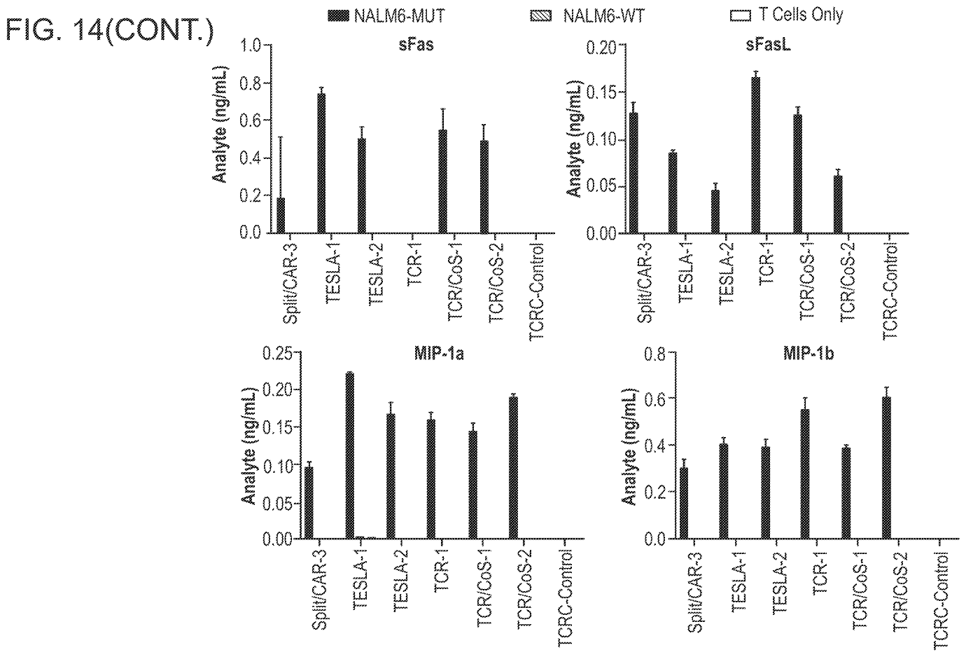

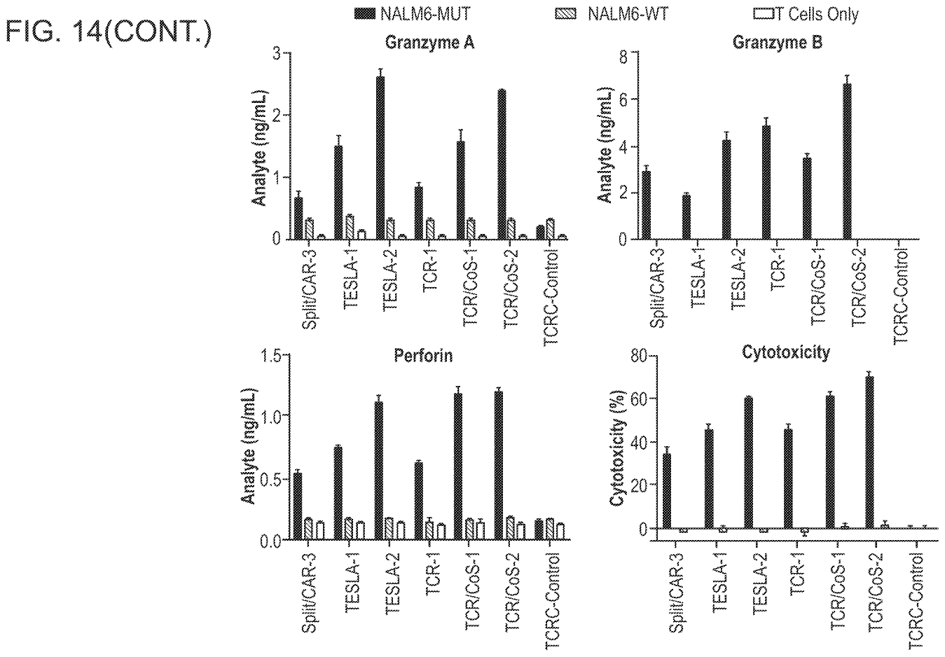

Based on these results, the most potent Tier 3 T cells (i.e., Split/CAR-3, FIGS. 3F-3I) were analogously engineered to express MC domains (FIG. 5A). The resulting T cells were named TESLA-1 and TESLA-2, with TESLA an acronym for TCR Embedded ScFv for Long-term Activation. After co-culture with KMS26-MUT or NALM6-MUT target cells, the MC domains in TESLA and TCR/CoS T cells enhanced IFN-γ production over that observed in the unmodified Split/CAR-3 and TCR-1 T cells (FIGS. 13H-13I). The MC domains also enhanced the secretion of 15 other proteins associated with T cell activation in addition to enhancing cytotoxicity (FIG. 14). Retained specificity was confirmed by co-culture with KMS26 or NALM6 cells devoid of the p53RH antigen (FIGS. 13H-13I, and FIG. 14).

### Example 5—Long-Term Tumor Control is Enabled by Co-Stimulation in TESLA T Cells

The purpose of including co-stimulatory domains in TESLA and TCR/CoS T cells was to prolong and enhance the ability of T cells to control cancer cell growth. One day long co-cultures of T cells with tumor cells (e.g., those described in FIGS. 1A-1F, 2A-2E, and 3A-3I) do not recapitulate the repeated antigen stimulation encountered in vivo. In the clinic as well as in experimental mice, the “co-culture” takes place over much longer time periods and represents a battle between expanding target cancer cells vs. persistently activated or exhausted engineered T cells. To better mimic this in vivo situation in vitro, multi-stimulation assays (MSAs) were used in which new GFP-labeled cancer cells (but not new T cells) were added to the co-cultures every other day and growth was monitored over relatively long periods of time. The data in FIG. 5B show that Split/CAR-3 T cells, as well as TCR-1, -2, -3, and -4 T cells, were all initially able to retard the growth of cancer cells. However, the T cells failed to control growth during re-challenge after six to eight days in culture. This result was expected given the failure of the same T cells to control the growth of tumors in mice for longer than 10 days (FIGS. 4A-4F). However, with the addition of MC co-stimulatory domains to the T cells, tumor cell growth could be controlled much longer, with failure evident only after three weeks of co-culture for three of the four T-cell types tested (FIG. 5C). This prolonged control was mirrored by the persistence of the T cells. Without the additional MC co-stimulatory domains, there were only cancer cells, and no T cells, left in the wells after 25 days of multi-stimulation (FIGS. 5D-5E). With the MC co-stimulatory domain, T cells were still present after 25 days. In general, the MC co-stimulatory domains attached to the extracellular and transmembrane domain of TCRβ (TESLA-1 and TCR/CoS-1) was more potent than the MC co-stimulatory domain attached to the extracellular and transmembrane domain of Fas (TESLA-2 and TCR/CoS-2; FIGS. 5C-5E).

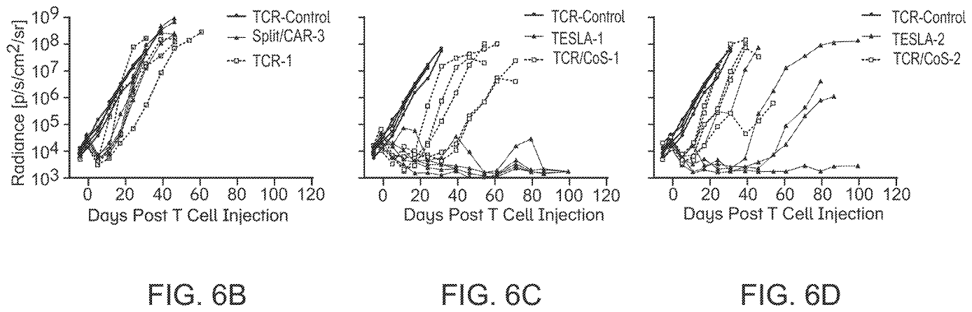

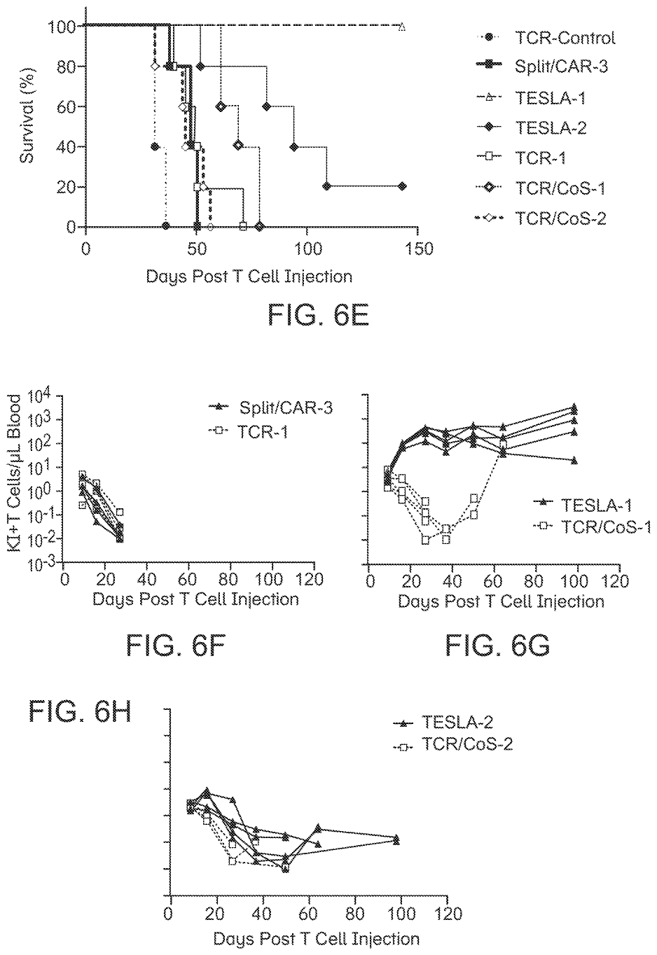

In mice, Tier 3 T cells (TCR-1 or Split/CAR-3) could not control KMS26-MUT tumor growth for longer than 10 days after administration of T cells (i.e., a total of 16 days after injection of cancer cells) (FIGS. 6A-6B). With MC co-stimulatory domains, tumor control was prolonged, with no tumors evident at the end of the experiment after treatment with TESLA-1 T cells (5 months, FIGS. 6C-6E). Additionally, no signs of graft versus host disease were observed for the duration of the experiment. The differences between the effectiveness of the various T-cell types were pronounced in these experiments. First, TESLA cells were considerably more effective than TCR/CoS T cells with the same MC domains. This can be appreciated by comparing TESLA-1 and -2 with TCR/CoS-1 and -2 in tumor growth kinetics (FIGS. 6C-6D) and mouse survival (FIG. 6E). Second, the MC domain was more effective when attached to TCRβ (TESLA-1) than when attached to Fas (TESLA-2) (FIGS. 6C-6E). A plausible explanation for the superiority of TESLA-1 cells over the other T-cell types was discovered by monitoring the number of modified human T cells in the mice during the course of this experiment. The degree of expansion and persistence of TESLA-1 cells was greater than those of any other type of T cells evaluated (FIGS. 6F-6H). The difference in expansion of T cells in vivo between TESLA-1 and the other types of T cells was two to three orders of magnitude.

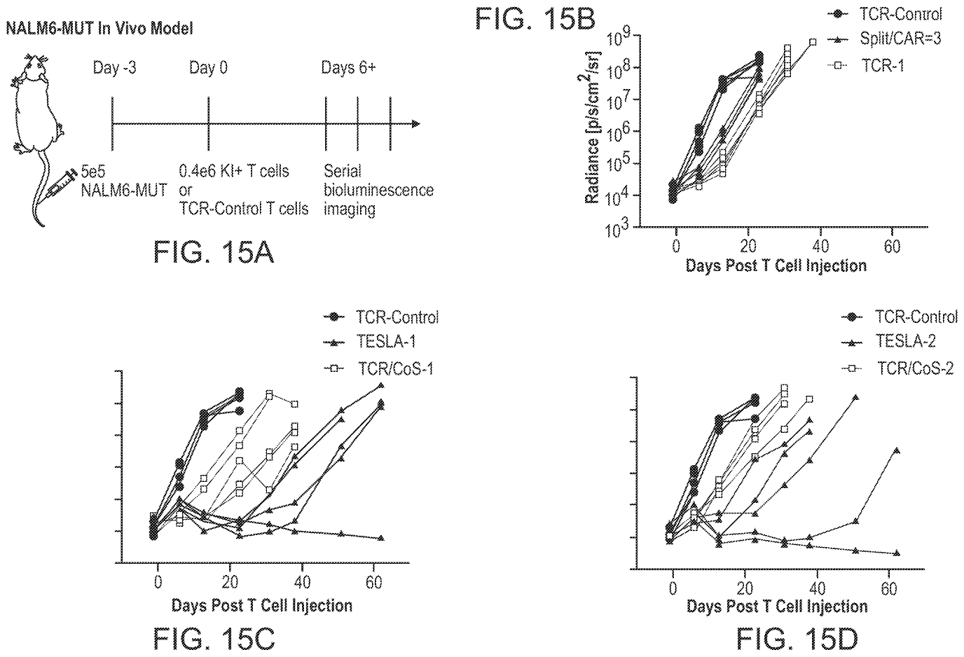

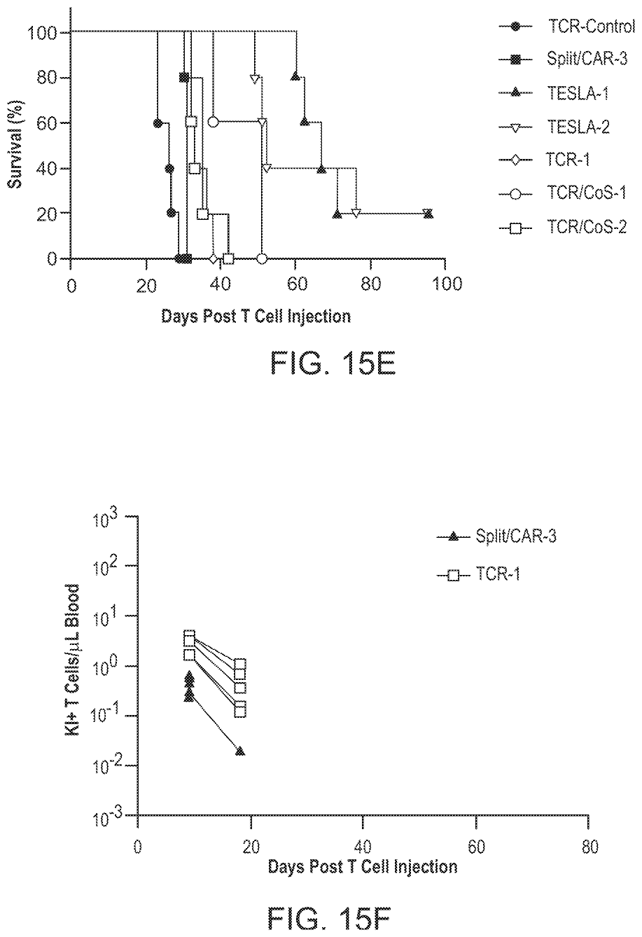

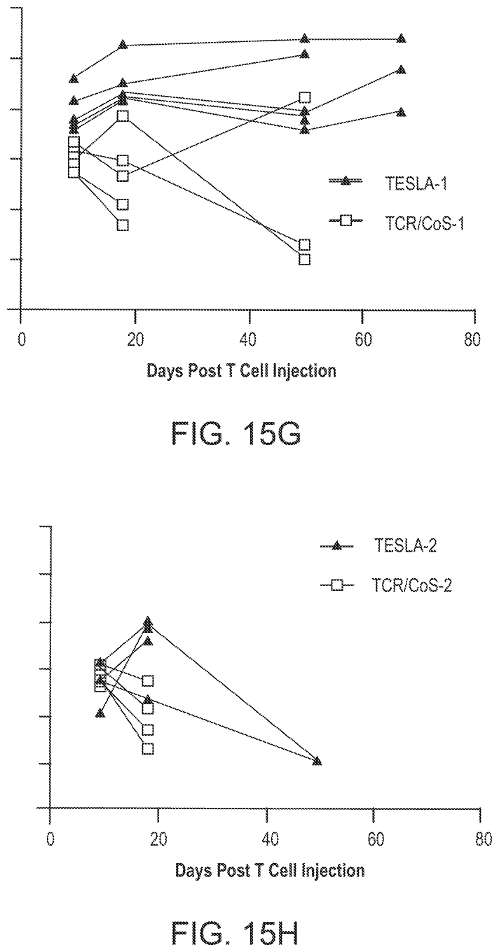

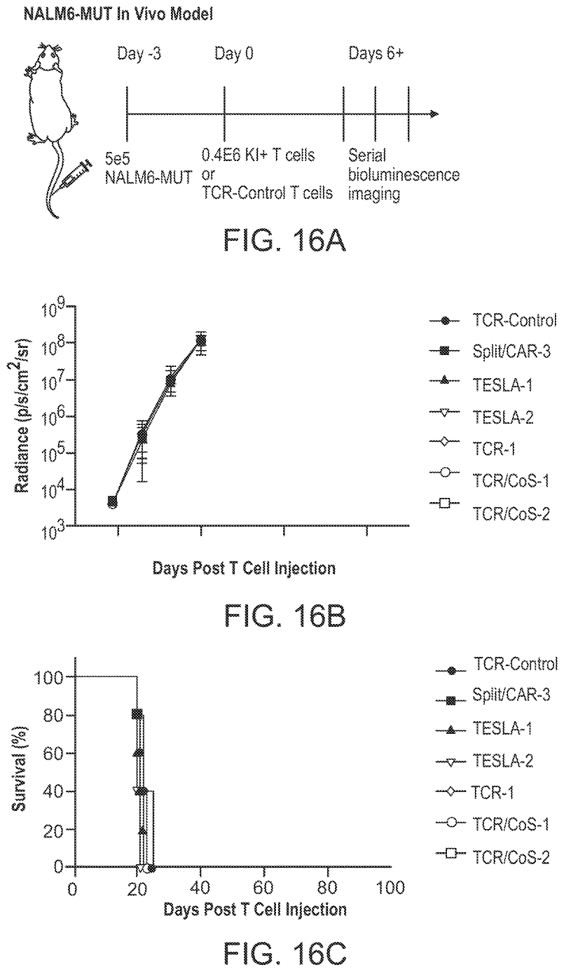

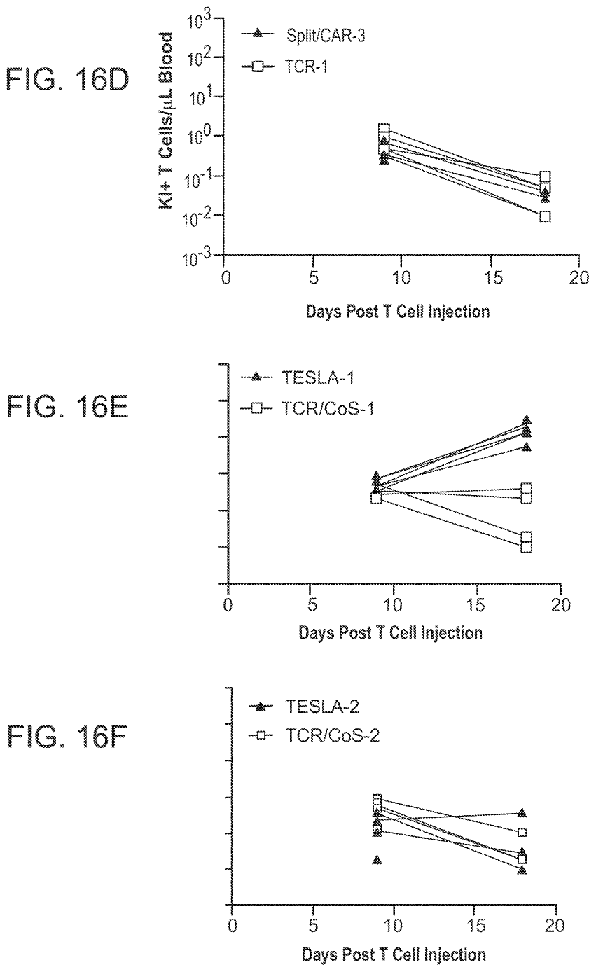

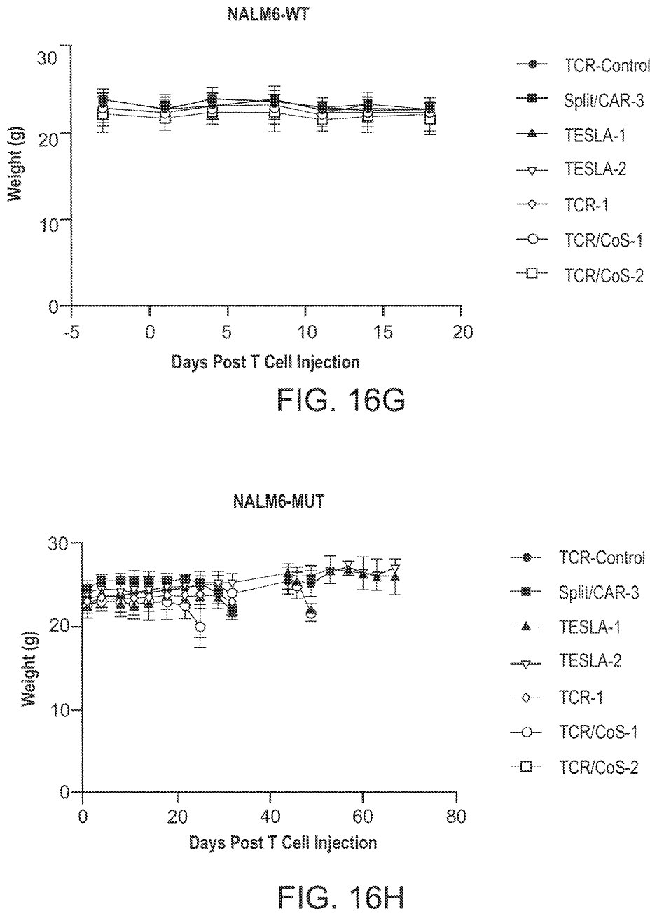

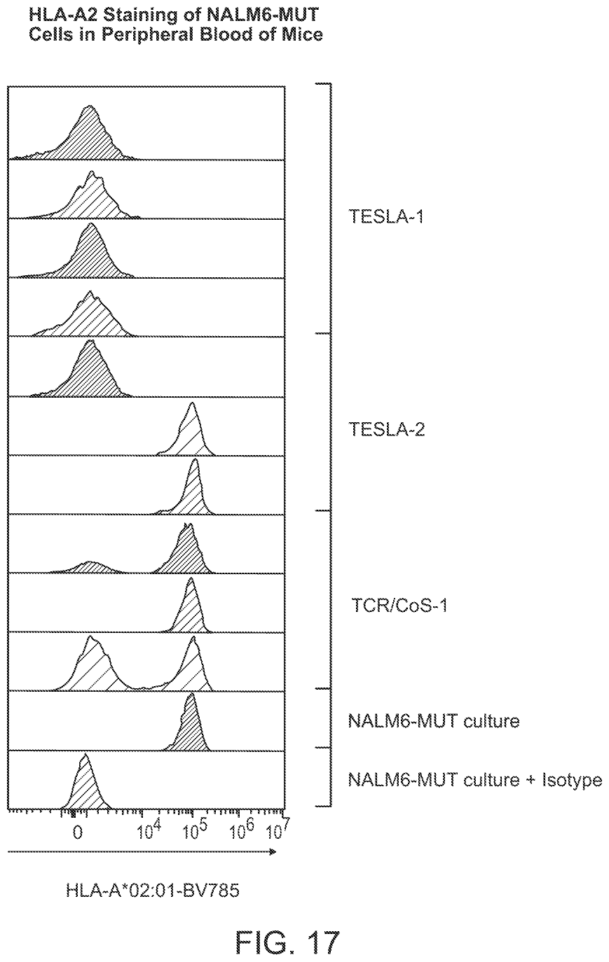

The ability of the engineered T cells to control the growth of a second tumor model in vivo was also evaluated (FIG. 15A). Overall, the relative performance of the various T-cell types was similar in mice bearing NALM6-MUT tumor cells compared to KMS26-MUT tumor cells. For example, TESLA T cells were again more effective than TCR/CoS T cells, though both T-cell types were equipped with the same MC domains (FIGS. 15B-15E). Moreover, in these TESLA T cells, the MC domain was more effective when it was attached to TCRβ than when attached to Fas (FIGS. 15C-15E). The degree of expansion and persistence of TESLA-1 cells was much greater than any other type of T cells evaluated (FIGS. 15F-15H). The specificity of TESLA-1 T cells was documented by experiments in mice bearing isogenic NALM6-WT tumor cells (harboring the normal arginine rather than the mutant histidine at amino acid 175 of p53) (FIG. 16A). Both the growth of NALM6-WT cells and mouse survival were unaffected by TESLA-1 or any other of the engineered T cells assessed (FIGS. 16B-16C). Moreover, the expansion of TESLA-1 cells in mice occurred even in the absence of p53RH stimulation in vivo (FIGS. 16D-16F). No toxicity, as assessed by weight loss, was observed in mice treated with TESLA-1 T cells, regardless of whether the tumor cells expressed the p53RH antigen (FIGS. 16G-16H).

However, there was one major difference between the therapeutic efficacy in the KMS26-MUT and NALM6-MUT models: in the KMS26-MUT model, TESLA-1 treatment induced complete remissions in every mouse, and these lasted throughout the entire duration of the experiment (5 months, FIG. 6E). However, in the NALM6-MUT model, mice began to succumb to their tumors two months following TESLA-1 treatment, with four of the five treated mice eventually dying (FIG. 15E). Based on prior studies on T-cell therapeutics in mice and humans, T-cell treatment failures are typically due to a lack of persistence of the T cells or loss of antigen on the target cells. Flow cytometry revealed that all four of the TESLA-1 treated mice that eventually died from their cancer had lost the HLA-A*02:01 component of the p53RH antigen on their circulating cancer cells (FIG. 17). By contrast, in mice treated with either TESLA-2 or TCR/CoS T cells, all of which succumbed to their tumors earlier than those treated with TESLA-1 T cells, the circulating cancer cells retained the expression of HLA-A*02:01 on their surface. In these mice, tumor relapse was apparently due to the disappearance of the therapeutic T cells (FIGS. 15G-15H).

### Example 6—TESLAs Protect Against Exhaustion and Support CD4 T Cell Expansion

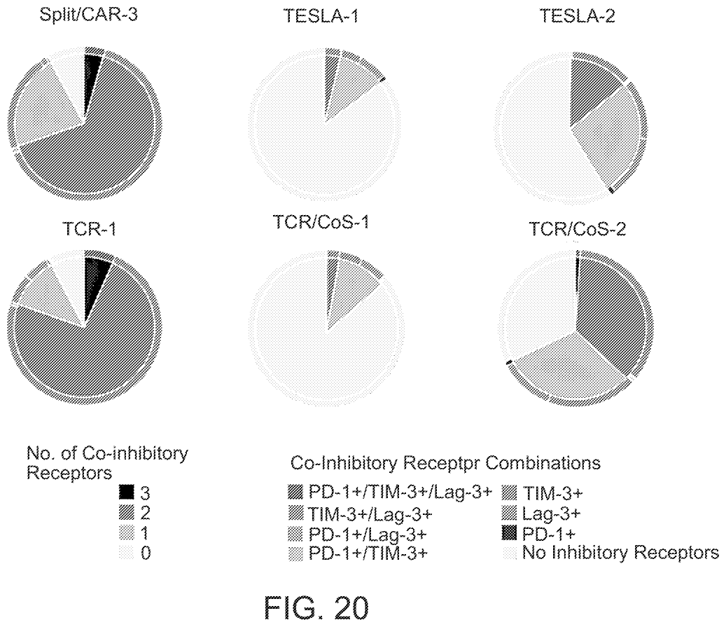

To further characterize the functional differences between TESLA and TCR/CoS cells, co-inhibitory receptor expression was examined after 12 days of multi-stimulation with NALM6-MUT cells, a time when all T cell types still persisted in the cultures. Tier 3 T cells that lacked co-stimulation (TCR-1 or Split/CAR-3) expressed the highest frequencies of PD-1, TIM-3, and LAG-3, with most cells dual positive for TIM-3 and LAG-3 (FIG. 20). Fas-MC co-stimulated TESLA-2 and TCR/CoS-2 T cells expressed lower frequencies of co-inhibitory receptors, with TESLA-2 demonstrating a greater portion of cells (50%) negative for co-inhibitory receptors. Finally, TESLA-1 and TCR/CoS-1 T cells expressed the lowest frequency of co-inhibitory receptors, with more than 80% of cells staining negative for co-inhibitory receptors after 12 days of multi-stimulation. The degree of co-inhibitory upregulation appeared to be inversely correlated with performance in the multi-stimulation assay.

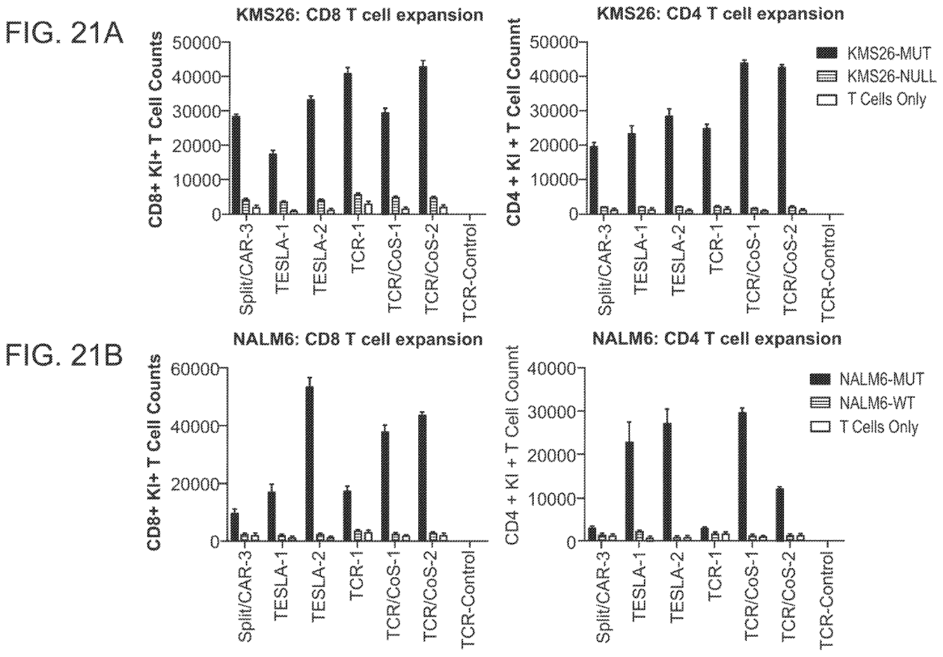

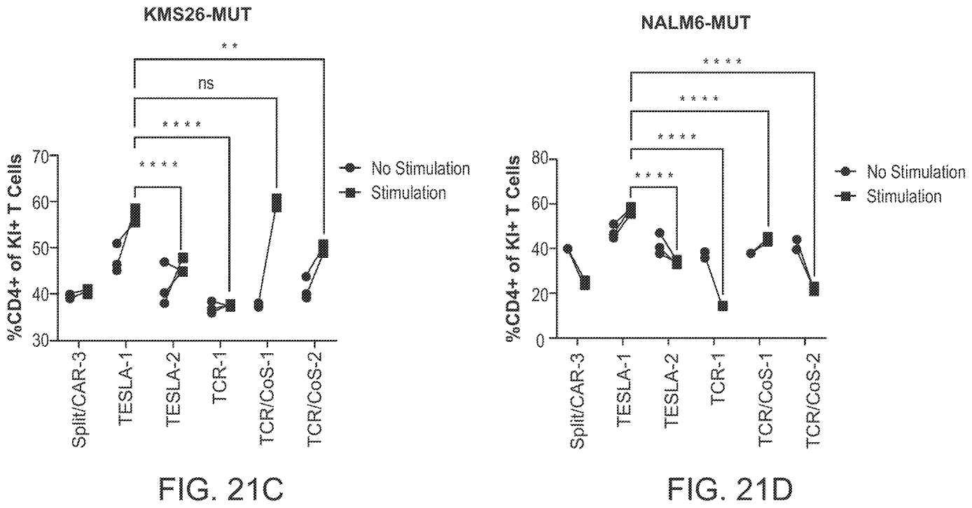

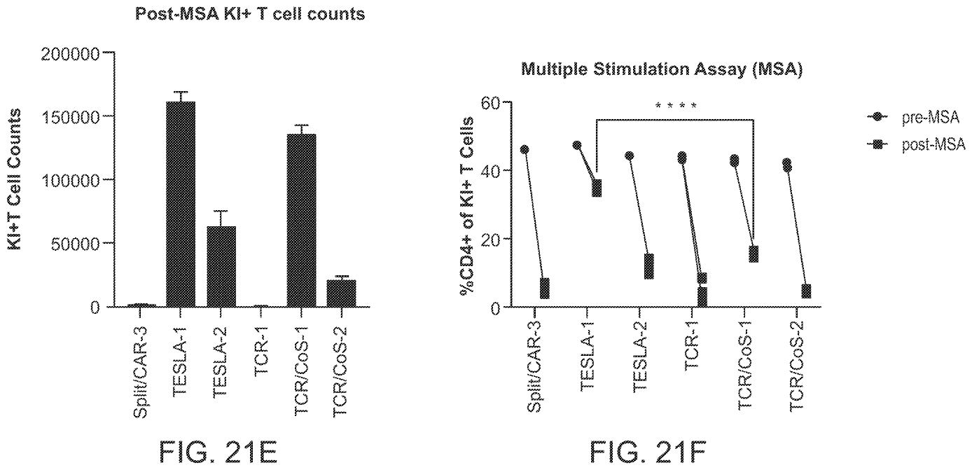

It was also asked whether TESLA or TCR/CoS receptors differed in their capacity to activate CD4 and CD8 T cells in co-culture, as the lower affinity of the TCR-1 binding domain may render it more dependent on CD8 co-receptor engagement. Five days after a single stimulation with either the KMS26-MUT cells or NALM6-MUT cells, both TESLA and TCR/CoS receptors induced expansion of both CD8 and CD4 T cell subsets (FIGS. 21A-21B). No expansion was observed after co-culture with isogenic KMS26-NULL and NALM6-WT cells or when T cells were cultured on their own. Inclusion of MC co-stimulation appeared to preferentially support the expansion of CD4 T cells, as demonstrated by the increased fraction of CD4 T cells observed after a single stimulation with cognate antigen positive cells, with the TCRβ linkage having a more pronounced effect than Fas linkage (FIGS. 21C-21D). The predominance of CD4 T cells was even more pronounced after a 12-day multiple stimulation assay, where the TESLA-1 construct preserved the highest percentage of CD4 T cells relative to all other constructs (FIGS. 21E-21F).

### Example 7—TESLA-1 Modulates Cytokine Secretion and Tonic Signaling

To examine the effects of the MC domain on TESLA-1 function, transcriptomic changes of unedited, Split/CAR-3, and TESLA-1 T cells were examined after an 18-hour co-culture with KMS26-MUT, KMS26-NULL, or no target cells. This allowed multiple comparisons, such as the differential expression of genes among T-cell types after stimulation with the KMS26-MUT target cells or after stimulation of the same T cells with KMS26-NULL cells. At the end of the co-cultures, CD4+ and CD8+ cells were purified, and transcriptome analyses performed separately, permitting other comparisons. In general, hundreds of genes were found to be differentially expressed in every comparison made.

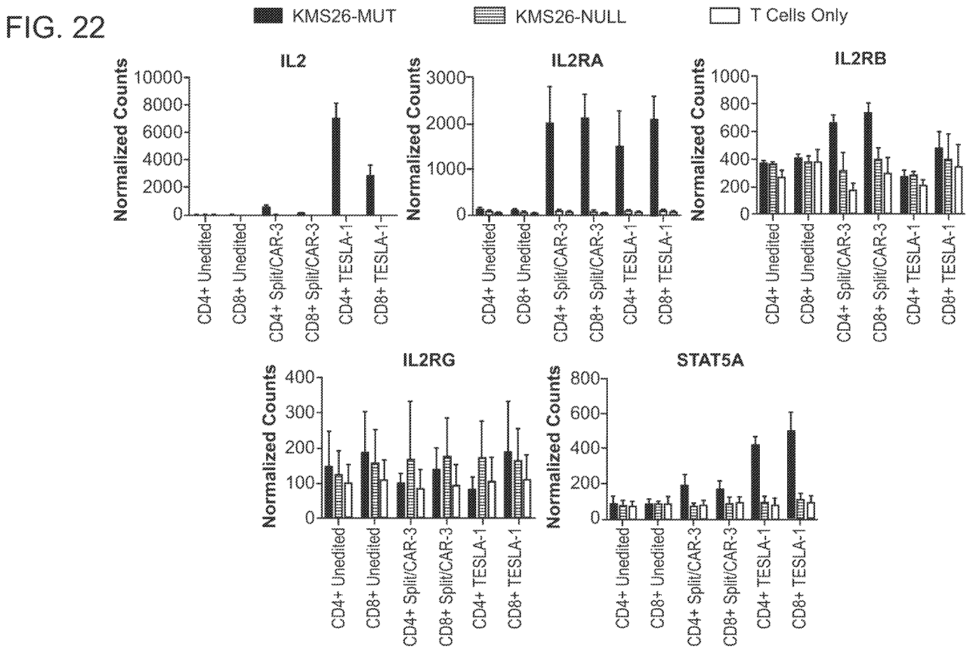

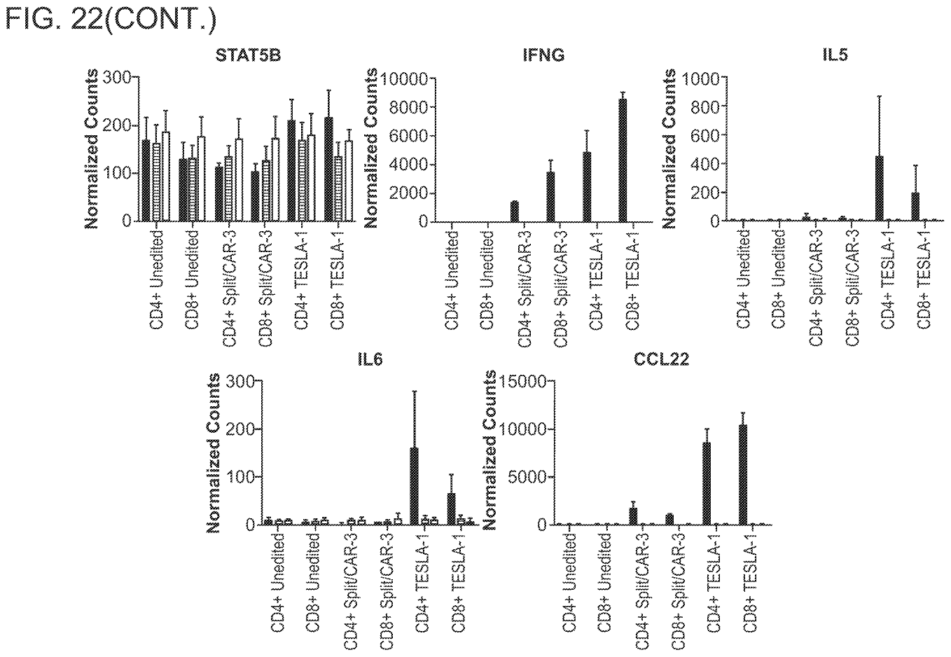

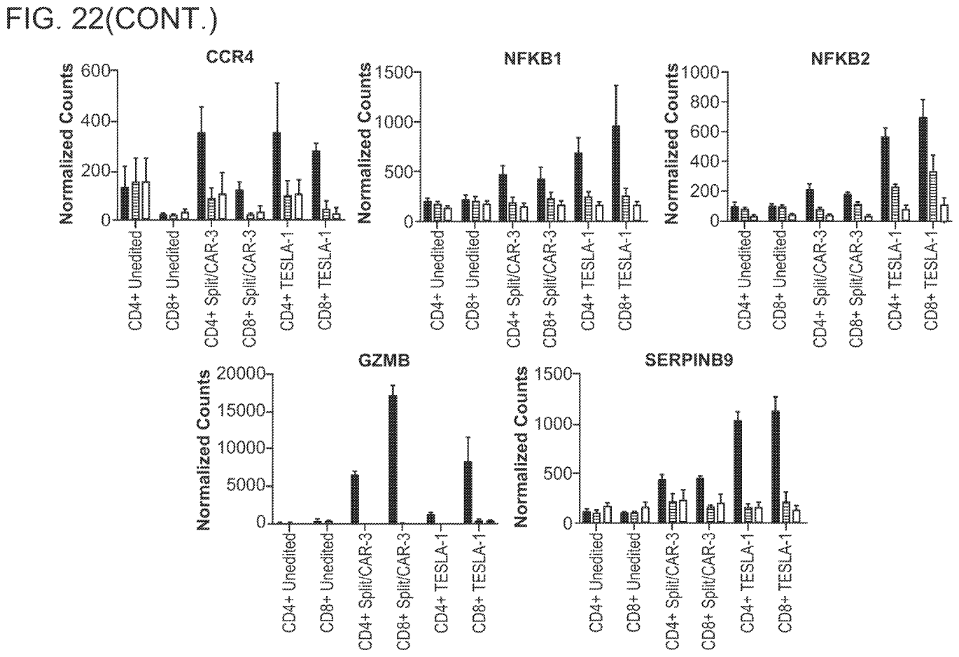

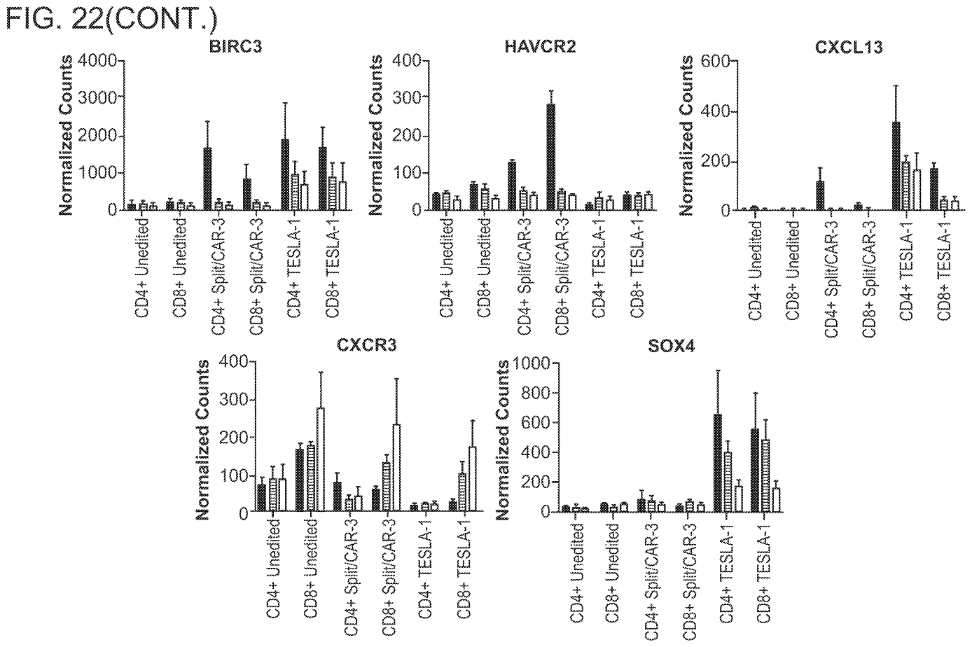

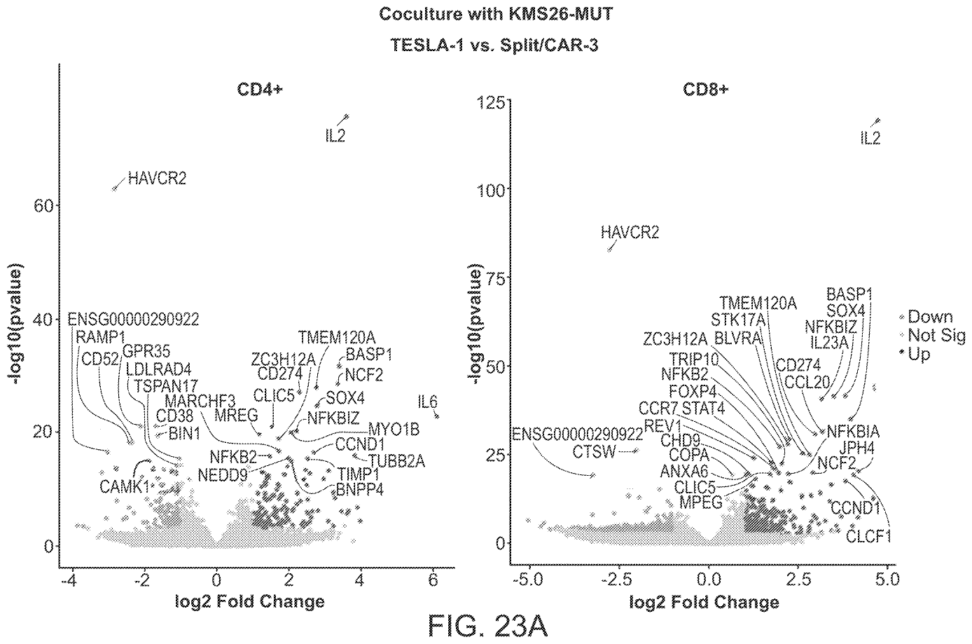

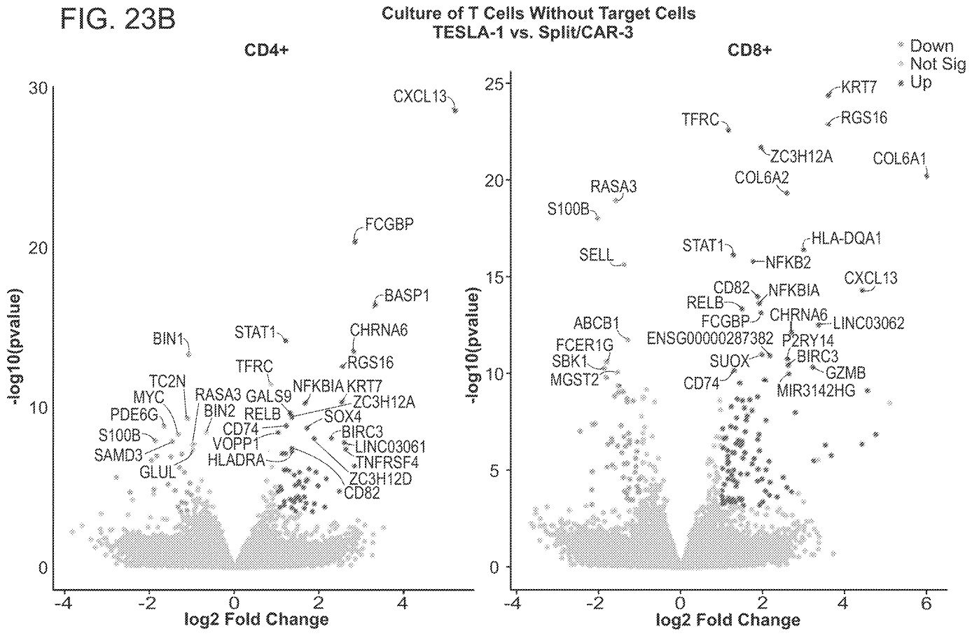

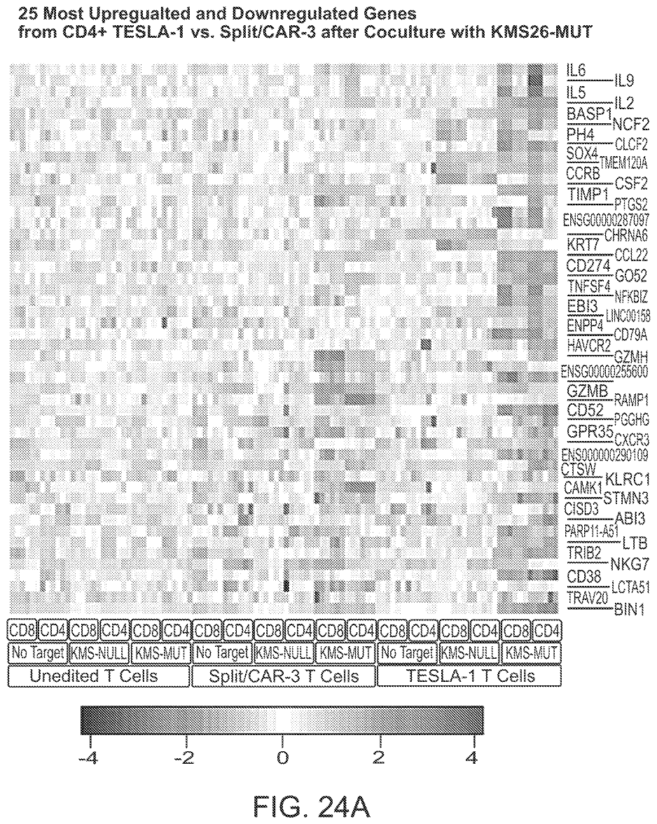

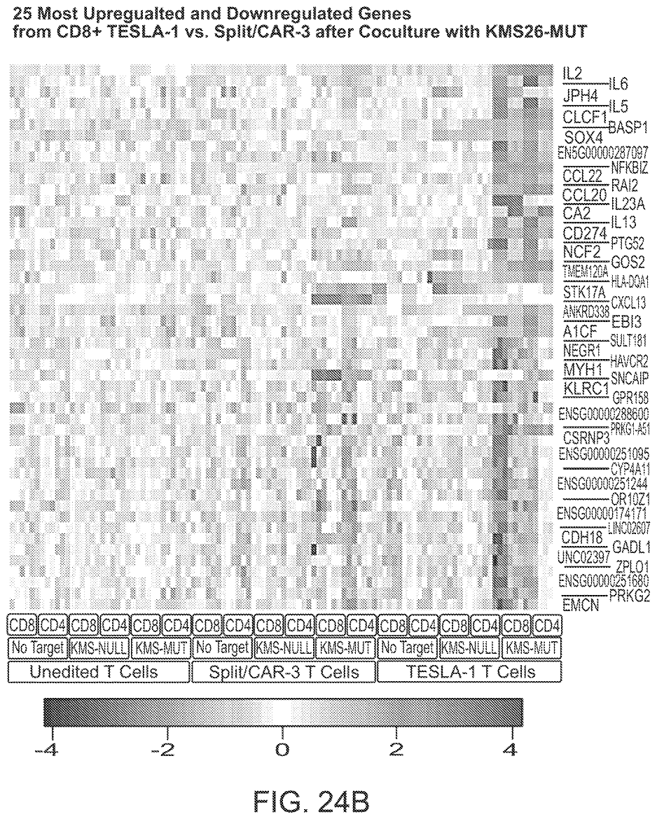

The gene encoding IL-2 (IL2) was the most upregulated gene in TESLA-1 T cells compared to Split/CAR-3 T cells after activation with KMS26-MUT cells, demonstrating a 22-fold increase in CD8+ T cells and 12-fold increase in CD4+ T cells (FIGS. 22 and 23A). Importantly, no upregulation of the gene encoding IL-2 was observed after culturing TESLA-1 T cells with KMS26-NULL target cells or in the absence of target cells (FIGS. 22 and 24A-24B). Concomitant upregulation in Split/CAR-3 and TESLA-1 T cells of the genes encoding IL-2Rα (IL2RA) and STAT5A (STAT5A), along with constitutive expression of the genes encoding IL-2Rβ (IL2RB), the common gamma chain (IL2RG), and STAT5B (STAT5B) suggest an augmented IL-2 autocrine signaling loop in TESLA-1 T cells (FIG. 22). Genes encoding additional cytokines including IFN-γ (IFNG), IL-5 (IL5), and IL-6 (IL6) were upregulated in TESLA-1 T cells compared to Split/CAR-3 T cells after exposure to KMS26-MUT cells and showed no upregulation after exposure to KMS26-NULL cells confirming the specificity of T-cell activation (FIGS. 22 and 23A-23B). Activation-induced upregulation of the genes encoding the chemokine CCL22 (CCL22) in TESLA-1 T cells and its cognate receptor CCR4 (CCR4) in Split/CAR-3 and TESLA-1 T cells highlight another autocrine signaling loop augmented by inclusion of the MC domain (FIG. 22).

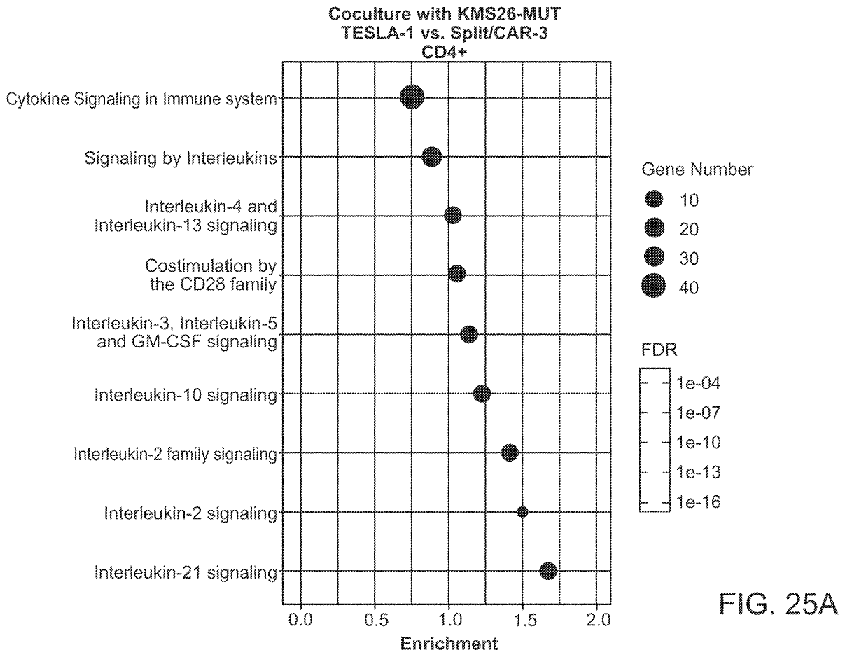

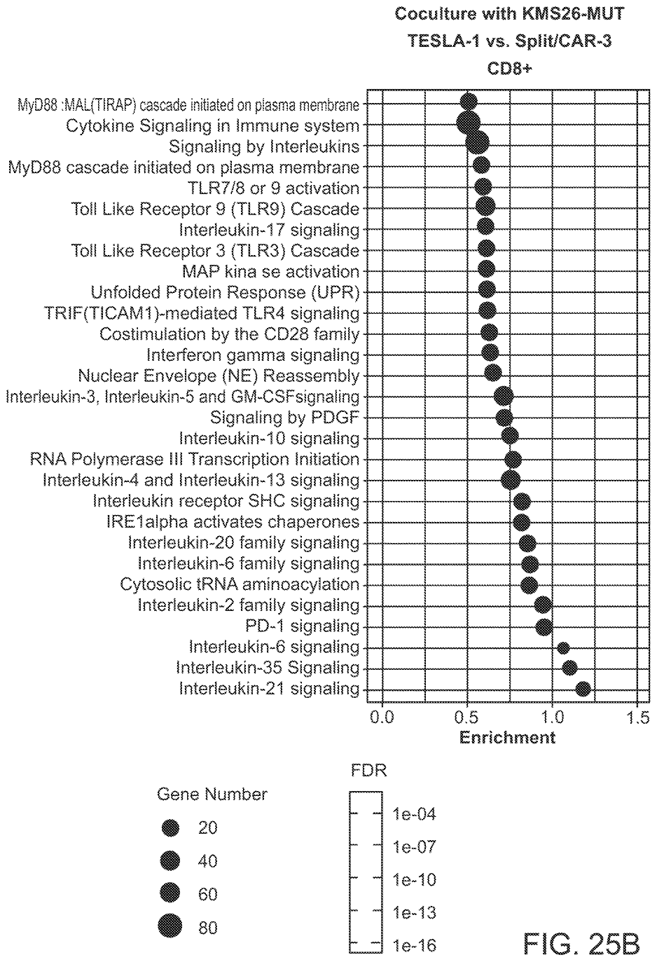

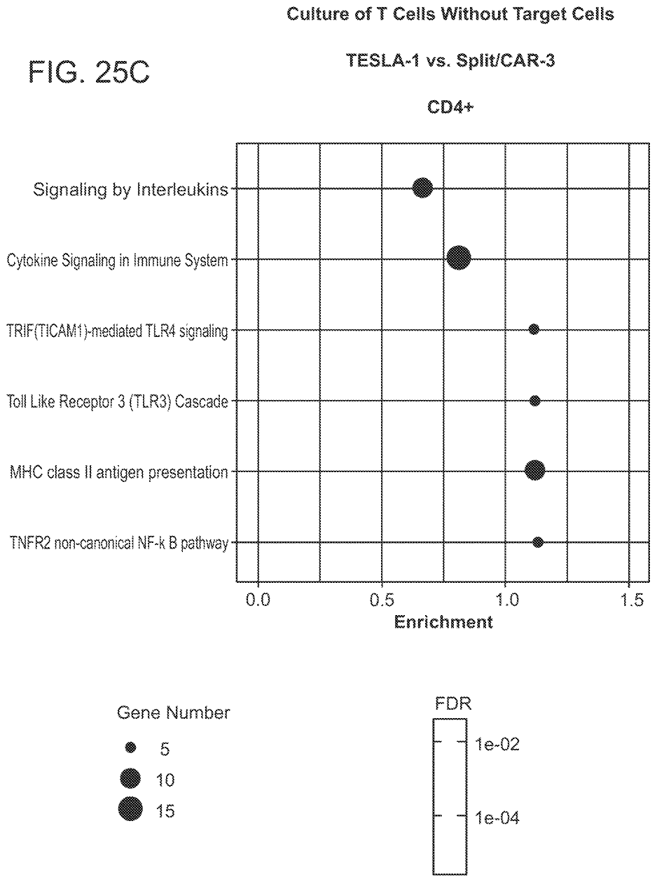

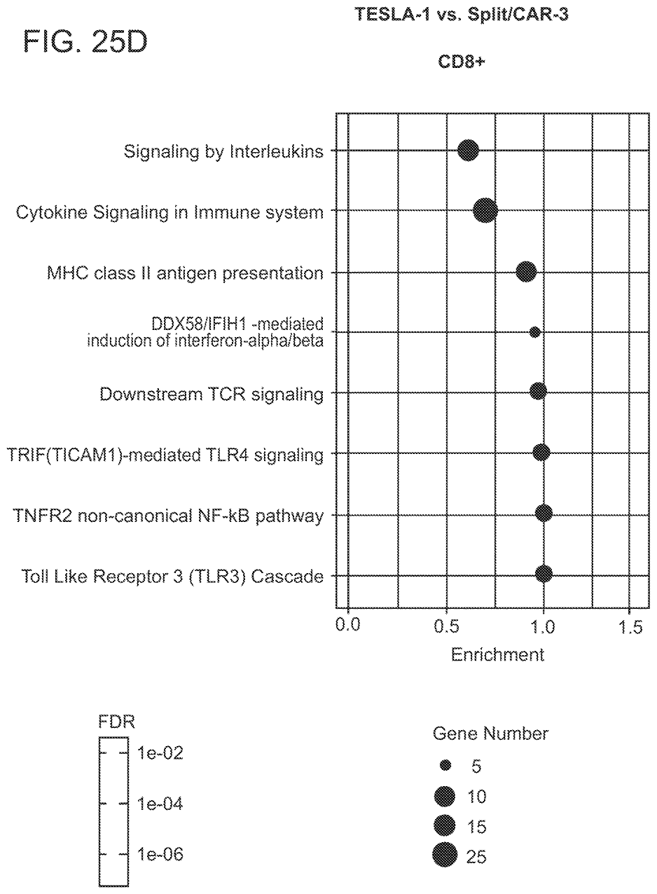

Stimulation with KMS26-MUT cells also upregulated the genes encoding nuclear factor kappa B (NFκB) subunits 1 and 2 (NFKB1 and NFKB2) in TESLA-1 T cells compared to Split/CAR-3 T cells, suggesting the MC domain modulates NFκB signaling within engineered T cell (FIG. 22). Network analysis of differentially upregulated genes in TESLA-1 T cells after KMS26-MUT stimulation confirmed the upregulation of multiple pathways in CD8+ TESLA-1 T cells that signal through NFκB, including toll-like receptor (TLR)- and MyD88-related pathways, as would be expected with the inclusion of a MyD88 domain within the T-cell receptor complex (FIGS. 25A-25B).

TESLA-1 T cells show gene expression changes associated with reduced apoptosis and exhaustion. After activation with KMS26-MUT cells, the gene SERPINB9 encoding proteinase inhibitor-9, a cytoplasmic protein that protects T cells from granzyme B-mediated activation-induced cell death, was upregulated in TESLA-1 T cells compared to Split/CAR-3 T cells (FIG. 22). The gene GZMB encoding granzyme B was less upregulated in TESLA-1 T cells compared to Split/CAR-3 T cells after culture with KMS26-MUT cells (FIG. 22). Additionally, the gene encoding cellular inhibitor of apoptosis 2 (BIRC3), an anti-apoptotic protein and positive regulator of canonical of NFκB signaling, was upregulated in TESLA-1 T cells compared to Split/CAR-3 T cells in non-stimulating conditions (i.e., culture with KMS26-NULL or no target cells), suggesting a role in supporting the homeostatic persistence of TESLA-1 T cells observed in mice (FIG. 22). The gene HAVCR2 encoding the co-inhibitory receptor TIM3 was among the most downregulated genes after stimulation of TESLA-1 cells with KMS26-MUT cells (FIGS. 22 and 23A). This was true for both CD4+ and CD8+ TESLA-1 cells. This gene was not downregulated after co-culture with KMS26-NULL cells or without target cells. Conversely, the expression of the gene encoding TIM3 was increased in Split/CAR-3 T cells after co-culture KMS26-MUT target cells.

Tonic-signaling of synthetic receptors within T cells in the absence of cognate antigen has been shown to dramatically modulate T-cell phenotypes and anti-tumor activity. When TESLA-1 and Split/CAR-3 T cells were cultured without target cells, 127 and 258 genes were differentially expressed in CD4+ and CD8+ T cells, suggesting antigen-independent transcriptomic changes induced by the inclusion of the MC domain in the TCR. The gene encoding CXCL13 (CXCL13) was among the most differentially upregulated genes for both CD4+ and CD8+ TESLA-1 cells in the absence of target cells (FIGS. 22 and 23B). By contrast, in Split/CAR-3 T cells, CXCL13 expression was dependent on antigen exposure with upregulation only observed after co-culture with KMS26-MUT cells (FIG. 22). The concomitant expression of the gene encoding CXCR3 (CXCR3), a receptor for CXCL13, in CD8+ TESLA-1 T cells under non-stimulating conditions (i.e., culture with KMS26-NULL or no target cells) suggests the presence of an autonomous chemokine signaling loop (FIG. 22). The gene encoding the transcription factor SOX4 (SOX4), which has been demonstrated to be upregulated in T cells by continuous CAR signaling and to drive expression of CXCL13, was also upregulated in TESLA-1 T cells in the absence of antigen, while it was not upregulated in Split/CAR-3 T cells under any condition (FIGS. 22 and 23B). Network analysis of genes upregulated in TESLA-1 T cells cultured without target cancer cells identified interleukin, TLR, and non-canonical NFκB pathways, suggesting tonic signaling mediated by both the MyD88 and CD40 domains within the chimeric receptor (FIGS. 25C-25D).

What is claimed is:

1. An immune cell comprising an engineered T cell receptor (TCR) comprising:
(a) a chimeric TCR alpha chain comprising a TCR alpha constant (TCR Cα) domain, wherein the chimeric TCR alpha chain lacks a TCR alpha variable (TCR Vα) domain;
(b) a chimeric TCR beta chain comprising a TCR beta constant (TCR Cβ) domain, wherein the chimeric TCR beta chain lacks a TCR beta variable (TCR Vβ) domain;
(c) a scFv VH domain;
(d) a scFv VL domain; and
(e) a co-stimulatory domain.

2. The immune cell of claim 1, wherein (i) the scFv VH domain is linked to the TCR alpha constant (TCR Cα) domain, and (ii) the scFv VL domain is linked to the TCR beta constant (TCR Cβ) domain.

3. The immune cell of claim 2, wherein the scFv VH domain is linked to the TCR alpha constant (TCR Cα) domain via a linker, and the scFv VL domain is linked to the TCR beta constant (TCR Cβ) domain via the linker.

4. The immune cell of claim 1, wherein (i) the scFv VL domain is linked to the TCR alpha constant (TCR Cα) domain, and (ii) the scFv VH domain is linked to the TCR beta constant (TCR Cβ) domain.

5. The immune cell of claim 4, wherein the scFv VL domain is linked to the TCR Cα domain via a linker, and the scFv VH domain is linked to the TCR Cβ domain via the linker.

6. The immune cell of claim 1, wherein the scFv VH domain and the scFv VL domain are linked to the TCR alpha constant (TCR Cα) domain.

7. The immune cell of claim 1, wherein the scFv VH domain and the scFv VL domain are linked to the TCR beta constant (TCR Cβ) domain.

8. The immune cell of claim 1, further comprising a TCR Cγ domain and a TCR Cδ domain.

9. An immune cell comprising an engineered T cell receptor (TCR) comprising:
(a) a chimeric TCR gamma chain comprising a TCR gamma constant (TCR Cγ) domain, wherein the chimeric TCR gamma chain lacks a TCR gamma variable (TCR Vγ) domain;
(b) a chimeric TCR delta chain comprising a TCR delta constant (TCR Cδ) domain, wherein the chimeric TCR delta chain lacks a TCR delta variable (TCR Vδ) domain;
(c) a scFv VH domain;
(d) a scFv VL domain; and
(e) a co-stimulatory domain.

10. The immune cell of claim 9, wherein (i) the scFv VH domain is linked to the TCR CY domain, and (ii) the scFv VL domain is linked to the TCR Cδ domain.

11. The immune cell of claim 10, wherein the scFv VH domain is linked to the TCR Cγ domain via a linker, and the scFv VL domain is linked to the TCR Cδ domain via the linker.

12. The immune cell of claim 9, wherein (i) the scFv VL domain is linked to the TCR Cγ domain, and (ii) the scFv VH domain is linked to the TCR Cδ domain.

13. The immune cell of claim 12, wherein the scFv VL domain is linked to the TCR Cγ domain via a linker, and the scFv VH domain is linked to the TCR Cδ domain via the linker.

14. The immune cell of claim 9, wherein the scFv VH domain and the scFv VL domain are linked to the TCR Cγ domain.

15. The immune cell of claim 9, wherein the scFv VH domain and the scFv VL domain are linked to the TCR Cδ domain.

16. The immune cell of claim 9, further comprising a TCR Cα domain and a TCR Cβ domain.

17. The immune cell of any one of claims 1-16, wherein the engineered TCR specifically binds to a neoantigen.

18. The immune cell of claim 17, wherein the neoantigen comprises EGFRvIII.

19. The immune cell of claim 17, wherein the neoantigen is presented on a HLA molecule.

20. The immune cell of claim 19, wherein the neoantigen comprises a peptide derived from a mutant oncogene.

21. The immune cell of claim 20, wherein the neoantigen comprises a p53RH antigen.

22. The immune cell of any one of claims 1-21, wherein the linker comprises SEQ ID NO: 1 or SEQ ID NO: 2.

23. The immune cell of any one of claims 1-22, wherein the co-stimulatory domain comprises a single co-stimulatory domain.

24. The immune cell of claim 23, wherein the co-stimulatory domain comprises a co-stimulatory domain of CD28, 4-1BB, OX40, ICOS, MYD88, 2B4, BTLA, CD2, CD22, CD27, CD30, CD4, CD40, CD79a, CD79b, CD84, CD8a, CD8b, CRACC, CRTAM, CTLA-4, DAP10, DNAM-1, DAP12, DR3, FCER1G, FCGR1A, FCGR2A, FCGR2B, FCGR3A, FCRL1, FCRL2, FCRL3, FCRL4, 5 FCRL5, FCRL6, GITR, HVEM, ITAM, KIR2DL1, KIR2DL2, KIR2DL3, KIR2DL4, KIR2DL5A, KIR2DL5B, KIR3DL1, KIR3DL2, KIR3DL3, LAG3, LAT, LILRB1, LILRB2, Ly9, NKG2A, NKG2C, NKG2D, NKp30, NKp44, NKp46, PD-1, PILRB, SIRPa, SLAMF1, SLAMF6, SLAMF7, TIGIT, TIM1, TIM3, TNFL6, TLR1, TLR10, TLR2, TLR3, TLR4, TLR5, TLR6, TLR7, TLR8, or TLR9.

25. The immune cell of claim 24, wherein the co-stimulatory domain comprises a co-stimulatory domain of MyD88 or a co-stimulatory domain of CD40.

26. The immune cell of any one of claims 1-22, wherein the co-stimulatory domain comprises a first co-stimulatory domain and a second co-stimulatory domain.

27. The immune cell of claim 26, wherein the first co-stimulatory domain and the second co-stimulatory domain are independently selected from CD28, 4-1BB, OX40, ICOS, MYD88, 2B4, BTLA, CD2, CD22, CD27, CD30, CD4, CD40, CD79a, CD79b, CD84, CD8a, CD8b, CRACC, CRTAM, CTLA-4, DAP10, DNAM-1, DAP12, DR3, FCER1G, FCGR1A, FCGR2A, FCGR2B, FCGR3A, FCRL1, FCRL2, FCRL3, FCRL4, 5 FCRL5, FCRL6, GITR, HVEM, ITAM, KIR2DL1, KIR2DL2, KIR2DL3, KIR2DL4, KIR2DL5A, KIR2DL5B, KIR3DL1, KIR3DL2, KIR3DL3, LAG3, LAT, LILRB1, LILRB2, Ly9, NKG2A, NKG2C, NKG2D, NKp30, NKp44, NKp46, PD-1, PILRB, SIRPa, SLAMF1, SLAMF6, SLAMF7, TIGIT, TIM1, TIM3, TNFL6, TLR1, TLR10, TLR2, TLR3, TLR4, TLR5, TLR6, TLR7, TLR8, or TLR9.

28. The immune cell of claim 27, wherein the first co-stimulatory domain comprises a co-stimulatory domain of MyD88 and the second co-stimulatory domain comprises a co-stimulatory domain of CD40.

29. The immune cell of any one of claims 1-28, wherein the co-stimulatory domain is linked to the TCR Cβ domain.

30. The immune cell of any one of claims 1-28, wherein the co-stimulatory domain is linked to the TCR Cγ domain.

31. The immune cell of any one of claims 1-30, wherein the co-stimulatory domain is linked to an extracellular and transmembrane domain of Fas.

32. The immune cell of any one of claims 1-31, wherein the engineered TCR is expressed from an expression cassette placed in an endogenous T cell receptor alpha constant (TRAC) locus of the immune cell.

33. The immune cell of any one of claims 1-32, wherein the immune cell is a human immune cell.

34. The immune cell of any one of claims 1-33, wherein the immune cell is a T cell.

35. A nucleic acid sequence encoding an engineered TCR, wherein the engineered TCR comprises:
(a) a chimeric TCR alpha chain comprising a TCR alpha constant (TCR Cα) domain, wherein the chimeric TCR alpha chain lacks a TCR alpha variable (TCR Vα) domain;
(b) a chimeric TCR beta chain comprising a TCR beta constant (TCR Cβ) domain, wherein the chimeric TCR beta chain lacks a TCR beta variable (TCR Vβ) domain;
(c) a scFv VH domain:
(d) a scFv VL domain; and
(e) a co-stimulatory domain.

36. The nucleic acid sequence of claim 35, wherein (i) the scFv VH domain is linked to the TCR alpha constant (TCR Cα) domain, and (ii) the scFv VL domain is linked to the TCR beta constant (TCR Cβ) domain.

37. The nucleic acid sequence of claim 36, wherein the scFv VH domain is linked to the TCR alpha constant (TCR Cα) domain via a linker, and the scFv VL domain is linked to the TCR beta constant (TCR Cβ) domain via the linker.

38. The nucleic acid sequence of claim 35, wherein (i) the scFv VL domain is linked to the TCR alpha constant (TCR Cα) domain, and (ii) the scFv VH domain is linked to the TCR beta constant (TCR Cβ) domain.

39. The nucleic acid sequence of claim 38, wherein the scFv VL domain is linked to the TCR Cα domain via a linker, and the scFv VH domain is linked to the TCR Cβ domain via the linker.

40. The nucleic acid sequence of claim 35, wherein the scFv VH domain and the scFv VL domain are linked to the TCR alpha constant (TCR Cα) domain.

41. The nucleic acid sequence of claim 35, wherein the scFv VH domain and the scFv VL domain are linked to the TCR beta constant (TCR Cβ) domain.

42. The nucleic acid sequence of claim 35, further comprising a TCR Cγ domain and a TCR Cδ domain.

43. A nucleic acid sequence encoding an engineered TCR, wherein the engineered TCR comprises:
(a) a chimeric TCR gamma chain comprising a TCR gamma constant (TCR Cγ) domain, wherein the chimeric TCR gamma chain lacks a TCR gamma variable (TCR Vγ) domain;
(b) a chimeric TCR delta chain comprising a TCR delta constant (TCR Cδ) domain, wherein the chimeric TCR delta chain lacks a TCR delta variable (TCR Vδ) domain;
(c) a scFv VH domain:
(d) a scFv VL domain; and
(e) a co-stimulatory domain.

44. The nucleic acid sequence of claim 43, wherein (i) the scFv VH domain is linked to the TCR Cγ domain, and (ii) the scFv VL domain is linked to the TCR Cδ domain.

45. The nucleic acid sequence of claim 44, wherein the scFv VH domain is linked to the TCR Cγ domain via a linker, and the scFv VL domain is linked to the TCR Cδ domain via the linker.

46. The nucleic acid sequence of claim 43, wherein (i) the scFv VL domain is linked to the TCR Cγ domain, and (ii) the scFv VH domain is linked to the TCR Cδ domain.

47. The nucleic acid sequence of claim 46, wherein the scFv VL domain is linked to the TCR Cγ domain via a linker, and the scFv VH domain is linked to the TCR Cδ domain via the linker.

48. The nucleic acid sequence of claim 43, wherein the scFv VH domain and the scFv VL domain are linked to the TCR Cγ domain.

49. The nucleic acid sequence of claim 43, wherein the scFv VH domain and the scFv VL domain are linked to the TCR Cδ domain.

50. The nucleic acid sequence of claim 43, further comprising a TCR Cα domain and a TCR Cβ domain.

51. The nucleic acid sequence of any one of claims 35-50, wherein the linker comprises SEQ ID NO: 1 or SEQ ID NO: 2.

52. The nucleic acid sequence of any one of claims 35-51, wherein the co-stimulatory domain comprises a single co-stimulatory domain.

53. The nucleic acid sequence of claim 52, wherein the co-stimulatory domain comprises a co-stimulatory domain of CD28, 4-1BB, OX40, ICOS, MYD88, 2B4, BTLA, CD2, CD22, CD27, CD30, CD4, CD40, CD79a, CD79b, CD84, CD8a, CD8b, CRACC, CRTAM, CTLA-4, DAP10, DNAM-1, DAP12, DR3, FCER1G, FCGR1A, FCGR2A, FCGR2B, FCGR3A, FCRL1, FCRL2, FCRL3, FCRL4, 5 FCRL5, FCRL6, GITR, HVEM, ITAM, KIR2DL1, KIR2DL2, KIR2DL3, KIR2DL4, KIR2DL5A, KIR2DL5B, KIR3DL1, KIR3DL2, KIR3DL3, LAG3, LAT, LILRB1, LILRB2, Ly9, NKG2A, NKG2C, NKG2D, NKp30, NKp44, NKp46, PD-1, PILRB, SIRPa, SLAMF1, SLAMF6, SLAMF7, TIGIT, TIM1, TIM3, TNFL6, TLR1, TLR10, TLR2, TLR3, TLR4, TLR5, TLR6, TLR7, TLR8, or TLR9.

54. The nucleic acid sequence of claim 53, wherein the co-stimulatory domain comprises a co-stimulatory domain of MyD88 or a co-stimulatory domain of CD40.

55. The nucleic acid sequence of any one of claims 35-51, wherein the co-stimulatory domain comprises a first co-stimulatory domain and a second co-stimulatory domain.

56. The nucleic acid sequence of claim 55, wherein the first co-stimulatory domain and the second co-stimulatory domain are independently selected from CD28, 4-1BB, OX40, ICOS, MYD88, 2B4, BTLA, CD2, CD22, CD27, CD30, CD4, CD40, CD79a, CD79b, CD84, CD8a, CD8b, CRACC, CRTAM, CTLA-4, DAP10, DNAM-1, DAP12, DR3, FCER1G, FCGR1A, FCGR2A, FCGR2B, FCGR3A, FCRL1, FCRL2, FCRL3, FCRL4, 5 FCRL5, FCRL6, GITR, HVEM, ITAM, KIR2DL1, KIR2DL2, KIR2DL3, KIR2DL4, KIR2DL5A, KIR2DL5B, KIR3DL1, KIR3DL2, KIR3DL3, LAG3, LAT, LILRB1, LILRB2, Ly9, NKG2A, NKG2C, NKG2D, NKp30, NKp44, NKp46, PD-1, PILRB, SIRPa, SLAMF1, SLAMF6, SLAMF7, TIGIT, TIM1, TIM3, TNFL6, TLR1, TLR10, TLR2, TLR3, TLR4, TLR5, TLR6, TLR7, TLR8, or TLR9.

57. The nucleic acid sequence of claim 56, wherein the first co-stimulatory domain comprises a co-stimulatory domain of MyD88 and the second co-stimulatory domain comprises a co-stimulatory domain of CD40.

58. The nucleic acid sequence of any one of claims 35-57, wherein the co-stimulatory domain is linked to the TCR Cβ domain.

59. The nucleic acid sequence of any one of claims 35-57, wherein the co-stimulatory domain is linked to the TCR Cγ domain.

60. The nucleic acid sequence of any one of claims 35-59, wherein the co-stimulatory domain is linked to an extracellular and transmembrane domain of Fas.

61. A vector comprising a nucleic acid sequence of any one of claims 35-60.

62. The vector of claim 61, further comprising a promoter.

63. The vector of claim 62, wherein the promoter is a TRAC promoter or EF1-alpha promoter.

64. The vector of any one of claims 61-63, wherein the vector is a viral vector.

65. A method of producing an engineered immune cell, the method comprising: introducing into an immune cell a nucleic acid sequence of any one of claims 35-60 or a vector of any one of claims 61-64, thereby producing the engineered immune cell.

66. The method of claim 65, wherein the nucleic acid sequence is introduced into the immune cell by using a gene-editing agent.

67. The method of claim 66, wherein the gene-editing agent comprises CRISPR components.

68. An engineered immune cell produced by any one of the methods of claims 65-67.

69. A pharmaceutical composition comprising an engineered immune cell of claim 68 and a pharmaceutically acceptable carrier.

70. A method of treating a disease in a subject, the method comprising administering to the subject the engineered immune cell of claim 68 or a pharmaceutical composition of claim 69.

71. The method of claim 70, wherein the disease is a cancer comprising a p53 mutation.

72. The method of claim 71, wherein the p53 mutation comprises a p53R175H mutation.

73. The method of claim 71, wherein the cancer is an ovary cancer, colorectum cancer, esophagus cancer, head and neck cancer, larynx cancer, lung cancer, skin cancer, pancreas cancer, stomach cancer, liver cancer, brain cancer, bladder cancer, breast cancer, uterus cancer, soft tissue cancer, lymph node cancer, prostate cancer, bone cancer, endocrine gland cancer, or cervix cancer.
